(19) United States
(12) Patent Application Publication
(10) Pub. No.: US 2020/0188528 A1
(43) Pub. Date: Jun. 18, 2020

(54) PEPTIDE-ANTIBODY COMPOSITIONS AND METHODS OF USE THEREOF

(71) Applicant: Fred Hutchinson Cancer Research Center, Seattle, WA (US)

(72) Inventors: James OLSON, Seattle, WA (US); Andrew J. MHYRE, Kenmore, WA (US); Colin CORRENTI, Seattle, WA (US)

(21) Appl. No.: 16/470,936

(22) PCT Filed: Dec. 19, 2017
(86) PCT No.: PCT/US2017/067432

§ 371 Date: Jun. 18, 2019

Related U.S. Application Data
(60) Provisional application No. 62/436,391, filed on Dec. 19, 2016.

(51) Int. Cl. A61K 47/68; C07K 16/22; C07K 16/30; C07K 16/40; A61P 25/28

(52) U.S. Cl. CPC A61K 47/6881; A61K 47/6811; A61K 47/6851; A61K 47/6871; A61K 38/00; C07K 16/30; C07K 16/40; A61P 25/28; C07K 16/22

(57) ABSTRACT

Peptide-antibody complexes, including fusions and conjugates, that target or accumulate in target cells, such as tumors and diseased cells, or enhance the immune system are disclosed. In some embodiments, such complexes function as platforms for attaching and targeting one or more therapeutic agents or detectable agents, including radioisotopes, detectable labels, and/or cytotoxic agents, to target cells in the central nervous system or across the blood brain barrier, cancerous cells, or target cells recognized by the antibody in such complexes. In some complexes, the peptide serves as the targeting mechanism, while other embodiments contemplate the antibody as the targeting mechanism. Peptides and/or antibodies can be engineered to deliver various effector functions in various embodiments. Peptide-antibody complexes that function as immunotherapies and enhance a body's immune system in detecting and/or destroying diseased cells or pathogens are also disclosed. Pharmaceutical compositions and uses of peptide-antibody complexes are further disclosed.

### CROSS REFERENCE

This application claims the benefit of U.S. Provisional Patent Application No. 62/436,391, filed Dec. 19, 2016, the entire contents of which are incorporated by reference.

### SEQUENCE LISTING

The instant application contains a Sequence Listing which has been submitted electronically in ASCII format and is hereby incorporated by reference in its entirety. Said ASCII copy, created on Dec. 19, 2017, is named 44189-717_601_SL.txt and is 251,970 bytes in size.

### BACKGROUND

Targeted therapies enable specific treatment of diseases/disorders with the advantages of lower dose-dependent toxicities and increased effective dose of the therapy at the location of interest. Targeted therapies can be beneficial for the treatment of central nervous system (CNS) diseases/disorders as well as for the treatment of cancers.

CNS diseases/disorders refer to a group of neurological diseases/disorders that affect the structure or function of the brain or spinal cord. Causes of CNS diseases include trauma, infections, degeneration, autoimmune disorders, structural defects, tumors, and stroke. Development of new products for treating CNS diseases/disorders has lagged behind other therapeutic areas due to the complexity of the CNS and challenges associated with delivery of therapeutics across the blood-brain barrier (BBB).

Cancer is a genetic disease and is caused by changes in the genes that control cellular function, such as how cells grow and divide. Such genetic changes can be caused by inheritance, environment factors, or random mutations in one's genome. Cancer occurs when there is abnormal cell growth, which has the potential to invade or spread to other parts of the body. Not all tumors are cancerous, as some tumors are benign and do not spread to other parts of the body. Cancerous tumors are malignant and can spread to other tissues. Metastatic cancer is cancer that spreads to another place in the body.

There is a need in the art for improved, targeted therapies for the treatment of CNS diseases/disorders as well as cancers.

### SUMMARY

The present disclosure provides compositions of peptide-antibody complexes and methods of use thereof.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In various aspects, the present disclosure provides a peptide-antibody complex comprising: (a) a peptide; and (b) an antibody or fragment thereof, wherein the peptide and antibody or fragment thereof are conjugated, linked, bound together by affinity, or fused, and an amino acid sequence of the peptide is any one of SEQ ID NO: 1-SEQ ID NO: 426, or a fragment thereof.

In various aspects, the present disclosure provides a peptide-antibody complex comprising: (a) a peptide; and (b) an antibody or fragment thereof, wherein the peptide and antibody or fragment thereof are conjugated, linked, bound together by affinity, or fused, and an amino acid sequence of the peptide has at least 80% sequence identity with any one of SEQ ID NO: 1-SEQ ID NO: 201, SEQ ID NO: 214-SEQ ID NO: 414, or a fragment thereof. In further aspects, the amino acid sequence of the peptide has at least 85%, at least 90%, or at least 95% sequence identity with any one of SEQ ID NO: 1-SEQ ID NO: 201, SEQ ID NO: 214-SEQ ID NO: 414, or a fragment thereof.

In some aspects, the antibody is a monoclonal antibody or Fc fusion protein. In further aspects, the antibody is a human or humanized monoclonal antibody. In some aspects, the peptide is a targeting or homing agent, or selectively accumulates in a target cell or tissue. In other aspects, the peptide targets central nervous system cells, or can cross a BBB or a CSF barrier. In further aspects, the peptide targets cancerous cells, tumors, or diseased or infected cells.

In some aspects, the antibody is a targeting or homing agent, or selectively accumulates in a target cell or tissue. In further aspects, the antibody targets cells expressing, secreting, or comprising a receptor or marker recognized by the antibody. In some aspects, the antibody triggers internalization, trafficking, or lysosomal processing of the peptide-antibody complex.

In other aspects, the peptide is a therapeutic agent. In still other aspects, the antibody is a therapeutic agent. In some aspects, the peptide is further conjugated, coupled, or attached to a therapeutic agent or detectable agent. In other aspects, the antibody is further conjugated, coupled, or attached to a therapeutic agent or detectable agent.

In some aspects, the peptide-antibody complex further comprises a therapeutic agent, or detectable agent. In some aspects, the peptide and the antibody are conjugated or linked using a cleavable or a non-cleavable linker. In other aspects, the peptide and the antibody are conjugated or linked using a flexible or a rigid linker.

In some aspects, the antibody or fragment thereof comprises an scFv, Fab, Fc, heavy chain, light chain, single chain, or complementarity-determining region, or any combination thereof. In some aspects, the antibody modulates pharmacokinetics, pharmacodynamics, serum half-life, stability, expression level, and/or biodistribution of the peptide.

In other aspects, the peptide modulates pharmacokinetics, pharmacodynamics, stability, expression level, and/or biodistribution of the antibody. In still other aspects, a linker between the peptide and the antibody modulates pharmacokinetics, pharmacodynamics, serum half-life, stability, expression level, and/or biodistribution of the peptide-antibody complex.

In some aspects, the antibody disrupts an abnormal cellular process or pathway. In some aspects, the antibody or the peptide has anti-cancer activity. In other aspects, the antibody or the peptide enhances a body's immune system against an infection, pathogen, or any diseased, dysregulated, or uncontrolled cell.

In other aspects, the antibody or the peptide is a neuroprotective agent. In some aspects, the antibody or the peptide is a chemotherapy agent. In further aspects, the antibody or the peptide modulates an immune response toward a target cell or a cellular factor. In some aspects, the antibody enhances T-cell mediated immune response. In other aspects, the antibody or the peptide induces or triggers apoptosis of a cancerous, abnormal, or diseased cell.

In some aspects, the antibody binds to or modulates an antigen or a marker indicative of a central nervous system disorder. In other aspects, the antibody binds to or modulates a receptor or a surface marker or a cancer cell in a brain. In some aspects, the antibody binds to or modulates a biomarker or a factor associated with a neurodegenerative disease.

In other aspects, the antibody binds to or modulates a pathogen or an infectious agent. In still other aspects, the antibody suppresses an autoimmune response. In still other aspects, the antibody suppresses or reduces angiogenesis. In some aspects, the antibody is selected from any one of the antibodies listed in TABLE 4.

In further aspects, the antibody is or has at least 80% sequence identity with an antibody selected from the group consisting of: Crenezumab, Urelumab, Utomilumab, Ensituximab, Tacatuzumab tetraxetan, Nesvacumab, Vanucizumab, Evinacumab, Vepalimomab, 8H9, Belimumab, Tabalumab, Bapineuzumab, Gantenerumab, Solanezumab, Aducanumab, Detumomab, Nacolomab tafenatox, Igovomab, Oregovomab, Sofituzumab vedotin, Abagovomab, Galcanezumab, Mogamulizumab, PRO 140, Tovetumab, Coltuximab ravtansine, Denintuzumab mafodotin, Inebilizumab, SGN-CD19A, Taplitumomab paptox, Afutuzumab, FBTA05, Moxetumomab pasudotox, Pinatuzumab vedotin, Varlilumab, Atezolizumab, Avelumab, Durvalumab, Enoblituzumab, Lilotomab satetraxetan, Naratuximab emtansine, Otlertuzumab, Tetulomab, Isatuximab, Daratumumab, Ibalizumab, Zanolimumab, Bleselumab, Dacetuzumab, Lucatumumab, Teneliximab, Bivatuzumab mertansine, Abituzumab, Intetumumab, Alemtuzumab, Lorvotuzumab mertansine, Itolizumab, Vorsetuzumab mafodotin, Milatuzumab, Polatuzumab vedotin, Galiximab, Altumomab pentetate, Arcitumomab, Labetuzumab, Besilesomab, Erenumab, Margetuximab, IMAB362, Actoxumab, Bezlotoxumab, Tefibazumab, Tisotumab vedotin, Cabiralizumab, Emactuzumab, Lenzilumab, Namilumab, Ticilimumab, tremelimumab, Ulocuplumab, Sevirumab, Regavirumab, Rovalpituzumab tesirine, Demcizumab, Enoticumab, Navicixizumab, Begelomab, Drozitumab, Parsatuzumab, Cetuximab, Depatuxizumab mafodotin, Futuximab, Imgatuzumab, Laprituximab emtansine, Matuzumab, Necitumumab, Nimotuzumab, Panitumumab, Zalutumumab, Carotuximab, Edobacomab, Nebacumab, Adecatumumab, Citatuzumab bogatox, Edrecolomab, Oportuzumab monatox, Solitomab, Tucotuzumab celmoleukin, Catumaxomab, Fibatuzumab, Epitumomab cituxetan, Sontuzumab, Duligotumab, Elgemtumab, Lumretuzumab, Patritumab, Seribantumab, Radretumab, Pasotuxizumab, Farletuzumab, Mirvetuximab soravtansine, Vantictumab, Crotedumab, 3F8, Ch.14.18, Dinutuximab, Derlotuximab biotin, Suvizumab, Apolizumab, Fasinumab, Ponezumab, Volociximab, Lirilumab, Monalizumab, cBR96-doxorubicin immunoconjugate, Carlumab, Amatuximab, Imalumab, Ublituximab, Anetumab ravtansine, Cantuzumab mertansine, Refanezumab, Fulranumab, Tanezumab, Racotumomab, Ozanezumab, Brontictuzumab, Tarextumab, Vesencumab, Nivolumab, Pidilizumab, Pembrolizumab, Olaratumab, Lifastuzumab vedotin, Bavituximab, Tosatoxumab, Icrucumab, Alacizumab pegol, Ramucirumab, and Pritumumab.

In some aspects, the antibody binds to or modulates a target or a target with at 80% sequence identity with a target selected from the group consisting of: beta amyloid, Tau, alpha synuclein, BACE, IL23, TGF beta, CD137, 5AC, alpha-fetoprotein, angiopoietin, anthrax toxin, AOC3, VAP-1, B7-H3, calcitonin, CD4, CD5, CD6, CD20, CD22, CD27, CD274, CD276, CD28, CD33, CD37, CD38, CD40, CD44, CD51, CD56, CD70, CD74, CD79B, CD80, CD137, CD140a, CD19, CGRP, ch4D5, CLDN18.2, coagulation factor III, CSF1R, CSF2, CTLA-4, EGFL7, EGFR, endotoxin, EpCAM, CD3, ephrin receptor A3, episialin, ERBB3, HER3, FAP, fibrin, fibronectin, folate hydrolase, folate receptor, Frizzled receptor, GCGR, GD2 ganglioside, GD3 ganglioside, glypican 3, GMCSF receptor, GPNMB, growth differentiation factor 8, GUCY2C, HER1, HER2/neu, HGF, HHGFR, histone complex, HIV-1, HLA-DR, HNGF, scatter factor receptor kinase, TNF, IGF-1 receptor, CD221, IGF1, IGF2, IL 17A, IL-13, IL-17, IL2, ILGF2, KIR2D, KLRC1, Lewis-Y antigen, MCP-1, mesothelin, MIF, MS4A1, MSLN, NGF, N-glycolylneuraminic acid, NOGO-A, Notch 1, Notch receptor, NRP1, PD-1, PD-L1, PDCD1, PDGF-R, phosphate-sodium co-transporter, RON, RTN4, SDC1, STEAP1, TAG-72, T-cell receptor, TEM1, tenascin C, TNFR, TNF-α, TRAIL-R1, tumor-associated calcium signal transducer 2, TWEAK receptor, VEGF, VEGFR1, VEGFR2, vimentin, CD20/MS4A1, 4-1BB, 5T4, activin receptor-like kinase 1, adenocarcinoma antigen, AGS-22M6, B7-H3, BAFF, B-lymphoma cell, C242 antigen, CCR4, CCR5, CEA, CGRP, CLDN18.2, Clostridium difficile, clumping factor A, coagulation factor III, CSF1R, CSF2, CTLA-4, CXCR4 (CD184), cytomegalovirus, cytomegalovirus glycoprotein B, DLL3, DLL4, DPP4, DR5, EGFL7, EGFR, endoglin, endotoxin, HNGF, integrin α5β1, MSLN, myelin-associated glycoprotein, NGF, phosphatidylserine, and Staphylococcus aureus.

In some aspects, the antibody binds to or modulates a target or a target with least 80% sequence with a target selected from the group consisting of: CD19, CD20, CD30, CD33, CD52, EpCAM, gpA33, CEA, mucins, TAG-72, carbonic anhydrase IX, PSMA, folate binding protein, gangliosides (e.g., GD2, GD3, GM2), Lewis-Y², VEGF, VEGFR, αVβ3, α5β1, ErbB1/EGFR, ErbB2/HER2, ErbB3, c-MET, IGF1R, EphA3, TRAIL-R1, TRAIL-R2, RANKL, FAP, tenascin, Tau, alpha synuclein, beta amyloid, and BACE.

In other aspects, the antibody binds to or modulates beta amyloid, Tau, or alpha synuclein. In some aspects, the antibody comprises an anti-BACE heavy chain, and anti-BACE light chain, or a fragment thereof.

In some aspects, the antibody has at least 80% sequence identity with SEQ ID NO: 427 or SEQ ID NO: 428, or a fragment thereof. In other aspects, the complex comprises peptide SEQ ID NO: 1 conjugated to SEQ ID NO: 427 or SEQ ID NO: 1 conjugated to SEQ ID NO: 428, or has at least 80% sequence identity with SEQ ID NO: 429 or SEQ ID NO: 430, or a fragment thereof.

In further aspects, the peptide has at least 85%, at least 90%, or at least 95% sequence identity with any one of SEQ ID NO: 1, SEQ ID NO: 2, SEQ ID NO: 3, SEQ ID NO: 124, SEQ ID NO: 128, SEQ ID NO: 132, SEQ ID NO: 141, SEQ ID NO: 147, SEQ ID NO: 149, SEQ ID NO: 161, SEQ ID NO: 179, SEQ ID NO: 215, SEQ ID NO: 216, SEQ ID NO: 345, or SEQ ID NO: 374, or a fragment thereof.

In some aspects, the peptide comprises at least 6, at least 8, at least 10, at least 12, at least 14, or at least 16 cysteine residues. In some aspects, the peptide comprises a plurality of disulfide bridges formed between cysteine residues. In further aspects, the peptide comprises at least 5% or more of the residues are cysteines forming intramolecular disulfide bonds. In some aspects, the peptide comprises a disulfide through disulfide knot.

In some aspects, the peptide comprises at least one amino acid residue in an L configuration, or wherein at least one amino acid residue of the peptide is in a D configuration. In some aspects, the peptide sequence is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues long.

In some aspects, the peptide is arranged in a multimeric structure with at least one other peptide. In some aspects, the peptide has a neutral net charge at physiological pH. In some aspects, the peptide has a positive net charge greater than +0.5 at physiological pH. In other aspects, the peptide has a negative net charge lower than −0.5 at physiological pH.

In other aspects, at least one residue of the peptide comprises a chemical modification. In some aspects, the chemical modification is blocking the N-terminus of the peptide prior to conjugating or linking with the antibody. In further aspects, the chemical modification is methylation, acetylation, or acylation. In still further aspects, the chemical modification is: methylation of one or more lysine residues or analogue thereof; methylation of an N-terminus; or methylation of one or more lysine residue or analogue thereof and methylation of the N-terminus.

In some aspects, the peptide is linked to an acyl adduct. In other aspects, the peptide is fused with the antibody at an N-terminus or a C-terminus of the peptide, at an N-terminus or a C-terminus of a heavy chain, a light chain, or a constant region of the antibody, or within the heavy chain, light chain, or constant region of the antibody.

In some aspects, the peptide-antibody complex further comprises a linker. In further aspects, the linker is cleavable. In other aspects, the linker is pH sensitive. In still other aspects, the linker comprises a sequence of (GxS)n, wherein x is 1, 2, 3, 4, 5, 6, 7, 8, 9, or 10, and wherein n is 1, 2, 3, 4, 5, 6, 7, 8, 9, or 10 (SEQ ID NO: 445).

In still other aspects, the linker comprises a sequence of GGGSGGGS (SEQ ID NO: 444). In some aspects, the linker comprises a dipeptide. In some aspects, the linker is linked to the antibody, the peptide, a therapeutic agent, a detectable agent, or any combination thereof. In further aspects, the linker is linked to the antibody, the peptide, the therapeutic agent, or the detectable agent by a covalent chemical bond.

In some aspects, the linker links the peptide to the antibody at an N-terminus or a C-terminus of the peptide. In some aspects, the linker releases the peptide, the antibody, the therapeutic agent, the detectable agent, or any combination thereof upon cleavage, a pH change, or cleavage by a protease upon internalization of the peptide-antibody complex. In some aspects, the peptide-antibody complex further comprises a therapeutic agent, wherein the therapeutic agent is a radiosensitizer or photo sensitizer.

In some aspects, the peptide-antibody complex further comprises a therapeutic agent, wherein the therapeutic agent is a microtubule inhibitor, DNA intercalator, DNA cleaver, or a modulator of cellular processes. In further aspects, the microtubule inhibitor, DNA intercalator, or DNA cleaver is selected from the group consisting of: Bleomycin A2, Calicheamicin g-1, Auristatins (e.g., MMAE, MMAF, PE, Dolastatin-10), Maytansines (e.g., DM-1, DM-4, and derivatives), Vinorelbine, Paclitaxel, Epothilone B, Tubulysins (IM-2, B), Doxorubicin, Epirubicin, PNU-159682, Duocarmycins, PBD dimers, Oligomycin C, Daunorubicin, Valrubicin, Topotecan and desmethyl-Topotecan, DNA Transcription Inhibitors, Dactinomycin, Akt inhibitor, Ipatasertib (GDC-0068), DNA cross-linker, Mitomycin C, and Dihydrofolate reductase (DHFR) inhibitor, and Methotrexate.

In some aspects, the peptide-antibody complex further comprises a therapeutic agent, wherein the therapeutic agent is a cytotoxic molecule selected from the group consisting of: auristatin, a maytansinoid, doxorubicin, a calicheamicin, a platinum compound a taxane, paclitaxel, a BACE inhibitor, a Bcl-xL inhibitor, WEHI-539, venetoclax, ABT-199, navitoclax, AT-101, obatoclax, a pyrrolobenzodiazepine, and dolastatin. In further aspects, the peptide-antibody complex further comprises a therapeutic agent conjugated to the complex at an N-terminus or a C-terminus of the complex.

In some aspects, 1, 2, 3, 4, 5, 6, 7, 8, 9, or 10 therapeutic agents are linked to the antibody or the peptide. In some aspects, the peptide is linked to the therapeutic agent via a cleavable, non-cleavable, or a pH sensitive linker. In other aspects, the peptide is linked to the therapeutic agent at an N-terminus, at the epsilon amine of an internal lysine residue, or a C-terminus of the peptide. In still other aspects, the antibody is linked to the therapeutic agent at an N-terminus or a C-terminus of a heavy chain, light chain, a variable region, a constant region, or any combination thereof, of the antibody.

In some aspects, the peptide-antibody complex further comprises a detectable agent selected from the group consisting of: a fluorophore, a near-infrared dye, a contrast agent, a nanoparticle, a metal-containing nanoparticle, a metal chelate, an X-ray contrast agent, a PET agent, a radioisotope, and a radionuclide chelator. In some aspects, the detectable agent is a fluorescent dye.

In some aspects, the peptide crosses a blood brain barrier of a subject. In other aspects, the peptide crosses a blood cerebrospinal fluid barrier of a subject. In some aspects, the peptide or the antibody, or both home, target, is directed to or migrates to a tumor or diseased region, tissue, structure, or cell of the subject after crossing the blood brain barrier or the cerebrospinal fluid barrier.

In other aspects, upon administration to a subject the peptide or the antibody, or both home, target, accumulate in or migrate to a specific region in CNS of the subject. In some aspects, the specific region of the CNS comprises the ventricles, the cerebrospinal fluid, the hippocampus, the meninges, the rostral migratory system, the dentate gyrus, the subventricular zone, or any combination thereof. In some aspects, the antibody or the peptide, or the peptide-antibody complex, is capable of affecting neurological disorders, lysosomal storage diseases, epilepsy, meningitis, infections in the brain, stroke, and multiple sclerosis.

In some aspects, the antibody or the peptide, or the peptide-antibody complex is capable of affecting aggregation of a protein associated with a neurodegenerative disease. In other aspects, the peptide or the antibody blocks a receptor or channel involved in neurological disorders. In still other aspects, the peptide or the antibody homes, targets, is directed to, or migrates to a tumor or cancerous cell.

In some aspects, the tumor is a solid tumor. In further aspects, the peptide penetrates the solid tumor. In still further aspects, the tumor or cancerous cell is from a brain cancer, a glioblastoma, a primary brain tumor, a metastatic brain tumor, a triple-negative breast cancer, a colon cancer, or a sarcoma.

In some aspects, the peptide has at least 80% sequence identity with SEQ ID NO: 433, SEQ ID NO: 219, SEQ ID NO: 230, SEQ ID NO: 238, SEQ ID NO: 245, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 258, SEQ ID NO: 259, SEQ ID NO: 260, SEQ ID NO: 262, SEQ ID NO: 263, SEQ ID NO: 264, SEQ ID NO: 265, SEQ ID NO: 266, SEQ ID NO: 295, and any fragment thereof.

In some aspects, the peptide or the antibody is conjugated or fused to an immunotherapy. In other aspects, the peptide is conjugated or fused to an anti-angiogenic antibody. In other aspects, the peptide crosses a blood brain barrier to target a tumor in the brain. In still other aspects, the peptide or the peptide-antibody complex crosses a blood brain barrier to reduce aggregation or plaque in the brain.

In some aspects, the antibody or peptide inhibits a cellular pathway associated with cancer. In other aspects, the antibody or peptide is capable of inhibiting or activating ion channels. In still other aspects, the antibody exhibits protease inhibitor activity. In some aspects, the peptide or the antibody has antibacterial, antifungal, or antiviral activity.

In some aspects, the peptide comprises or is derived from the group consisting of: chlorotoxins, brazzeins, circulins, stecrisps, hanatoxins, midkines, hefutoxins, potato carboxypeptidase inhibitors, bubble proteins, attractins, α-GI, α-GID, μ-pIIIA, ω-MVIIA, ω-CVID, χ-MrIA, ρ-TIA, conantokin G, contulakin G, GsMTx4, margatoxins, shK, toxin K, chymotrypsin inhibitors (CTI), EGF epiregulin core, hainantoxins, theraphotoxins, hexatoxins, opicalcins, imperatoxins, defensins, and insectotoxins.

In various aspects, the present disclosure provides a peptide comprising a sequence of any one of SEQ ID NO: 193-SEQ ID NO: 201, SEQ ID NO: 406-SEQ ID NO: 414, or a fragment thereof.

In various aspects, the present disclosure provides a peptide comprising a sequence that has at least 80% sequence identity with any one of SEQ ID NO: 193-SEQ ID NO: 201, SEQ ID NO: 406-SEQ ID NO: 414, or a fragment thereof. In some aspects, the sequence has at least 85%, at least 90%, or at least 95% sequence identity with any one of SEQ ID NO: 193-SEQ ID NO: 201, SEQ ID NO: 406-SEQ ID NO: 414, or a fragment thereof. In some aspects, at least one residue of the peptide comprises a chemical modification. In other aspects, the peptide is linked to a therapeutic agent. In still other aspects, the peptide is linked to a detectable agent.

Also disclosed herein are pharmaceutical compositions comprising any of the peptide-antibody complexes described herein, or a salt thereof, and a pharmaceutically acceptable carrier. The pharmaceutical composition can be formulated for administration to a subject, e.g., for inhalation, intranasal administration, oral administration, topical administration, intravenous administration, subcutaneous administration, intra-articular administration, intramuscular administration, intrathecal, intraperitoneal administration, or a combination thereof. The peptide-antibody complex or pharmaceutical composition can be administered by inhalation, intranasally, orally, topically, intravenously, subcutaneously, intra-articularly, intramuscularly administration, intraperitoneally, or a combination thereof.

Also described herein are methods of using peptide-antibody complexes as described herein to treat a disease, disorder, or condition, wherein the peptide or the antibody of the complex, or a pharmaceutical composition thereof, homes, targets, migrates to, or is directed to a cancerous or diseased region, tissue, structure, or cell of the subject following administration. The condition includes, but not limited to, a tumor or cancer, solid tumor, a metastatic cancer, a brain tumor, triple-negative breast cancer, colon cancer, breast or colon cancer metastases, or sarcoma, wherein the brain tumor is inoperable in some cases. In some embodiments, the complex comes into proximity with the tumor after crossing a blood brain barrier, or is used to treat the subject with an additional treatment, wherein the additional treatment comprises chemotherapy, radiation therapy, immunomodulatory therapy, or a combination thereof. In some cases, the peptide-antibody complex can cross the blood brain barrier of the subject following administration, or the blood cerebrospinal fluid barrier of the subject following administration. In other aspects, the peptide-antibody complex, or the peptide or the antibody of the complex homes, targets, is directed to or migrates to the ventricles, cerebrospinal fluid, meninges, rostral migratory system, or hippocampus of the subject following administration, wherein the condition is a brain disorder, or wherein the condition is associated with a function of the ventricles, cerebrospinal fluid, or hippocampus, or wherein the brain disorder is associated with a function of the brain. In some cases, the peptide-antibody complex can be used to diagnose, prevent, or treat the brain disorder, wherein the brain disorder includes, but not limited to, memory loss or memory function, Alzheimer's disease, Parkinson's disease, multiple system atrophy (MSA), schizophrenia, epilepsy, progressive multifocal leukoencephalopathy, fungal infection, depression, bipolar disorder, post-traumatic stress disorder, stroke, traumatic brain injury, infection, or multiple sclerosis.

In some embodiments, the peptide in the peptide-antibody complexes, including compositions and methods of use thereof, comprise or are derived from the group consisting of: chlorotoxins, brazzeins, circulins, stecrisps, hanatoxins, midkines, hefutoxins, potato carboxypeptidase inhibitors, bubble proteins, attractins, α-GI, α-GID, μ-pIIIA, ω-MVIIA, ω-CVID, χ-MrIA, ρ-TIA, conantokin G, contulakin G, GsMTx4, margatoxins, shK, toxin K, chymotrypsin inhibitors (CTI), EGF epiregulin core, hainantoxins, theraphotoxins, hexatoxins, opicalcins, imperatoxins, defensins, and insectotoxins.

In various aspects, the present disclosure provides a peptide-antibody complex comprising: (a) a peptide; and (b) an antibody or fragment thereof, wherein the peptide and antibody or fragment thereof are conjugated, linked, bound together by affinity, or fused, and an amino acid sequence of the peptide is any one of SEQ ID NO: 202-SEQ ID NO: 213 or SEQ ID NO: 415-SEQ ID NO: 426, or a fragment thereof, or a fragment thereof.

In some aspects, the amino acid sequence of the peptide has at least 80%, at least 85%, at least 90%, at least 95%, at least 97%, at least 99%, or at least 100% sequence identity with any one of SEQ ID NO: 1-SEQ ID NO: 201, SEQ ID NO: 214-SEQ ID NO: 414, or a fragment thereof.

In some aspects, the antibody is a human or humanized monoclonal antibody, a monoclonal antibody or Fc fusion protein or wherein the antibody is an antibody fragment comprising scFv, Fab, Fc, heavy chain, light chain, single chain, or complementarity-determining region, or any combination thereof.

In some aspects, the peptide is a targeting or homing agent, or selectively accumulates in a target cell or tissue. In further aspects, the peptide-antibody complex targets central nervous system cells, or can cross a BBB or a CSF barrier, or targets cancerous cells, tumors, or diseased or infected cells, or targets cells expressing, secreting, or comprising a receptor or marker recognized by the antibody.

In still further aspects, the antibody triggers internalization, trafficking, or lysosomal processing of the peptide-antibody complex. In some aspects, the peptide-antibody complex further comprises a therapeutic agent, or detectable agent. In some aspects, the peptide and the antibody are conjugated or linked using a cleavable or a non-cleavable linker or a flexible or a rigid linker.

In some aspects, the antibody or the peptide or the linker modulates pharmacokinetics, pharmacodynamics, serum half-life, stability, expression level, and/or biodistribution of the complex. In some aspects, the antibody or the peptide or both disrupts an abnormal cellular process or pathway, has anti-cancer activity, enhances a body's immune system against an infection, pathogen, or any diseased, dysregulated, or uncontrolled cell, is a neuroprotective agent, is a chemotherapy agent, modulates an immune response toward a target cell or a cellular factor, enhances T-cell mediated immune response, induces or triggers apoptosis of a cancerous, abnormal, or diseased cell, binds to or modulates an antigen or a marker indicative of a central nervous system disorder, binds to or modulates a receptor or a surface marker or a cancer cell in a brain, binds to or modulates a biomarker or a factor associated with a neurodegenerative disease, binds to or modulates a pathogen or an infectious agent, suppresses an autoimmune response, suppresses or reduces angiogenesis.

In some aspects, the antibody is selected from any one of the antibodies listed in TABLE 4. In further aspects, the antibody is or has at least 80% sequence identity with an antibody selected from the group consisting of: Crenezumab, Urelumab, Utomilumab, Ensituximab, Tacatuzumab tetraxetan, Nesvacumab, Vanucizumab, Evinacumab, Vepalimomab, 8H9, Belimumab, Tabalumab, Bapineuzumab, Gantenerumab, Solanezumab, Aducanumab, Detumomab, Nacolomab tafenatox, Igovomab, Oregovomab, Sofituzumab vedotin, Abagovomab, Galcanezumab, Mogamulizumab, PRO 140, Tovetumab, Coltuximab ravtansine, Denintuzumab mafodotin, Inebilizumab, SGN-CD19A, Taplitumomab paptox, Afutuzumab, FBTA05, Moxetumomab pasudotox, Pinatuzumab vedotin, Varlilumab, Atezolizumab, Avelumab, Durvalumab, Enoblituzumab, Lilotomab satetraxetan, Naratuximab emtansine, Otlertuzumab, Tetulomab, Isatuximab, Daratumumab, Ibalizumab, Zanolimumab, Bleselumab, Dacetuzumab, Lucatumumab, Teneliximab, Bivatuzumab mertansine, Abituzumab, Intetumumab, Alemtuzumab, Lorvotuzumab mertansine, Itolizumab, Vorsetuzumab mafodotin, Milatuzumab, Polatuzumab vedotin, Galiximab, Altumomab pentetate, Arcitumomab, Labetuzumab, Besilesomab, Erenumab, Margetuximab, IMAB362, Actoxumab, Bezlotoxumab, Tefibazumab, Tisotumab vedotin, Cabiralizumab, Emactuzumab, Lenzilumab, Namilumab, Ticilimumab, tremelimumab, Ulocuplumab, Sevirumab, Regavirumab, Rovalpituzumab tesirine, Demcizumab, Enoticumab, Navicixizumab, Begelomab, Drozitumab, Parsatuzumab, Cetuximab, Depatuxizumab mafodotin, Futuximab, Imgatuzumab, Laprituximab emtansine, Matuzumab, Necitumumab, Nimotuzumab, Panitumumab, Zalutumumab, Carotuximab, Edobacomab, Nebacumab, Adecatumumab, Citatuzumab bogatox, Edrecolomab, Oportuzumab monatox, Solitomab, Tucotuzumab celmoleukin, Catumaxomab, Fibatuzumab, Epitumomab cituxetan, Sontuzumab, Duligotumab, Elgemtumab, Lumretuzumab, Patritumab, Seribantumab, Radretumab, Pasotuxizumab, Farletuzumab, Mirvetuximab soravtansine, Vantictumab, Crotedumab, 3F8, Ch.14.18, Dinutuximab, Derlotuximab biotin, Suvizumab, Apolizumab, Fasinumab, Ponezumab, Volociximab, Lirilumab, Monalizumab, cBR96-doxorubicin immunoconjugate, Carlumab, Amatuximab, Imalumab, Ublituximab, Anetumab ravtansine, Cantuzumab mertansine, Refanezumab, Fulranumab, Tanezumab, Racotumomab, Ozanezumab, Brontictuzumab, Tarextumab, Vesencumab, Nivolumab, Pidilizumab, Pembrolizumab, Olaratumab, Lifastuzumab vedotin, Bavituximab, Tosatoxumab, Icrucumab, Alacizumab pegol, Ramucirumab, and Pritumumab.

In other aspects, the antibody binds to or modulates a target or a target with at least 80% sequence identity with a target selected from the group consisting of: beta amyloid, Tau, alpha synuclein, BACE or BACE fragment (anti-BACE heavy chain, and anti-BACE light chain, or a fragment thereof), IL23, TGF beta, CD137, 5AC, alpha-fetoprotein, angiopoietin, anthrax toxin, AOC3, VAP-1, B7-H3, calcitonin, CD4, CD5, CD6, CD20, CD22, CD27, CD30, CD52, CD274, CD276, CD28, CD33, CD37, CD38, CD40, CD44, CD51, CD56, CD70, CD74, CD79B, CD80, CD137, CD140a, CD19, CGRP, ch4D5, CLDN18.2, coagulation factor III, CSF1R, CSF2, CTLA-4, EGFL7, EGFR, endotoxin, EpCAM, gpA3, CD3, ephrin receptor A3, episialin, αVβ3, α5β1, ErbB1/EGFR, ErbB2/HER2, ErbB3, ERBB3, HERS, FAP, fibrin, fibronectin, carbonic anhydrase IX, PSMA, folate binding protein, folate hydrolase, folate receptor, Frizzled receptor, GCGR, gangliosides (e.g., GD2, GD3, GM2) glypican 3, GMCSF receptor, GPNMB, growth differentiation factor 8, GUCY2C, HER1, HER2/neu, HGF, HHGFR, histone complex, HIV-1, HLA-DR, HNGF, scatter factor receptor kinase, TNF, IGF-1 receptor, CD221, IGF1, IGF2, c-MET, IGF1R, IL 17A, IL-13, IL-17, IL2, ILGF2, KIR2D, KLRC1, Lewis-Y antigen, Lewis-Y², MCP-1, mesothelin, MIF, MS4A1, MSLN, NGF, N-glycolylneuraminic acid, NOGO-A, Notch 1, Notch receptor, NRP1, PD-1, PD-L1, PDCD1, PDGF-R, phosphate-sodium co-transporter, RON, RTN4, SDC1, STEAP1, TAG-72, T-cell receptor, TEM1, tenascin, tenascin C, EphA3, TNFR, TNF-α, TRAIL-R1, TRAIL-R2, RANKL, FAP, alpha synuclein tumor-associated calcium signal transducer 2, TWEAK receptor, VEGF, VEGFR1, VEGFR2, vimentin, CD20/MS4A1, 4-1BB, 5T4, activin receptor-like kinase 1, adenocarcinoma antigen, AGS-22M6, B7-H3, BAFF, B-lymphoma cell, C242 antigen, CCR4, CCR5, CEA, CGRP, CLDN18.2, mucins, TAG-72, Clostridium difficile, clumping factor A, coagulation factor III, CSF1R, CSF2, CTLA-4, CXCR4 (CD184), cytomegalovirus, cytomegalovirus glycoprotein B, DLL3, DLL4, DPP4, DR5, EGFL7, EGFR, endoglin, endotoxin, HNGF, integrin α5β1, MSLN, myelin-associated glycoprotein, NGF, phosphatidylserine, and Staphylococcus aureus.

In some aspects, the antibody has at least 80% at least 85%, at least 90%, or at least 95% sequence identity with SEQ ID NO: 427 or SEQ ID NO: 428, or a fragment thereof. In some aspects, the peptide has at least 85%, at least 90%, or at least 95% sequence identity with any one of SEQ ID NO: 1, SEQ ID NO: 2, SEQ ID NO: 3, SEQ ID NO: 5, SEQ ID NO: 35, SEQ ID NO: 36, SEQ ID NO: 37, SEQ ID NO: 39, SEQ ID NO: 124, SEQ ID NO: 128, SEQ ID NO: 132, SEQ ID NO: 141, SEQ ID NO: 147, SEQ ID NO: 149, SEQ ID NO: 161, SEQ ID NO: 179, SEQ ID NO: 212, SEQ ID NO: 214, SEQ ID NO: 215, SEQ ID NO: 216, SEQ ID NO: 345, or SEQ ID NO: 374, SEQ ID NO: 244, SEQ ID NO: 245, SEQ ID NO:246, SEQ ID NO: 248, or a fragment thereof.

In some aspects, the peptide sequence is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues long.

In some aspects, at least one residue of the peptide comprises a chemical modification. In further aspects, the chemical modification is blocking the N-terminus of the peptide prior to conjugating or linking with the antibody. In still further aspects, the chemical modification is: methylation of one or more lysine residues or analogue thereof; methylation of an N-terminus; or methylation of one or more lysine residue or analogue thereof and methylation of the N-terminus.

In still further aspects, the peptide is fused with the antibody at one or more of the following: an N-terminus or a C-terminus of the peptide, at an N-terminus or a C-terminus of a heavy chain, a light chain, or a constant region of the antibody, or within the heavy chain, light chain, or constant region of the antibody.

In some aspects, the peptide-antibody complex further comprises a cleavable, non-cleavable, or a pH sensitive linker. In further aspects, the linker is cleavable, is pH sensitive, is sensitive to reduction, is cleavable by an enzyme, or is cleavable by cathepsin or matrix metalloprotease, or is a dipeptide. In still further aspects, the linker comprises a sequence of GGGSGGGS (SEQ ID NO: 444).

In further aspects, the linker is linked to the antibody, the peptide, a therapeutic agent, a detectable agent, or any combination thereof. In still further aspects, the linker links the peptide to the antibody at an N-terminus or a C-terminus of the peptide. In some aspects, the linker releases the peptide, the antibody, the therapeutic agent, the detectable agent, or any combination thereof upon cleavage, reduction, a pH change, or cleavage by a protease upon internalization of the peptide-antibody complex.

In some aspects, the peptide-antibody complex further comprises a therapeutic agent, wherein the therapeutic agent is a radiosensitizer or photo sensitizer, a microtubule inhibitor, DNA intercalator, DNA cleaver, or cytotoxic agent, or a modulator of cellular processes.

In further aspects, the microtubule inhibitor, DNA intercalator, or DNA cleaver is selected from the group consisting of: Bleomycin A2, Calicheamicin g-1, Auristatins (e.g., MMAE, MMAF, PE, Dolastatin-10), Maytansines (e.g., DM-1, DM-4, and derivatives), Vinorelbine, Paclitaxel, Epothilone B, Tubulysins (IM-2, B), Doxorubicin, Epirubicin, PNU-159682, Duocarmycins, PBD dimers, Oligomycin C, Daunorubicin, Valrubicin, Topotecan and desmethyl-Topotecan, DNA Transcription Inhibitors, Dactinomycin, Akt inhibitor, Ipatasertib (GDC-0068), DNA cross-linker, Mitomycin C, and Dihydrofolate reductase (DHFR) inhibitor, and Methotrexate.

In some aspects, the peptide-antibody complex further comprises a therapeutic agent, wherein the therapeutic agent is a cytotoxic molecule selected from the group consisting of: auristatin, a maytansinoid, doxorubicin, a calicheamicin, a platinum compound a taxane, paclitaxel, a BACE inhibitor, a Bcl-xL inhibitor, WEHI-539, venetoclax, ABT-199, navitoclax, AT-101, obatoclax, a pyrrolobenzodiazepine, and dolastatin.

In some aspects, the peptide-antibody complex further comprises a therapeutic agent conjugated to the complex at an N-terminus or a C-terminus of the complex, or wherein the peptide is linked to the therapeutic agent via a cleavable, non-cleavable, or a pH sensitive linker, or via the N-terminus, or via the epsilon amine of an internal lysine residue, or via the C-terminus of the peptide, or wherein the antibody is linked to the therapeutic agent at an N-terminus or a C-terminus of a heavy chain, light chain, a variable region, a constant region, or any combination thereof, of the antibody.

In some aspects, the peptide-antibody complex further comprises a detectable agent selected from the group consisting of: a fluorophore, a fluorescent dye, a near-infrared dye, a contrast agent, a nanoparticle, a metal-containing nanoparticle, a metal chelate, an X-ray contrast agent, a PET agent, a radioisotope, and a radionuclide chelator. In some aspects, the peptide crosses a blood brain barrier or a blood cerebrospinal fluid barrier of a subject.

In further aspects, the peptide or the antibody, or both home, target, is directed to or migrates to a tumor or diseased region, tissue, structure, or cell of the subject after crossing the blood brain barrier or the cerebrospinal fluid barrier, or a specific region in CNS, ventricles, the cerebrospinal fluid, the hippocampus, the meninges, the rostral migratory system, the dentate gyrus, the subventricular zone, or any combination thereof.

In some aspects, the antibody or the peptide, or the peptide-antibody complex, is capable of affecting neurological disorders, lysosomal storage diseases, epilepsy, meningitis, infections in the brain, stroke, and multiple sclerosis, aggregation of a protein associated with a neurodegenerative disease, or blocks a receptor or channel involved in neurological disorders.

In some aspects, the peptide or the antibody homes, targets, is directed to, or migrates to or penetrates a tumor or cancerous cell. In further aspects, the tumor or cancerous cell is a solid tumor, a brain cancer, a glioblastoma, a primary brain tumor, a metastatic brain tumor, a triple-negative breast cancer, a colon cancer, or a sarcoma.

In some aspects, the peptide or the antibody is conjugated or fused to an immunotherapy or an anti-angiogenic antibody. In some aspects, the peptide crosses a blood brain barrier to target a tumor in the brain or to reduce aggregation or plaque in the brain.

In further aspects, the antibody or peptide inhibits a cellular pathway associated with cancer.

In various aspects, the present disclosure provides a peptide comprising a sequence of any one of SEQ ID NO: 193-SEQ ID NO: 201, SEQ ID NO: 406-SEQ ID NO: 414, or a fragment thereof.

In various aspects, the present disclosure provides a peptide comprising a sequence that has at least 80%, at least 85%, at least 90%, or at least 95% sequence identity with any one of SEQ ID NO: 193-SEQ ID NO: 201, SEQ ID NO: 406-SEQ ID NO: 414, or a fragment thereof.

In some aspects, at least one residue of the peptide comprises a chemical modification, wherein the peptide is linked to a therapeutic agent, or wherein the peptide is linked to a detectable agent.

In various aspects, the present disclosure provides a pharmaceutical composition comprising any peptide-antibody complex described above or a salt thereof, and a pharmaceutically acceptable carrier. In some aspects, the pharmaceutical composition is formulated for inhalation, intranasal administration, oral administration, topical administration, intravenous administration, subcutaneous administration, intra-articular administration, intramuscular administration, intrathecal, intraperitoneal administration, or a combination thereof.

In various aspects, the present disclosure provides a method of treating a condition in a subject in need thereof, the method comprising: administering to the subject any peptide-antibody complex described above or any pharmaceutical composition described above. In some aspects, the peptide-antibody complex, the peptide or the antibody of the complex, or a pharmaceutical composition thereof, homes, targets, migrates to, or is directed to a cancerous or diseased region, tissue, structure, or cell of the subject following administration.

In some aspects, the condition is a tumor or cancer, a solid tumor, a brain tumor, triple-negative breast cancer, colon cancer, sarcoma, or a metastatic cancer of any of the foregoing. In some aspects, the peptide-antibody complex crosses the blood brain barrier or the blood cerebrospinal fluid barrier of the subject following administration or comes into proximity with the tumor after crossing the barrier.

In some aspects, the peptide-antibody complex, or the peptide or the antibody of the complex homes, targets, is directed to or migrates to the ventricles, cerebrospinal fluid, meninges, rostral migratory system, or hippocampus of the subject following administration. In some aspects, the condition is a brain disorder, is associated with a function of the ventricles, cerebrospinal fluid, the hippocampus, or is associated with a function of the brain. In some aspects, the peptide-antibody complex diagnoses, prevents, or treats the condition, wherein the condition is memory loss or memory function, Alzheimer's disease, Parkinson's disease, multiple system atrophy (MSA), schizophrenia, epilepsy, progressive multifocal leukoencephalopathy, fungal infection, depression, bipolar disorder, post-traumatic stress disorder, stroke, traumatic brain injury, infection, or multiple sclerosis.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic of a method of manufacturing and purifying a peptide as described herein.

FIG. 2 shows biodistribution of ¹⁴C radiolabeled SEQ ID NO: 1 in the brain and other tissues, wherein radiolabeled peptides are represented by darkened regions.

FIG. 3 shows the elution profile of anti-BACE1 antibody-peptide fusion comprising SEQ ID NO: 428 and SEQ ID NO: 429, wherein the peak represents the elution of the purified intact antibody-peptide fusion at the expected molecular weight. The gel inset shows a sample of the purified antibody-peptide fusion composing SEQ ID NO: 428 and SEQ ID NO: 429 (right lane) in comparison to a molecular weight standard (left lane) under non-reducing conditions.

FIG. 4 shows modulation of amyloid β-protein Aβ1-40 secretion modulated by a peptide-anti-BACE antibody fusion. Bioactivity of a peptide-antibody complex (solid line with black squares) comprising a peptide of SEQ ID NO: 1 fused to an anti-BACE antibody heavy chain of SEQ ID NO: 427 (SEQ ID NO: 429) and an anti-BACE antibody light chain (SEQ ID NO: 428) at various concentrations was tested. Bioactivity of the peptide-antibody complex was compared to a control comprising an anti-BACE antibody heavy chain (SEQ ID NO: 427) and an anti-BACE antibody light chain (SEQ ID NO: 428). The peptide-antibody complex displayed a similar dose-dependent effect as the antibody control.

FIG. 5 shows a diagram of various embodiments of peptide-antibody complexes. FIG. 5A shows a diagram of an antibody without a peptide. FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M, FIG. 5N, FIG. 5O, FIG. 5P, and FIG. 5Q show peptide-antibody complexes including different configurations and/or combinations of one or more peptides (represented by a circle) conjugated to, fused, linked, or embedded in an antibody at various regions of the antibody or antibody fragment. The variable heavy chain (V_{H}), variable light chain (V_{L}), constant heavy chain (C_{H}), and the constant light chain (C_{L}) of an antibody are represented by boxes.

### DETAILED DESCRIPTION

The present disclosure describes compositions and methods of use thereof comprising peptide-antibody complexes, including conjugates and fusions that can target specific cells or tissue in vivo. In some embodiments, peptide-antibody complexes can serve as a platform or carrier for attaching and delivering one or more therapeutic agents or detectable agents to target cells or tissue, such as cancerous cells, tumor, infected or disease cells. In various embodiments wherein a peptide-antibody complex serves as the platform or carrier of a therapeutic agent, the present disclosure contemplates either the peptide or the antibody, or both the peptide and the antibody of the peptide-antibody complex providing targeting or homing functions, such that the peptide-antibody complex targets or accumulates in or near specific cells or tissues or deliver the therapeutic agent to a specific cell or tissue of interest. Examples of specific cells or tissue include cancerous cells, tumors, nerve cells within the central nervous system or the peripheral nervous system, cells with dysregulated pathways or uncontrolled growth, cells with specific surface markers or receptors indicative of cellular dysfunction, infection, and/or disease, or any diseased, unhealthy, or dysfunction cell or tissue.

Also contemplated in the present disclosure are compositions and methods of use thereof comprising peptide-antibody complexes that exert a therapeutic effect on a target cell or tissue. In such embodiments, the present disclosure contemplates embodiments wherein either the peptide or the antibody, or both, provide the targeting or homing function. In some embodiments, either the peptide or the antibody, or both, can provide therapeutic effect on a target cell or tissue in vivo. In some embodiments, the peptide can cross the blood brain barrier (BBB) and/or the cerebrospinal barrier to target cells or tissues of the central nervous system (CNS), can target cells of the peripheral nervous system, can target tissue of a specific organ, can penetrate target cells to deliver an antibody or any therapeutic agent attached to the peptide-antibody complex inside, near, or at the site of the target cell or tissue, can target or cause the peptide-antibody complex to accumulate in a target cell or tissue, can serve as a platform or a means for attaching another moiety or molecule to the peptide-antibody complex, or can modulate the biodistribution, pharmacodynamics, and/or pharmacokinetics of the peptide-antibody complex. In other embodiments, the antibody of the peptide-antibody complex can provide various effector functions, such as inducing or mediating an immune response, triggering apoptosis, extending the half-life of the peptide or any moiety or molecule attached, bound, associated with, conjugated, or fused to the peptide-antibody complex, modulating the biodistribution, pharmacodynamics, and/or pharmacokinetics of the peptide-antibody complex, targeting certain cells or tissue to deliver the peptide of the peptide-antibody complex or any therapeutic agent attached to the peptide-antibody complex inside, near, or at the site of the target cell or tissue, targeting or causing the peptide-antibody complex to accumulate in a target cell or tissue, or serving as a platform or a means for attaching, conjugating, or fusing one or more peptides as described herein and/or another moiety or molecule to the peptide-antibody complex.

The peptide-antibody complexes as described herein can be used to treat or adapted to treat any disease, disorder, or condition wherein a therauptic agent, including a protein, DNA, RNA, a cytokine, a small molecule, a peptide, an antibody, a radioisotope, a toxin, and a drug, that can be attached, conjugated, bound, or fused to a peptide-antibody complex, which can target and deliver the therapeutic agent, inside, near, or at a site of a target cell or tissue, such as cancerous cells, cells with uncontrolled growth, cells with overexpression of one or more oncogenes, cells presenting a certain antigen, marker, or receptor recognized by the peptide-antibody complex, tumors, or any diseased, unhealthy, or infected cells of a body. The peptide-antibody complexes described herein can be used to treat or slow the progression of cancer, tumor growth, cellular damage, or spread of an infection. In some embodiments, the peptide-antibody complexes are adapted to enhance a body's immune response against an infection, pathogen, or any diseased, dysregulated, or uncontrolled cells in vivo. In other embodiments, the peptide-antibody complexes described herein can be used to provide a restorative or a therapeutic agent that helps to repair or restore a cell, or enhance a cellular function. Of the various disease, disorders, or conditions, various diseases, disorders, or conditions of the nervous system, especially those of the CNS or neurological conditions, and cancer are important categories, as these types of diseases, disorders, or conditions often can require a high specificity targeting mechanism, and ability to cross the BBB and/or the blood cerebral spinal fluid (CSF) barrier to reach a target cell or tissue of interest.

Treatment of neurological conditions, including, but not limited to, CNS disorders or conditions, cancer, brain tumors, neurodegenerative diseases, epilepsy, dementia, cerebrovascular diseases, stroke, multiple sclerosis, infections, autoimmune disease, and metabolic diseases, can be challenging and complicated to treat. One challenge associated with conditions affecting the CNS can that many drugs administered into the circulatory system of patients fail to cross the BBB or the CSF barrier, which are selective barriers that separate the circulating blood from the brain extracellular fluid and the central nervous system tissue. Another challenge is that many drugs lack sufficient specificity to one or more target regions, tissues, structures or cells in the brain. Treatment of CNS disorders often requires the use of high concentrations of non-specific drugs, leading to suboptimal efficacy and systemic side effects. Thus, there is a need for improved targeted therapies or pharmaceuticals for CNS disorders and cancers.

Some therapeutic agents also have short half-lives and/or poor biodistribution to target cells or tissue. One way to deliver drugs or therapeutics to the target cells or tissue is to apply them directly in conjunction with surgical procedures, or applying therapeutics in situ. Another way is to identify specific carriers that can target the cells or tissue or interest, or cross barriers to reach the target cells or tissue of interest, such as the BBB or the CSF barrier. Specific carriers conjugated to or complexed to potent drugs that are capable of crossing such barriers can counteract the non-specificity of many treatments by selectively targeting and delivering compounds to specific tissues, cells, structures and regions. Such drugs can also be useful to modulate ion channels, protein-protein interactions, extracellular matrix remodeling (i.e., protease inhibition), intracellular signaling pathways, neurotransmitter signaling, immune response, or other cellular proceses. Such targeted therapy can allow for lower dosing, higher efficacy, reduced off-target side effects, and improvement in therapeutic outcomes.

The present disclosure provides compositions and methods for treating various cancers and neurological disorders, including tumor in the brain, CNS disorders, infections, degenerative conditions, and functional disorders, such as epilepsy or Tourette's syndrome, in a subject by administering a peptide-antibody complex, including peptide-antibody fusion or conjugate formed using a linker or non-covalent interactions. Described herein are different combinations and/or configurations of peptide-antibody complexes, including fusion and conjugates, wherein the peptide is a derived from knottins, or cysteine knot peptides, that can home, distribute to, target, be directed to, accumulate in, migrate to, and/or bind to cancerous or diseased cells. This targeting function of peptides allows peptides to serve as carriers of antibodies, or any suitable therapeutic agent, to treat or confer a therapeutic effect on cancerous or diseased cells, such as a cytotoxic effect. It is advantageous to use a peptide that homes, distributes to, targets, migrates to, or accumulates in one or more specific cancerous or diseased regions, tissues, structures or cells to reduce off-target and potentially negative effect.

The present disclosure also provides compositions and methods of using immune-conjugates or immune-oncology therapies, including antibodies conjugated or joined to a peptide, which itself can be a toxin or trigger a therapeutic effect on a target cell or a cancerous/diseased cell. Peptide-antibody complexes can be further joined or conjugated to a toxin, radioisotope, or label. In some embodiments, the antibody portion of the peptide-antibody complex can serve as the carrier that can home, distribute to, target, be directed to, accumulate in, migrate to, and/or bind to cancerous or diseased cells, or to deliver a toxin or a therapeutic agent complexed, fused, or conjugated to the antibody to a specific target or cell recognized by the antibody. Some antibodies can also extend the half-life or improve the stability of a peptide or another molecule complexed, fused, bound, conjugated, or associated with the antibody. In some instances, association or complexing with an antibody alters the pharmacokinetics, pharmacodynamics, and/or biodistribution of a peptide or a therapeutic molecule associated with, attached to, linked to, conjugated to, bound to, or fused to the antibody. In some aspects, association of a peptide with an antibody can increase therapeutic window of a therapeutic agent attached to the peptide-antibody complex.

Also described herein are peptide-antibody complexes, including fusions, conjugates, and complexes that are bound together due to affinity, wherein the peptide component selectively homes, distributes to, targets, is directed to, migrates to, or accumulates in specific regions, tissues, structures or cells of the brain. In some cases, peptides target cancerous or diseased cells anywhere in the body, or solid tumors anywhere in the body, while some peptides target cancerous or diseased cells in the nervous system, CNS, or the brain. In some cases, peptide-antibody complexes accumulate in one or more areas of the brain targeted by the peptide, such as the hippocampus, the center of memory and learning and spatial navigation; the cerebrospinal fluid (CSF), which is found in the brain and spine; the ventricular system, the site of CSF production and circulation; the rostral migratory stream; the dentate gyrus; neural stem cells; or neuronal precursors. The dentate gyrus of the hippocampus and the subventricular zone are two locations of neurogenesis in the adult brain, and the rostral migratory stream is one mechanism for migration of new neurons. Thus, targeting those regions could allow for modulation of various aspects of neurogenesis, including repair or regeneration. A peptide that homes, distributes to, targets, migrates to, or accumulates in one or more specific regions, tissues, structures or cells of the brain can have fewer off-target and potentially negative effects. For example, side effects that often limit use and efficacy of drugs for neurological conditions or disorders. In some cases, peptides of peptide-antibody complexes home, distribute to, target, migrate to, or accumulate in one or more specific cell types in the CNS, including, but not limited to, sensory neurons, motor neurons, multipolar neurons, bipolar neurons, interneurons, excitatory interneurons, inhibitory interneurons, astrocytes, and glial cells. Peptides in peptide-antibody complexes can increase the efficacy and effective local dose of antibodies by directly targeting them to a specific region, tissue, structure or cell of the brain and helping the antibody cross the blood brain barrier or blood CSF barrier. The present disclosure provides compositions and methods for treating a CNS disorder in a subject by administering a peptide-antibody complex or a peptide-antibody complex that is further conjugated to a therapeutic agent.

In some embodiments, peptide-antibody fusions target or bind to an antigen associated with a CNS disorder or a biomarker of a CNS disorder. Examples of an antigen associated with a CNS disorder or a biomarker of a CNS disorder include, but are not limited to, Tau, alpha synuclein, beta amyloid, and prion. In some embodiments, peptide-antibody fusions target or bind to a CNS cell or a CNS antigen associated with any one of the CNS disorders described herein, including, but not limited to, Alzheimer's disease, Parkinson's disease, multiple system atropy (MSA), transmissible spongiform encephalopathies (TSEs) (e.g., Creutzfeldt-Jakob disease (CJD), bovine spongiform encephalopathies (BSE) and the like) Huntington's disease, Amyotrophic lateral sclerosis (ALS or Lou Gehrig's Disease), dementia, multiple sclerosis, meningitis, and epilepsy. For instance, Alzheimer's disease is a brain disorder that is associated with the aggregation of amyloid beta peptide fragment. The accumulation of the amyloid beta peptide fragment is a result of proteolytic cleavage of the amyloid precursor protein (APP) by an enzyme known as beta-secretase, or beta-site APP cleaving enzyme (BACE). A peptide-antibody fusion that can cross the BBB to interact with and inhibit the beta-secretase protease could be used in the treatment and prevention of Alzheimer's disease by reducing aggregation of the amyloid beta fragment through, for example, binding or inhibiting the protease using an anti-BACE antibody that antagonizes, reduces, or interferes with APP cleavage, thus regulating the amyloid beta fragment pathway and reducing the amount of amyloid beta fragment produced. Such anti-BACE antibody can be engineered based on the crystal structure of the human antibody localized with BACE. See J. K. Atwal et al., A Therapeutic Antibody Targeting BACE1 Inhibits Amyloid-Production in vivo. Science Translational Medicine. 3, 84ra43-84ra43 (2011). Many types of tumors are difficult to treat. Often, the prognosis of the patient is directly influenced by the ability drug therapies can effectively kill the cancerous cells and on the precision with which the cancer cells can be surgically resected. For example, one challenge in treating tumors is that many drugs treatments are systemic, and therefore, the efficacy of their use is constrained by the toxicity of systemic use.

In some embodiments, peptide-antibody complexes, including fusions and conjugates, target or bind to an antigen associated with cancer or a tumor biomarker. Examples of cancer antigen or tumor biomarker include, but are not limited to, CA-125, MUC16, AFP (liver cancer), BCR-ABL (chronic myeloid leukemia), BRCA1/BRCA2 (breast/ovarian cancer), BRAF V600 (melanoma/colorectal cancer), CA-125 (ovarian cancer), CA19.9 (pancreatic cancer), CEA, EGFR, KRAS, UGT1A1 (colorectal cancer), EGFR (non-small-cell lung carcinoma), HER-2/neu (breast cancer), KIT (gastrointestinal stromal tumor), PSA (prostate cancer), BRAF, S100 (melanoma), KRAS, p53, erbB2 (colorectal, esophageal, liver, and pancreatic cancers), abnormal methylation of tumor suppressor genes p16, CDKN2B, and p14ARF (brain cancer), hypermethylation of MYOD1, CDH1, and CDH13 (cervical cancer), and hypermethylation of p16, p14, and RB1 (oral cancer), TIMP1, CD137, CTLA-4, CD40 (multiple myeloma), CD20, CD30, PDGFR, PML/RAR alpha, TPMT, UGT1A1 (leukemia/lymphoma), and a mutant or variant thereof. Other tumor markers include, but not limited to, ALK, AFP, B2M, beta-hCG, BCR-ABL, c-kit/CD117, CA15-3/CA27.29, CA19-9, CA-125, calcitonin, carcinoembryonic antigen (CEA), CD20, chromogranin A (CgA), cytokeratin fragment 21-1, estrogen receptor (ER)/progesterone receptor (PR), fibrin/fibrinogen, HE4, lactate dehydrogenase, neuron-specific enolase (NSE), nuclear matrix protein 22, programmed death ligand 1 (PD-L1), thyroglobulin, urokinase plasminogen activator (uPA) and plasminogen activator inhibitor (PAI-1).

In some embodiments, peptide-antibody complexes are specific for cancerous cells and function to deliver or target the conjugated antibodies to cancerous cells anywhere in the body. In some aspects, peptide-antibody complexes are targeted to a solid tumor in the body. In other aspects, peptide-antibody complexes are targeted to a specific cancerous cell type. Examples of cancerous cell types include, but are not limited to, carcinoma formed by epithelial cells, sarcoma formed in bone and soft tissues, leukemia formed from blood-forming tissue of the bone marrow, lymphoma formed from lymphocytes (T or B cells), multiple myeloma formed from plasma cells, melanoma formed from melanocytes, and brain and spinal cord tumors (e.g., astrocytic tumor formed from astrocytes).

In some embodiments, peptide-antibody complexes are used in targeted therapies, which interfere with cancer cell growth or survival. Targeted therapies can involve small molecules or monoclonal antibodies, which can be conjugated or fused to a peptide. Several types of targeted therapies are available, including hormone therapies, signal transduction inhibitors, gene expression modulators, apoptosis inducers, angiogenesis inhibitors, immunotherapies, and toxin delivery molecules. In some embodiments, immunotherapies trigger a subject's immune system to destroy or attack cancer cells. Some immunotherapies comprise monoclonal antibodies that recognize specific molecules or markers on the surface of cancer cells. Some monoclonal antibodies bind to their target molecules to trigger the immune system to destroy cells that express the target molecules. In other aspects, monoclonal antibodies can bind to immune cells to help the immune cells kill cancer or diseases cells expressing a certain antigen. In some embodiments, monoclonal antibodies can deliver toxic molecules that cause the death of cancer cells or diseased cells that come in contact with the toxic molecules, or internalize the toxic molecules.

In some embodiments, peptide-antibody fusions or conjugates cross the BBB and target cancerous cells in the brain. In some embodiments, peptide-antibody fusions or conjugates target cancerous cells of any one of the following types of brain tumor: astrocytoma, atypical teratoid rhaboid tumor (ATRT), chondrosarcoma, choroid plexus, craniopharyngioma, cysts, ependymoma, germ cell tumor, glioblastoma, glioma, hemangioma, juvenile pilocytic astrocytoma, lipoma, lymphoma, medulloblastoma, meningioma, neurofibroma, neuronal and mixed neuronal-glial tumor, oligoastrocytoma, oligodendroglioma, pineal tumor, pituitary tumor, PNET, and Schwannoma, and tumors that have metastasized to the brain.

In certain embodiments, a peptide-antibody fusion or conjugate is further conjugated to a fluorophore or a label for imagining. In other embodiments, a peptide-antibody fusion is further conjugated to neurotensin peptide ELYENKPRRPYIL (SEQ ID NO: 441), which is a tridecapeptide found in the CNS and the gastrointestinal (GI) tract. For example, in some embodiments an antibody is conjugated to any one of the peptide-neurotensin fusions shown in SEQ ID NO: 431-SEQ ID NO: 435 (with N-terminal GS before peptide and neurotensin sequence) or SEQ ID NO: 436-SEQ ID NO: 440 (without N-terminal GS before peptide or neurotensin sequence), as shown below in TABLE 1.

TABLE 1
Neurotensin Sequences

| SEQ ID NO | Sequence |
|---|---|
| SEQ ID NO: 431 | GSRCLPPGRPCYGATQRIPCCGVCSHNNCTGSSELYENKPRRPYIL |
| SEQ ID NO: 432 | GSVGCEECPMHCRGRNANPTCDDGVCNCNVGSSELYENKPRRPY |
| | IL |
| SEQ ID NO: 433 | GSMCMPCFTTDTQMQERCDRCCGGGGRGRCWGPQCLCIGSSELY |
| | ENKPRRPYIL |
| SEQ ID NO: 434 | GSMCMPCFTTDPNMAKKCRDCCGGNGKCFGPQCLCNRGSSELYE |
| | NKPRRPYIL |
| SEQ ID NO: 435 | GSSEKDCIKHLQRCRENKDCCSKKCSRRGTNPEKRCRGSSELYEN |
| | KPRRPYIL |
| SEQ ID NO: 436 | RCLPPGRPCYGATQRIPCCGVCSHNNCTELYENKPRRPYIL |
| SEQ ID NO: 437 | VGCEECPMHCRGRNANPTCDDGVCNCNVELYENKPRRPYIL |
| SEQ ID NO: 438 | MCMPCFTTDTQMQERCDRCCGGGGRGRCWGPQCLCIELYENKPR |
| | RPYIL |
| SEQ ID NO: 439 | MCMPCFTTDPNMAKKCRDCCGGNGKCFGPQCLCNRELYENKPR |
| | RPYIL |
| SEQ ID NO: 440 | EKDCIKHLQRCRENKDCCSKKCSRRGTNPEKRCRELYENKPRRPY |
| | IL |

Neurotensin can function like a neurotransmitter in the brain and a hormone in the GI tract. Neurotensin can also act as a neuromodulator in various neurotransmitter systems, and has been implicated in pathophysiology of various CNS disorders, such as pain, Parkinson's disease, eating disorders, cancer, and inflammation.

In other embodiments, a therapeutic agent can be a cytotoxic agent. For example, a peptide-antibody complex can be further conjugated to one or more cytotoxic agents that kill target cells, such as when cytotoxic agents are released from the peptide-antibody complex or internalized by target cells. Peptide-antibody complexes can function as a targeting carrier for attaching and delivering such cytotoxic agents to specific target cells recognized by the peptide-antibody complexes. Such cytotoxic agents can be synthetic, naturally occurring, or derivatives of naturally occurring toxins. In some embodiments, stable linkers can be used to attach the cytotoxic agents to the peptide-antibody complex. In other embodiments, the linker may be cleavable and releases the cytotoxic agent at the target cell or a target site, such as in response to a pH change or in response to another molecule that cleaves the linker. Cytotoxic agent can be any agent that is able to kill a cell or trigger cellular processes that lead to cell death. Examples of cytotoxic agents include pyrrolobenzodiazepine dimers (PBD) (DNA minor groove cross-linker), antibiotic, chemotherapy drugs, maytansines (tubulin depolymerization), calicheamicins (DNA cleavage), duocarymycins (DNA minor groove alkylating agent), alpha-amanitin (RNA polymerase inhibitor), auristatins (tubulin polymerase inhibitors), including, but not limited to, monomethyl auristatin E (MMAE) and monomethyl auristatin F (MMAF).

In some embodiments, brain-reactive antibodies can be used to target specific antigens or cells in the brain, allowing such antibodies to serve as the homing or targeting components in peptide-antibody complexes. In such complexes, the peptide can serve as a substrate for further attaching or conjugating therapeutic agents to the peptide-antibody complexes. Examples of antibody-related disorders of the CNS and antigens recognized by brain-reactive antibodies include hnRNP A1 antigen (HAM/tropical spastic paraparesis), AQP4 antigen (neuromyelitits optica), MOG antigen (acute disseminated encephalomyelitis), NR2A/NR2B antigen and neuroncal surface P antigen (systematic lupus erythematosus), lysoganglio side dopamine D2 receptor or tubulin antigens (post-streptococcal movement disorders, Sydenham's chorea), synapsin 1 or transglutaminase antigens (celiac disease), AMPAR, GluR1, GluR2, NMDAR, NR1/NR2B, Lgi1 (limbic encephalitis), GluR3 antigen (Rasmussen encephalitis), Aldehyde reductase or thyroglobulin (Hashimoto's encephalitis), GAD, Gephryin, GABA(B) receptor, or amphiphysin (Stiff-person syndrome). The abbreviations are: AMPAR, α-amino-3-hydroxy-5-methyl-4-isoxazolepropionic acid receptor; mGluR, metabotropic glutamate receptor; AQP4, astrocytic aquaporin-4 water channels; CSF, cerebrospinal fluid; GABA, γ-aminobutyric acid; GAD, glutamic acid decarboxylase; HAM, human T-lymphotropic virus type 1-associated myelopathy; hnRNP A1, heterogeneous ribonucleoprotein A1; Lgi1, leucine-rich, glioma-inactivated 1; NMDAR, N-methyl-d-aspartate receptor, NR1, NR2A, and NR2B, subunits of the NMDAR; MOG; myelin oligodendrocyte glycoprotein.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N, Asn), glutamine (Q, Gln), histidine (H, His), lysine (K. Lys), or arginine (R, Arg).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

### Peptide Compositions

Knottins are a class of peptides, usually ranging from about 11 to about 81 amino acids in length that are often folded into a compact structure. Knottins are typically assembled into a complex tertiary structure that is characterized by a number of intramolecular disulfide crosslinks and may contain beta strands and other secondary structures. The presence of the disulfide bonds gives knottins remarkable environmental stability, allowing them to withstand extremes of temperature and pH and to resist the proteolytic enzymes of the blood stream. The rigidity of knottins also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The knotted peptides can bind targets with antibody-like affinity. A wider examination of the sequence structure and sequence identity or homology of knottins reveals that they have arisen by convergent evolution in all kinds of animals and plants. In animals, they are typically found in venoms, for example, the venoms of spiders and scorpions and have been implicated in the modulation of ion channels. The knottin proteins of plants can inhibit the proteolytic enzymes of animals or have antimicrobial activity, suggesting that knottins can function in the native defense of plants.

The present disclosure provides peptides that comprise or are derived from these knotted peptides (or knottins). As used herein, the term “knotted peptide” is considered to be interchangeable with the terms “knottin” and “optide.” Knotted peptides are a class of peptides, usually ranging from about 11 to about 81 amino acids in length, that are often folded into a compact structure. In certain embodiments, knotted peptides are assembled into a complex tertiary structure that is characterized by a number of intramolecular disulfide crosslinks, and optionally contain beta strands and other secondary structures such as an alpha helix.

The peptides of the present disclosure can comprise cysteine amino acid residues. In some cases, the peptide has at least 6 cysteine amino acid residues. In some cases, the peptide has at least 8 cysteine amino acid residues. In other cases, the peptide has at least 10 cysteine amino acid residues, at least 12 cysteine amino acid residues, at least 14 cysteine amino acid residues or at least 16 cysteine amino acid residues.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the disulfide bridges form a knot. A disulfide bridge can be formed between cysteine residues, for example, between cysteines 1 and 4, 2 and 5, or, 3 and 6. In some cases, one disulfide bridge passes through a loop formed by the other two disulfide bridges, for example, to form the knot. In other cases, the disulfide bridges can be formed between any two cysteine residues.

The present disclosure further includes peptide scaffolds that, e.g., can be used as a starting point for generating additional peptides. In some embodiments, these scaffolds can be derived from a variety of knotted peptides (or knottins). In certain embodiments, knotted peptides are assembled into a complex tertiary structure that is characterized by a number of intramolecular disulfide crosslinks, and optionally contain beta strands and other secondary structures such as an alpha helix. For example, knotted peptides include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures include peptide having two parallel helices linked by two disulfide bridges without β-sheets (e.g., hefutoxin).

A knotted peptide can comprise at least one amino acid residue in an L configuration. A knotted peptide can comprise at least one amino acid residue in a D configuration. In some embodiments, a knotted peptide is 15-40 amino acid residues long. In other embodiments, a knotted peptide is 11-57 amino acid residues long. In still other embodiments, a knotted peptide is 11-81 amino acid residues long. In further embodiments, a knotted peptide is at least 20 amino acid residues long.

Knotted peptides can be derived from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Ornithoctonus huwena, Hadronyche versuta, Atrax robustus, Angelenopsis aperta, Psalmopoeus cambridgei, Hadronyche infensa, Paracoelotes luctosus, and Chilobrachys jingzhaoor another suitable genus or species of scorpion or spider. In some cases, a peptide can be derived from a Buthus martensii Karsh (scorpion) toxin. In some embodiments, a peptide can be derived from a member of the pfam005453: Toxin_6 class.

TABLE 2 lists exemplary peptide sequences that can be linked, conjugated, fused, or complexed to an antibody to form a peptide-antibody complex.

TABLE 2
Exemplary Peptide Sequences

| SEQ ID NO | Amino Acid Sequence |
|---|---|
| SEQ ID NO: 1 | GSMCIPCFTTNPNMAAKCNACCGSRRGSCRGPQCIC |
| SEQ ID NO: 2 | GSGCLEFWWKCNPNDDKCCRPKLKCSKLFKLCNFSFG |
| SEQ ID NO: 3 | GSECRYWLGTCSKTGDCCSHLSCSPKHGWCVWDWTFRK |
| SEQ ID NO: 4 | GSMCMPCFTTDHQMARRCDDCCGGRGRGRCYGPQCLCR |
| SEQ ID NO: 5 | GSMCMPCFTTHHRMAENCDICCGGDGRGKCYGPQCLCR |
| SEQ ID NO: 6 | GSMCMPCFTTDHRMAENCDICCGGDGRGKCYGPQCLCR |
| SEQ ID NO: 7 | GSMCMPCFTTHHQMAENCDICCGGDGRGKCYGPQCLCR |
| SEQ ID NO: 8 | GSMCMPCFTTHHRMARNCDICCGGDGRGKCYGPQCLCR |
| SEQ ID NO: 9 | GSMCMPCFTTHHRMAERCDICCGGDGRGKCYGPQCLCR |
| SEQ ID NO: 10 | GSMCMPCFTTHHRMAENCDDCCGGDGRGKCYGPQCLCR |
| SEQ ID NO: 11 | GSRCMPCFTTDHQMARRCDDCCGGRGRGKCYGPQCLCR |
| SEQ ID NO: 12 | GSICIPCFTTDHQIARRCDDCCGGRGRGKCYGPQCLCR |
| SEQ ID NO: 13 | GSMCLPCFTTDHQLARRCDDCCGGRGRGKCYGPQCLCR |
| SEQ ID NO: 14 | GSMCMPCFTTEHQMARRCEECCGGRGRGKCYGPQCLCR |
| SEQ ID NO: 15 | GSMCIPCFTTDHQMARRCEECCGGRGRGKCYGPQCLCR |
| SEQ ID NO: 16 | GSICIPCFTTDHQMARRCDDCCGGRGDGKCYGPQCLCR |
| SEQ ID NO: 17 | GSICIPCFTTDHQIARRCDDCCGGRGRGKCYGPQCICR |
| SEQ ID NO: 18 | GSRCMPCFTTDHFMARFCDFCCGGRGRGKCYGPQCLCR |
| SEQ ID NO: 19 | GSRCMPCFTTDHYMARYCDYCCGGRGRGKCYGPQCLCR |
| SEQ ID NO: 20 | GSRCMPCFTTDHRMARRCDRCCGGRGRGKCYGPQCLCR |
| SEQ ID NO: 21 | GSRCMPCFTTDHEMARECDECCGGRGRGKCYGPQCLCR |
| SEQ ID NO: 22 | GSRCMPCFTTDHHMARHCDHCCGGRGRGKCYGPQCLCR |
| SEQ ID NO: 23 | GSLCLPCFTTHHRLADQCDICCGGDGRGKCYGPQCLCR |
| SEQ ID NO: 24 | GSICIPCFTTEHQIARRCEECCGGRGRGKCYGPQCLCR |
| SEQ ID NO: 25 | GSMCMPCFTTDTQMQERCDRCCGGGGRGKCWGPQCLCI |
| SEQ ID NO: 26 | GSMCMPCFTTIYRMAHECDECCGGRGRGKCYGPQCLCR |
| SEQ ID NO: 27 | GSMCMPCFTTGYRMAEYCDICCGGRGRGKCYGPQCLCR |
| SEQ ID NO: 28 | GSMCMPCFTTHRRMANTCDACCGGRSRGKCYGPQCLCR |
| SEQ ID NO: 29 | GSHCMPCFTTDHQMIRRCDDCCGGGSYGKCDGPQCLCF |
| SEQ ID NO: 30 | GSDCMPCFTTDHRMADHCDICCGGDDRGKCYGPQCLCR |
| SEQ ID NO: 31 | GSMCMPCFTTDHEMERRCDDCCGIGGGGKCHGPQCLCG |
| SEQ ID NO: 32 | GSMCMPCFTTEQRMAIICDDCCGGFGRGKCYGPQCLCR |
| SEQ ID NO: 33 | GSMCMPCFTTSEQMFRRCDDCCGGWGDGKCNGPHCLCR |
| SEQ ID NO: 34 | GSGVPINVKCRGSRDCLDPCKKAGMRFGKCINSKCHCTP |
| SEQ ID NO: 35 | GSMCMPCFTTEQRMAIICDDCCGGFGRGRCYGPQCLCR |
| SEQ ID NO: 36 | GSICIPCFTTDHQIARRCDDCCGGRGRGRCYGPQCICR |
| SEQ ID NO: 37 | GSMCMPCFTTDTQMQERCDRCCGGGGRGRCWGPQCLCI |
| SEQ ID NO: 38 | GSMCMPCFTTDTQMQERCDRCCGGGGRGRCWGPQCLC |
| SEQ ID NO: 39 | GSMCMPCFTTDHRMAENCDICCGGDGRGRCYGPQCLCR |
| SEQ ID NO: 40 | GSMCMPCFTTEQRMAIICDDCCGGFGRGKCYGPQCLCI |
| SEQ ID NO: 41 | GSMCMPCFTTEQRMAIICDDCCGGFGRGRCYGPQCLCI |
| SEQ ID NO: 42 | GSICIPCFTTDHQIARRCDDCCGGRGRGKCYGPQCICI |
| SEQ ID NO: 43 | GSICIPCFTTDHQIARRCDDCCGGRGRGRCYGPQCICI |
| SEQ ID NO: 44 | GSMCMPCFTTDTQMQEKCDRCCGGGGRGRCWGPQCLCI |
| SEQ ID NO: 45 | GSMCMPCFTTEQRMAIKCDDCCGGFGRGRCYGPQCLCR |
| SEQ ID NO: 46 | GSICIPCFTTDHQIARKCDDCCGGRGRGRCYGPQCICR |
| SEQ ID NO: 47 | GSMCMPCFTTDHRMAEKCDICCGGDGRGRCYGPQCLCR |
| SEQ ID NO: 48 | GSMCMPCFTTDTQMQERCDRCCGGKGRGRCWGPQCLCI |
| SEQ ID NO: 49 | GSMCMPCFTTEQRMAIICDDCCGGKGRGRCYGPQCLCR |
| SEQ ID NO: 50 | GSICIPCFTTDHQIARRCDDCCGGKGRGRCYGPQCICR |
| SEQ ID NO: 51 | GSMCMPCFTTDHRMAENCDICCGGKGRGRCYGPQCLCR |
| SEQ ID NO: 52 | GSMCMPCFTTDHRMAENCDICCGGDGRGKCYGPQCLCI |
| SEQ ID NO: 53 | GSMCMPCFTTDHRMAENCDICCGGDGRGRCYGPQCLCI |
| SEQ ID NO: 54 | GSMCMPCFTTHHRMAENCDICCGGDGRGRCYGPQCLCR |
| SEQ ID NO: 55 | GSVGCEECPMHCKGKNANPTCDDGVCNCNV |
| SEQ ID NO: 56 | GSVGCEECPMHCKGKNAKPTCDDGVCNCNV |
| SEQ ID NO: 57 | GSVGCEECPMHCKGKNAKPTCDNGVCNCNV |
| SEQ ID NO: 58 | GSVGCEECPMHCKGKHAVPTCDDGVCNCNV |
| SEQ ID NO: 59 | GSVGCEECPAHCKGKNAKPTCDDGVCNCNV |
| SEQ ID NO: 60 | GSVGCEECPAHCKGKNAIPTCDDGVCNCNV |
| SEQ ID NO: 61 | GSVGCEECPMHCKGKMAKPTCDDGVCNCNV |
| SEQ ID NO: 62 | GSVGCEECPMHCKGKNAVPTCDNGVCNCNA |
| SEQ ID NO: 63 | GSVGCEECPMHCKGKMAKPTCYDGVCNCNV |
| SEQ ID NO: 64 | GSVGCEECPMYCKGKNAVPTCDGGVCNCNA |
| SEQ ID NO: 65 | GSVGCEECPKYCKGKNAVPTCDGGVCNCNA |
| SEQ ID NO: 66 | GSVGCEECPVYCKGKKALPTCDGGVCNCNA |
| SEQ ID NO: 67 | GSVGCEEDPMHCKGKQAKPTCCNGVCNCNV |
| SEQ ID NO: 68 | GSVGCAECPMHCKGKMAKPTCENEVCKCNIGKKD |
| SEQ ID NO: 69 | GSVGCEECPMHCKGKKALPTCDYGCECND |
| SEQ ID NO: 70 | GSIVCKVCKIICGMQGKKVNICKAPIKCKCKKG |
| SEQ ID NO: 71 | GSVSCEDCPDHCSTQKARAKCDNDKCVCEPK |
| SEQ ID NO: 72 | GSVSCEDCPEHCSTQKARAKCDNDKCVCESV |
| SEQ ID NO: 73 | GSVSCEDCPEHCSTQKAQAKCDNDKCVCEPI |
| SEQ ID NO: 74 | GSATCEDCPEHCATQNARAKCDNDKCVCEPK |
| SEQ ID NO: 75 | GSVSCEDCPEHCATKDQRAKCDNDKCVCEPK |
| SEQ ID NO: 76 | GSVGCEDCPEHCSQQNARAKCENDKCVCEPK |
| SEQ ID NO: 77 | GSVSCEDCPEHCATKDQRAKCDNDRCVCEPK |
| SEQ ID NO: 78 | GSVSCEDCPPHCATKDQRAKCENDKCVCEPK |
| SEQ ID NO: 79 | GSVSCEDCPEHCSTQKARAKCDNDKCVCEAI |
| SEQ ID NO: 80 | GSMCMPCFTTEQRMAIICDDCCGGFGRGRCYGPQCLC |
| SEQ ID NO: 81 | GSICIPCFTTDHQIARRCDDCCGGRGRGRCYGPQCIC |
| SEQ ID NO: 82 | GSMCMPCFTTDHRMAENCDICCGGDGRGRCYGPQCLC |
| SEQ ID NO: 83 | GSVGCEECPMHCRGRNANPTCDDGVCNCNV |
| SEQ ID NO: 84 | GSVGCEECPMHCRGRNANPTCDDGVCNC |
| SEQ ID NO: 85 | GSCGPCFTTDHQMEQKCAECCGGIGKCYGPQCLCNR |
| SEQ ID NO: 86 | GSRCGPCFTTDPQTQAKCSECCGRKGGVCKGPQCICGIQY |
| SEQ ID NO: 87 | GSMCMPCFTTDPNMAKKCRDCCGGNGKCFGPQCLCNR |
| SEQ ID NO: 88 | GSMCMPCFTTDHNMAKKCNDCCGGYGKCFGPQCLCR |
| SEQ ID NO: 89 | GSRCPPCFTTNPNMEADCRKCCGGRGYCASYQCICPGG |
| SEQ ID NO: 90 | GSMCMPCFTTDPNMANKCRDCCGGGKKCFGPQCLCNR |
| SEQ ID NO: 91 | GSMKFLYGVILIALFLTVMTATLSEARCGPCFTTDPQTQAKCSEC |
| | CGRKGGVCKGPQCICGIQY |
| SEQ ID NO: 92 | GSMCMPCFTTRPDMAQQCRACCKGRGKCFGPQCLCGYD |
| SEQ ID NO: 93 | GSMKFLYGIVFIALFLTVMTATLSDAMCMPCFTTDHNMAKKCR |
| | DCCGGNGKCFGPQCLCNRG |
| SEQ ID NO: 94 | GSMCMPCFTTDHNMAKKCRDCCGGNGKCFGPQCLCNR |
| SEQ ID NO: 95 | GSMKFLYGIVFITLFLTVMIATHTEAMCMPCFTTRPNMAQQCRD |
| | CCRGRGKCFGPQCLCGYD |
| SEQ ID NO: 96 | GSMKFLYGIVFIALFLTVMIATHTEAMCMPCFTTRPNMAQQCRD |
| | CCRGRGKCFGPQCLCGYD |
| SEQ ID NO: 97 | GSRCKPCFTTDPQMSKKCADCCGGKGKGKCYGPQCLC |
| SEQ ID NO: 98 | GSMKFLYGIVFITLFLTVMIATHTEAAMCMPCFTTNLNMEQECR |
| | DCCGGTGRCFGPQCLCGYD |
| SEQ ID NO: 99 | GSRCSPCFTTDQQMTKKCYDCCGGKGKGKCYGPQCICAPY |
| SEQ ID NO: 100 | GSCGPCFTTDPYTESKCATCCGGRGKCVGPQCLCNRI |
| SEQ ID NO: 101 | GSTEAMCMPCFTTDHNMAKKCRDCCGGNGKCFGYQCLCNRG |
| SEQ ID NO: 102 | GSMKFLYGIVFIALFLTVMFATQTDGCGPCFTTDANMARKCREC |
| | CGGIGKCFGPQCLCNRI |
| SEQ ID NO: 103 | GSMKFLYGIVFIALFLTVMFATQTDGCGPCFTTDANMARKCREC |
| | CGGNGKCFGPQCLCNRE |
| SEQ ID NO: 104 | GSMKFLYGTILIAFFLTVMIATHSEARCPPCFTTNPNMEADCRKC |
| | CGGRGYCASYQCICPGG |
| SEQ ID NO: 105 | GSTEAMCMPCFTTRPDMAQQCRDCCGGNGKCFGYQCLCNRG |
| SEQ ID NO: 106 | GSMKFLYGIVFIALFLTVMIATLTEAMCMPCFTTRPDMAQQCRD |
| | CCGGNGKCFGYQCLCNRG |
| SEQ ID NO: 107 | GSMKFLYGIVFIALFLTVMIATHTEAMCMPCFTTRPDMAQQCRD |
| | CCGGNGKCFGYQCLCNRG |
| SEQ ID NO: 108 | GSMKFLYGIILIALFLTVMIATHSEARCPNCFTTNPNAEADCKKC |
| | CGNRWGKCAGYQCVCPMK |
| SEQ ID NO: 109 | GSMKFLYGIVFIALFLTGMIATHTEAMCMPCFTTRPDMAQQCRD |
| | CCGGNGKCFGYQCLCNRGRIVIMYT |
| SEQ ID NO: 110 | GSMCMPCFTTRPGMAQQCRDCCGGNGKCFGYQCLCNR |
| SEQ ID NO: 111 | GSMCIPCFTTNPNMAAKCNACCGSRRGSCRGPQCICR |
| SEQ ID NO: 112 | GSMCIPCFTTNPNMAAKCNACCGSRRGSCRGPQCICN |
| SEQ ID NO: 113 | GSMCIPCFTTNPNMAAKCNACCGGNGSCRGPQCICN |
| SEQ ID NO: 114 | GSMCIPCFTTNPNMAAKCNACCGSRGRGSCRGPQCICN |
| SEQ ID NO: 115 | GSMCIPCFTTNPNMAAKCNACCGSRGRGKCRGPQCICN |
| SEQ ID NO: 116 | GSMCIPCFTTDHQMAAKCNACCGSRRGSCRGPQCICN |
| SEQ ID NO: 117 | GSMCIPCFTTNHQMAAKCNACCGSRRGSCRGPQCICN |
| SEQ ID NO: 118 | GSMCIPCFTTNPNMARKCNACCGSRGRGSCRGPQCICN |
| SEQ ID NO: 119 | GSMCIPCFTTNPNMAAKCNACCGGKGRGSCRGPQCICN |
| SEQ ID NO: 120 | GSMCIPCFTTNPNMAAKCNACCGSRRGSCFGPQCICN |
| SEQ ID NO: 121 | GSMCIPCFTTNPNMAAKCNACCGSRGRGKCFGPQCICN |
| SEQ ID NO: 122 | GSMCIPCFTTNPNMAAKCNACCGSRGRGSCFGPQCICN |
| SEQ ID NO: 123 | GSMCIPCFTTNPNMAAKCNACCGSRGRGSCYGPQCICN |
| SEQ ID NO: 124 | GSMCIPCFTTNPNMAAKCDACCGSRRGSCRGPQCICN |
| SEQ ID NO: 125 | GSMCIPCFTTNHQMAAKCDACCGSRRGSCRGPQCICN |
| SEQ ID NO: 126 | GSMCIPCFTTNHNMAAKCDACCGGRGRGSCRGPQCICN |
| SEQ ID NO: 127 | GSMCIPCFTTNPNMAAKCDACCGSRGRGSCRGPQCICN |
| SEQ ID NO: 128 | GSMCIPCFTTNPNMAAKCDACCGGKGRGSCRGPQCICN |
| SEQ ID NO: 129 | GSMCIPCFTTNHNMAAKCDACCGSRGRGSCRGPQCICN |
| SEQ ID NO: 130 | GSMCIPCFTTNPNMAAKCRDCCGGRGSCRGPQCICN |
| SEQ ID NO: 131 | GSMCMPCFTTNPNMAAKCDDCCGSRGRGSCRGPQCICN |
| SEQ ID NO: 132 | GSMCIPCFTTNPNMAARCNACCGSRRGSCRGPQCIC |
| SEQ ID NO: 133 | GSMCIPCFTTNPNMAAKCNACCGSRRGSCRGPQCICI |
| SEQ ID NO: 134 | GSGCLQFMWKCNPDNDKCCRPNLKCNTYHKWCEFVTGK |
| SEQ ID NO: 135 | GSDCLGFLWKCNPSNDKCCRPNLVCSRKDKWCKYQI |
| SEQ ID NO: 136 | GSDCLGFMRKCIPDNDKCCRPNLVCSRTHKWCKYVFGK |
| SEQ ID NO: 137 | GSECLEIFKACNPSNDQCCKSSKLVCSRKTRACKYQI |
| SEQ ID NO: 138 | GSECGGFWWKCGSGKPACCPKYVCSPKWGLCNFPMP |
| SEQ ID NO: 139 | GSGCLERWWKCNPNDDKCCRPKLKCSKLFKLCNRSRG |
| SEQ ID NO: 140 | GSGCLEEWWKCNPNDDKCCRPKLKCSKLFKLCNESEG |
| SEQ ID NO: 141 | GSGCLEIWWKCNPNDDKCCRPKLKCSKLFKLCNYSIG |
| SEQ ID NO: 142 | GSGCLEFWWKCNPNDDKCCRPKLKCSKLGKLCNFSFG |
| SEQ ID NO: 143 | GSGCLEFWWKCNPNDDKCCRPKLKCSPLGKLCNFSFG |
| SEQ ID NO: 144 | GSGCLEFWWKCNPNDDKCCRPKLKCSPNGKLCNFSFG |
| SEQ ID NO: 145 | GSGCLEFWWKCNPNDDKCCRPKLKCSRKTKLCNFSFG |
| SEQ ID NO: 146 | GSGCLEFWWKCNPNDDKCCRPKLKCGSNFKLCNFSFG |
| SEQ ID NO: 147 | GSGCLEFWWKCNPNDDKCCRPKLKCSTKHKLCNFSFG |
| SEQ ID NO: 148 | GSGCLEFWWKCNPNDDKCCRPKLKCSNDGKLCNFSFG |
| SEQ ID NO: 149 | GSGCLEFWWKCNPNDDKCCRPKLKCSKKTKLCNFSFG |
| SEQ ID NO: 150 | GSGCLEFWWKCNPNDDKCCRPKLKCHSNFKLCNFSFG |
| SEQ ID NO: 151 | GSGCLEFWWKCNPNDDKCCRPKLKCSKKFTACNFSFG |
| SEQ ID NO: 152 | GSGCLEIFKACNPNDDKCCRPKLKCSKLFKLCNFSFG |
| SEQ ID NO: 153 | GSGCLKFGWKCNPNDDKCCRPKLKCSKLFKLCNFSFG |
| SEQ ID NO: 154 | GSGCLEFWWKCNPNDDKCCKSSKLKCSKLFKLCNFSFG |
| SEQ ID NO: 155 | GSGCLEFWWKCNPNDDKCCRPKLKCNKLFKLCNISIG |
| SEQ ID NO: 156 | GSGCLEFWWKCNPNDDCCRKLKCSKLFKLCNFSFG |
| SEQ ID NO: 157 | GSGCLEFWWKCNPSNDQCCRPKLKCSKLFKLCNFSFG |
| SEQ ID NO: 158 | GSGCLEFWWKCNPNDDKCCRPSKLVCSKLFKLCNFSFG |
| SEQ ID NO: 159 | GSGCLEFLGECNPNDDKCCRPKLKCSKLFKLCNFSFG |
| SEQ ID NO: 160 | GSGCLWYLWKCNPNDDKCCRPKLKCSKLFKLCNFSFG |
| SEQ ID NO: 161 | GSGCLEFWWRCNPNDDRCCRPRLRCSRLFRLCNFSFG |
| SEQ ID NO: 162 | GSGCLEFWWRCNPNDDRCCRPRLRCSRLFRLC |
| SEQ ID NO: 163 | GSCRYFLGECKKTSECCEHLACHDKHKWCAWDWTIGK |
| SEQ ID NO: 164 | GSECRYWLGGCSAGQTCCKHLVCSRRHGWCVWDGTF |
| SEQ ID NO: 165 | GSECRWYLGECSQDGDCCKHLQCHSNYEWCIWDGTFSK |
| SEQ ID NO: 166 | GSECRWYLGGCSQDGDCCKHLQCHSNYEWCVWDGTFSK |
| SEQ ID NO: 167 | GSDCRKFLGACTQTSDCCKHLACHNKHKWCAWDWTI |
| SEQ ID NO: 168 | GSECRYLMGGCSKDGDCCEHLVCRTKWPYHCVWDWTFGK |
| SEQ ID NO: 169 | GSECRYRLGTCSKTGDCCSHLSCSPKHGWCVRDRTFRK |
| SEQ ID NO: 170 | GSECRYELGTCSKTGDCCSHLSCSPKHGWCVEDETFRK |
| SEQ ID NO: 171 | GSECRYILGTCSKTGDCCSHLSCSPKHGWCVYDITFRK |
| SEQ ID NO: 172 | GSECRYWLGTCSKTGDCCSHLSCSPKGGWCVWDWTFRK |
| SEQ ID NO: 173 | GSECRYWLGTCSKTGDCCSHLSCSPNHGWCVWDWTFRK |
| SEQ ID NO: 174 | GSECRYWLGTCSKTGDCCSHLSCSRKTGWCVWDWTFRK |
| SEQ ID NO: 175 | GSECRYWLGTCSKTGDCCSHLSCGSNHGWCVWDWTFRK |
| SEQ ID NO: 176 | GSECRYWLGTCSKTGDCCSHLSCSTKHGWCVWDWTFRK |
| SEQ ID NO: 177 | GSECRYWLGTCSKTGDCCSHLSCSSKHGWCVWDWTFRK |
| SEQ ID NO: 178 | GSECRYWLGTCSKTGDCCSHLSCSNDGGWCVWDWTFRK |
| SEQ ID NO: 179 | GSECRYWLGTCSKTGDCCSHLSCSPKTRACVWDWTFRK |
| SEQ ID NO: 180 | GSECRYWLGTCSKTGDCCSHLSCHSNHGWCVWDWTFRK |
| SEQ ID NO: 181 | GSECRYWLGTCSKTGDCCSHLSCSRKHRACVWDWTFRK |
| SEQ ID NO: 182 | GSECRYWLGTCSKTGDQCCKSSHLSCSPKHGWCVWDWTFRK |
| SEQ ID NO: 183 | GSECRYWLGTCSAGQDCCSHLSCSPKHGWCVWDWTFRK |
| SEQ ID NO: 184 | GSECRYWLGGCSATGDCCSHLSCSPKHGWCVWDWTFRK |
| SEQ ID NO: 185 | GSECRYWLGTCSKTGDCCKSSHLVCSPKHGWCVWDWTFRK |
| SEQ ID NO: 186 | GSECLEILGTCSKTGDCCSHLSCSPKHGWCVWDWTFRK |
| SEQ ID NO: 187 | GSECRYWFKACSKTGDCCSHLSCSPKHGWCVWDWTFRK |
| SEQ ID NO: 188 | GSECRYWLGTCSKTGDCCSHLSCSDGHGWCVWDWTFRK |
| SEQ ID NO: 189 | GSECRYWLGTCSKTGDCCSHLSCSKLHGWCVWDWTFRK |
| SEQ ID NO: 190 | GSECRYWLGTCSKTGDCCSHLQCHSKHGWCVWDWTFRK |
| SEQ ID NO: 191 | GSECRYWLGTCSRTGDCCSHLSCSPRHGWCVWDWTFRR |
| SEQ ID NO: 192 | GSECRYWLGTCSRTGDCCSHLSCSPRHGWC |
| SEQ ID NO: 193 | GSMCIPCFTTDHQMAAKCNACCGGKRGSCRGPQCICN |
| SEQ ID NO: 194 | GSMCIPCFTTDHQMAAKCNACCGGKGSCRGPQCICN |
| SEQ ID NO: 195 | GSMCIPCFTTDHQMAAKCNACCGGKRGSCRGPQCICI |
| SEQ ID NO: 196 | GSGCLEIWWKCNPNDDKCCRPKLKCSTKHFKLCNYSIG |
| SEQ ID NO: 197 | GSDCLGILWKCNPSNDKCCRPNLVCSTKHKWCKYQI |
| SEQ ID NO: 198 | GSGCLEIWWKCNPNDDKCCRPKLKCSTKHFKLCN |
| SEQ ID NO: 199 | GSDCLGILWKCNPSNDKCCRPNLVCSTKHKWCKYSIG |
| SEQ ID NO: 200 | GSGCLEIWWKCSKTGDCCSHLSCSPKTRACVWDWTFRK |
| SEQ ID NO: 201 | GSECRYWLGTCSKTGDCCSHLSCSPKTRACSKLFKLCNYSIG |
| SEQ ID NO: 214 | MCIPCFTTNPNMAAKCNACCGSRRGSCRGPQCIC |
| SEQ ID NO: 215 | GCLEFWWKCNPNDDKCCRPKLKCSKLFKLCNFSFG |
| SEQ ID NO: 216 | ECRYWLGTCSKTGDCCSHLSCSPKHGWCVWDWTFRK |
| SEQ ID NO: 217 | MCMPCFTTDHQMARRCDDCCGGRGRGRCYGPQCLCR |
| SEQ ID NO: 218 | MCMPCFTTHHRMAENCDICCGGDGRGKCYGPQCLCR |
| SEQ ID NO: 219 | MCMPCFTTDHRMAENCDICCGGDGRGKCYGPQCLCR |
| SEQ ID NO: 220 | MCMPCFTTHHQMAENCDICCGGDGRGKCYGPQCLCR |
| SEQ ID NO: 221 | MCMPCFTTHHRMARNCDICCGGDGRGKCYGPQCLCR |
| SEQ ID NO: 222 | MCMPCFTTHHRMAERCDICCGGDGRGKCYGPQCLCR |
| SEQ ID NO: 223 | MCMPCFTTHHRMAENCDDCCGGDGRGKCYGPQCLCR |
| SEQ ID NO: 224 | RCMPCFTTDHQMARRCDDCCGGRGRGKCYGPQCLCR |
| SEQ ID NO: 225 | ICIPCFTTDHQIARRCDDCCGGRGRGKCYGPQCLCR |
| SEQ ID NO: 226 | MCLPCFTTDHQLARRCDDCCGGRGRGKCYGPQCLCR |
| SEQ ID NO: 227 | MCMPCFTTEHQMARRCEECCGGRGRGKCYGPQCLCR |
| SEQ ID NO: 228 | MCIPCFTTDHQMARRCEECCGGRGRGKCYGPQCLCR |
| SEQ ID NO: 229 | ICIPCFTTDHQMARRCDDCCGGRGDGKCYGPQCLCR |
| SEQ ID NO: 230 | ICIPCFTTDHQIARRCDDCCGGRGRGKCYGPQCICR |
| SEQ ID NO: 231 | RCMPCFTTDHFMARFCDFCCGGRGRGKCYGPQCLCR |
| SEQ ID NO: 232 | RCMPCFTTDHYMARYCDYCCGGRGRGKCYGPQCLCR |
| SEQ ID NO: 233 | RCMPCFTTDHRMARRCDRCCGGRGRGKCYGPQCLCR |
| SEQ ID NO: 234 | RCMPCFTTDHEMARECDECCGGRGRGKCYGPQCLCR |
| SEQ ID NO: 235 | RCMPCFTTDHHMARHCDHCCGGRGRGKCYGPQCLCR |
| SEQ ID NO: 236 | LCLPCFTTHHRLADQCDICCGGDGRGKCYGPQCLCR |
| SEQ ID NO: 237 | ICIPCFTTEHQIARRCEECCGGRGRGKCYGPQCLCR |
| SEQ ID NO: 238 | MCMPCFTTDTQMQERCDRCCGGGGRGKCWGPQCLCI |
| SEQ ID NO: 239 | MCMPCFTTIYRMAHECDECCGGRGRGKCYGPQCLCR |
| SEQ ID NO: 240 | MCMPCFTTGYRMAEYCDICCGGRGRGKCYGPQCLCR |
| SEQ ID NO: 241 | MCMPCFTTHRRMANTCDACCGGRSRGKCYGPQCLCR |
| SEQ ID NO: 242 | HCMPCFTTDHQMIRRCDDCCGGGSYGKCDGPQCLCF |
| SEQ ID NO: 243 | DCMPCFTTDHRMADHCDICCGGDDRGKCYGPQCLCR |
| SEQ ID NO: 244 | MCMPCFTTDHEMERRCDDCCGIGGGGKCHGPQCLCG |
| SEQ ID NO: 245 | MCMPCFTTEQRMAIICDDCCGGFGRGKCYGPQCLCR |
| SEQ ID NO: 246 | MCMPCFTTSEQMFRRCDDCCGGWGDGKCNGPHCLCR |
| SEQ ID NO: 247 | GVPINVKCRGSRDCLDPCKKAGMRFGKCINSKCHCTP |
| SEQ ID NO: 248 | MCMPCFTTEQRMAIICDDCCGGFGRGRCYGPQCLCR |
| SEQ ID NO: 249 | ICIPCFTTDHQIARRCDDCCGGRGRGRCYGPQCICR |
| SEQ ID NO: 250 | MCMPCFTTDTQMQERCDRCCGGGGRGRCWGPQCLCI |
| SEQ ID NO: 251 | MCMPCFTTDTQMQERCDRCCGGGGRGRCWGPQCLC |
| SEQ ID NO: 252 | MCMPCFTTDHRMAENCDICCGGDGRGRCYGPQCLCR |
| SEQ ID NO: 253 | MCMPCFTTEQRMAIICDDCCGGFGRGKCYGPQCLCI |
| SEQ ID NO: 254 | MCMPCFTTEQRMAIICDDCCGGFGRGRCYGPQCLCI |
| SEQ ID NO: 255 | ICIPCFTTDHQIARRCDDCCGGRGRGKCYGPQCICI |
| SEQ ID NO: 256 | ICIPCFTTDHQIARRCDDCCGGRGRGRCYGPQCICI |
| SEQ ID NO: 257 | MCMPCFTTDTQMQEKCDRCCGGGGRGRCWGPQCLCI |
| SEQ ID NO: 258 | MCMPCFTTEQRMAIKCDDCCGGFGRGRCYGPQCLCR |
| SEQ ID NO: 259 | ICIPCFTTDHQIARKCDDCCGGRGRGRCYGPQCICR |
| SEQ ID NO: 260 | MCMPCFTTDHRMAEKCDICCGGDGRGRCYGPQCLCR |
| SEQ ID NO: 261 | MCMPCFTTDTQMQERCDRCCGGKGRGRCWGPQCLCI |
| SEQ ID NO: 262 | MCMPCFTTEQRMAIICDDCCGGKGRGRCYGPQCLCR |
| SEQ ID NO: 263 | ICIPCFTTDHQIARRCDDCCGGKGRGRCYGPQCICR |
| SEQ ID NO: 264 | MCMPCFTTDHRMAENCDICCGGKGRGRCYGPQCLCR |
| SEQ ID NO: 265 | MCMPCFTTDHRMAENCDICCGGDGRGKCYGPQCLCI |
| SEQ ID NO: 266 | MCMPCFTTDHRMAENCDICCGGDGRGRCYGPQCLCI |
| SEQ ID NO: 267 | MCMPCFTTHHRMAENCDICCGGDGRGRCYGPQCLCR |
| SEQ ID NO: 268 | VGCEECPMHCKGKNANPTCDDGVCNCNV |
| SEQ ID NO: 269 | VGCEECPMHCKGKNAKPTCDDGVCNCNV |
| SEQ ID NO: 270 | VGCEECPMHCKGKNAKPTCDNGVCNCNV |
| SEQ ID NO: 271 | VGCEECPMHCKGKHAVPTCDDGVCNCNV |
| SEQ ID NO: 272 | VGCEECPAHCKGKNAKPTCDDGVCNCNV |
| SEQ ID NO: 273 | VGCEECPAHCKGKNAIPTCDDGVCNCNV |
| SEQ ID NO: 274 | VGCEECPMHCKGKMAKPTCDDGVCNCNV |
| SEQ ID NO: 275 | VGCEECPMHCKGKNAVPTCDNGVCNCNA |
| SEQ ID NO: 276 | VGCEECPMHCKGKMAKPTCYDGVCNCNV |
| SEQ ID NO: 277 | VGCEECPMYCKGKNAVPTCDGGVCNCNA |
| SEQ ID NO: 278 | VGCEECPKYCKGKNAVPTCDGGVCNCNA |
| SEQ ID NO: 279 | VGCEECPVYCKGKKALPTCDGGVCNCNA |
| SEQ ID NO: 280 | VGCEEDPMHCKGKQAKPTCCNGVCNCNV |
| SEQ ID NO: 281 | VGCAECPMHCKGKMAKPTCENEVCKCNIGKKD |
| SEQ ID NO: 282 | VGCEECPMHCKGKKALPTCDYGCECND |
| SEQ ID NO: 283 | IVCKVCKIICGMQGKKVNICKAPIKCKCKKG |
| SEQ ID NO: 284 | VSCEDCPDHCSTQKARAKCDNDKCVCEPK |
| SEQ ID NO: 285 | VSCEDCPEHCSTQKARAKCDNDKCVCESV |
| SEQ ID NO: 286 | VSCEDCPEHCSTQKAQAKCDNDKCVCEPI |
| SEQ ID NO: 287 | ATCEDCPEHCATQNARAKCDNDKCVCEPK |
| SEQ ID NO: 288 | VSCEDCPEHCATKDQRAKCDNDKCVCEPK |
| SEQ ID NO: 289 | VGCEDCPEHCSQQNARAKCENDKCVCEPK |
| SEQ ID NO: 290 | VSCEDCPEHCATKDQRAKCDNDRCVCEPK |
| SEQ ID NO: 291 | VSCEDCPPHCATKDQRAKCENDKCVCEPK |
| SEQ ID NO: 292 | VSCEDCPEHCSTQKARAKCDNDKCVCEAI |
| SEQ ID NO: 293 | MCMPCFTTEQRMAIICDDCCGGFGRGRCYGPQCLC |
| SEQ ID NO: 294 | ICIPCFTTDHQIARRCDDCCGGRGRGRCYGPQCIC |
| SEQ ID NO: 295 | MCMPCFTTDHRMAENCDICCGGDGRGRCYGPQCLC |
| SEQ ID NO: 296 | VGCEECPMHCRGRNANPTCDDGVCNCNV |
| SEQ ID NO: 297 | VGCEECPMHCRGRNANPTCDDGVCNC |
| SEQ ID NO: 298 | CGPCFTTDHQMEQKCAECCGGIGKCYGPQCLCNR |
| SEQ ID NO: 299 | RCGPCFTTDPQTQAKCSECCGRKGGVCKGPQCICGIQY |
| SEQ ID NO: 300 | MCMPCFTTDPNMAKKCRDCCGGNGKCFGPQCLCNR |
| SEQ ID NO: 301 | MCMPCFTTDHNMAKKCNDCCGGYGKCFGPQCLCR |
| SEQ ID NO: 302 | RCPPCFTTNPNMEADCRKCCGGRGYCASYQCICPGG |
| SEQ ID NO: 303 | MCMPCFTTDPNMANKCRDCCGGGKKCFGPQCLCNR |
| SEQ ID NO: 304 | MKFLYGVILIALFLTVMTATLSEARCGPCFTTDPQTQAKCSECCG |
| | RKGGVCKGPQCICGIQY |
| SEQ ID NO: 305 | MCMPCFTTRPDMAQQCRACCKGRGKCFGPQCLCGYD |
| SEQ ID NO: 306 | MKFLYGIVFIALFLTVMTATLSDAMCMPCFTTDHNMAKKCRDC |
| | CGGNGKCFGPQCLCNRG |
| SEQ ID NO: 307 | MCMPCFTTDHNMAKKCRDCCGGNGKCFGPQCLCNR |
| SEQ ID NO: 308 | MKFLYGIVFITLFLTVMIATHTEAMCMPCFTTRPNMAQQCRDCC |
| | RGRGKCFGPQCLCGYD |
| SEQ ID NO: 309 | MKFLYGIVFIALFLTVMIATHTEAMCMPCFTTRPNMAQQCRDCC |
| | RGRGKCFGPQCLCGYD |
| SEQ ID NO: 310 | RCKPCFTTDPQMSKKCADCCGGKGKGKCYGPQCLC |
| SEQ ID NO: 311 | MKFLYGIVFITLFLTVMIATHTEAAMCMPCFTTNLNMEQECRDC |
| | CGGTGRCFGPQCLCGYD |
| SEQ ID NO: 312 | RCSPCFTTDQQMTKKCYDCCGGKGKGKCYGPQCICAPY |
| SEQ ID NO: 313 | CGPCFTTDPYTESKCATCCGGRGKCVGPQCLCNRI |
| SEQ ID NO: 314 | TEAMCMPCFTTDHNMAKKCRDCCGGNGKCFGYQCLCNRG |
| SEQ ID NO: 315 | MKFLYGIVFIALFLTVMFATQTDGCGPCFTTDANMARKCRECCG |
| | GIGKCFGPQCLCNRI |
| SEQ ID NO: 316 | MKFLYGIVFIALFLTVMFATQTDGCGPCFTTDANMARKCRECCG |
| | GNGKCFGPQCLCNRE |
| SEQ ID NO: 317 | MKFLYGTILIAFFLTVMIATHSEARCPPCFTTNPNMEADCRKCCG |
| | GRGYCASYQCICPGG |
| SEQ ID NO: 318 | TEAMCMPCFTTRPDMAQQCRDCCGGNGKCFGYQCLCNRG |
| SEQ ID NO: 319 | MKFLYGIVFIALFLTVMIATLTEAMCMPCFTTRPDMAQQCRDCC |
| | GGNGKCFGYQCLCNRG |
| SEQ ID NO: 320 | MKFLYGIVFIALFLTVMIATHTEAMCMPCFTTRPDMAQQCRDCC |
| | GGNGKCFGYQCLCNRG |
| SEQ ID NO: 321 | MKFLYGIILIALFLTVMIATHSEARCPNCFTTNPNAEADCKKCCG |
| | NRWGKCAGYQCVCPMK |
| SEQ ID NO: 322 | MKFLYGIVFIALFLTGMIATHTEAMCMPCFTTRPDMAQQCRDCC |
| | GGNGKCFGYQCLCNRGRIVIMYT |
| SEQ ID NO: 323 | MCMPCFTTRPGMAQQCRDCCGGNGKCFGYQCLCNR |
| SEQ ID NO: 324 | MCIPCFTTNPNMAAKCNACCGSRRGSCRGPQCICR |
| SEQ ID NO: 325 | MCIPCFTTNPNMAAKCNACCGSRRGSCRGPQCICN |
| SEQ ID NO: 326 | MCIPCFTTNPNMAAKCNACCGGNGSCRGPQCICN |
| SEQ ID NO: 327 | MCIPCFTTNPNMAAKCNACCGSRGRGSCRGPQCICN |
| SEQ ID NO: 328 | MCIPCFTTNPNMAAKCNACCGSRGRGKCRGPQCICN |
| SEQ ID NO: 329 | MCIPCFTTDHQMAAKCNACCGSRRGSCRGPQCICN |
| SEQ ID NO: 330 | MCIPCFTTNHQMAAKCNACCGSRRGSCRGPQCICN |
| SEQ ID NO: 331 | MCIPCFTTNPNMARKCNACCGSRGRGSCRGPQCICN |
| SEQ ID NO: 332 | MCIPCFTTNPNMAAKCNACCGGKGRGSCRGPQCICN |
| SEQ ID NO: 333 | MCIPCFTTNPNMAAKCNACCGSRRGSCFGPQCICN |
| SEQ ID NO: 334 | MCIPCFTTNPNMAAKCNACCGSRGRGKCFGPQCICN |
| SEQ ID NO: 335 | MCIPCFTTNPNMAAKCNACCGSRGRGSCFGPQCICN |
| SEQ ID NO: 336 | MCIPCFTTNPNMAAKCNACCGSRGRGSCYGPQCICN |
| SEQ ID NO: 337 | MCIPCFTTNPNMAAKCDACCGSRRGSCRGPQCICN |
| SEQ ID NO: 338 | MCIPCFTTNHQMAAKCDACCGSRRGSCRGPQCICN |
| SEQ ID NO: 339 | MCIPCFTTNHNMAAKCDACCGGRGRGSCRGPQCICN |
| SEQ ID NO: 340 | MCIPCFTTNPNMAAKCDACCGSRGRGSCRGPQCICN |
| SEQ ID NO: 341 | MCIPCFTTNPNMAAKCDACCGGKGRGSCRGPQCICN |
| SEQ ID NO: 342 | MCIPCFTTNHNMAAKCDACCGSRGRGSCRGPQCICN |
| SEQ ID NO: 343 | MCIPCFTTNPNMAAKCRDCCGGRGSCRGPQCICN |
| SEQ ID NO: 344 | MCMPCFTTNPNMAAKCDDCCGSRGRGSCRGPQCICN |
| SEQ ID NO: 345 | MCIPCFTTNPNMAARCNACCGSRRGSCRGPQCIC |
| SEQ ID NO: 346 | MCIPCFTTNPNMAAKCNACCGSRRGSCRGPQCICI |
| SEQ ID NO: 347 | GCLQFMWKCNPDNDKCCRPNLKCNTYHKWCEFVTGK |
| SEQ ID NO: 348 | DCLGFLWKCNPSNDKCCRPNLVCSRKDKWCKYQI |
| SEQ ID NO: 349 | DCLGFMRKCIPDNDKCCRPNLVCSRTHKWCKYVFGK |
| SEQ ID NO: 350 | ECLEIFKACNPSNDQCCKSSKLVCSRKTRACKYQI |
| SEQ ID NO: 351 | ECGGFWWKCGSGKPACCPKYVCSPKWGLCNFPMP |
| SEQ ID NO: 352 | GCLERWWKCNPNDDKCCRPKLKCSKLFKLCNRSRG |
| SEQ ID NO: 353 | GCLEEWWKCNPNDDKCCRPKLKCSKLFKLCNESEG |
| SEQ ID NO: 354 | GCLEIWWKCNPNDDKCCRPKLKCSKLFKLCNYSIG |
| SEQ ID NO: 355 | GCLEFWWKCNPNDDKCCRPKLKCSKLGKLCNFSFG |
| SEQ ID NO: 356 | GCLEFWWKCNPNDDKCCRPKLKCSPLGKLCNFSFG |
| SEQ ID NO: 357 | GCLEFWWKCNPNDDKCCRPKLKCSPNGKLCNFSFG |
| SEQ ID NO: 358 | GCLEFWWKCNPNDDKCCRPKLKCSRKTKLCNFSFG |
| SEQ ID NO: 359 | GCLEFWWKCNPNDDKCCRPKLKCGSNFKLCNFSFG |
| SEQ ID NO: 360 | GCLEFWWKCNPNDDKCCRPKLKCSTKHKLCNFSFG |
| SEQ ID NO: 361 | GCLEFWWKCNPNDDKCCRPKLKCSNDGKLCNFSFG |
| SEQ ID NO: 362 | GCLEFWWKCNPNDDKCCRPKLKCSKKTKLCNFSFG |
| SEQ ID NO: 363 | GCLEFWWKCNPNDDKCCRPKLKCHSNFKLCNFSFG |
| SEQ ID NO: 364 | GCLEFWWKCNPNDDKCCRPKLKCSKKFTACNFSFG |
| SEQ ID NO: 365 | GCLEIFKACNPNDDKCCRPKLKCSKLFKLCNFSFG |
| SEQ ID NO: 366 | GCLKFGWKCNPNDDKCCRPKLKCSKLFKLCNFSFG |
| SEQ ID NO: 367 | GCLEFWWKCNPNDDKCCKSSKLKCSKLFKLCNFSFG |
| SEQ ID NO: 368 | GCLEFWWKCNPNDDKCCRPKLKCNKLFKLCNISIG |
| SEQ ID NO: 369 | GCLEFWWKCNPNDDCCRKLKCSKLFKLCNFSFG |
| SEQ ID NO: 370 | GCLEFWWKCNPSNDQCCRPKLKCSKLFKLCNFSFG |
| SEQ ID NO: 371 | GCLEFWWKCNPNDDKCCRPSKLVCSKLFKLCNFSFG |
| SEQ ID NO: 372 | GCLEFLGECNPNDDKCCRPKLKCSKLFKLCNFSFG |
| SEQ ID NO: 373 | GCLWYLWKCNPNDDKCCRPKLKCSKLFKLCNFSFG |
| SEQ ID NO: 374 | GCLEFWWRCNPNDDRCCRPRLRCSRLFRLCNFSFG |
| SEQ ID NO: 375 | GCLEFWWRCNPNDDRCCRPRLRCSRLFRLC |
| SEQ ID NO: 376 | CRYFLGECKKTSECCEHLACHDKHKWCAWDWTIGK |
| SEQ ID NO: 377 | ECRYWLGGCSAGQTCCKHLVCSRRHGWCVWDGTF |
| SEQ ID NO: 378 | ECRWYLGECSQDGDCCKHLQCHSNYEWCIWDGTFSK |
| SEQ ID NO: 379 | ECRWYLGGCSQDGDCCKHLQCHSNYEWCVWDGTFSK |
| SEQ ID NO: 380 | DCRKFLGACTQTSDCCKHLACHNKHKWCAWDWTI |
| SEQ ID NO: 381 | ECRYLMGGCSKDGDCCEHLVCRTKWPYHCVWDWTFGK |
| SEQ ID NO: 382 | ECRYRLGTCSKTGDCCSHLSCSPKHGWCVRDRTFRK |
| SEQ ID NO: 383 | ECRYELGTCSKTGDCCSHLSCSPKHGWCVEDETFRK |
| SEQ ID NO: 384 | ECRYILGTCSKTGDCCSHLSCSPKHGWCVYDITFRK |
| SEQ ID NO: 385 | ECRYWLGTCSKTGDCCSHLSCSPKGGWCVWDWTFRK |
| SEQ ID NO: 386 | ECRYWLGTCSKTGDCCSHLSCSPNHGWCVWDWTFRK |
| SEQ ID NO: 387 | ECRYWLGTCSKTGDCCSHLSCSRKTGWCVWDWTFRK |
| SEQ ID NO: 388 | ECRYWLGTCSKTGDCCSHLSCGSNHGWCVWDWTFRK |
| SEQ ID NO: 389 | ECRYWLGTCSKTGDCCSHLSCSTKHGWCVWDWTFRK |
| SEQ ID NO: 390 | ECRYWLGTCSKTGDCCSHLSCSSKHGWCVWDWTFRK |
| SEQ ID NO: 391 | ECRYWLGTCSKTGDCCSHLSCSNDGGWCVWDWTFRK |
| SEQ ID NO: 392 | ECRYWLGTCSKTGDCCSHLSCSPKTRACVWDWTFRK |
| SEQ ID NO: 393 | ECRYWLGTCSKTGDCCSHLSCHSNHGWCVWDWTFRK |
| SEQ ID NO: 394 | ECRYWLGTCSKTGDCCSHLSCSRKHRACVWDWTFRK |
| SEQ ID NO: 395 | ECRYWLGTCSKTGDQCCKSSHLSCSPKHGWCVWDWTFRK |
| SEQ ID NO: 396 | ECRYWLGTCSAGQDCCSHLSCSPKHGWCVWDWTFRK |
| SEQ ID NO: 397 | ECRYWLGGCSATGDCCSHLSCSPKHGWCVWDWTFRK |
| SEQ ID NO: 398 | ECRYWLGTCSKTGDCCKSSHLVCSPKHGWCVWDWTFRK |
| SEQ ID NO: 399 | ECLEILGTCSKTGDCCSHLSCSPKHGWCVWDWTFRK |
| SEQ ID NO: 400 | ECRYWFKACSKTGDCCSHLSCSPKHGWCVWDWTFRK |
| SEQ ID NO: 401 | ECRYWLGTCSKTGDCCSHLSCSDGHGWCVWDWTFRK |
| SEQ ID NO: 402 | ECRYWLGTCSKTGDCCSHLSCSKLHGWCVWDWTFRK |
| SEQ ID NO: 403 | ECRYWLGTCSKTGDCCSHLQCHSKHGWCVWDWTFRK |
| SEQ ID NO: 404 | ECRYWLGTCSRTGDCCSHLSCSPRHGWCVWDWTFRR |
| SEQ ID NO: 405 | ECRYWLGTCSRTGDCCSHLSCSPRHGWC |
| SEQ ID NO: 406 | MCIPCFTTDHQMAAKCNACCGGKRGSCRGPQCICN |
| SEQ ID NO: 407 | MCIPCFTTDHQMAAKCNACCGGKGSCRGPQCICN |
| SEQ ID NO: 408 | MCIPCFTTDHQMAAKCNACCGGKRGSCRGPQCICI |
| SEQ ID NO: 409 | GCLEIWWKCNPNDDKCCRPKLKCSTKHFKLCNYSIG |
| SEQ ID NO: 410 | DCLGILWKCNPSNDKCCRPNLVCSTKHKWCKYQI |
| SEQ ID NO: 411 | GCLEIWWKCNPNDDKCCRPKLKCSTKHFKLCN |
| SEQ ID NO: 412 | DCLGILWKCNPSNDKCCRPNLVCSTKHKWCKYSIG |
| SEQ ID NO: 413 | GCLEIWWKCSKTGDCCSHLSCSPKTRACVWDWTFRK |
| SEQ ID NO: 414 | ECRYWLGTCSKTGDCCSHLSCSPKTRACSKLFKLCNYSIG |

In some instances, a peptide of the disclosure can comprise the sequence GSX¹CX²PCFTTX³X⁴X⁵X⁶X⁷X⁸X⁹CX¹⁰X¹¹CCGX¹²X¹³X¹⁴X¹⁵GX¹⁶CX¹⁷GPX¹⁸CX¹⁹CX²⁰ (SEQ ID NO: 202) or a fragment thereof, where X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, and X²⁰ are each individually any amino acid or amino acid analogue.

In some instances, a peptide of the disclosure can comprise the sequence X¹CX²PCFTTX³X⁴X⁵X⁶X⁷X⁸X⁹C¹⁰X¹¹CCGX¹²X¹³X¹⁴X¹⁵GX¹⁶CX¹⁷GPX¹⁸CX¹⁹CX²⁰ (SEQ ID NO: 415) or a fragment thereof, where X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, and X²⁰ are each individually any amino acid or amino acid analogue.

In some instances, a peptide of the disclosure can comprise the sequence GSX¹CX²PCFTTX³X⁴X⁵X⁶X⁷X⁸X⁹CX¹⁰X¹¹CCGX¹²X¹³X¹⁴X¹⁵GX¹⁶CX¹⁷GPX¹⁸CX¹⁹CX²⁰ (SEQ ID NO: 203) or a fragment thereof, where: X¹ is selected from M, R, I, D, H, or L; X² is selected from M, I or L; X³ is selected from D, H, E, S, G, or I; X⁴ is selected from H, E, Q, R, Y, or T; X⁵ is selected from Q, R, H, E, Y, or F; X⁶ is selected from M, I, or L; X⁷ is selected from A, F, E, I, or Q; X⁸ is selected from R, E, I, D, N, or H; X⁹ is selected from R, N, H, E, Y, F, I, T, or Q; X¹⁰ is selected from D or E; X¹¹ is selected from D, I H, E, R, Y, F, or A; X¹² is selected from G or I; X¹³ is selected from R, D, W, F, or G; X¹⁴ is selected from G, D, or S; X¹⁵ is selected from R, D, G, or Y; X¹⁶ is selected from K or R; X¹⁷ is selected from Y, N, H, D, or W; X¹⁸ is selected from Q or H; X¹⁹ is selected from L or I; and X²⁰ is selected from R, G, F, or I.

In some instances, the peptides of the disclosure can comprise the sequence X¹CX²PCFTTX³X⁴X⁵X⁶X⁷X⁸X⁹C X¹⁰X¹¹CCGX¹²X¹³X¹⁴X¹⁵GX¹⁶CX¹⁷GPX¹⁸CX¹⁹CX²⁰ (SEQ ID NO: 416) or a fragment thereof, where: X¹ is selected from M, R, I, D, H, or L; X² is selected from M, I or L; X³ is selected from D, H, E, S, G, or I; X⁴ is selected from H, E, Q, R, Y, or T; X⁵ is selected from Q, R, H, E, Y, or F; X⁶ is selected from M, I, or L; X⁷ is selected from A, F, E, I, or Q; X⁸ is selected from R, E, I, D, N, or H; X⁹ is selected from R, N, H, E, Y, F, I, T, or Q; X¹⁰ is selected from D or E; X¹¹ is selected from D, I H, E, R, Y, F, or A; X¹² is selected from G or I; X¹³ is selected from R, D, W, F, or G; X¹⁴ is selected from G, D, or S; X¹⁵ is selected from R, D, G, or Y; X¹⁶ is selected from K or R; X¹⁷ is selected from Y, N, H, D, or W; X¹⁸ is selected from Q or H; X¹⁹ is selected from L or I; and X²⁰ is selected from R, G, F, or I.

In some instances, a peptide of the disclosure can comprise the sequence GSVGCEECPX¹HCX²GX³X⁴AX⁵PTCDX⁶GVCNCNV (SEQ ID NO: 204) or a fragment thereof, wherein X¹, X², X³, X⁴, X⁵, and X⁶ are each individually any amino acid or amino acid analogue.

In some instances, a peptide of the disclosure can comprise the sequence VGCEECPX¹HCX²GX³X⁴AX⁵PTCDX⁶GVCNCNV (SEQ ID NO: 417) or a fragment thereof, wherein X¹, X², X³, X⁴, X⁵, and X⁶ are each individually any amino acid or amino acid analogue.

In other cases, a peptide can comprise the sequence GSVGCEECPX¹HCX²GX³X⁴AX⁵PTCDX⁶GVCNCNV (SEQ ID NO: 205) or a fragment thereof, where X¹ is selected from M, A, V, I, or L, wherein X² is selected from K or R, wherein X³ is selected from K or R, wherein X⁴ is selected from N, H, M, K, or Q, wherein X⁵ is selected from N, K, V, I, L, R or Q, and wherein X⁶ is selected from D, N, G, Y, or E.

In other cases, a peptide can comprise the sequence VGCEECPX¹HCX²GX³X⁴AX⁵PTCDX⁶GVCNCNV (SEQ ID NO: 418) or a fragment thereof, where X¹ is selected from M, A, V, I, or L, wherein X² is selected from K or R, wherein X³ is selected from K or R, wherein X⁴ is selected from N, H, M, K, or Q, wherein X⁵ is selected from N, K, V, I, L, R or Q, and wherein X⁶ is selected from D, N, G, Y, or E.

In some instances, a peptide of the disclosure can comprise the sequence GSVGCX¹EX²PX³X⁴CKGKX⁵AX⁶PTCX⁷X⁸X⁹X¹⁰CX¹¹CNX¹² (SEQ ID NO: 206) or a fragment thereof, where X¹, X², X³, X⁴, X⁵, X⁶, X⁷, X⁸, X⁹, X¹⁰, X¹¹, and X¹² are each individually any amino acid or amino acid analogue.

In some instances, a peptide of the disclosure can comprise the sequence VGCX¹EX²PX³X⁴CKGKX⁵AX⁶PTCX⁷X⁸X⁹X¹⁰CX¹¹CNX¹² (SEQ ID NO: 419) or a fragment thereof, where X¹, X², X³, X⁴, X⁵, X⁶, X⁷, X⁸, X⁹, X¹⁰, X¹¹, and X¹² are each individually any amino acid or amino acid analogue.

In some instances, a peptide of the disclosure can comprise the sequence GSVGCX¹EX²PX³X⁴CKGKX⁵AX⁶PTCX⁷X⁸X⁹X¹⁰CX¹¹CNX¹² (SEQ ID NO: 207) or a fragment thereof, where: X¹ is selected from A or E; X² is selected from C or D; X³ is selected from M, A, K, or V; X⁴ is selected from H or Y; X⁵ is selected from N, H, M, K, or Q; X⁶ is selected from N, K V, I, or L; X is selected from D, Y, C, or E; X is selected from D, N, G, or Y; X⁹ is selected from G or E; X¹⁰ is selected from V or is absent; X¹¹ is selected from N, K, or E; and X¹² is selected from V, A, I, or D.

In some instances, a peptide of the disclosure can comprise the sequence VGCX¹EX²PX³X⁴CKGKX⁵AX⁶PTCX⁷X⁸X⁹X¹⁰CX¹¹CNX¹² (SEQ ID NO: 420) or a fragment thereof, where: X¹ is selected from A or E; X² is selected from C or D; X³ is selected from M, A, K, or V; X⁴ is selected from H or Y; X⁵ is selected from N, H, M, K, or Q; X⁶ is selected from N, K V, I, or L; X⁷ is selected from D, Y, C, or E; X⁸ is selected from D, N, G, or Y; X⁹ is selected from G or E; X¹⁰ is selected from V or is absent; X¹¹ is selected from N, K, or E; and X¹² is selected from V, A, I, or D.

In some instances, a peptide of the disclosure can comprise the sequence GSX¹X²CEDCPX³HCX⁴X⁵X⁶X⁷X⁸X⁹AKCX¹⁰NDX¹¹CVCEX¹²X¹³ (SEQ ID NO: 208) or a fragment thereof, where X¹, X², X³, X⁴, X⁵, X⁶, X⁷, X⁸, X⁹, X¹⁰, X¹¹, X¹², and X¹³ are each individually any amino acid or amino acid analogue.

In some instances, a peptide of the disclosure can comprise the sequence X¹X²CEDCPX³HCX⁴X⁵X⁶X⁷X⁸X⁹AKCX¹⁰NDX¹¹CVCEX¹²X¹³ (SEQ ID NO: 421) or a fragment thereof, where X¹, X², X³, X⁴, X⁵, X⁶, X⁷, X⁸, X⁹, X¹⁰, X¹¹, X¹², and X¹³ are each individually any amino acid or amino acid analogue.

In some instances, a peptide of the disclosure can comprise the sequence GSX¹X²CEDCPX³HCX⁴X⁵X⁶X⁷X⁸X⁹AKCX¹⁰NDX¹¹CVCEX¹²X¹³ (SEQ ID NO: 209) or a fragment thereof, where: X¹ is selected from V or A; X² is selected from S, T, or G; X³ is selected from D or E; X⁴ is selected from S or A; X⁵ is selected from T or Q; X⁶ is selected from Q or K; X⁷ is selected from K, N, or D; X⁸ is selected from A or Q; X⁹ is selected from R or Q; X¹⁰ is selected from D or E; X¹¹ is selected from K or R; X¹² is selected from P, S, or A; and X¹³ is selected from K, V, or I.

In some instances, a peptide of the disclosure can comprise the sequence X¹X²CEDCPX³HCX⁴X⁵X⁶X⁷X⁸X⁹AKCX¹⁰NDX¹¹CVCEX¹²X¹³ (SEQ ID NO: 422) or a fragment thereof, where: X¹ is selected from V or A; X² is selected from S, T, or G; X³ is selected from D or E; X⁴ is selected from S or A; X⁵ is selected from T or Q; X⁶ is selected from Q or K; X⁷ is selected from K, N, or D; X⁸ is selected from A or Q; X⁹ is selected from R or Q; X¹⁰ is selected from D or E; X¹¹ is selected from K or R; X¹² is selected from P, S, or A; and X¹³ is selected from K, V, or I.

In some instances, a peptide of the disclosure can comprise the sequence GSX¹CX²PCFTTDHQX²ARRCDDCCGGRGRGX³CYGPQCX²CX⁴ (SEQ ID NO: 210) or a fragment thereof, where: X¹ is any amino acid or amino acid analogue except P or C; X² is independently selected from A, L, V, I, or M; X³ is selected from K or R; and X⁴ is any amino acid or amino acid analogue except C.

In some instances, a peptide of the disclosure can comprise the sequence X¹CX²PCFTTDHQX²ARRCDDCCGGRGRGX³CYGPQCX²CX⁴ (SEQ ID NO: 423) or a fragment thereof, where: X¹ is any amino acid or amino acid analogue except P or C; X² is independently selected from A, L, V, I, or M; X³ is selected from K or R; and X⁴ is any amino acid or amino acid analogue except C.

In some instances, a peptide of the disclosure can comprise the sequence GSMCMPCFTTDHRMAENCDICCGGDGRGXCYGPQCLCR (SEQ ID NO: 211) or a fragment thereof, where X is R or K.

In some instances, a peptide of the disclosure can comprise the sequence MCMPCFTTDHRMAENCDICCGGDGRGXCYGPQCLCR (SEQ ID NO: 424) or a fragment thereof, where X is R or K.

In some instances, a peptide of the disclosure can comprise the sequence GSXCMPCFTTXXXMXXXCDXCCGXXXXGXCXGPXCLCX (SEQ ID NO: 212) or a fragment thereof, where X can independently be any amino acid or amino acid analogue.

In some instances, a peptide of the disclosure can comprise the sequence XCMPCFTTXXXMXXXCDXCCGXXXXGXCXGPXCLCX (SEQ ID NO: 425) or a fragment thereof, where X can independently be any amino acid or amino acid analogue.

In some embodiments, a peptide of the present disclosure comprises a sequence having cysteine residues at one or more of positions 4, 5, 7, 8, 12, 18, 21, 22, 26, 28, 30, 35, or 37. For example, in certain embodiments, a peptide comprises a sequence having a cysteine residue at position 4. In certain embodiments, a peptide comprises a sequence having a cysteine residue at position 5. In certain embodiments, a peptide comprises a sequence having a cysteine residue at position 7. In certain embodiments, a peptide comprises a sequence having a cysteine residue at position 8. In certain embodiments, a peptide comprises a sequence having a cysteine residue at position 12. In certain embodiments, a peptide comprises a sequence having a cysteine residue at position 18. In certain embodiments, a peptide comprises a sequence having a cysteine residue at position 21. In certain embodiments, a peptide comprises a sequence having a cysteine residue at position 22. In certain embodiments, a peptide comprises a sequence having a cysteine residue at position 26. In certain embodiments, a peptide comprises a sequence having a cysteine residue at position 28. In certain embodiments, a peptide comprises a sequence having a cysteine residue at position 30. In certain embodiments, a peptide comprises a sequence having a cysteine residue at position 35. In certain embodiments, a peptide comprises a sequence having a cysteine residue at position 37. In some embodiments, the first cysteine residue in the sequence is disulfide bonded with the 4^{th} cysteine residue in the sequence, the 2^{nd} cysteine residue in the sequence is disulfide bonded to the 5 cysteine residue in the sequence, and the 3^{rd} cysteine residue in the sequence is disulfide bonded to the 6^{th} cysteine residue in the sequence. In some embodiments, the 1^{st} cysteine residue in the sequence is disulfide bonded to the 4^{th} cysteine residue in the sequence, the second cysteine residue in the sequence is disulfide bonded to the 6^{th} cysteine residue in the sequence, the 3^{rd} cysteine residue in the sequence is disulfide bonded to the 7^{th} cysteine residue in the sequence, and the 5^{th} cysteine residue in the sequence is disulfide bonded to the 8^{th} cysteine residue in the sequence. Optionally, a peptide can comprise one disulfide bridge that passes through a ring formed by two other disulfide bridges, also known as a “two-and-through” structure system.

In some embodiments, a peptide of the present disclosure can comprise the sequence GSCXXCXXXXXXXXXXCXXCCXXXXXXXCXXXXCXC (SEQ ID NO: 213), where at least some or all of the cysteine residues form intramolecular disulfide bridges and X is any amino acid or amino acid analogue.

In some embodiments, a peptide of the present disclosure can comprise the sequence CXXCXXXXXXXXXXCXXCCXXXXXXXCXXXXCXC (SEQ ID NO: 426), where at least some or all of the cysteine residues form intramolecular disulfide bridges and X is any amino acid or amino acid analogue.

In some instances, a peptide can contain only one lysine residue, or no lysine residues. In some instances, some or all of the lysine residues in the peptide are replaced with arginine residues. In some instances, some or all of the methionine residues in the peptide are replaced by leucine or isoleucine. In some instances, some or all of the tryptophan residues in the peptide are replaced by phenylalanine or tyrosine. In some instances, some or all of the asparagine residues in the peptide are replaced by glutamine. In some cases, the N-terminus of the peptide is blocked, such as by an acetyl group. Alternatively or in combination, in some instances, the C-terminus of the peptide is blocked, such as by an amide group. In some embodiments, the peptide is modified by methylation on free amines. For example, full methylation may be accomplished through the use of reductive methylation with formaldehyde and sodium cyanoborohydride.

In some cases, the first two N-terminal amino acids of the peptide sequence are GS, as shown in SEQ ID NO: 1-SEQ ID NO: 213. In some cases, the N-terminus of peptide sequences lack GS, or substituted by any other one or two amino acids, as shown in SEQ ID NO: 214-SEQ ID NO: 426. In some cases, the GS residues in the N-terminus can be part of a linker sequence.

In some cases, the C-terminal Arg residues of a peptide is modified to another residue such as Ala, Asn, Asp, Gln, Glu, Gly, His, Leu, Lys, Met, Phe, Pro, Ser, Thr, Trp, Tyr, or Val. For example, the C-terminal Arg residue of a peptide can be modified to Ile. Alternatively, the C-terminal Arg residue of a peptide can be modified to any non-natural amino acid. This modification can prevent clipping of the C-terminal residue during expression, synthesis, processing, storage, in vitro, or in vivo including during treatment, while still allowing maintenance of a key hydrogen bond. A key hydrogen bond can be the hydrogen bond formed during the initial folding nucleation and is critical for forming the initial hairpin.

Generally, the NMR solution structures of related structural homologs can be used to inform mutational strategies that may improve the folding, stability, manufacturability, while maintaining a particular biological function. They can be used to predict the 3D pharmacophore of a group of structurally homologous scaffolds, as wells as to predict possible graft regions of related proteins to create chimeras with improved properties. For example, this strategy was used to identify critical amino acid positions and loops that may be used to design drugs with improved properties or to correct deleterious mutations that complicate folding and manufacturability for peptides of SEQ ID NO: 5, SEQ ID NO: 1, SEQ ID NO: 2, and SEQ ID NO: 3.

TABLE 3 summarizes key amino acid positions and loops that have been used with some success as learned from SEQ ID NO: 5. In some aspects, the amino acids listed in the table below may be retained while other residues in the peptide sequences may be mutated to improve, change, remove, or otherwise modify function, homing, and activity of the peptide.

TABLE 3
Exemplary Key Amino Acid Positions and Loops
According to the Present Disclosure

| Amino Acid Position | Residues |
|---|---|
| T10 | H11, H12 |
| D19 | C22, G23, G24, G26, R27 |
| R38 | R27 |

With respect to the above residues in TABLE 3, it is understood that the positions and interacting residues above describe different but corresponding positions within any peptide sequence described herein. For example, the first two N-terminal amino acids shown (GS) in SEQ ID NO: 1-SEQ ID NO: 213 can be absent, or substituted by any other one or two amino acids, as shown in SEQ ID NO:214-SEQ ID NO: 426, and in such peptides where the N-terminal amino acids (GS) are absent, amino acid position T10 would correspond to T8 with the interacting residues H11, H12 corresponding to H9, H10; amino acid position D19 would correspond to D17 with interacting residues C22, G23, G24, G26, and R27 corresponding to C20, G21, G22, G24, and R25, and amino acid position R38 would correspond to R36 with interacting residue R27 corresponding to R25. Additionally, the interacting residue at position 11 can be substituted with aspartic acid. Similarly, any variants of the peptides described herein for SEQ ID NO: 1-SEQ ID NO: 213 would have similarly corresponding residues.

Additionally, the comparison of the primary sequences and the tertiary sequences of two or more peptides can be used to reveal sequence and 3D folding patterns that can be leveraged to improve the peptides and parse out biological activity of these peptides. For example, comparing two different peptide scaffolds that cross the BBB or enter the CSF can lead to the identification of conserved pharmacophores that can guide engineering strategies, such as designing variants with improved folding properties. Important pharmacores, for example, can comprise aromatic residues, which can be important for protein-protein binding interactions.

In some instances, a peptide used to conjugate to an antibody is any one of SEQ ID NO: 1-SEQ ID NO: 426, or a functional fragment thereof. In other embodiments, the peptide of the disclosure further comprises a peptide with 99%, 95%, 90%, 85%, or 80% sequence identity or homology to any one of SEQ ID NO: 1-SEQ ID NO: 426, or fragment thereof.

In other instances, a peptide is homologous to any one of SEQ ID NO: 1-SEQ ID NO: 426, or a functional fragment thereof. The term “homologous” is used herein to denote peptides having at least 70%, at least 80%, at least 90%, at least 95%, or greater than 95% sequence identity or homology to a sequence of any one of SEQ ID NO: 1-SEQ ID NO: 426, or a functional fragment thereof.

In still other instances, the variant nucleic acid molecules of a peptide of any one of SEQ ID NO: 1-SEQ ID NO: 426, can be identified by either a determination of the sequence identity or homology of the encoded peptide amino acid sequence with the amino acid sequence of any one of SEQ ID NO: 1-SEQ ID NO: 426, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 426 (or any complement of the previous sequences) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 426. Alternatively, peptide variants of any one of SEQ ID NO: 1-SEQ ID NO: 426, can be characterized as nucleic acid molecules (1) that remain hybridized with a nucleic acid molecule having the nucleotide sequence of any one of SEQ ID NO: 1-SEQ ID NO: 426 (or any complement of the previous sequences) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 426.

Percent sequence identity or homology is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments determination of structure can typically be accompanied by evaluating activity of modified molecules.

In further embodiments, a peptide fragment comprises a contiguous fragment of any one of SEQ ID NO: 1-SEQ ID NO: 426 that is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46 residues long, wherein the peptide fragment is selected from any portion of the peptide.

In various embodiments, the peptides of the present disclosure comprise positively charged amino acid residues. In some embodiments, the peptide has at least 1 positively charged residue, at least 2 positively charged residues, at least 3 positively charged residues, at least 4 positively charged residues, at least 5 positively charged residues, at least 6 positively charged residues, at least 7 positively charged residues, at least 8 positively charged residues, at least 9 positively charged residues, at least 10 positively charged residues, at least 11 positively charged residues, at least 12 positively charged residues, at least 13 positively charged residues, at least 14 positively charged residues, at least 15 positively charged residues, at least 16 positively charged residues, or at least 17 positively charged residues. While the positively charged residues can be selected from any positively charged amino acid residues, in certain embodiments, the positively charged residues are either K, or R or a combination of K and R.

In various embodiments, the peptides of the present disclosure comprise negative amino acid residues. In some embodiments, the peptide has 1 or fewer negative amino acid residues, 2 or fewer negative amino acid residues, 3 or fewer negative amino acid residues, or 4 or fewer negative amino acid residues, 5 or fewer negative amino acid residues, 6 or fewer negative amino acid residues, 7 or fewer negative amino acid residues, 8 or fewer negative amino acid residues, 9 or fewer negative amino acid residues, or 10 or fewer negative amino acid residues. While negative amino acid residues can be selected from any negative charged amino acid residues, in certain embodiments, the negative amino acid residues are either E, or D or a combination of both E and D.

In various embodiments, the peptides of the present disclosure comprise neutral amino acid residues. In some embodiments, the peptide has 1 or fewer neutral amino acid residues, 2 or fewer neutral amino acid residues, 3 or fewer neutral amino acid residues, 4 or fewer neutral amino acid residues, 5 or fewer neutral amino acid residues, 6 or fewer neutral amino acid residues, 7 or fewer neutral amino acid residues, 8 or fewer neutral amino acid residues, 9 or fewer neutral amino acid residues, 10 or fewer neutral amino acid residues, 15 or fewer neutral amino acid residues, 20 or fewer neutral amino acid residues, 25 or fewer neutral amino acid residues, 30 or fewer neutral amino acid residues, 35 or fewer neutral amino acid residues, 40 or fewer neutral amino acid residues, or 60 or fewer neutral amino acid residues.

At physiological pH, peptides can have a net charge, for example, of −5, −4, −3, −2, −1, 0, +1, +2, +3, +4, or +5. When the net charge is zero, the peptide can be uncharged or zwitterionic. In some embodiments, the peptide contains one or more disulfide bonds and has a positive net charge at physiological pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10.

In some cases, the engineering of one or more mutations within a peptide yields a peptide with an altered isoelectric point, charge, surface charge, or rheology at physiological pH. Such engineering of a mutation to a peptide derived from a scorpion or spider can change the net charge of the complex, for example, by decreasing the net charge by 1, 2, 3, 4, or 5, or by increasing the net charge by 1, 2, 3, 4, or 5. In such cases, the engineered mutation may facilitate the ability of the peptide to cross the blood brain barrier. Suitable amino acid modifications for improving the rheology and potency of a peptide can include conservative or non-conservative mutations.

In some embodiments, the pI (the pH at which the net charge of the peptide is zero) of the peptides of this disclosure can be calculated by the EMBOSS method. The pI value is that of the folded protein or can be the isoelectric point of fully reduced form of protein sequences. The value can be calculated with the Henderson-Hasselbalch equation using EMBOSS scripts and a pKa table provided by the European Bioinformatics Institute. The EMBOSS method of calculating pI has been described by Rice et al. (EMBOSS: the European Molecular Biology Open Software Suite. Trends Genet. 2000 June; 16(6):276-7) and Carver et al. (The design of Jemboss: a graphical user interface to EMBOSS. Bioinformatics. 2003 Sep. 22; 19(14):1837-43). In some embodiments, peptides of the present disclosure with a pI value greater than 9 can have higher accumulation in the kidneys.

In some embodiments, the pI of the peptide influences its localization within the kidney. For example, in certain embodiments, higher pI values (e.g., greater than or equal to about 7.5) promote localization and/or binding to the glomerulus, while lower pI values (e.g., lower than 7.5) promote localization and/or binding to the proximal tubule. Accordingly, different localization patterns within the kidney can be achieved by varying the pI of the peptide. In certain embodiments, the osmotic concentration of the urine and/or urine flow rates have an impact on intratubular localization.

A peptide can comprise at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin component that the peptide is derived from. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom or toxin component that the peptide is derived from. In some embodiments, mutations can be engineered within a peptide to provide a peptide that has a desired charge or stability at physiological pH.

The present disclosure also encompasses multimers of the various peptides described herein. Examples of multimers include dimers, trimers, tetramers, pentamers, hexamers, heptamers, and so on. A multimer may be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The present disclosure further includes peptide scaffolds that, e.g., can be used as a starting point for generating additional peptides. In some embodiments, these scaffolds can be derived from a variety of knotted peptides or knottins. Some suitable peptides for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, μ-PIIIA, ω-MVIIA, ω-CVID, [χ] chi-MrIA, chi-MrIB, [ρ] rho-TIA, conantokin G, contulakin G, GsMTx4, margatoxin, shK, toxin K, chymotrypsin inhibitor (CTI), and EGF epiregulin core.

In some cases the peptide comprises the sequence of any one of SEQ ID NO: 1-SEQ ID NO: 426. In some embodiments, the peptide sequence is flanked by additional amino acids. One or more additional amino acids can, for example, confer a desired in vivo charge, isoelectric point, chemical conjugation site, stability, or physiologic property to a peptide.

Two or more peptides can share a degree of sequence identity or homology and share similar properties in vivo. For instance, a peptide can share a degree of sequence identity or homology with any one of the peptides of SEQ ID NO: 1-SEQ ID NO: 426. In some cases, one or more peptides of the disclosure can have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. In some cases, one or more peptides of the disclosure can have at least about 20% pairwise sequence identity or homology, at least about 25% pairwise sequence identity or homology, at least about 30% pairwise sequence identity or homology, at least about 35% pairwise sequence identity or homology, at least about 40% pairwise sequence identity or homology, at least about 45% pairwise sequence identity or homology, at least about 50% pairwise sequence identity or homology, at least about 55% pairwise sequence identity or homology, at least about 60% pairwise sequence identity or homology, at least about 65% pairwise sequence identity or homology, at least about 70% pairwise sequence identity or homology, at least about 75% pairwise sequence identity or homology, at least about 80% pairwise sequence identity or homology, at least about 85% pairwise sequence identity or homology, at least about 90% pairwise sequence identity or homology, at least about 95% pairwise sequence identity or homology, at least about 96% pairwise sequence identity or homology, at least about 97% pairwise sequence identity or homology, at least about 98% pairwise sequence identity or homology, at least about 99% pairwise sequence identity or homology, at least about 99.5% pairwise sequence identity or homology, at least about 99.9% pairwise sequence identity or homology with a second peptide. Various methods and software programs can be used to determine the homology between two or more peptides, such as NCBI BLAST, Clustal W, MAFFT, Clustal Omega, AlignMe, Praline, or another suitable method or algorithm.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

Additionally, more than one peptide sequence derived from a toxin or venom can be present on or fused with a particular antibody. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

### Peptide Stability

A peptide of the present disclosure can be stable in various biological conditions. In some cases, such stable peptides are used to make peptide-antibody fusions that are stable in various biological conditions, such as resistant to reducing agents, oxidative conditions, pH changes, acidic conditions, and/or proteases.

In some cases, biologic molecules (such as peptides and fusion proteins) can provide therapeutic functions, but such therapeutic functions are decreased or impeded by instability caused by the in vivo environment. (Moroz et al. Adv Drug Deliv Rev 101:108-21 (2016), Mitragotri et al. Nat Rev Drug Discov 13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g. pH ˜1), a reducing environment, or a protease-rich environment that can degrade various proteins or protein complexes. Proteolytic activity in other areas of the body, such as the mouth, eye, lung, intranasal cavity, joint, skin, vaginal tract, mucous membranes, and serum, can also be an obstacle to the delivery of functionally active protein complexes, such as peptide-antibody fusions described herein. Proteolytic activity in cellular compartments such as lysosomes and reduction activity in lysosomes and the cytosol can degrade protein complexes such that they may be unable to provide a therapeutic function on intracellular targets. Therefore, peptides that are resistant to reducing agents, proteases, and low pH may be able to provide enhanced therapeutic effects or enhance the therapeutic efficacy of co-formulated or conjugated active agents in vivo.

Additionally, oral delivery of drugs can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function.

Peptide Resistance to Reducing Agents.

In some embodiments, chemical moieties can provide linkers or serve to link or conjugate a peptide to an antibody or a peptide-antibody complex to another molecule, such as a therapeutic agent or a cytotoxin. An example of a chemical moiety include sulfhydryl-reactive crosslinker reaction groups, such as maleimide reagent, that reacts with the sulfhydryl groups of Cys to form a crosslinker, or thioester bond, or a stable conjugate. Other chemical moieties that can serve as linkers include disulfides and hydrazones or peptides. a knotted peptide of the present disclosure can be reduction resistant. Peptides of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body and in cells, and can reduce disulfide bonds. As another example, a peptide can become reduced upon cellular internalization during trafficking of a peptide across the gastrointestinal epithelium after oral administration. A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In some embodiments, a peptide can be activated with maleimide by using bi-functional NHS-linker-maleimide conjugated onto N-terminus of a Lys-free peptide. The combined maleimide-activated peptide can then react with free thiol groups on an antibody to form a crosslinked peptide-antibody conjugate. In such methods, diafiltration can be used to remove free reducing agents from a sample.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001M-0.0001M, 0.0001M-0.001M, 0.001M-0.01M, 0.01 M-0.05 M, 0.05 M-0.1 M, for greater 15 minutes or more. In some embodiments, the reducing agent used to determine peptide stability can be dithiothreitol (DTT), Tris(2-carboxyethyl)phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent.

Peptide Resistance to Proteases.

The stability of peptides of this disclosure can be determined by resistance to degradation by proteases. In some embodiments, a knotted peptide of the present disclosure can be resistant to protease degradation. Proteases, also referred to as peptidases or proteinases, can be enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, esterases, serum proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, the knotted peptides of this disclosure can resist degradation by any class of protease. In certain embodiments, the knotted peptides of this disclosure resist degradation by pepsin (which can be found in the stomach), trypsin (which can be found in the duodenum), serum proteases, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, elafin), or any combination thereof. In some embodiments, the proteases used to determine peptide stability can be pepsin, trypsin, chymotrypsin, or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a protease.

Peptide Stability in Acidic Conditions.

Peptides of this disclosure can be administered in biological environments that are acidic. For example, after oral administration, peptides can experience acidic environmental conditions in the gastric fluids of the stomach and gastrointestinal (GI) tract. The pH of the stomach can range from −1-4 and the pH of the GI tract ranges from acidic to normal physiological pH descending from the upper GI tract to the colon. In addition, the vagina, late endosomes, and lysosomes can also have acidic pH values, such as less than pH 7. The pH of various compartments of the kidney can also vary. These acidic conditions can lead to denaturation of peptides and proteins into unfolded states. Unfolding of peptides and proteins can lead to increased susceptibility to subsequent digestion by other enzymes as well as loss of biological activity of the peptide.

In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5-10%, at least 10%-20%, at least 20%-30%, at least 30%-40%, at least 40%-50%, at least 50%-60%, at least 60%-70%, at least 70%-80%, at least 80%-90%, or at least 90-100% of the peptide remains intact after exposure to simulated gastric fluid. In some embodiments, low pH solutions such as simulated gastric fluid or citrate buffers can be used to determine peptide stability.

Peptide Stability at High Temperatures.

In some embodiments, the knotted peptides of the present disclosure are resistant to an elevated temperature. Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

### Peptide-Antibody Complexes

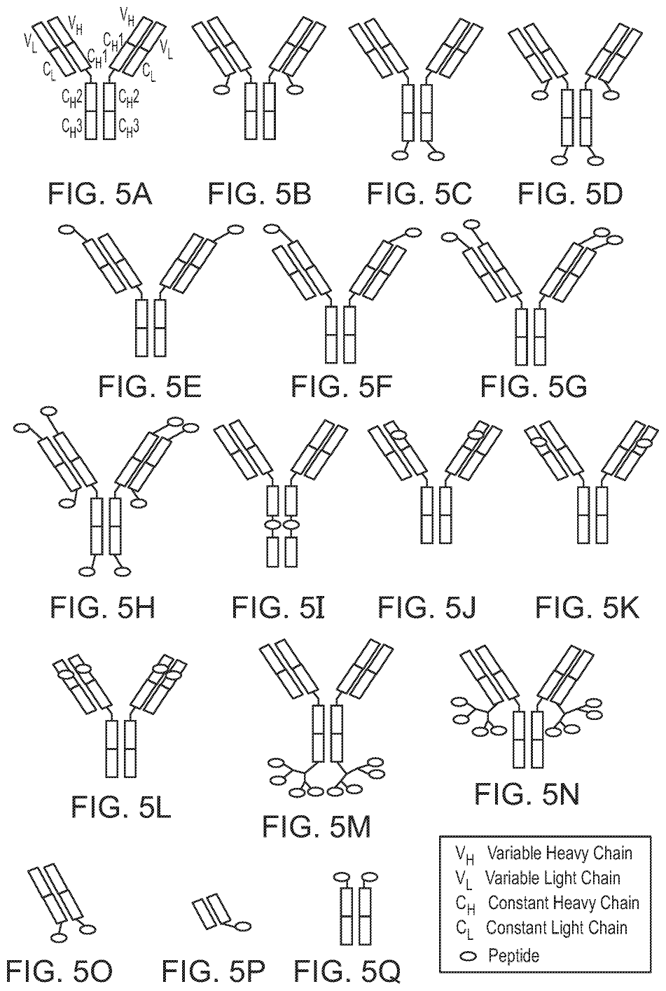

As described herein, peptide-antibody complexes can be formed in a number of configurations and combinations. In some embodiments, up to eight peptides can be fused or conjugated to a single antibody. The multiple peptides can be fused, linked, or conjugated to the heavy chain, the light chain, Fc region, variable fragment (Fv), antigen-binding fragment (Fab), or any combination thereof. The peptides can be fused to the N-terminus or C-terminus of a heavy chain, light chain, or a fragment of an antibody. Amongst other methods, fusion can mean joining the DNA for the antibody chain with the DNA for the peptide, such that when the protein is expressed in a recombinant expression system using that DNA, one protein is expressed that contains the amino acids for the antibody and for the peptide all in one sequence. Amongst other methods, conjugation can mean using a chemical reaction to chemically link the antibody with the peptide, such as by activating the peptide with an NHS ester and then reacting the peptide-NHS ester with the lysine residues in the antibody to form an antibody-peptide conjugate. It is understood that one or more peptides may be associated with, attached to, linked to, conjugated to, bound to, or fused to the antibody using various methods. A linker between a peptide and an antibody can be positioned at the N-terminus or C-terminus of the peptide to form a peptide-antibody conjugate. Some embodiments of the peptide-antibody complexes are shown in FIG. 5. In FIG. 5, FIG. 5A shows a general structure of an antibody comprising the variable heavy chain, variable light chain, constant heavy chain, and constant light chain. FIG. 5B-FIG. 5Q of FIG. 5 show various embodiments of peptide-antibody complexes, including fusions and conjugates, including peptide-antibody fragment complexes such as fusion to Fc region or Fab antibody fragments.

In some aspects, one or more peptide can be joined, conjugated, linked, attached, or fused to an antibody at the constant light chain, or at the CH3 region, or at both the constant light chain and the constant heavy chain CH3 regions, or at the variable light chain, or at the variable heavy chain, or at both the variable heavy chain and the variable light chain, or at both the variable and constant regions of the light chains and the constant regions of the heavy chains. In some cases, a peptide can be embedded or integrated in the constant heavy chain regions, or embedded or integrated in the various regions of heavy chain, or embedded or integrated in the variable region of the light chain, or embedded or integrated in the variable regions of both the heavy chain and the light chain to form peptide-antibody fusions.

In some embodiments, multiple peptides, such as 2 or more, 3 or more, 4 or more, 5 or more, 6 or more, 7 or more, or 8 or more peptides, can be conjugated, fused, or linked to the heavy chain, such as at the constant region of the heavy chain, or the light chain, such as at the constant region of the light chain.

Immunoglobulin fragment crystallization (Fc) regions dimerize, and mutations at the CH3 domain interface can be engineered to form Fc heterodimers, which can serve as a platform for generating bispecific antibodies. Fc based IgG and IgG-like antibodies can impart high stability, longer serum half-life, lower immunogenicity, and immune effector functions to molecules that bind or conjugated to such Fc-based IgG antibodies. Heterodimeric Fc can also be used to create Fc-fusion proteins with therapeutic agents. In some embodiments, peptides having therapeutic properties can be conjugated to or fused with Fc regions to form IgG-like antibodies to impart stability, longer serum half-life, lower immunogenicity, and immune effector functions to the peptide-antibody complex. In other embodiments, such peptide-Fc-based antibody complexes can serve as a platform for conjugating and delivering therapeutic agents to a target cell or tissue. In some cases, Fc-fusions with peptides can be engineered to form stable polymers that can serve as platform for forming Fc-fusion immune-complexes with therapeutic agents.

The term “antibody” herein is used in the broadest sense and includes polyclonal and monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

As described herein, antibodies include monoclonal antibodies, polyclonal antibodies, multispecific antibodies (for example, bispecific antibodies and polyreactive antibodies), and antibody fragments. Antibodies can be synthetic, naturally occurring, or modified, including chimeric receptors with one or more stimulatory, signaling, and/or costimulatory domains; and chimeric antigen receptors (CARs). Antibody, as contemplated herein, includes, but is not limited to, full-length and native antibodies, as well as fragments and portions with binding specificities, such as any specific binding portion thereof, including those having any number of, immunoglobulin classes and/or isotypes, such as IgG1, IgG2, IgG3, IgG4, IgM, IgA, IgD, IgE and IgM; and biologically relevant (antigen-binding) fragments or specific binding portions thereof, including but not limited to Fab, F(ab′)2, Fv, and scFv (single chain or related entity). A monoclonal antibody can also contain engineered or naturally occurring mutations that improve its effector functions. A polyclonal antibody includes different antibodies of varying sequences that generally are directed against two or more different determinants (epitopes).

Monoclonal antibodies (MAbs) as described herein can be complexed to one or more peptides through various covalent or non-covalent interactions to form conjugates, fusions, embedded fusions, linked conjugates, weakly or tightly bound complexes through affinity or non-covalent interactions, or any combination thereof. Such peptide-antibody complexes can serve as platforms for various therapeutic agents, including drugs and cytotoxic agents, using either or both the antibody and the peptide to target a specific cell or tissue. Effector functions of MAbs can be altered by amino acid mutations in heavy chain constant regions or through glycol-modification of Fc-linked oligosaccharides. In some cases, effector functions include antibody-dependent cell-medicated cytotoxicity and complement-dependent cytotoxicity can be triggered. Such effector functions can be improved by fucose depletion from Fc-linked oligosaccharides and by IgG1 and IgG3 isotype shuffling in heavy chains. Ways to improve effector functions of antibodies for cancer treatment are further described in Natsume et al., Drug Des. Devel. Ther., 2009, 3: 7-16. The Fc fragment of an antibody can be used as a partner to make fusions with other therapeutic proteins. Fc fragments can be found in various isotypes, such as IgG₁, IgG₂, IgG₃, IgG₄, IgA₁, IgA₂, IgD, IgE, and IgM. Fc-based fusion proteins can be produced as bivalent homodimeric proteins due to the inherent dimeric nature of the Fc fragment. The Fc domain can provide beneficial biological and pharmacological properties. For example, the Fc domain can increase the plasma half-life of the fusion protein and therefore can increase therapeutic activity. The Fc domain of the fusion protein can interact with Fc-receptors on immune cells and can elicit an immune system response. The Fc domain can improve solubility and stability because of its ability to fold independently.

In some embodiments, the antibody has been glycoengineered to modify the oligosaccharides in the Fc region and wherein the antibody has increased effector function as compared to a non-glycoengineered antibody. In some embodiments, the antibody is a monoclonal antibody. In some embodiments, the antibody is a human, humanized, or chimeric antibody. In some embodiments, the antibody is a full-length IgG class antibody. In other embodiments, the antibody is an antibody fragment. In further embodiments, the antibody is a single chain variable fragment (scFv). An antibody, as described herein, also includes a single domain antibody (sdAb or nanobody), which is an antibody fragment consisting of a single monomeric variable antibody domain. sdAb can be engineered from heavy-chain antibodies, sdAB can be heat resistant and have greater stability in various conditions. sdAb's lower molecular mass gives them greater permeability in tissues and shorter plasma half-life since they can be eliminated renally. sdAb can be conjugated, linked, or fused to peptides to form complexes that can carry various therapeutic agents for delivering a therapeutic agent to a specific target cell or tissue recognized by the antibody. In some cases, the peptide can serve as that targeting component. In other aspects, the antibody serves as the targeting component through antigen/epitope-antibody interaction. In some embodiments, the Fc regions can mediate half-life and effector functions, such as complement-dependent cytotoxicity or antibody-dependent cellular cytotoxicity, and antibody-dependent cell phagocytosis. Fc regions can be engineered to increase half-life. Mutations located at the interface between the CH2 and CH3 domains, such as T250Q/M428L, M252Y/S254T/T256E, and H433K/N434F, can increase the binding affinity to FcRn and the half-life of IgG1 in vivo. For IgG, one or more mutations in the Fc can be used to increase half-life (e.g., T250, M428, M252, S254 T256, H433, N434); increase cytotoxicity effector function (e.g., E333, S239, A330, I332, K326), or increase macrophase phagocytosis (e.g., S239, I332, G236).

In some embodiments, complexing an antibody with a peptide modifies the immunogenicity, processing, and trafficking of the complex. There are known amino acid mutations and glycosylation variations that can alter the effector functions of antibodies in the complex. For example, leucine to alanine mutations in the Fc region in some embodiments can reduce cytotoxicity, including antibody-dependent cellular cytotoxicity and/or complement activation or complement-dependent cytotoxicity. Mutations in Fc can also alter how a peptide-antibody complex binds to the surface of a target cell, internalized or trafficked by a target cell, or processed by a target cell. Antibodies have various modes of action or effector functions, including blocking ligand-receptor interactions, cause cell lysis or cell death through activation of the complement dependent cytotoxicity (CDC), interact with Fc receptors on effector cells that engage in antibody dependent cellular cytotoxicity, or trigger phagocytosis by phagocyte. These effector functions can be modified to increase or decrease effector functions or to extend serum half life. Mutations in the Fc domain that can increase cellular cytotoxicity include S239D, A330L, I332E, F243L, and G236A. Mutations that can enhance serum half-life through Fc engineering include M252Y, S254T, T256E, T250Q, or N428L of the Fc domain. In some embodiments, the Fc regions in peptide-Fc fusions contain one or more of the mutations described herein to increase cellular cytotoxicity and/or serum half life of the peptide-antibody fusion or conjugate.

In some cases, monoclonal antibodies (mAbs) and Fc-fusion proteins have a glycosylation site in the Fc region at amino acid position 297 and, in some cases, in the Fab region. Glycosylation of fusion partners in Fc-fusion proteins can also occur. Glycosylation patterns can impact the pharmacokinetics and pharmacodynamics of antibodies and fusion proteins, such as the peptide-antibody complexes described herein. Glycans that impact pharmacokinetics and pharmacodynamics include mannose, sialic acids, fucose (Fuc), and galactose (Gal). Mannosylated glycans can impact the pharmacokinetics of a peptide-antibody complex, leading to reduced exposure and potentially lower efficacy. The level of sialic acid, N-acetylneuraminic acid (NANA), can also affect the pharmacokinetics of Fc-fusion proteins. Glycosylation patterns can also affect antibody-dependent cell-mediated cytotoxicity (ADCC) activities. Antibodies and peptide-antibodies can be engineered to bypass glycosylation. In some embodiments, peptide-antibody complexes described herein comprise antibodies that are glycosylation variants, such as afucosylated antibodies and aglycosylated and glyco-engineered antibodies.

In some embodiments, a peptide-antibody complex with or without a therapeutic agent exerts a cytotoxic effect on a target cell, such as a chemotherapy conjugated to a peptide-antibody complex intended to kills cancer cells selectively. In such cases, either the peptide or antibody can serve as a targeting mechanism to direct the complex to cancerous cells to expose target cells to the therapeutic agent. In some embodiments, the antibody of the peptide-antibody complexes interact with a target protein, antigen, or receptor on a target cell, such as a cancerous cell, which triggers a signal in the cell that causes the cell to absorb or internalize the antibody along with the peptide and/or therapeutic agent attached or coupled to the complex. The internalization of the peptide-antibody complex thus provides a means for delivering a therapeutic agent inside a target cell. After the peptide-antibody has been internalized, its therapeutic agent can be released to exert an effect on the cell. Targeting using an antibody, which triggers such trafficking and internalization process, lowers off-target side effects and provides a wider therapeutic window. In some embodiments, a peptide-antibody complex is trafficked to lysosomes of a target cell, wherein lysosome-specific proteases and/or hydrolases can cleave a cleavable linker between a drug and the peptide-antibody complex or between a cytotoxic peptide and an antibody to release the drug or the cytototic peptide in the target cell.

In some embodiments, antibody is complexed or bound to peptides through weak affinity so that the peptide-antibody complex can dissociate in the target cell or tissue to release the therapeutic agent, such as a toxin or a drug. In some embodiments, the affinity between a peptide and an antibody is not weak, and replaces the need to use a linker to form a peptide-antibody complex.

Affinity is the strength of the sum total of noncovalent interactions between a single binding site of a molecule (e.g., an antibody) and its binding partner (e.g., an antigen, a target cell surface marker, or a peptide). The affinity of a molecule X for its partner Y can generally be represented by the dissociation constant (kd). In some embodiments, an antibody provided herein has a dissociation constant (K_{D}) of about 1 μM, 100 nM, 10 nM, 5 nM, 2 nM, 1 nM, 0.5 nM, 0.1 nM, 0.05 nM, 0.01 nM, or 0.001 nM or less (e.g., 10-8 M or less, e.g., from 10-8 M to 10-13 M, e.g., from 10-9 M to 10-13 M). In some embodiments, the K_{D} values ranged from 0.1 to 1.0 mM. An affinity matured antibody is an antibody with one or more alterations in one or more hypervariable regions (HVRs), compared to a parent antibody which does not possess such alterations, such alterations resulting in an improvement in the affinity of the antibody for antigen. Kd can be measured using standard methods in the art, such as surface plasmon resonance assays (e.g., using a BIACORE®-2000 or a BIACORE®-3000).

In some embodiments, peptide-antibody complexes include fusions and conjugates, which can be formed using various chemical bonds, crosslinking moieties, cloning methods, and recombinant tools. Peptide-antibody complexes include complexes wherein one or more peptides are joined or associated with one or more antibodies using one or more covalent bonds, cleavable or non-cleavable linkers, and/or non-covalent interactions. In some cases, a peptide is weakly bound to an antibody through non-covalent interaction. In some cases, a peptide can be embedded in or is part of an antibody. In other embodiments, one or more, two or more, three or more, four or more, five or more, six or more, seven or more, eight or more, nine or more, ten or more peptides can be associated with, fused, linked, or conjugated to an antibody. In some peptide-antibody complexes, the ratio of peptide:antibody is 1:1, 1:2, 1:3, 1:4, 1:5, 1:6, 1:7, 1:8, 1:9, 1:10; or 10:1, 9:1, 8:1, 7:1, 6:1, 5:1, 4:1, 3:1, 2:1; or anywhere in between, such as 2:3 or 3:2.

In some embodiments, an antibody is linked, conjugated, or fused to any one of the peptide sequences described herein, e.g., SEQ ID NO: 1-SEQ ID NO: 426. An antibody can be conjugated, fused, or linked to a peptide at the N-terminus or the C-terminus of the peptide. Peptide-antibody fusions, including peptides embedded in an antibody, can be made using various cloning and recombinant tools. For example, a single DNA sequence can be used to express a heavy chain and a light chain, either or both may further comprise a linker sequence linked to a peptide sequence such that they are expressed together recombinantly. In some cases, a linker sequence can be inserted at the DNA sequence level between a peptide and an antibody so that they are expressed together. In other cases, a linker is added chemically after each the peptide or antibody is synthesized. Short peptides can also be synthesized chemically using solid phase peptide synthesis.

Examples of antibodies that can be linked, conjugated, fused, or complexed to a peptide as described herein, e.g., any one of SEQ ID NO: 1-SEQ ID NO: 426, are described in TABLE 2. In some embodiments, antibodies that can be linked, fused, conjugated, or bound to a peptide described herein include therapeutic monoclonal antibodies, including, but not limited to, abciximab, adalimumab, alemtuzumab, Atezolizumab, basiliximab, belimumab, bevacizumab, brentuximab vedotin, canakinumab, catumaxomab, certolizumab pegol, cetuximab, daclizumab, daratumumab, denosumab, eculizumab, efalizumab, golimumab, ibritumomab tioxetan, infliximab, ipilimumab, muromonab-CD3, natalizumab, nivolumab, ofatumumab, omalizumab, palivizumab, panitumumab, pembrolizumab, ranibizumab, rituximab, tocilizumab, tositumomab, trastuzumab, ustekinumab, and vedolizumab. In other aspects, antibodies that can be linked, fused, conjugated, or bound to a peptide described herein include, but not limited to, chimeric anti-tenascin antibody 81C6, adecatumumab (MT201), anti-HGF monoclonal antibody (AMG 102), anti-insulin-like growth factor 1 receptor antibody (ave1642), Figitumumab (CP 751871), tigatuzumab (CS-1008), eteracizumab, F19, Lexatumumab (HGS-ETR2), huA33, IIIA4, IM-2C6 and CDP791, IMC-A12, KB005, labetuzumab, mapatumumab, metmab, MK-0646, MM-121, nimotuzumab, oregovomab, pemtumomab, pertuzumab, F1507, raxibacumab, SCH 900105, sibrotuzumab, and volociximab.

In some embodiments, a peptide described herein can be conjugated to an antibody conjugated to a therapeutic agent, such as Gemtuzumab ozogamicin, Brentuximab vedotin, and Trastuzumab emtansine, to form a peptide-antibody complex to further target the peptide-antibody complex to a target cell or tissue, or across the BBB or CSF barrier.

In some embodiments, antibodies linked, fused, conjugated, bound, or complexed to any one of the peptides described herein are reactive to a cell, a pathogen, or a cancer antigen in the CNS, including, but not limited to, an unhealthy or abnormal neuron, a tumor cell, beta amyloid, a surface molecule of an infectious agent, a gene product or cellular factor that contributes to tumor growth in the brain. In other embodiments, antibodies linked, fused, conjugated, or bound to any one of the peptides described herein are reactive to a cancerous cell anywhere in the body, solid tumors, or a tumor biomarker.

In some embodiments, any one of the peptides described herein, e.g., SEQ ID NO: 1-SEQ ID NO: 426, is linked, fused, bound, or conjugated to an anti-BACE1 antibody with or without a linker to target the anti-BACE1 antibody to the brain or to carry the anti-BACE1 antibody across the BBB. In other embodiments, a peptide is linked, fused, bound, or conjugated to an anti-NMDAR (glutamate receptor) antibody or an antibody that targets or binds to or modulates a biomarker of a brain tumor cell. Biomarkers of brain tumor include, but are not limited to, various genes or their mutant variants that are overexpressed in cancerous cells. For example, gene expression profiles of metastatic brain tumors can be determined using a number of microarray or sequencing techniques to identify genes that are consistently altered in tumors, including upregulation of invasion-related gene neurofibromatosis 1 (NF1), angiogenesis-related genes vascular endothelial growth factor-B (VEGF-B) and placental growth factor (PGF). Antibodies engineered to recognize such biomarkers of brain tumor cells can be linked, fused, bound, or conjugated to any one of the peptides describes herein, e.g., SEQ ID NO: 1-SEQ ID NO: 426, to deliver such antibodies across the BBB so that the peptide-antibody fusions can bind to their target molecules, confer a therapeutic effect, and/or trigger an immune-cell-mediated apoptosis of target tumor cells or immune-cell-mediated clearance of target molecules recognized by the peptide-antibody fusions.

In some embodiments, peptide-antibody fusion, conjugate, or complex comprises one or more peptides selected from: SEQ ID NO: 1, SEQ ID NO: 2, SEQ ID NO: 3, SEQ ID NO: 124, SEQ ID NO: 128, SEQ ID NO: 132, SEQ ID NO: 141, SEQ ID NO: 147, SEQ ID NO: 149, SEQ ID NO: 161, SEQ ID NO: 179, SEQ ID NO: 215, SEQ ID NO: 216, SEQ ID NO: 345, and SEQ ID NO: 374.

In some embodiments, a peptide is linked, fused, bound or conjugated to an antibody identified for use in Alzheimer's disease. For example, adacunuab and aolanezumab are monoclonal antibodies that target beta amyloid and facilitate the removal of plaques by microglial cells or immune cells in the brain. In other aspects, a peptide can be linked, fused, bound, or conjugated to an antibody that binds to or modulates beta-secretase (BACE) to inhibit the enzyme, which cleaves amyloid precursor protein (APP) and contributes to the production of beta amyloid in the brain. As demonstrated in FIG. 5, a peptide-anti-BACE antibody fusion, comprising SEQ ID NO: 428 and SEQ ID NO: 429 reduced amyloid β-protein (Aβ1-40) in a dose dependent manner, similar to the control with anti-BACE antibody without peptide fusion (SEQ ID NO: 427 and SEQ ID NO: 428).

In some embodiments, a peptide-antibody fusion targets Tau proteins or alpha synuclein in the brain. Tau proteins stabilize microtubules, while defective Tau is associated with Alzheimer's disease and Parkinson's disease. Alpha synuclein is associated with various neurodegenerative disorders, including Parkinson's disease.

In some cases, peptide-antibody fusions used for glioma are reactive to any of the following targets: EGFR, VEGFR, PDGFR, and c-kit. Additional examples of peptide-antibody fusions include, but are not limited to, biomarkers associated with gliomas, SCdc42, extracellular signal-regulated kinase (ERK), mammalian target of rapamycin (mTOR), phosphatidylinositol 3-kinase (PI3K), epidermal growth factor receptor (EGFR), growth factor receptor-bound protein 2 (Grb2), c-Jun N-terminal kinase (JNK), mitogen-activated protein kinase kinases (MEK/MKK), platelet derived growth factor receptor (PDGFR), son of sevenless (SOS), TGFβ-activated kinase (TAK), transforming growth factor (TGF), and vascular endothelial growth factor receptor (VEGFR).

T or B cell receptors can be engineered to recognize new antigens presented on cancer cells or peptide-antibody fusions bound to a target molecule, antigen, or cell. In some cases, antibodies against cancer cells recruit or target a subject's immune system to destroy cancer cells bound to such antibodies of peptide-antibody fusions. In some embodiments, antibodies can block checkpoint inhibitors in order to allow the subject's immune system to destroy cancer cells. In some embodiments, monoclonal antibodies are fused to a peptide described herein to form a peptide-antibody fusion for use in cancer immunotherapy, wherein the antibodies include, but are not limited to, alemtuzumab, atezolizumab, ipilimumab, ofatumumab, nivolumab, pembrolizumab, and rituximab, and wherein any one of such antibodies can be linked, fused, bound, or conjugated to any one of the peptides of TABLE 2. In some embodiments, a peptide for linking, conjugating, binding, or fusing to antibodies is selected from the group consisting of: SEQ ID NO: 1, SEQ ID NO: 124, SEQ ID NO: 141, SEQ ID NO: 147, and SEQ ID NO: 179.

In some embodiments, any one of the antibodies described herein, e.g., TABLE 4, is linked, fused, bound, or conjugated or fused to a peptide of TABLE 2 for targeting the antibody to a cancer cell, an antigen, or a diseased cell anywhere in the body, or across the BBB.

In some cases, a person's own T cells can be engineered to express artificial chimeric antigen receptors (CARs), such as CARs that recognize an antigen or a neoantigen presented on a cancer or tumor cells. Such modified T cells can be used in a cell-based therapy, or T cells can be modified ex vivo before transplanting the modified T cells back to a subject. In some cases, CARs are used to graft specificity of a monoclonal antibody onto a T cell, such as any one of the antibodies described herein, including the list of antibodies described in TABLE 4, or a variant, homolog, derivative, or an analog thereof. In some embodiments, CARs are designed to recognize a part of the peptide-antibody fusion or conjugate described herein to recruit immune cells to cancer cells so that a subject's own immune system can used to destroy cancer cells or other cells bounded by the peptide-antibody complexes described herein.

TABLE 4
List of Exemplary Antibodies that can be Linked, Conjugated,
Bound, or Fused to Peptides Described Herein

| Name | Target | Therapeutic Use |
|---|---|---|
| Fresolimumab | TGF beta | Cancer, renal diseases, systemic |
| | | sclerosis |
| Ustekinumab | IL-12/IL-23 | Autoimmune, inflammation |
| Risankizumab | IL-23 | Autoimmune, inflammation |
| Bevacizumab | VEGF-A | Cancer |
| Crenezumab | 1-40-β-amyloid | Alzheimer's disease |
| Urelumab | 4-1BB (CD137) | Cancer |
| Utomilumab | 4-1BB (CD137) | Cancer |
| Ensituximab | 5AC | Cancer |
| Tacatuzumab tetraxetan | alpha- fetoprotein | Cancer |
| Nesvacumab | angiopoietin 2 | Cancer |
| Vanucizumab | angiopoietin 2 | Cancer |
| Raxibacumab | anthrax toxin | Infection |
| Timolumab | AOC3 | Inflammation |
| Vapaliximab | AOC3 (VAP-1) | Inflammation |
| Vepalimomab | AOC3 (VAP-1) | Inflammation |
| 8H9 | B7-H3 | Neuroblastoma, sarcoma, |
| | | metastatic brain cancers |
| Bapineuzumab | beta amyloid | Alzheimer's disease |
| Gantenerumab | beta amyloid | Alzheimer's disease |
| Solanezumab | beta amyloid | Alzheimer's disease |
| Aducanumab | beta-amyloid | Alzheimer's disease |
| Galcanezumab | calcitonin | Migraine |
| Varlilumab | CD27 | Solid tumors and hematologic |
| | | malignancies |
| Atezolizumab | CD274 | Cancer |
| Avelumab | CD274 | Cancer |
| Durvalumab | CD274 | Cancer |
| Enoblituzumab | CD276 | Cancer |
| Lulizumab pegol | CD28 | Autoimmune diseases |
| TGN1412 | CD28 | Chronic lymphocytic leukemia, |
| | | rheumatoid arthritis |
| Lintuzumab | CD33 | Cancer |
| SGN-CD33A | CD33 | Cancer |
| Vadastuximab talirine | CD33 | Acute myeloid leukemia |
| Lilotomab satetraxetan | CD37 | Cancer |
| Naratuximab emtansine | CD37 | B cell malignancies |
| Otlertuzumab | CD37 | Cancer |
| Tetulomab | CD37 | Cancer |
| Isatuximab | CD38 | Cancer |
| Daratumumab | CD38 | Cancer |
| Lorvotuzumab mertansine | CD56 | Cancer |
| Vorsetuzumab mafodotin | CD70 | Cancer |
| Milatuzumab | CD74 | Multiple myeloma and other |
| | | hematological malignancies |
| Polatuzumab vedotin | CD79B | Cancer |
| Galiximab | CD80 | B-cell lymphoma |
| Erenumab | CGRP | Migraine |
| Margetuximab | ch4D5 | Cancer |
| IMAB362 | CLDN18.2 | Gastrointestinal |
| | | adenocarcinomas and pancreatic |
| | | tumor |
| Tisotumab vedotin | coagulation factor III | Cancer |
| Cabiralizumab | CSF1R | Macrophage-dependent |
| | | disorders |
| Emactuzumab | CSF1R | Cancer |
| Lenzilumab | CSF2 | Myelomonocytic leukemia |
| Namilumab | CSF2 | Inflammation; rheumatoid |
| | | arthritis |
| Ticilimumab | CTLA-4 | Cancer |
| (=tremelimumab) | | |
| Tremelimumab | CTLA-4 | Cancer |
| Parsatuzumab | EGFL7 | Cancer |
| Cetuximab | EGFR | Metastatic colorectal cancer and |
| | | head and neck cancer |
| Depatuxizumab mafodotin | EGFR | Cancer |
| Futuximab | EGFR | Cancer |
| Imgatuzumab | EGFR | Cancer |
| Laprituximab emtansine | EGFR | Cancer |
| Edobacomab | endotoxin | Sepsis caused by Gram-negative |
| | | bacteria |
| Nebacumab | endotoxin | Sepsis |
| Adecatumumab | EpCAM | Prostate and breast cancer |
| Citatuzumab bogatox | EpCAM | Ovarian cancer and other solid |
| | | tumors |
| Edrecolomab | EpCAM | Colorectal carcinoma |
| Oportuzumab monatox | EpCAM | Cancer |
| Solitomab | EpCAM | Cancer |
| Tucotuzumab celmoleukin | EpCAM | Cancer |
| Catumaxomab | EpCAM, CD3 | Ovarian cancer, malignant |
| | | ascites, gastric cancer |
| Fibatuzumab | ephrin receptor A3 | Cancer |
| Epitumomab cituxetan | episialin | Epithelial ovarian cancers |
| Sontuzumab | episialin | Breast cancer |
| Duligotumab | ERBB3 (HER3) | Cancer |
| Elgemtumab | ERBB3 (HER3) | Cancer |
| Lumretuzumab | ERBB3 (HER3) | Cancer |
| Patritumab | ERBB3 (HER3) | Cancer |
| Seribantumab | ERBB3 (HER3) | Cancer |
| Sibrotuzumab | FAP | Cancer |
| Biciromab | fibrin II, beta chain | Thromboembolism (diagnosis) |
| Radretumab | fibronectin extra domain-B | Cancer |
| Pasotuxizumab | folate hydrolase | Cancer |
| Farletuzumab | folate receptor 1 | Ovarian cancer |
| Mirvetuximab soravtansine | folate receptor alpha | Cancer |
| Vantictumab | Frizzled receptor | Cancer |
| Crotedumab | GCGR | Diabetes |
| 3F8 | GD2 ganglioside | Neuroblastoma |
| Ch.14.18 | GD2 ganglioside | Neuroblastoma |
| Dinutuximab | GD2 ganglioside | Neuroblastoma |
| TRBS07 | GD2 ganglioside | Melanoma |
| Ecromeximab | GD3 ganglioside | Malignant melanoma |
| Mitumomab | GD3 ganglioside | Small cell lung carcinoma |
| Codrituzumab | glypican 3 | Cancer |
| Mavrilimumab | GMCSF receptor α-chain | Rheumatoid arthritis |
| Glembatumumab vedotin | GPNMB | Melanoma, breast cancer |
| Trevogrumab | growth differentiation | Muscle atrophy due to |
| | factor 8 | orthopedic disuse and |
| | | sarcopenia |
| Indusatumab vedotin | GUCY2C | Cancer |
| Zatuximab | HER1 | Cancer |
| Pertuzumab | HER2/neu | Cancer |
| Trastuzumab | HER2/neu | Breast cancer |
| Trastuzumab emtansine | HER2/neu | Breast cancer |
| Ertumaxomab | HER2/neu, CD3 | Breast cancer |
| Ficlatuzumab | HGF | Cancer |
| Rilotumumab | HGF | Solid tumors |
| Emibetuzumab | HHGFR | Cancer |
| Derlotuximab biotin | histone complex | Recurrent glioblastoma |
| | | multiforme |
| Suvizumab | HIV-1 | Viral infections |
| Apolizumab | HLA-DR | Hematological cancers |
| Fasinumab | HNGF | Acute sciatic pain |
| Ponezumab | human beta-amyloid | Alzheimer's disease |
| Onartuzumab | human scatter factor | Cancer |
| | receptor kinase | |
| Placulumab | human TNF | Pain and inflammatory diseases |
| Cixutumumab | IGF-1 receptor (CD221) | Solid tumors |
| Dalotuzumab | IGF-1 receptor (CD221) | Cancer |
| Figitumumab | IGF-1 receptor (CD221) | Adrenocortical carcinoma, non- |
| | | small cell lung carcinoma etc. |
| Ganitumab | IGF-1 receptor (CD221) | Cancer |
| Robatumumab | IGF-1 receptor (CD221) | Cancer |
| Teprotumumab | IGF-1 receptor (CD221) | Hematologic tumors |
| Xentuzumab | IGF1, IGF2 | Cancer |
| Ixekizumab | IL 17A | Autoimmune diseases |
| TNX-650 | IL-13 | Hodgkin's lymphoma |
| Brodalumab | IL-17 | Inflammatory diseases |
| Cergutuzumab amunaleukin | IL2 | Cancer |
| Dusigitumab | ILGF2 | Cancer |
| Lirilumab | KIR2D | Solid and hematological cancers |
| Monalizumab | KLRC1 | Head and neck cancer |
| cBR96-doxorubicin | Lewis-Y antigen | Cancer |
| immunoconjugate | | |
| Carlumab | MCP-1 | Oncology/immune indications |
| Amatuximab | mesothelin | Cancer |
| Imalumab | MIF | Cancer |
| Ublituximab | MS4A1 | Cancer |
| Anetumab ravtansine | MSLN | Cancer |
| Fulranumab | NGF | Pain |
| Tanezumab | NGF | Pain |
| Racotumomab | N-glycolylneuraminic acid | Cancer |
| Ozanezumab | NOGO-A | ALS and multiple sclerosis |
| Brontictuzumab | Notch 1 | Cancer |
| Tarextumab | Notch receptor | Cancer |
| Vesencumab | NRP1 | Solid malignancies |
| Nivolumab | PD-1 | Cancer |
| Pidilizumab | PD-1 | Cancer and infectious diseases |
| Lambrolizumab | PDCD1 | Antineoplastic agent |
| Pembrolizumab | PDCD1 | Cancer |
| Olaratumab | PDGF-R α | Cancer |
| Lifastuzumab vedotin | phosphate-sodium co- | Cancer |
| | transporter | |
| Narnatumab | RON | Cancer |
| Atinumab | RTN4 | Spinal cord injury |
| Indatuximab ravtansine | SDC1 | Cancer |
| Vandortuzumab vedotin | STEAP1 | Cancer |
| Anatumomab mafenatox | TAG-72 | Non-small cell lung carcinoma |
| Minretumomab | TAG-72 | Tumor |
| Satumomab pendetide | TAG-72 | Cancer (diagnosis) |
| Maslimomab | T-cell receptor | Immunosuppression |
| Ontuxizumab | TEM1 | Cancer |
| Tenatumomab | tenascin C | Cancer |
| Pogalizumab | TNFR superfamily member | Solid tumors |
| | 4 | |
| Nerelimomab | TNF-α | Cancer |
| Ozoralizumab | TNF-α | Inflammation |
| Mapatumumab | TRAIL-R1 | Cancer |
| Conatumumab | TRAIL-R2 | Cancer |
| Lexatumumab | TRAIL-R2 | Cancer |
| Tigatuzumab | TRAIL-R2 | Cancer |
| Sacituzumab govitecan | tumor-associated calcium | Cancer |
| | signal transducer 2 | |
| Enavatuzumab | TWEAK receptor | Cancer |
| Icrucumab | VEGFR-1 | Cancer |
| Alacizumab pegol | VEGFR2 | Cancer |
| Ramucirumab | VEGFR2 | Solid tumors |
| Pritumumab | vimentin | Brain cancer |
| Nofetumomab merpentan | EpCAM , CD20/MS4A1 | Cancer (diagnosis) |
| Telimomab aritox | CD5 | Immunosuppression |
| Crenezumab | 1-40-β-amyloid | Alzheimer's disease |
| Urelumab | 4-1BB (CD137) | Cancer |
| Utomilumab | 4-1BB (CD137) | Cancer |
| Ensituximab | 5AC | Cancer |
| Naptumomab estafenatox | 5T4 | Non-small cell lung carcinoma, |
| | | renal cell carcinoma |
| Ascrinvacumab | activin receptor-like kinase | Cancer |
| | 1 | |
| Pintumomab | adenocarcinoma antigen | Adenocarcinoma (imaging) |
| Enfortumab vedotin | AGS-22M6 | Cancer expressing Nectin-4 |
| Tacatuzumab tetraxetan | alpha-fetoprotein | Cancer |
| Nesvacumab | angiopoietin 2 | Cancer |
| Vanucizumab | angiopoietin 2 | Cancer |
| Evinacumab | angiopoietin 3 | Dyslipidemia |
| Vepalimomab | AOC3 (VAP-1) | Inflammation |
| 8H9 | B7-H3 | Neuroblastoma, sarcoma, |
| | | metastatic brain cancers |
| Belimumab | BAFF | Non-Hodgkin lymphoma etc. |
| Tabalumab | BAFF | B-cell cancers |
| Bapineuzumab | beta amyloid | Alzheimer's disease |
| Gantenerumab | beta amyloid | Alzheimer's disease |
| Solanezumab | beta amyloid | Alzheimer's disease |
| Aducanumab | beta-amyloid | Alzheimer's disease |
| Detumomab | B-lymphoma cell | Lymphoma |
| Nacolomab tafenatox | C242 antigen | Colorectal cancer |
| Igovomab | CA-125 | Ovarian cancer (diagnosis) |
| Oregovomab | CA-125 | Ovarian cancer |
| Sofituzumab vedotin | CA-125 | Ovarian cancer |
| Abagovomab | CA-125 (imitation) | Ovarian cancer |
| Galcanezumab | calcitonin | Migraine |
| Mogamulizumab | CCR4 | Cancer |
| PRO 140 | CCR5 | HIV infection |
| Tovetumab | CD140a | Cancer |
| Coltuximab ravtansine | CD19 | Cancer |
| Denintuzumab mafodotin | CD19 | Cancer |
| Inebilizumab | CD19 | Cancer, systemic sclerosis, |
| | | multiple sclerosis |
| SGN-CD19A | CD19 | Acute lymphoblastic leukemia |
| | | and B-cell non-Hodgkin |
| | | lymphoma |
| Taplitumomab paptox | CD19 | Cancer |
| Afutuzumab | CD20 | Lymphoma |
| FBTA05 | CD20 | Chronic lymphocytic leukemia |
| Moxetumomab pasudotox | CD22 | Cancer |
| Pinatuzumab vedotin | CD22 | Cancer |
| Varlilumab | CD27 | Solid tumors and hematologic |
| | | malignancies |
| Atezolizumab | CD274 | Cancer |
| Avelumab | CD274 | Cancer |
| Durvalumab | CD274 | Cancer |
| Enoblituzumab | CD276 | Cancer |
| Lilotomab satetraxetan | CD37 | Cancer |
| Naratuximab emtansine | CD37 | Non-Hodgkin's lymphoma, |
| | | chronic lymphocytic leukemia, |
| | | B-cell lymphomas |
| Otlertuzumab | CD37 | Cancer |
| Tetulomab | CD37 | Cancer |
| Isatuximab | CD38 | Cancer |
| Daratumumab | CD38 (cyclic ADP ribose | Cancer |
| | hydrolase) | |
| Ibalizumab | CD4 | HIV infection |
| Zanolimumab | CD4 | Rheumatoid arthritis, psoriasis, |
| | | T-cell lymphoma |
| Bleselumab | CD40 | Cancer |
| Dacetuzumab | CD40 | Hematologic cancers |
| Lucatumumab | CD40 | Multiple myeloma, non- |
| | | Hodgkin's lymphoma, Hodgkin's |
| | | lymphoma |
| Teneliximab | CD40 | Autoimmune diseases and |
| | | prevention of organ transplant |
| | | rejection |
| Bivatuzumab mertansine | CD44 v6 | Squamous cell carcinoma |
| Abituzumab | CD51 | Cancer |
| Intetumumab | CD51 | Cancer |
| Alemtuzumab | CD52 | Multiple sclerosis |
| Lorvotuzumab mertansine | CD56 | Cancer |
| Itolizumab | CD6 | Cancer |
| Vorsetuzumab mafodotin | CD70 | Cancer |
| Milatuzumab | CD74 | Cancer |
| Polatuzumab vedotin | CD79B | Cancer |
| Galiximab | CD80 | Cancer |
| Altumomab pentetate | CEA | Cancer |
| Arcitumomab | CEA | Cancer |
| Labetuzumab | CEA | Cancer |
| Besilesomab | CEA-related antigen | Cancer |
| Erenumab | CGRP | Migraine |
| Margetuximab | ch4D5 | Cancer |
| IMAB362 | CLDN18.2 | Cancer |
| Actoxumab | Clostridium difficile | Infection |
| Bezlotoxumab | Clostridium difficile | Infection |
| Tefibazumab | clumping factor A | Infection |
| Tisotumab vedotin | coagulation factor III | Cancer |
| Cabiralizumab | CSF1R | Cancer |
| Emactuzumab | CSF1R | Cancer |
| Lenzilumab | CSF2 | Cancer |
| Namilumab | CSF2 | Cancer |
| Ticilimumab | CTLA-4 | Cancer |
| (=tremelimumab) | | |
| Tremelimumab | CTLA-4 | Cancer |
| Ulocuplumab | CXCR4 (CD184) | Cancer |
| Sevirumab | cytomegalovirus | Infection |
| Regavirumab | cytomegalovirus | Infection |
| | glycoprotein B | |
| Rovalpituzumab tesirine | DLL3 | Cancer |
| Demcizumab | DLL4 | Cancer |
| Enoticumab | DLL4 | Cancer |
| Navicixizumab | DLL4 | Cancer |
| Begelomab | DPP4 | Cancer |
| Drozitumab | DR5 | Cancer |
| Parsatuzumab | EGFL7 | Cancer |
| Cetuximab | EGFR | Cancer |
| Depatuxizumab mafodotin | EGFR | Cancer |
| Futuximab | EGFR | Cancer |
| Imgatuzumab | EGFR | Cancer |
| Laprituximab emtansine | EGFR | Cancer |
| Matuzumab | EGFR | Cancer |
| Necitumumab | EGFR | Cancer |
| Nimotuzumab | EGFR | Cancer |
| Panitumumab | EGFR | Cancer |
| Zalutumumab | EGFR | Cancer |
| Carotuximab | endoglin | Cancer |
| Edobacomab | endotoxin | Infection |
| Nebacumab | endotoxin | Infection |
| Adecatumumab | EpCAM | Cancer |
| Citatuzumab bogatox | EpCAM | Cancer |
| Edrecolomab | EpCAM | Cancer |
| Oportuzumab monatox | EpCAM | Cancer |
| Solitomab | EpCAM | Cancer |
| Tucotuzumab celmoleukin | EpCAM | Cancer |
| Catumaxomab | EpCAM, CD3 | Cancer |
| Fibatuzumab | ephrin receptor A3 | Cancer |
| Epitumomab cituxetan | episialin | Cancer |
| Sontuzumab | episialin | Cancer |
| Duligotumab | ERBB3 (HER3) | Cancer |
| Elgemtumab | ERBB3 (HER3) | Cancer |
| Lumretuzumab | ERBB3 (HER3) | Cancer |
| Patritumab | ERBB3 (HER3) | Cancer |
| Seribantumab | ERBB3 (HER3) | Cancer |
| Radretumab | fibronectin extra domain-B | Cancer |
| Pasotuxizumab | folate hydrolase | Cancer |
| Farletuzumab | folate receptor 1 | Ovarian cancer |
| Mirvetuximab soravtansine | folate receptor alpha | Cancer |
| Vantictumab | Frizzled receptor | Cancer |
| Crotedumab | GCGR | Diabetes |
| 3F8 | GD2 ganglioside | Neuroblastoma |
| Ch.14.18 | GD2 ganglioside | Neuroblastoma |
| Dinutuximab | GD2 ganglioside | Neuroblastoma |
| Derlotuximab biotin | histone complex | Glioblastoma |
| Suvizumab | HIV-1 | Viral infections |
| Apolizumab | HLA-DR | Hematological cancers |
| Fasinumab | HNGF | Acute sciatic pain |
| Ponezumab | human beta-amyloid | Alzheimer's disease |
| Volociximab | integrin α₅β₁ | Solid tumors |
| Lirilumab | KIR2D | Solid and hematological cancers |
| Monalizumab | KLRC1 | Cancer |
| cBR96-doxorubicin | Lewis-Y antigen | Cancer |
| immunoconjugate | | |
| Carlumab | MCP-1 | Oncology/immune indications |
| Amatuximab | mesothelin | Cancer |
| Imalumab | MIF | Cancer |
| Ublituximab | MS4A1 | Cancer |
| Anetumab ravtansine | MSLN | Cancer |
| Cantuzumab mertansine | mucin CanAg | Colorectal cancer, etc. |
| Refanezumab | myelin-associated | Recovery of motor function |
| | glycoprotein | after stroke |
| Fulranumab | NGF | Pain |
| Tanezumab | NGF | Pain |
| Racotumomab | N-glycolylneuraminic acid | Cancer |
| Ozanezumab | NOGO-A | ALS and multiple sclerosis |
| Brontictuzumab | Notch 1 | Cancer |
| Tarextumab | Notch receptor | Cancer |
| Vesencumab | NRP1 | Solid malignancies |
| Nivolumab | PD-1 | Cancer |
| Pidilizumab | PD-1 | Cancer and infectious diseases |
| Pembrolizumab | PDCD1 | Cancer etc. |
| Olaratumab | PDGF-R α | Cancer |
| Lifastuzumab vedotin | phosphate-sodium co- | Cancer |
| | transporter | |
| Bavituximab | phosphatidylserine | Cancer, viral infections |
| Tosatoxumab | Staphylococcus aureus | Immunomodulation in spinal |
| | | cord injury |
| Icrucumab | VEGFR-1 | Cancer |
| Alacizumab pegol | VEGFR2 | Cancer |
| Ramucirumab | VEGFR2 | Solid tumors |
| Pritumumab | vimentin | Brain cancer |

In some embodiments, any one of the antibodies described herein in TABLE 4 is linked, fused, bound, or conjugated to a peptide described herein to target delivery of the antibody to the brain or across the BBB, to cancer cells, to an antigen, or to a diseased cell. In some embodiments, any of the antibodies described herein, e.g. TABLE 4, is modified to linked, fused, bound, or conjugate to a peptide sequence described herein, wherein the modification includes, but not limited to, deletion, addition, or chemical modification of one or more amino acids. In some embodiments, an antibody linked, fused, bound, or conjugated to a peptide described herein binds to, modulates, or targets any of the following antigens associated with cancer cells: CD20, CD30, CD33, CD52, EpCAM, gpA33, CEA, mucins, TAG-72, carbonic anhydrase IX, PSMA, folate binding protein, gangliosides (e.g., GD2, GD3, GM2), Lewis-Y², VEGF, VEGFR, αVβ3, α5β1, ErbB1/EGFR, ErbB2/HER2, ErbB3, c-MET, IGF1R, EphA3, TRAIL-R1, TRAI-R2, RANKL, FAP, and tenascin.

In some embodiments, the peptide-antibody complexes or fusions can consist of Fc-based antibody fusions such as belatacept, aflibercept, rilonacept, romiplostim, abatacept, alefacept, or etanercept. The Fc-based antibody fusions can be engineered for specific applications such as targeting cancer cells and killing cancer cells via antibody-dependent cell-mediated cytotoxicity or apoptosis. Peptide-antibody fusions consisting of Fc-based antibody fusions can also be modified with drugs to improve efficacy of tumor killing. For some antibody fusions, it may be necessary to include a chaperone protein whose presence stabilizes the desired partner such as a Toll-like receptor 4 Fc-fusion. The Fc domains can be engineered to demonstrate multiple specificities according to a process called strange-exchange engineered domains (SEED) wherein the Fc domains comprises of segments of human IgA and IgG sequences in the CH3 region of the Fc domain. Multiple avidities of Fc-fusions for binding of up to twelve target molecules can be created with tandem Fc repeated homodimers and star-shaped hexameric constructs.

In some embodiments, FcRn recycling plays an important role in the processing of Fc-fusion proteins. Fc-fusion protein drugs bind to FcRn on the endothelium. When protein is internalized by a cell, the addition of Fc fusion to a peptide or Fc-peptide-antibody complex can extend the half-life of the peptide fusion since receptor-bound proteins are internalized into endocytic vesicles and are not degraded in the lysosomal compartment. Receptor-bound proteins are recycled back to the cell membrane. Protein therapeutics are thus released back into the blood. FcRn-mediated recycling leads to prolonged circulation of Fc-fusion protein therapeutics. In some embodiments, Fc is fused to a peptide or peptide-antibody fusion to extend the half life of the peptide or a therapeutic agent conjugated to the peptide-antibody fusion. In some embodiments, internalization of a peptide-antibody fusion having a pH sensitive or cleavable linker can cause a drug, toxin, or other therapeutic agent conjugated to the fusion to break or to release the therapeutic agent attached, thereby delivering the therapeutic agent into a target cell or tissue.

In some embodiments, cytokines can be targeted to the appropriate cells or tissue using the peptide-antibody conjugate, fusion, or complex. In some cases, a peptide is directly fused to or conjugated to a cytokine, such as IL2, IL21, IL15, IL18, interferon alpha, and interferon beta. In other aspects, a checkpoint inhibitor, such as CD28, PD1, PDL1, and CTLA4, can also be conjugated to a peptide-antibody complex.

### Chemical Modifications of Peptide-Antibody Complexes

A peptide-antibody complex can be chemically modified. In some embodiments, the peptide-antibody complex can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, a peptide-antibody complex is modified by methylation on free amines. For example, full methylation may be accomplished through the use of reductive methylation with formaldehyde and sodium cyanoborohydride.

In some embodiments, the first two N-terminal amino acids (GS) of SEQ ID NO: 1-SEQ ID NO: 213 serve as a spacer or linker in order to facilitate conjugation or fusion to an antibody, as well as to facilitate cleavage of the peptide from such conjugated or fused molecules. In some embodiments, peptide-antibody complexes can be further modified, including addition, deletion, substitution, or chemical modification of one or more amino acids.

A chemical modification can, for instance, can change the biodistribution or pharmacokinetic profile. A chemical modification can comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region can be a fusion Fc-peptide. A polyamino acid can include, for example, a polyamino acid sequence with repeated single amino acids (e.g., polyglycine), and a polyamino acid sequence with mixed polyamino acid sequences (e.g., gly-ala-gly-ala (SEQ ID NO: 446)) that may or may not follow a pattern, or any combination of the foregoing. In some embodiments, a peptide-antibody complex is further modified chemically to alter or modulate its biodistribution or pharmacokinetic profile.

### Conjugates to Peptide-Antibody Complexes

In some embodiments, peptide-antibody complexes of the present disclosure can be further conjugated or bound to one or more moieties that can modify the properties, e.g., stability, improve expression, improved solubility, pharmacokinetics, extended serum half-life, immunogenicity, and biodistribution, or functionalities of the peptide-antibody fusion or conjugate, e.g., cytotoxic effect or a therapeutic effect. In some embodiments, a therapeutic agent can be linked or conjugated to a peptide described herein to increase targeting, stability, biodistribution, and other properties of the peptide-antibody complex. In some embodiments, therapeutic agents are conjugated to one or more peptides described herein to impart a cytotoxic effect to or in target cells, such as peptides derived from toxins.

Any therapeutic agent can be conjugated, linked, bound, or attached to a peptide-antibody complex as described herein. A therapeutic agent can be attached to the peptide or the antibody, or both, in a peptide-antibody complex. In some embodiments, conjugation between a peptide-antibody and a therapeutic agent can occur through a thiobridge, lysine conjugation (e.g., using a bi-functional reagent containing both amine- and thiol-reactive functional groups to react with an e-amino groups of lysine (i.e., acylation), followed by reaction with a thiol-reactive group on the antibody), cysteine-based conjugation, conjugation based on one or more non-natural amino acids incorporated in the protein or protein complex, or Transglutamine based approach.

In some cases, a peptide-antibody complex is further conjugated or fused to another molecule at the N-terminus or the C-terminus of the peptide-antibody complex, such as a neurotensin, a fluorophore, or any other suitable molecule or moiety for detection or visualization or to enhance a property or effector function of the peptide-antibody complex.

A peptide-antibody complex can be further conjugated to an agent used in imaging, research, therapeutics, theranostics, pharmaceuticals, chemotherapy, chelation therapy, targeted drug delivery, and radiotherapy. In some embodiments, a peptide-antibody complex is further conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, or 10 detectable agents can be linked to a peptide-antibody complex. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine green, rhodamine red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US 14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Other embodiments of the present disclosure provide peptide-antibody fusions or conjugates further conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide-antibody fusion is fused with, or covalently or non-covalently linked to the agent, e.g., directly or via a linker. Exemplary linkers suitable for use with the embodiments herein are discussed in further detail below.

In some embodiments, peptide-antibody fusions or conjugates are further modified or conjugated to a therapeutic agent. Examples of a therapeutic agent that can be further conjugated, linked, or fused to a peptide-antibody fusion or conjugate include, but not limited to a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, an second antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, a chemokine, a neurotransmitter, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc region, or an active fragment or a modification thereof. In some embodiments, the peptide-antibody complex is further conjugated to an active agent via a covalent or non-covalent linker, e.g., directly or via a linker. For example, cytotoxic molecules that can be used include auristatins, MMAE, MMAF, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, and BACE inhibitors. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). The peptide-antibody fusion or conjugates disclosed herein can be used to home, distribute to, target, directed to, accumulate in, migrate to, and/or bind to cancerous cells, and thus also be used for localizing the attached or fused active agent.

In other embodiments, a cytotoxic drug can be conjugated to an antibody in the context of a peptide-antibody complex to target the delivery of the cytotoxic drug. Cytotoxic drugs that can be coupled to, conjugated, linked to, or attached to a peptide-antibody complex as described herein include microtubule inhibitors, DNA intercalators, DNA cleavers, and various other inhibitors or modulators of cellular processes, including, but not limited to, Bleomycin A2, Calicheamicin g-1, Auristatins (e.g., MMAE, MMAF, PE, Dolastatin-10), Maytansines (e.g., DM-1, DM-4, and derivatives), Vinorelbine, Paclitaxel, Epothilone B, Tubulysins (IM-2, B), Doxorubicin, Epirubicin, PNU-159682, Duocarmycins, PBD dimers, Oligomycin C, Daunorubicin, Valrubicin, Topotecan and desmethyl-Topotecan, DNA Transcription Inhibitors, e.g., Dactinomycin, Akt inhibitor, e.g., Ipatasertib (GDC-0068), DNA cross-linker, e.g., Mitomycin C, and Dihydrofolate reductase (DHFR) inhibitor, e.g., Methotrexate.

In some embodiments, peptide-antibody complexes of the present disclosure can also be conjugated to other moieties that can serve other roles, such as providing an affinity handle (e.g., biotin) for retrieval of the peptide-antibody complexes from tissues or fluids. For example, peptide-antibody complexes of the present disclosure can also be conjugated to biotin. In addition to extension of half-life, biotin could also act as an affinity handle for retrieval of peptide-antibody complexes from tissues or other locations. In some embodiments, fluorescent biotin can be further conjugated to a peptide-antibody complex, which acts both as a detectable label and an affinity handle. Non limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, further conjugates to peptide-antibody complexes could include chemiluminescent compounds, colloidal metals, luminescent compounds, enzymes, radioisotopes, and paramagnetic labels. In some embodiments, peptide-antibody complexes described herein can also be attached to another molecule using a covalent or a non-covalent linked linker, or a spacer.

In additional embodiments, site-specific conjugation of a cytotoxic drug to a peptide-antibody complex can improve the therapeutic index. Cysteine substitutions can be engineered in the antibody heavy and light chains during cloning to expose free thiol groups for conjugation to a cytotoxic agent. Up to 10, up to 9, up to 8, up to 7, up to 6, up to 5, up to 4, up to 3, or up to 2 drugs, including therapeutic to cytotoxic agents or moieties can be conjugated to a peptide-antibody complex. Additional ratios of drug to peptide-antibody complex can be prepared to determine the effect of drug loading. Methods of site-specific conjugation of a cytotoxic drug to an antibody to improve the therapeutic index can be found at Junutula et al, Nature Biotechnology 26, 925-932 (2008). Antibody-drug conjugate formed with monomethyl auristatin E (MMAE) conjugated to the anti-CD30 monoclonal antibody (mAb) cAC10 with eight drug moieties per mAb and the effects of drug loading on antitumor activity of mAb-drug conjugate is described at Hamblett et al., Clinical Cancer Research, 2004, 7063-7070.

### Linkers

In some embodiments, a linker is added between the peptide and the antibody of a peptide-antibody complex to reduce the risk of misfolding of the complex or impaired bioactivity. In some embodiments, a linker can be synthetic or natural. Synthetic linkers include polymers, such as polyethylene glycol (PEG) linkers. Natural linkers can comprise amino acids. Linkers can be engineered to be flexible, rigid, or cleavable. Linkers can be of varying lengths. In some cases, linkers can be solven exposed or hydrophobic. Flexible linkers allow for a certain degree of movement and can comprise small, non-polar amino acids, such as glycine or polar amino acids, or threonine and alanine, or polar amino acids lysine and glutamate. Examples of a flexible linker include (GGGGS)n (SEQ ID NO: 447) where n is the copy number, GS, GGS, or any variant thereof. Other flexible linkers include, but are not limited to, KESGSVSSEQLAQFRSLD (SEQ ID NO: 448), EGKSSGSGSESKST (SEQ ID NO: 449), (G)8 (SEQ ID NO: 450), and GSAGSAAGSGEF (SEQ ID NO: 451). Rigid linkers can improve the effectiveness of protein bioactivity. For example, rigid linkers can include alpha helix-forming linkers such as (EAAAK)n (SEQ ID NO: 452), empirical rigid linkers such as A(EAAAK)nA (SEQ ID NO: 453), or proline-rich linkers such as (XP)n where X is any amino acid, or preferably alanine, lysine, or glutamate. Rigid and flexible linkers can impart greater stability in a peptide-antibody complex or fusion protein. In some cases, a linker as described herein can prolong the half-life of a peptide-antibody complex in vivo. Cleavable linkers can be cleaved in the presence of reducing agents or proteases. For example, a reduction of disulfide bonds can create a break in a linker. Proteases can also cleave linkers. In some cases, cleavable linkers can be engineered to be cleaved by a specific protease present only in a target cell or tissue, thus ensuring specificity and/or targeting of release of a therapeutic agent from a peptide-antibody complex in the applicable cell or tissue.

In some embodiments, cleavable or non-cleavable linkers can be used to conjugate or link peptides and antibodies for forming peptide-antibody complexes. In some embodiments, the linker is pH sensitive or can be cleaved at the target tissue or cell, allowing release of a peptide, antibody, or therapeutic agent, when the complex reaches the target cell or tissue, or becomes internalized by the target cell. In some cases, one or more linkers are used to further conjugate peptide-antibody complexes to a therapeutic agent or another moiety, such as a detectable agent. In some embodiments, a linker serves to physically separate an antibody and a peptide or a therapeutic agent in order to sterically access binding sites. In some embodiments, a cemadotin derivative containing a sulfhydryl group can be used to link or conjugate a peptide to an antibody or a peptide-antibody complex to a drug or a therapeutic agent. In other cases, a cemadotin derivative containing an aldehyde group can be used to link or conjugate a peptide to an antibody or a peptide-antibody complex to a drug or a therapeutic agent via a thiazolidine linkage.

In some cases, a linker comprises GGGSGGGS (SEQ ID NO: 444), C5 hydrocarbon linkers, or short PEG linkers. In some embodiments, the peptide, the antibody, or the peptide-antibody undergoes PEGylation, or the addition of ethylene glycol or ethylene oxide polymers. PEG spacer arm is hydrophilic and non-immunogenic. HO-PEG peglyation tail can also reduce aggregation or immunogenicity. PEGylation reagents that can be used add PEG linkers include, but not limited to, m-PEG36-alcohol, m-PEG4-thiol, m-PEG2-acid, m-PEG3-acid, m-PEG10-alkyne, azido-PEG11-alcohol, azido-PEG7-alcohol, m-PEG12-alkyne, and m-PEG8-alkyne. PEG linkers can be homobifunctional crosslinkers or heterobifunctional cross linkers, including, but not limited to, amino-PEG5-amine, amino-PEG7-amine, PEG11 bis-Maleimide, PEG7 Bis-PFP ester, Boc-amino-PEG 11-amine, tetrazine-PEG9-NHS, DBCO-PEG4-acid, and BCN-PEG5-amine.

In various embodiments, linkers can impart different functionalities to proteins or domains that are linked. Flexible linkers that improve stability and/or folding include (GGGGS)₃ (SEQ ID NO: 454), (Gly)s (SEQ ID NO: 450), (Gly)₆ (SEQ ID NO: 455), (EAAAK)n (SEQ ID NO: 456), wherein n is 1, 2, or 3. Rigid linkers, such as A(EAAAK)₄ALEA(EAAAK)₄A (SEQ ID NO: 457), can increase expression of a recombinant peptide-antibody complex. Other linkers can also improve biological activity, such as PAPAP (SEQ ID NO: 458), (AP)n, wherein n is 5-17 (SEQ ID NO: 459), GGGGS (SEQ ID NO: 447), (GGGGS)₃ (SEQ ID NO: 454), and disulfide cleavable linkers. In some embodiments, cleavable linkers can be used to enable targeting by a specific protease or impart specificity through protease cleavage, such as AGNRVRRSVG (SEQ ID NO: 460) and GFLG (SEQ ID NO: 461). In some embodiments, a linker can alter the pharmacokinetics of a peptide-antibody complex, such as LE dipeptide linker, rigid linker A(EAAAK)₄ALEA(EAAAK)₄A (SEQ ID NO: 457), and cleave disulfide linker. Adv Drug Deliv Rev. 2013 Oct. 15; 65(10): 1357-1369. Another useful linker is a valine-citrulline (vc) dipeptide linker, which can increase stability of a peptide-antibody complex in serum and provide conditional cleavage and release of a therapeutic agent by lysosomal cathepsins upon lysosomal trafficking of the peptide-antibody complex.

Peptide-antibody complexes or conjugates according to the present disclosure that home, distribute to, target, migrate to, accumulate in, or are directed to cancerous or diseased cells or a specific brain region (e.g., the hippocampus, ventricular system, CSF) or that cross the blood brain barrier can be further attached to another moiety (e.g., an active agent or an detectable agent), such as a small molecule, a second peptide, a protein, another antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent or detectable agent described herein through a linker, or directly in the absence of a linker. In the absence of a linker, for example, the antibody can be fused to the N-terminus or the C-terminus of a peptide to create a peptide-antibody complex.

In other embodiments, the linker can be made by a peptidic fusion via reductive alkylation. Direct attachment is possible by covalent attachment of a peptide to a region of the other molecule, such as an antibody. For example, an antibody can be fused or linked to the N-terminus or the C-terminus of a peptide to create a peptide-antibody fusion or conjugate. As another example, the peptide can be attached at the N-terminus, an internal lysine residue, or the C-terminus to a terminus of the amino acid sequence of the other molecule by a linker. If the attachment is at an internal lysine residue, the other molecule can be linked to the peptide at the epsilon amine of the internal lysine residue. For example, the internal lysine residues can be located at a position corresponding to amino acid residue 17 of SEQ ID NO: 37, amino acid residue 25 of SEQ ID NO: 37, or amino acid residue 29 of SEQ ID NO: 37 or similar residues of the disclosed peptide(s), such as any of the corresponding lysine residues in any one of SEQ ID NO: 1-SEQ ID NO: 213. As another example, the internal lysine residues can be located at a position corresponding to amino acid residue 15 of SEQ ID NO: 250, amino acid residue 23 of SEQ ID NO: 250, or amino acid residue 27 of SEQ ID NO: 250 or similar residues of the disclosed peptide(s), such as any of the corresponding lysine residues in any one of SEQ ID NO: 214-SEQ ID NO: 426. In some further examples, the peptide can be attached to the other molecule by a side chain, such as the side chain of a lysine, serine, threonine, cysteine, tyrosine, aspartic acid, a non-natural amino acid residue, or glutamic acid residue. A linker can be an amide bond, an ester bond, an ether bond, a carbamate bond, a carbonate bond, a carbon-nitrogen bond, a triazole, a macrocycle, an oxime bond, a hydrazone bond, a carbon-carbon single, double, or triple bond, a disulfide bond, a two carbon bridge between two cysteines, a three carbon bridge between two cysteines, or a thioether bond. In still other embodiments, the peptide comprises a non-natural amino acid, wherein the non-natural amino acid is an insertion, appendage, or substitution for another amino acid, and the peptide is linked to the active agent at the non-natural amino acid by a linker. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single, double, or triple bond, a disulfide bond, a thioether bond, or other linker as described herein) may be used to link other molecules.

Attachment via a linker involves incorporation of a linker moiety between an antibody and a peptide to form a peptide-antibody conjugate or between a peptide-antibody complex and another molecule. The peptide and the antibody, or a peptide-antibody complex and another molecule, can both be covalently attached to the linker. The linker can be cleavable, non-cleavable, self-immolating, hydrophilic, or hydrophobic. The linker has at least two functional groups, one bonded to the other molecule, and one bonded to the peptide, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment include functional groups capable of forming, for example, an amide bond, an ester bond, an ether bond, a carbonate bond, a carbamate bond, a carbon-nitrogen bond, a triazole, a macrocycle, an oxime bond, a hydrazone bond, a carbon-carbon single, double, or triple bond, a disulfide bond or a thioether bond. Non-limiting examples of functional groups capable of forming such bonds include amino groups; carboxyl groups; aldehyde groups; azide groups; alkyne and alkene groups; ketones; hydrazides; hydrazines; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; maleimides; linkers containing maleimide groups that are designed to hydrolyze; maleimidocaproyl; MCC ([N-maleimidomethyl]cyclohexane-1-carboxylate); N-ethylmaleimide; maleimide alkane; mc-vc-PABC; DUBA (DuocarmycinhydroxyBenzamide-Azaindole linker); SMCC Succinimidyl-4-(N-maleimidomethyl) cyclohexane-1-carboxylate; SPDP (N-succinimidyl-3-(2-pyridyldithio) propionate); SPDB N-succinimidyl-4-(2-pyridyldithio) butanoate; sulfo-SPDB N-succinimidyl-4-(2-pyridyldithio)-2-sulfo butanoate; SPP N-succinimidyl 4-(2-pyridyldithio)pentanoate; a dithiopyridylmaleimide (DTM); a hydroxylamine, a vinyl-halo group; haloacetamido groups; bromoacetamido; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion include alkylene, alkenylene, alkynylene, polyether, such as polyethylene glycol (PEG), polyester, polyamide, polyamino acids, polypeptides, cleavable peptides, Val-Cit, Phe-Lys, Val-Lys, Val-Ala, other peptide linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link molecules together include click chemistry, copper-free click chemistry, HIPS ligation, Staudinger ligation, and hydrazine-iso-Pictet-Spengler.

Non-limiting examples of linkers include:

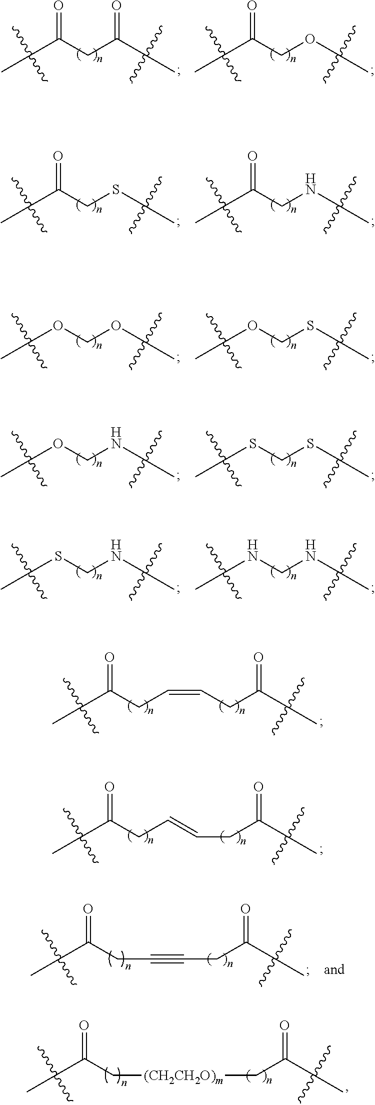

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid. In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, a linker used to conjugate or fuse a peptide to an antibody or a therapeutic agent is (GGGS)n, wherein n is 1, 2, 3, 4, 5, 6, 7, 8, 9, or 10 (SEQ ID NO: 462). In some cases, one or more Gly in GGGS (SEQ ID NO: 463) is replaced with Ala, Val, or Pro. In other aspects, Ser in GGGS (SEQ ID NO: 463) is replaced with Thr. In other embodiments, a linker is (GxS)n, wherein X is 1, 2, 3, 4, 5, 6, 7, 8, 9, or 10, and wherein n is 1, 2, 3, 4, 5, 6, 7, 8, 9, or 10 (SEQ ID NO: 445). In other aspects, a linker is (GxSy)n, wherein x is 1, 2, 3, 4, 5, 6, 7, 8, 9, or 10; y is 1, 2, 3, 4, 5, 6, 7, 8, 9, or 10, and wherein n is 1, 2, 3, 4, 5, 6, 7, 8, 9, or 10 (SEQ ID NO: 464). In some cases, the linker is GS, GGGS (SEQ ID NO: 463), or GGGSGGGS (SEQ ID NO: 444).

The linker between a peptide and antibody or between a peptide-antibody complex and another moiety, a detectable agent, or a therapeutic agent may be a noncleavable linker or a cleavable linker. In some embodiments, the noncleavable linker can slowly release the conjugated moiety by an exchange of the conjugated moiety onto the free thiols on serum albumin. In some embodiments, the use of a cleavable linker can permit release of the conjugated moiety (e.g., a therapeutic agent or an antibody) from the peptide, e.g., after targeting to the tumor or cancerous cell. In other embodiments, the use of a cleavable linker can permit the release of the conjugated therapeutic from the peptide after crossing the BBB and optionally after targeting to the specific brain region. In some cases the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, cathepsins, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters is faster compared to the hydrolysis of linkers with bulky groups next an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when conjugated via its carboxylic acid. The rate of hydrolysis of the linker can be tuned according to the residency time of the conjugate in the target location. For example, when a peptide-antibody complex is cleared from a tumor, or the brain, relatively quickly, the linker can be tuned to rapidly hydrolyze. When a peptide-antibody complex has a longer residence time in the target location, a slower hydrolysis rate would allow for extended delivery of a therapeutic agent, such as an antibody. “Programmed hydrolysis in designing paclitaxel prodrug for nanocarrier assembly” Sci Rep 2015, 5, 12023 Fu et al., provides an example of modified hydrolysis rates.

In some embodiments, chemical moieties can provide linkers or serve to link or conjugate a peptide to an antibody or a peptide-antibody complex to another molecule, such as a therapeutic agent or a cytotoxin. An example of a chemical moiety include sulfhydryl-reactive crosslinker reaction groups, such as maleimide reagent, that reacts with the sulfhydryl groups of Cys to form a crosslinker, or thioester bond, or a stable conjugate. Other chemical moieties that can serve as linkers include disulfides and hydrazones or peptides.

In some embodiments, a peptide can be activated with maleimide by using bi-functional NHS-linker-maleimide conjugated onto N-terminus of a Lys-free peptide. The combined maleimide-activated peptide can then react with free thiol groups on an antibody to form a crosslinked peptide-antibody conjugate. In such methods, diafiltration can be used to remove free reducing agents from a sample.

### Methods of Manufacture

Various expression vector/host systems can be utilized for the recombinant expression of peptide-antibody complexes described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines).

In some embodiments, mammalian systems or cell lines, such as CHO and HEK293 cells, are used to express, assemble, and purify antibodies. In some cases, the heavy chain is expressed in the same cells as the light chain. In some cases, the heavy chain and the light chain are express in different cells, and are purified and assembled in vitro. In some cases, the heavy chain and the light chain are expressed from the same expression vector or DNA sequence. In some cases, the heavy chain and the light chain are expressed from different plasmids, vector, or DNA sequence, which can be expressed in different cells or co-transfected into the same cells. After expression in cells, the heavy chain and the light chain can be purified separately or together using standard purification methods, such as affinity column, Protein A or G columns, column with target molecules for affinity purification, ion exchange chromatography, hydrophobic interaction columns, size exclusion chromatography, HPLC, FPLC, etc.

Other methods of fractionation or protein purification include separation based on physical characteristics such as charge (i.e., ion exchange chromatography, electrophoresis, isoelectric focusing), polarity (i.e., adsorption chromatography, reverse phase chromatography), size (i.e., dialysis, gel electrophoresis, gel filtration chromatography, ultracentrifugation), specificity (i.e., affinity chromatography, immunopurification), and solubility (i.e., salt precipitation, detergent solubilization). Diafiltration can be performed to remove free reducing agents in a sample.

Disulfide bond formation and folding of the peptide-antibody fusion can occur during expression or after expression or both.

A host cell can be adapted to express one or more peptide-antibody fusions described herein. In some cases, the peptide and the antibody are manufactured separately before they are fused or conjugated to form peptide-antibody fusions or conjugates. In other cases, peptide-antibody fusions are manufactured or expressed as a fusion protein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide-antibody fusion products can be important for the function of the peptide-antibody fusion. Host cells can have characteristic and specific mechanisms for the post-translational processing and modification of a peptide or peptide-antibody fusion. In some cases, the host cells that express the peptides secrete minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, peptide-antibody fusions can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptide-antibody fusions can also be synthesized in a cell-free system prior to extraction using a variety of known techniques employed in protein and peptide synthesis.

In some cases, a host cell produces a peptide or peptide-antibody fusion that has an attachment point for a drug or another moiety. An attachment point could comprise a lysine residue, an N-terminus, a cysteine residue, a cysteine disulfide bond, or a non-natural amino acid. The peptide-antibody fusion could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly.

Peptide fragments can be then be joined together enzymatically or synthetically.

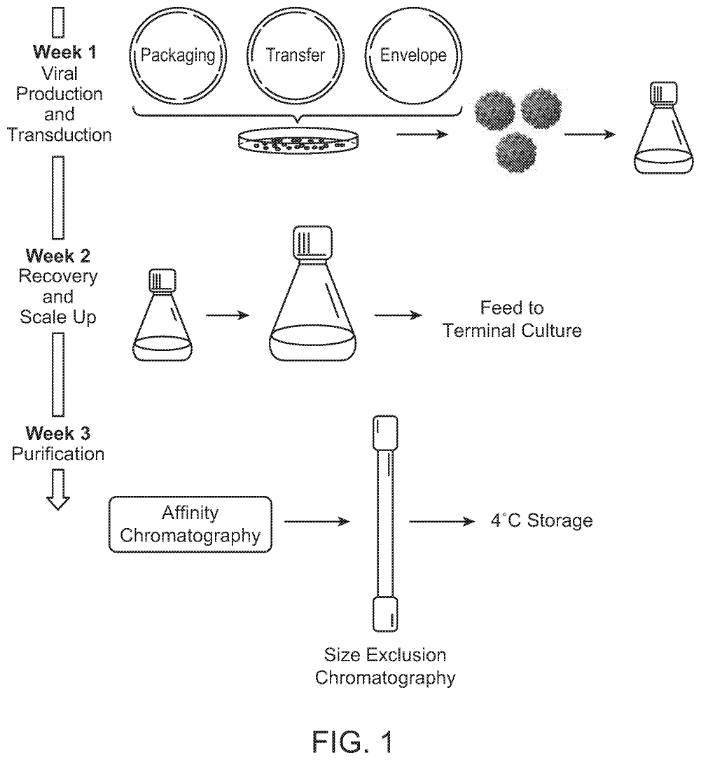

FIG. 1 illustrates a schematic of a method of manufacturing a construct that expresses a peptide or peptide-antibody fusion of the disclosure, such as the constructs as described throughout the disclosure expressing SEQ ID NO: 1-SEQ ID NO: 426 provided herein. Viral expression vectors comprising a peptide or peptide-antibody fusion can be cloned and packaged in viral particles that infect cells, such as CHO or 293 cells. Viral transduction of cells causes cells to express the peptide, antibody, or peptide-antibody fusions. The cells are then harvested, lysed, and subjected to purification methods to isolate and purify the protein expressed. Purification can include, but not limited to affinity chromatography and size exclusion chromatography.

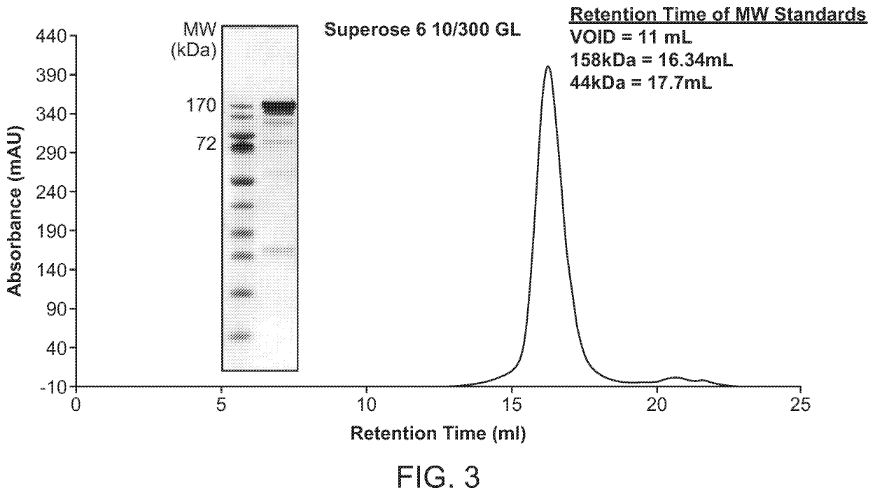

FIG. 3 provides an example of a purification profile of a peptide-antibody complex, with the human anti-BACE1 antibody light chain (SEQ ID NO: 428) bound to or assembled with human anti-BACE1 antibody heavy chain fused to peptide SEQ ID NO: 1 (SEQ ID NO: 429) eluted from a size exclusion chromatography column at the expected molecular weight, as represented by the peak in the chromatograph. Gel inset in FIG. 3 shows the purified peptide-antibody fusion complex under non-reducing conditions.

In other aspects, the peptides of the present disclosure can be prepared by conventional solid phase chemical synthesis techniques and purified before conjugating to an antibody to make a peptide-antibody fusion, for example according to the Fmoc solid phase peptide synthesis method (“Fmoc solid phase peptide synthesis, a practical approach,” edited by W. C. Chan and P. D. White, Oxford University Press, 2000).

### Peptide-Antibody Complexes in Pharmaceutical Compositions

A pharmaceutical composition of the disclosure can be a combination of any peptide-antibody complex, such as a peptide-antibody fusion or conjugate, as described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide-antibody complex described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, and topical administration. A pharmaceutical composition can be administered in a local or systemic manner, for example, via injection of the peptide-antibody complex described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide-antibody complex described herein in water-soluble form. Suspensions of peptide-antibody complexes described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances, which increase the viscosity of the suspension, such as sodium carboxymethyl cellulose, sorbitol, or dextran. The suspension can also contain suitable stabilizers or agents, which increase the solubility and/or reduce the aggregation of such peptide-antibody complexes described herein to allow for the preparation of highly concentrated solutions. Alternatively, the peptide-antibody complex described herein can be lyophilized or in powder form for re-constitution with a suitable vehicle, e.g., sterile pyrogen-free water, before use. In some embodiments, a purified peptide-antibody fusion is administered intravenously. A peptide-antibody complex described herein can be administered to a subject, home, target, migrate to, or be directed to an organ, e.g., the hippocampus and cross the blood brain barrier of a subject.

A peptide-antibody complex of the disclosure can be applied directly to an organ, or an organ tissue or cells, such as brain or brain tissue or cells, during a surgical procedure. The recombinant peptide-antibody fusion described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of a peptide-antibody complex described herein can be administered in pharmaceutical compositions to a subject suffering from a condition that affects the immune system. In some embodiments, the subject is a mammal such as a human or a primate. A therapeutically-effective amount can vary widely depending on the severity of the disease, the age and relative health of the subject, the potency of the compounds used, and other factors.

In some embodiments, a peptide-antibody fusion is cloned into a viral or non-viral expression vector, which expresses the fusion in vivo. Such expression vector can be packaged in a viral vector or a non-viral carrier, which is administered to patients in the form of gene therapy. In other embodiments, patient cells are extracted and modified to express a peptide-antibody fusion ex vivo before the modified cells are returned back to the patient in the form of a cell-based therapy, such that the modified cells will express the peptide-antibody fusion in the patient.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide-antibody complex described herein can be manufactured, for example, by expressing the peptide-antibody fusion in a recombinant system, purifying the peptide-antibody fusion, lyophilizing the peptide-antibody fusion, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptide-antibody complex described herein comprising the compounds described herein include formulating peptide-antibody complexes described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A pharmaceutical composition of the disclosure can be a combination of any peptide-antibody complex, including conjugates and fusions, described herein, or a salt thereof, with other chemical components, such as carriers, stabilizers, diluents, dispersing agents, suspending agents, thickening agents, and/or excipients. In some embodiments, the pharmaceutical composition facilitates administration of a peptide-antibody complex described herein to an organism.

Pharmaceutical compositions can be formulated for administration to a subject by various routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, or topical administration, or a combination thereof. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents, which increase the solubility and/or reduce the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously.

A peptide-antibody complex of the disclosure can be applied directly to an organ, or an organ tissue or cells, during a surgical procedure. The peptide-antibody complexes described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide-antibody complexes described herein described herein can be administered in pharmaceutical compositions to a subject suffering from a condition. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide-antibody complex described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of the pharmaceutical compositions described herein include formulating the peptide-antibody complex described herein, or a salt thereof, with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

### Use of Peptide-Antibody Complexes in Imaging and Surgical Methods

The present disclosure relates to peptide-antibody complexes, including fusions or conjugates, that home, distribute to, target, migrate to, accumulate in, or are directed to target cells, such as cancerous or diseased cells. The present disclosure relates to peptide-antibody complexes that home, target, migrate to, accumulate in, or are directed to specific regions, tissues, structures, or cells within the body and methods of using such peptide-antibody complexes. These -antibody complexes have the ability to bind to cross the blood brain barrier or blood CSF barrier, which makes them useful for a variety of applications. These abilities make them useful for a variety of applications. In particular, the peptide-antibody complexes have applications in site-specific modulation of biomolecules to which the peptide-antibody complexes are directed. End uses of such peptide-antibody complexes include, for example, imaging, research, therapeutics, theranostics, pharmaceuticals, chemotherapy, chelation therapy, targeted drug delivery, and radiotherapy. Some uses can include targeted drug delivery and imaging.

In some embodiments, peptide-antibody complexes of the disclosure deliver a metal, a radioisotope, a dye, fluorophore, or another suitable material that can be used in imaging.

Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, ZW800, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, F1AsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US 14/56177 or another suitable material that can be used in imaging.

The present invention provides methods for intraoperative imaging and resection of a cancer, cancerous tissue, tumor tissue, cancerous cells, or diseased tissue using peptide-antibody complexes of the present disclosure conjugated with a detectable agent. In some aspects, the cancer, cancerous tissue, tumor tissue, or diseased tissue or cells of the foregoing is detectable by fluorescence imaging that allows for intraoperative visualization of the cancer, cancerous tissue, tumor tissue, cancerous cells, or diseased tissue using a peptide-antibody complex of the present disclosure. In some aspects, the peptide-antibody complex of the present disclosure is further conjugated to one or more detectable agents. In a further embodiment, the detectable agent comprises a fluorescent moiety coupled to the peptide-antibody complex. In another embodiment, the detectable agent comprises a radionuclide. In some aspects, imaging is pre-operative imaging. In other aspects, imaging is achieved during open surgery. In further aspects, imaging is accomplished while using endoscopy or other non-invasive surgical techniques. In yet further aspects, imaging is performed after surgical removal of the cancer, cancerous tissue, tumor tissue, or diseased tissue or cells of the foregoing. In some embodiments, peptide-antibody complexes confer both a therapeutic effect as well as a detectable signal.

### Treatment of Cancer

In one embodiment, the method includes administering an effective amount of a peptide-antibody complex of the present disclosure to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide-antibody complex of the disclosure. The disease may be a cancer or tumor. In treating the disease, the peptide-antibody complex may contact the tumor or cancerous cells. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment may be provided to the subject before clinical onset of disease. Treatment may be provided to the subject after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition, such as one or more of the pharmaceutical compositions described throughout the disclosure. A treatment can comprise delivering a peptide-antibody complex of the disclosure to a subject, either intravenously, subcutaneously, intramuscularly, by inhalation, dermally, topically, orally, sublingually, intrathecally, transdermally, intranasally, via a peritoneal route, or directly into the brain, e.g., via and intracerebral ventrical route.

In some embodiments, the present disclosure provides a method for treating a cancer or tumor, the method comprising administering to a subject in need thereof an effective amount of a peptide-antibody complex of the present disclosure. One example of cancers or conditions that can be treated with a peptide-antibody complex of the disclosure is solid tumors. Further examples of cancers or conditions that can be treated with a peptide-antibody complex of the disclosure include triple negative breast cancer, breast cancer, breast cancer metastases, metastases of any cancers described herein, colon cancer, colon cancer metastases, sarcomas, acute lymphoblastic leukemia, acute myeloid leukemia, adrenocortical carcinoma, AIDS-related cancers such as Kaposi sarcoma, AIDS-related lymphoma, primary CNS lymphoma, anal cancer, appendix cancer, childhood astrocytomas, astrocytomas, childhood atypical teratoid/rhabdiod tumor, CNS atypical teratoid/rhabdiod tumor, atypical teratoid/rhabdiod tumor, basal cell carcinoma, skin cancer, bile duct cancer, bladder cancer, bone cancer, Ewing sarcoma family of tumors, osteosarcoma, chondroma, chondrosarcoma, primary and metastatic bone cancer, malignant fibrous histiocytoma, childhood brain stem glioma, brain stem glioma, brain tumor, brain and spinal cord tumors, central nervous system embryonal tumors, childhood central nervous system embryonal tumors, central nervous system germ cell tumors, childhood central nervous system germ cell tumors, craniopharyngioma, childhood craniopharyngioma, ependymoma, childhood ependymoma, breast cancer, bronchial tumors, childhood bronchial tumors, burkitt lymphoma, carcinoid tumor, gastrointestinal cancer, carcinoma of unknown primary, cardiac tumors, childhood cardiac tumors, primary lymphoma, cervical cancer, cholangiocarcinoma, chordoma, childhood chordoma, chronic lymphocytic leukemia, chronic myelogenous leukemia, chronic myeloproliferative neoplasms, colon cancer, colorectal cancer, cutaneous T cell lymphoma, ductal carcinoma in situ, endometrial cancer, esophageal cancer, esthesioneuroblastoma, childhood esthesioneuroblastoma, ewing sarcoma, extracranial germ cell tumor, childhood extracranial germ cell tumor, extragonadal germ cell tumor, eye cancer, intraocular melanoma, retinoblastoma, fallopian tube cancer, fibrous histiocytoma of bone, gallbladder cancer, gastric cancer, gastrointestinal carcinoid tumor, gastrointestinal stromal tumors, ovarian cancer, testicular cancer, gestational trophoblastic disease, glioma, hairy cell leukemia, head and neck cancer, hepatocellular cancer, histiocytosis, Langerhans cell histiocytosis, hodgkin lymphoma, hypopharyngeal cancer, intraocular melanoma, melanoma, melanoma metastases, islet cell tumors, pancreatic neuroendocrine tumors, kidney cancer, renal cell tumors, Wilms tumor, childhood kidney tumors, lip and oral cavity cancer, liver cancer, lung cancer, nonhodgkin lymphoma, macroglodulinemia, Waldenstrom macroglodulinemia, male breast cancer, merkel cell carcinoma, metastatic squamous neck cancer with occult primary, midline tract carcinoma involving NUT gene, mouth cancer, multiple endocrine neoplasia syndromes, childhood multiple endocrine neoplasia syndromes, multiple myeloma/plasma cell neoplasm, mycosis fungoides, myelodysplastic syndromes, myelodysplastic/myeloproliferative neoplasms, multiple myeloma, myloproliferative neoplasms, chronic myeloproliferative neoplasms, nasal cavity and paranasal sinus cancer, nasopharyngeal cancer, neuorblastoma, non-small cell lung cancer, oropharyngeal cancer, low malignant potential tumor, pancreatic cancer, pancreatic neuroendocrine tumors, papillomatosis, childhood papillomatosis, paraganglioma, paranasal sinus and nasal cavity cancer, parathyroid cancer, penile cancer, pheochromocytoma, pharyngeal cancer, pituitary tumor, pleuropulmonary blastoma, childhood pleuropulmonary blastoma, primary peritoneal cancer, prostate cancer, rectal cancer, pregnancy-related cancer, rhabdomyosarcoma, childhood rhabdomyosarcoma, salivary gland cancer, Sezary syndrome, small cell lung cancer, small intestine cancer, soft tissue sarcoma, squamous cell carcinoma, testicular cancer, throat cancer, thymoma, thymic carcinoma, thyroid cancer, transitional cell cancer of the renal, pelvis, and ureter, uterine cancer, urethral cancer, endometrial cancer, uterine sarcoma, vaginal cancer, vascular tumors, and vulvar cancers.

In some embodiments, the peptide-antibody complex binds to an ion channel, such as potassium or sodium channels. In some embodiments, the peptide-antibody complex blocks potassium channels and/or sodium channels, in some embodiments the peptide-antibody complex activates of potassium channels and/or sodium channels. In some embodiments, the peptide-antibody complex interacts with ion channels or chloride channels or calcium channels. In some embodiments the peptide-antibody complex interacts with nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors. In some embodiments, the peptide-antibody complex interacts with matrix metalloproteinase, inhibits cancer cell migration or metastases, or has antitumor activity. In some embodiments, the peptide-antibody complex interacts with calcium activated potassium channels. In some embodiments, the peptide-antibody complex has antibacterial, antifungal, or antiviral activity. In some embodiments, the peptide-antibody complex inhibits proteases. In some embodiments, the peptide-antibody complex interacts with channels that influence pain. In some embodiments, the peptide-antibody complex has other therapeutic effects on the tissue of an effected organ or structures thereof.

In some embodiments, peptide-antibody complexes of the present disclosure exhibit protease inhibitor activity. In certain embodiments, peptide-antibody complexes are used to inhibit proteases of interest, such as coagulation-associated proteases (e.g., thrombin, factor 10a), metabolism-associated proteases (e.g., DPP-IV), cancer-associated proteases (e.g., matrix metalloproteinases, cathepsins), viral infection-associated proteases (e.g., HIV protease), and inflammation-associated proteases (e.g., tryptase, kallikrein).

In some embodiments, the peptide-antibody complexes of the present disclosure can be modified to be anti-inflammatory, such as by incorporating properties of Immune Selective Anti-Inflammatory Derivatives (ImSAIDs). In certain embodiments, ImSAIDs are incorporated into or added onto peptide-antibody complexes capable of targeting cancerous cells as described herein. FEG is an example of a key sequence that confers anti-inflammatory properties. Alternatively or in combination, peptide-antibody complexes of the present disclosure can be further conjugated to immune regulatory molecules to reverse, reduce, or limit inflammation.

In some embodiments, the peptide-antibody complexes of the present disclosure are used to treat cancers. For example, in certain embodiments, the peptide-antibody complexes provided herein are used to directly inhibit critical cancer-associated pathways such as RAS, MYC, PHF5A, BubR1, PKMYT1, or BuGZ.

In some aspects, the peptide-antibody complexes of the present disclosure involve one or more therapeutic agents, or is further conjugated to one or more therapeutic agents. In certain aspects, the therapeutic agent is a chemotherapeutic, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. For example, in certain embodiments, a peptide-antibody complex of the present disclosure is conjugated to palbociclib, a CDK 4/6 inhibitor with limited ability to cross the blood brain barrier. As another example, in certain embodiments, a peptide-antibody complex of the present disclosure is conjugated to monomethyl auristatine E (MMAE), MMAF, auristatin, dolostatin, auristatin F, monomethylauristatin D, maytansinoid (e.g., DM-1, DM4, maytansine), pyrrolobenzodiazapine dimer, calicheamicin, N-acetyl-y-calicheamicin, duocarmycin, anthracycline, a microtubule inhibitor, or a DNA damaging agent.

Optionally, certain embodiments of the present disclosure provide peptide-antibody complexes conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cancer cells using both a therapeutic agent (e.g., drug) and electromagnetic energy (e.g., radiation or light) concurrently.

In certain embodiments, the peptide-antibody complexes of the disclosure are mutated to home, distribute to, target, migrate to, accumulate in, or is directed to certain tissues but not to others, to change the strength or specificity of its function, or to gain or lose function, such as agonizing an ion channel or inhibiting a protease.

The present disclosure also encompasses the use of “tandem” peptides in which two or more peptides are conjugated or fused to one or more antibodies. In certain embodiments, a peptide-antibody complex comprises two or more knotted peptides conjugated or fused together, where at least one knotted peptide is capable of targeting to a specific region, while at least one other knotted peptide provides a specific therapeutic activity, such as a BIM analogue, as discussed above and herein.

In some embodiments, the present disclosure provides a method for treating a cancer, the method comprising administering to a subject in need thereof an effective amount of a peptide-antibody complex of the present disclosure.

In some embodiments, the present disclosure provides a method for treating a cancer, the method comprising administering to a patient in need thereof an effective amount of a pharmaceutical composition comprising a peptide-antibody complex of the present disclosure and a pharmaceutically acceptable carrier.

In some embodiments, the present disclosure provides a method for inhibiting invasive activity of cells, the method comprising administering an effective amount of a peptide-antibody complex of the present disclosure to a subject.

A peptide-antibody complex comprising the sequence of any of SEQ ID NO: 1-SEQ ID NO: 426, and any peptide derivative or peptide-active agent as described herein, can be used to target upper GI disease and cancers (e.g., throat, oral, esophageal cancer, salivary glands, tonsils, pharynx, adenosarcomas, oral malignant melanoma head and neck cancer). A peptide-antibody complex comprising the sequence of any of SEQ ID NO: 1-SEQ ID NO: 426, and any derivative thereof fused to or conjugated an antibody, an antibody fragment, or a derivative thereof, can be used to additionally target gall bladder disease and cancers.

Venom or toxin derived peptide(s), peptides, modified peptides, labeled peptides, peptide-antibody complexes and pharmaceutical compositions described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Such peptide-antibody complexes described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the calculations of the treating physician.

In some embodiments, the present disclosure provides a method of treating a tumor or cancerous cells of a subject, the method comprising administering to the subject a peptide-antibody complex comprising the sequence of any of SEQ ID NO: 1-SEQ ID NO: 426, or SEQ ID NO: 1, SEQ ID NO: 2, SEQ ID NO: 3, SEQ ID NO: 124, SEQ ID NO: 128, SEQ ID NO: 132, SEQ ID NO: 141, SEQ ID NO: 147, SEQ ID NO: 149, SEQ ID NO: 161, SEQ ID NO: 179, SEQ ID NO: 215, SEQ ID NO: 216, SEQ ID NO: 345, and SEQ ID NO: 374 or a functional fragment thereof, conjugated or fused to an antibody, or a fragment, derivative, or variant thereof. Multiple peptide-antibody complexes described herein can be administered in any order or simultaneously.

Peptide-antibody fusions or conjugates can be packaged as a kit. In some embodiments, a kit includes written instructions on the use or administration of the peptide-antibody fusions or conjugates.

### Treatment of Brain Tumors and Other Brain Diseases and Disorders

In one embodiment, the method includes administering an effective amount of a peptide-antibody complex of the present disclosure to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide-antibody complex of the disclosure. The disease may be a brain or spinal cord disease. In treating the disease, the peptide-antibody complex may cross the blood brain barrier or blood cerebrospinal fluid barrier of a subject. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment may be provided to the subject before clinical onset of disease. Treatment may be provided to the subject after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition, such as one or more of the pharmaceutical compositions described throughout the disclosure. A treatment can comprise delivering a peptide-antibody complex of the disclosure to a subject, either intravenously, subcutaneously, intramuscularly, by inhalation, dermally, topically, orally, sublingually, intrathecally, transdermally, intranasally, via a peritoneal route, or directly into the brain, e.g., via and intracerebral ventrical route.

The activity of a plurality of brain regions, tissues, structures or cells can be modulated by a peptide-antibody complex of the disclosure. Some of the brain regions, tissues, structures include: a) the cerebrum, including cerebral cortex, basal ganglia (striatum), and olfactory bulb; b) the cerebellum, including dentate nucleus, interposed nucleus, fastigial nucleus, and vestibular nuclei; c) diencephalon, including thalamus, hypothalamus, and the posterior portion of the pituitary grand; and d) the brain-stem, including pons, substantia nigra, medulla oblongata; e) the temporal lobe, including the hippocampus and the dentate gyrus (including the subgranular zone); f) the ventricular system, including the lateral ventricles (right and left ventricles), third ventricle, fourth ventricle, intraventricular foramina, cerebral aqueduct, median aperture, right and left lateral apertures, choroid plexus, and the subventricular zone; g) the CSF and associated tissues, including the subarachnoid space, cisterns, sulci; h) the meninges, including the dura mater, arachnoid mater, and pia mater; i) the rostral migratory stream; j) neural stem cells, neural progenitor cells, and new neural cells; and k) any cells or cell types in (a)-(j) above. In some embodiments, the peptide-antibody complexes of the present disclosure are capable of crossing the BBB or blood CSF barrier and accumulating in one or more specific brain regions, tissue, structures, or cells. For example, in certain embodiments, the peptide-antibody complexes described herein home, target, are directed to, migrate to, or accumulate in the hippocampus, the CSF, the ventricular system, the meninges, or the rostral migratory stream, or combinations thereof.

In some embodiments, the present disclosure provides a method for treating a brain disease or condition, the method comprising administering to a subject in need thereof an effective amount of a peptide-antibody complex of the present disclosure. A brain disease or condition can be any neurodegenerative disease or lysosomal storage disease. A neurodegenerative disease can be any disease, state, or condition relating to the loss of structure or function of the central nervous system, including any disease, state or condition relating to the loss of structure or function of the central nervous system, including without limitation Alzheimer's disease, Parkinson's disease, Huntington's disease, Amyotrophic Lateral Sclerosis, Frontotemporal Dementia, Progressive Supranuclear Palsy, multiple system atropy (MSA), Corticobasal Degeneration, Amyotrophic lateral sclerosis (ALS or Lou Gehrig's Disease), dementia, multiple sclerosis, meningitis, epilepsy, and prion diseases, such as transmissible spongiform encephalopathies (TSEs) (e.g., Creutzfeldt-Jakob disease (CJD), bovine spongiform encephalopathies (BSE) and the like).

A lysosomal storage disease can be any disease, state, or condition relating to defects in lysosomal function, including, without limitation, Krabbe disease, Gaucher disease, Tay-Sachs disease, Niemann-Pick disease, Pompe disease, Hurler syndrome, and Hunter syndrome, Sphingolipidoses, Ceramidase, Farber disease, Krabbe disease, Galactosialidosis, Gangliosides, Fabry disease, Schindler disease, Beta-galactosidase/GM1 gangliosidosis, GM2 Gangliosidosis, Sandhoff disease, Tay-sachs, Gaucher disease, Sphingomyelinase, Lysosomal acid lipase deficiency, Nieman-pick disease, Sulfatisdosis, Metachromatic leukodystrophy, Multiple sulfatase deficiency, Mucopolysaccharidose, Mucolipidoses, Lipidoses, Neuronal ceroid lipofuscinoses, Wolman disease, Alpha-mannosidosis, Aspartylglucosaminuria, Fucosidosis, Lysosomal transport diseases, Cystinosis, Pycnodysostosis, Salla disease, Sialic Acid Storage Disease, Infantile free sialic acid storage disease, Glycogen storage disease, Chloesteryl ester storage disease.

Brain disease due to the automimmune system or due to an infection can be any disease, state, or condition relating to defects in the autoimmune system including autoimmune brain disorders (ABD), multiple sclerosis, and System Lupus Erythematosus. Brain disease due to an infection can be any disease, state, or condition relating to an infection including meningitis, brain abscess, neuromyelitis optica, late-stage neurological trypanosomiasis, HIV encephalitis, Hashimoto's thyroiditis, prion and prion-like disease, rabies, and systemic inflammation.

Further examples of brain diseases or disorder that can be treated with peptide-antibody complexes of the disclosure include Acoustic Neuroma (Vestibular Schwannoma), Acute Subdural Hematomas, Addictions (e.g., alcoholism, drug addiction, nicotine or tobacco, etc.), Alzheimer's disease, Amyotrophic Lateral Sclerosis (ALS, or Lou Gehrig's Disease), Anaplastic Astrocytoma (AA), Anxiety and related disorders, Anorexia, Antisocial Personality Disorder, Aqueductal Stenosis, Arachnoid Cysts, Arnold Chiari Malformation, Arteriovenous Malformation (AVM), Astrocytoma, Autism, Ballism, bipolar disorders, Brain Aneurysm, Autism, Brain Attack, Brain Metastases, Brainstem Glioma, Bulimia, Carotid Stenosis, Catastrophic Epilepsy in Children, Cavernous Angioma, Cerebral Aneurysms, Cerebral Contusion and Intracerebral Hematoma, Cerebral Edema, Cerebral Hemorrhage, Chiari Malformation, Chordomas, Chorea, Choroid Plexus Cyst, Chronic Subdural Hematomas, Colloid Cyst, Coma, Concussion, Cranial Gun Shot Wounds, Corticobasal Degeneration, Craniopharyngioma, Craniosynostosis, Cushing's Disease, Cyst (Epidermoid Tumor), Dementia, Depression and related disorders, eating disorders, weight loss and satiety, Diabetes, Dravet Syndrome, Encephalopathy, Ependymoma, Epilepsy, Epidural Hematomas Epilepsy, Essential Tremor, Extratemporal Lobe Epilepsies, Facet Joint Syndrome, Frontotemporal Dementia, Ganglioglioma, Gaucher disease, Germinoma, Glioblastoma Multiforme (GBM), Glioma, Glomus Jugulare Tumor, Glossopharyngeal Neuralgia, Hemangioblastomas, Hemi-Facial Spasm, Hydrocephalus, Huntington's disease, immune system disorders, Intracerebral Hemorrhage, Hurler syndrome, Hunter syndrome, Intracranial Hypotension, JPA (Juvenile Pilocytic Astrocytoma), Krabbe disease, Lennox-Gestaut Syndrome, Lipomyelomeningocele, Low-Grade Astocytoma (LGA), Lymphocytic Hypophysitis, Lymphoma, Medulloblastoma, Meningioma, Meningitis, Mesial Temporal Lobe Epilepsy, Metastatic Brain Tumors, Migraine, Mitochondrial Disease, Moyamoya Disease, multiple sclerosis, Multiple system atrophy (MSA), Niemann-Pick disease, Nelson's Syndrome, Neurocysticercosis, Neurodegenerative Disorders, Neurofibroma, neuropathic pain, Nonfunctional Pituitary Adenoma, Normal Pressure Hydrocephalus, obsessive-compulsive disorders, Oligodendroglioma, Optic Nerve Glioma, Osteomyelitis, Parkinson's disease, Paranoia and related disorders, Pediatric Hydrocephalus, Phantom Limb Pain, Pilocytic Astrocytoma, Pineal Tumor, Pineoblastoma, Pineocytoma, Pituitary Adenoma (Tumor), Pituitary Apoplexy, Pituitary Failure, Pompe disease, Postherpetic Neuralgia, Post-Traumatic Seizures, Post-Traumatic Stress Disorder, Primary CNS Lymphoma, Prolactinoma, Pseudotumor Cerebri, Progressive Supranuclear Palsy, Rathke's Cleft Cyst, Recurrent Adenomas, Rheumatoid Arthritis, Schizophrenia, Schwannomas, Scoliosis, Skull Fracture, Slit Ventricle Syndrome, Spasticity, Spontaneous Intracranial Hypotension, Stroke (Brain Attack, TIA), Subarachnoid Hemorrhage, Syrinx, Tay-Sachs disease, Thyrotroph (TSH) Secreting Adenomas, Torticollis, Transient Ischemic Attacks (TIA), Traumatic Brain Injury, Traumatic Hematomas, Trigeminal Neuralgia, Ventriculitis, Vestibular Schwannoma, depression, mood disorders, lysosomal storage diseases, memory disorders, learning disorders, disorders of spatial memory or navigation, stress-related disorders, post-traumatic stress disorder, pain, aging, hippocampal atrophy, brain infections including fungal infections and progressive Multifocal Leukoencepalopathy, or another brain disease or disorder. In other cases, a peptide-antibody complex of the disclosure can be used to treat alcoholism, cigarette addiction, drug addiction, or anxiety.

In some embodiments, the peptide-antibody complex binds to potassium channels in the brain. In some embodiments the peptide-antibody complex binds to sodium channels in the brain. In some embodiments, the peptide-antibody complex blocks potassium channels and/or sodium channels, in some embodiments the peptide-antibody complex activates of potassium channels and/or sodium channels. In some embodiments, a peptide-antibody complex interacts with ion channels or chloride channels or calcium channels. In some embodiments the peptide-antibody complex interacts with nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors. In some embodiments, the peptide-antibody complex interacts with matrix metalloproteinase, inhibits cancer cell migration or metastases, or has antitumor activity. In some embodiments, the peptide-antibody complex interacts with calcium activated potassium channels. In some embodiments, the peptide-antibody complex has antibacterial, antifungal, or antiviral activity. In some embodiments, the peptide-antibody complex inhibits proteases. In some embodiments, the peptide-antibody complex interacts with channels that influence pain. In some embodiments, the peptide-antibody complex has other therapeutic effects on the brain or structures thereof.

In some embodiments, the peptide-antibody fusions of the present disclosure are used to diagnose or treat a disease or disorder associated with the hippocampus. The hippocampus is a critical brain structure involved in learning, memory, mood, and cognition. Changes in the hippocampus, including reduced volume and cellularity, reduced neuronal density, and defects in neurotransmitter function, are associated with initiation, persistence, and/or progression of disorders including late-life depression (Taylor); major depression and bipolar disorder (Drevets); post-traumatic stress disorder (PTSD) (Schmidt); Alzheimer disease (Nava-Mesa); and schizophrenia (Perez). Peptide-antibody fusions of the current invention that target the hippocampus can be used to treat these diseases or to target therapeutically-active substances to treat these diseases amongst others. In some embodiments, the peptide-antibody fusions are used to treat these diseases by acting on receptors such as GABA, NMDA, AMPA, dopamine, or serotonin receptors. The dentate gyrus in the hippocampus can also be a site of neurogenesis.

In some embodiments, the peptide-antibody complexes of the present disclosure are used to diagnose or treat a disease or condition associated with the CSF or ventricular system. The CSF is a fluid that surrounds and circulates in the brain and spine that provides mechanical protection for the brain and plays a role in the homeostasis and metabolism of the central nervous system. CSF is produced by and circulated within the ventricular system. Diseases and conditions that are associated with the CSF or ventricular system include but are not limited to: antisocial personality disorder, cerebral hemorrhage, choroid plexus cyst, dementia, ependymoma, hydrocephalus, meningitis, multiple system atrophy (MSA), neurodegenerative disease (such as amyotrophic lateral sclerosis, Parkinson's disease, Alzheimer's disease, Huntington's disease) post-traumatic stress disorder, schizophrenia, subarachnoid hemorrhage, traumatic brain injury, and ventriculitis.

Peptide-antibody complexes of the current disclosure that target the CSF or ventricular system can be used to treat these diseases or to target therapeutically-active substances to treat these diseases, amongst others. For example, in certain embodiments, the peptide-antibody complexes of the present disclosure are used to modulate targets associated with a disease, such as mitochondrial deubiquitinase USP30 (e.g., for the treatment of Parkinsons' disease) or dual leucine zipper kinase (e.g., for the treatment of neurodegeneration). As another example, in certain embodiments, the peptide-antibody complexs are further conjugated to a therapeutic agent used to treat a neurodegenerative disease, such as Alzheimer's disease. Such drugs could also include galantamine, donzepil, tacrine, or even neurotoxins generally thought to be too toxic, such as sarin. Examples of therapeutic agents useful for treating neurodegenerative disease include but are not limited to: acetylcholinesterase inhibitors (e.g., rivastigimine), galantamine, donzepil, tacrine, and neurotoxins (e.g., sarin). This approach allows for treatment with lower dosages and reduced side effects in the periphery, compared to prior methods which utilize untargeted systemic delivery. In yet another example, in certain embodiments, peptide-antibody complexes that home, distribute to, target, migrate to, accumulate in, or are directed to the ventricular space are used as radioprotectant (e.g., alone or as a conjugate to a radioprotective compound such as amifostine) during treatment of brain metastases with radiation.

In some embodiments, the peptide-antibody complexes of the present disclosure are used to inhibit small-conductance, calcium-activated potassium channels (SK channels). Peptide-antibody complex that inhibit SK channels include members of the Toxin_6 class, for example. Optionally, such peptide-antibody complexes may exhibit homing to specific brain regions, such as the ventricles. In certain embodiments, the peptide-antibody complexes of the present disclosure have specificity for one or more SK channel subtypes, such as one or more of the SK1, SK2, SK3, or SK4 channel subtypes. In certain embodiments, inhibition of the SK3 subtype increases the frequency of firing in dopaminergic neurons, thus increasing levels of dopamine, which may ameliorate the physical symptoms of Parkinson's disease.

In some embodiments, the peptide-antibody complexes of the present disclosure are used to affect (e.g., reduce, slow, or inhibit) the aggregation of proteins associated with neurodegenerative disease, such as tau, prion protein, amyloid beta, alpha synuclein, parkinin, or huntingtin.

In some embodiments, the peptide-antibody complexes of the present disclosure are used to inhibit or activate one or more specific ion channels, and the inhibition or activation of the ion channels alleviates the symptoms of a range of diseases. TABLE 5 illustrates exemplary ion channels and associated diseases that may be treated in accordance with the compositions and methods presented herein.

TABLE 5
Exemplary Ion Channels and Associated Diseases
According to the Present Disclosure.

| Ion | | Gain (G) or | |
|---|---|---|---|
| Channel | | Loss (L) of | |
| Family | Channel | Function | Disease |
| Kᵢᵣ | Kᵢᵣ1.1 | L | Bartter's syndrome |
| Kᵢᵣ | Kᵢᵣ2.1 | L | Andersen's syndrome |
| Kᵥ | Kᵢᵣ6.2 | L | congenital hyperinsulinism |
| | Kᵢᵣ6.2 | G | neonatal diabetes |
| | SUR2 | L | dilated cardiomyopathy |
| | Kᵥ1.1 | L | episodic ataxia type 1 |
| Kᵥ | KCNQ1 | L | long QT syndrome |
| TRP | KCNQ1 | G | short QT syndrom |
| | KCNQ2 | L | benign neonatal febrile convulsions |
| | KCNQ4 | L | nonsyndromic deafness |
| | hERG | L | long QT syndrome |
| | hERG | G | short QT syndrome |
| | TRPP2 | | polycystic kidney disease |
| TRP | TRPA1 | G | familial episodic pain syndrome |
| CNG | TRPC6 | G | focal segmental glomerulosclerosis |
| | CNGA1 | L | retinitis pigmentosa |
| K_{Ca} | BK | G | epilepsy |
| NAᵥ | NAᵥ1.1 | G | epilepsy |
| NAᵥ | NAᵥ1.1 | L | severe myoclonic epilepsy |
| Caᵥ | NAᵥ1.5 | G | long QT syndrome |
| | NAᵥ1.6 | L | cerebellar ataxia |
| | NAᵥ1.7 | G | erythromelalgia, paroxysmal |
| | | | extreme pain disorder |
| | NAᵥ1.7 | L | congenital indifference to pain |
| | NAᵥ2.1 | G | benign familial neonatal seizures |
| | Caᵥ1.2 | G | timothy syndrome |
| | Caᵥ2.1 | L | episodic ataxia type 2 |
| glycine | GLRA1 | L | stiff baby syndrome |
| receptors | | | |
| GABA | GABA_{A} | L | juvenile myoclonic epilepsy |
| AChR | CHRNA4 | L | autosomal dominant nocturnal |
| | | | frontal lobe epilepsy |

In some embodiments, the peptide-antibody complexes of the present disclosure exhibit protease inhibitor activity. In certain embodiments, peptide-antibody complexes capable of crossing the BBB are used to inhibit Alzheimer's associated proteases such as beta and gamma secretase. In alternative embodiments, peptide-antibody complexes that may or may not be capable of crossing the BBB are used to inhibit other proteases of interest, such as coagulation-associated proteases (e.g., thrombin, factor 10a), metabolism-associated proteases (e.g., DPP-IV), cancer-associated proteases (e.g., matrix metalloproteinases, cathepsins), viral infection-associated proteases (e.g., HIV protease), and inflammation-associated proteases (e.g., tryptase, kallikrein).

In some embodiments, the peptide-antibody complexe of the present disclosure can be modified to be anti-inflammatory, such as by incorporating properties of Immune Selective Anti-Inflammatory Derivatives (ImSAIDs). In certain embodiments, ImSAIDs are incorporated into or added onto peptide-antibody complexes capable of targeting specific brain regions as described herein. FEG is an example of a key sequence that confers anti-inflammatory properties. Alternatively or in combination, peptide-antibody complexes of the present disclosure can be conjugated to immune regulatory molecules to reverse, reduce, or limit inflammation.

In some aspects, the peptide-antibody complexes of the present disclosure are further conjugated to one or more therapeutic agents. In certain embodiments, the peptide-antibody complexes described herein are used as conjugates to deliver therapeutic agents across the BBB or blood CSF barrier and optionally into specific regions, tissues, structures, or cells in the brain. Examples of such therapeutic agents include anti-inflammatory molecules (e.g., dexamethasone, prednisone, prednisolone, methyl prednisolone, or traimcinolone), antifungal agents (e.g., fluconazole, amphotericin B, ketoconazole, or abafungin), antiviral agents (e.g., acyclovir, cidofovir), growth factors (e.g., NGF or EGF), or anti-infective agents (e.g., ciprofloxacin, tetracycline, erythromycin, or streptomycin). For instance, in certain embodiments, peptide-antibody complex of the present disclosure is conjugated to an antifungal agent in order to treat a fungal infection of the brain, which is otherwise highly difficult to treat using prior methods and compositions. As another example, in certain embodiments, a BBB-penetrating peptide-antibody complex of the present disclosure is conjugated to cidofovir in order to treat progressive multifocal leucoencephalopathy (PML) caused by the JC virus, which otherwise has no reliable treatment.

In some embodiments, the peptide-antibody complexes of the present disclosure are used to treat brain cancer. For example, in certain embodiments, the peptide-antibody complexes provided herein are used to directly inhibit critical cancer-associated pathways such as RAS, MYC, PHF5A, BubR1, PKMYT1, or BuGZ. Alternatively or in combination, the peptide-antibody complexes of the present disclosure are used to carry a conjugated therapeutic agent across the BBB in order to treat brain cancer.

In further aspects, the therapeutic agent is a chemotherapeutic agent, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. For example, in certain embodiments, a peptide-antibody complex of the present disclosure is conjugated to palbociclib, a CDK 4/6 inhibitor with limited ability to cross the BBB. As another example, in certain embodiments, a peptide-antibody complex of the present disclosure is conjugated to monomethyl auristatine E (MMAE), MMAF, an auristatin, dolostatin, auristatin F, monomethylauristatin D, a maytansinoid (e.g., DM-1, DM4, maytansine), a pyrrolobenzodiazapine dimer, N-acetyl-y-calicheamicin, a calicheamicin, a duocarmycin, an anthracycline, a microtubule inhibitor, or a DNA damaging agent.

Optionally, certain embodiments of the present disclosure provide peptide-antibody complexes further conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cancer cells using both a therapeutic agent (e.g., drug) and electromagnetic energy (e.g., radiation or light) concurrently.

In certain embodiments, the peptide-antibody complex of the disclosure is mutated to retain ability to cross the BBB or the blood CSF barrier and home, distribute to, target, migrate to, accumulate in, or are directed to certain tissues, but to gain or lose function, such as agonizing an ion channel or inhibiting a protease. In other embodiments, the peptide-antibody complex of the disclosure is mutated to home, distribute to, target, migrate to, accumulate in, or is directed to certain tissues but not others, to change the strength or specificity of its function, or to gain or lose function.

In some embodiments, the present disclosure provides a method for treating a cancer, the method comprising administering to a subject in need thereof an effective amount of a peptide-antibody complex of the present disclosure.

In some embodiments, the present disclosure provides a method for treating a cancer, the method comprising administering to a patient in need thereof an effective amount of a pharmaceutical composition comprising a peptide-antibody complex of the present disclosure and a pharmaceutically acceptable carrier.

In some embodiments, the present disclosure provides a method for inhibiting invasive activity of cells, the method comprising administering an effective amount of a peptide-antibody complex of the present disclosure to a subject.

In some aspects, the present disclosure provides a method for detecting a cancer, cancerous tissue, or tumor tissue, the method comprising the steps of contacting a tissue of interest with a peptide-antibody complex of the present disclosure, wherein the peptide-antibody complex is conjugated to a detectable agent and measuring the level of binding of the peptide-antibody complex, wherein an elevated level of binding, relative to normal tissue, is indicative that the tissue is a cancer, cancerous tissue or tumor tissue.

The present invention provides methods for intraoperative imaging and resection of a cancer, cancerous tissue, or tumor tissue using a peptide-antibody complex of the present disclosure conjugated with a detectable agent. In some aspects, the cancer, cancerous tissue, or tumor tissue is detectable by fluorescence imaging that allows for intraoperative visualization of the cancer, cancerous tissue, or tumor tissue using a peptide-antibody complex of the present disclosure. In some aspects, the peptide-antibody complex of the present disclosure is conjugated to one or more detectable agents. In a further embodiment, the detectable agent comprises a fluorescent moiety coupled to the peptide-antibody complex. In another embodiment, the detectable agent comprises a radionuclide. In some aspects, imaging is achieved using open surgery. In further aspects, imaging is accomplished using endoscopy or other non-invasive surgical techniques.

In some cases, the peptide-antibody complex can be used to target cancer in the brain by crossing the BBB or blood CSF barrier and then having antitumor function, targeted toxicity, inhibiting metastases, etc. In other cases, the peptide-antibody complex can be used to label, detect, or image such brain lesions, including tumors and metastases amongst other lesions, which may be removed through various surgical techniques.

In addition, certain peptide-antibody complexes of the disclosure can have additional applicability in diseases and conditions outside the brain. A peptide-antibody complex comprising the sequence of any of SEQ ID NO: 1-SEQ ID NO: 426, and any antibody as described herein, e.g., TABLE 4, can be used to additionally target upper GI disease and cancers (e.g., throat, oral, esophageal cancer, salivary glands, tonsils, pharynx, adenosarcomas, oral malignant melanoma, head and neck cancer), gall bladder disease and cancer. Venom or toxin derived peptide-antibody complexes and pharmaceutical compositions thereof as described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Such complexes described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the calculations of the treating physician.

In some embodiments, the present disclosure provides a method of treating a brain disorder of a subject, the method comprising administering to the subject a peptide-antibody complex comprising the sequence of any of SEQ ID NO: 1-SEQ ID NO: 426, or a functional fragment thereof, conjugated to an antibody described herein.

### EXAMPLES

The following examples are included to further describe some aspects of the present disclosure, and should not be used to limit the scope of the invention.

### Example 1

### Method of Manufacturing/Purification of a Peptide

This example describes the method of manufacturing and purifying a peptide. FIG. 1 shows a schematic, in one embodiment, of making a recombinant peptide, e.g., SEQ ID NO: 1, using virus, e.g., lentivirus, to transduces cells, e.g., HEK293 cells, with an expression vector comprising a sequence encoding amino acids of the peptide. After viral production, transduction, and cell culture growth, cell cultures are harvested for isolation and purification of the peptide using affinity chromatography and size exclusion chromatography followed by storage at 4° C.

Once purified, such peptide can be linked or conjugated to a purified antibody to make a peptide-antibody conjugate as described herein.

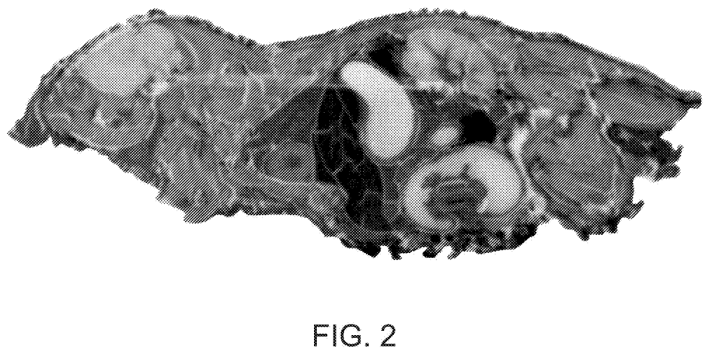

FIG. 2 illustrates the ability of SEQ ID NO: 1 to enter the CNS, showing the distribution of ¹⁴C radiolabeled SEQ ID NO: 1 in the brain and various other tissues.

### Example 2

### Purification of an Anti-BACE1 Peptide-Antibody Fusion

This example describes the treatment of Alzheimer's disease in a subject using a peptide-antibody fusion. The C-terminus of human anti-BACE1 antibody heavy chain (SEQ ID NO: 427) and/or the light chain (SEQ ID NO: 428) (TABLE 7) were fused to peptide SEQ ID NO. 1, wherein the antibody was fused to the N-terminus of the peptide sequence, giving antibody-peptide fusions of SEQ ID NO: 429 and SEQ ID NO: 430 (TABLE 6). J. K. Atwal et al., A Therapeutic Antibody Targeting BACE1 Inhibits Amyloid-Production in Vivo. Science Translational Medicine. 3, 84ra43-84ra43 (2011).

SEQ ID NO: 1 was reverse-translated into DNA, synthesized, and cloned in-frame with either the antibody heavy (SEQ ID NO: 427) or light chain (SEQ ID NO: 428) using standard molecular biology techniques. (M. R. Green, Joseph Sambrook. Molecular Cloning. 2012 Cold Spring Harbor Press). The resulting construct was packaged into a lentivirus, transduced into HEK293 cells with the partner heavy or light chain, expanded, isolated by immobilized affinity chromatography, and purified to homogeneity using size exclusion chromatography (SEC) using media supplemented with amino acid. Following purification, the peptide-anti-BACE1 antibody fusions were stored at 4° C.

A linker sequence of GGGSGGGS (SEQ ID NO: 444) was cloned between the antibody and the peptide. The resulting peptide-antibody fusions are SEQ ID NO: 429, wherein the antibody heavy chain was fused to the peptide, and SEQ ID NO: 430, wherein the antibody light chain was fused to SEQ ID NO: 1. The heavy chain and the light chain were expressed together with the peptide fused to each component of the antibody. After purification of the heavy chain and the light chain peptide fusions, they were assembled in vitro to form the full antibody and peptide fusion protein complex.

FIG. 3 shows the size exclusion chromatograph of antibody fusion composed of SEQ ID NO: 428 and SEQ ID NO: 429 as a purified intact antibody of the expected molecular weight. Gel inset shows the purified antibody fusion composed of SEQ ID NO: 428 and SEQ ID NO: 429 under non-reducing conditions.

In some embodiments, the heavy chain and the light chains can be co-expressed, allow purification of an intact or assembled peptide-antibody fusion complex. In some cases, four peptides can be complexed to an antibody. For embodiments wherein the heavy chain and the light chain are assembled after expression or purification, the assembled antibody would be further purified using affinity purification, such as affinity purification column with the antibody's target immobilized on a column, or other methods. The purified antibody, comprising the heavy chains and the light chains, can then be used for therapeutics or for formulation as a therapeutic agent.

### Example 3

### Bioactivity of Anti-BACE1 Peptide-Antibody Complexes

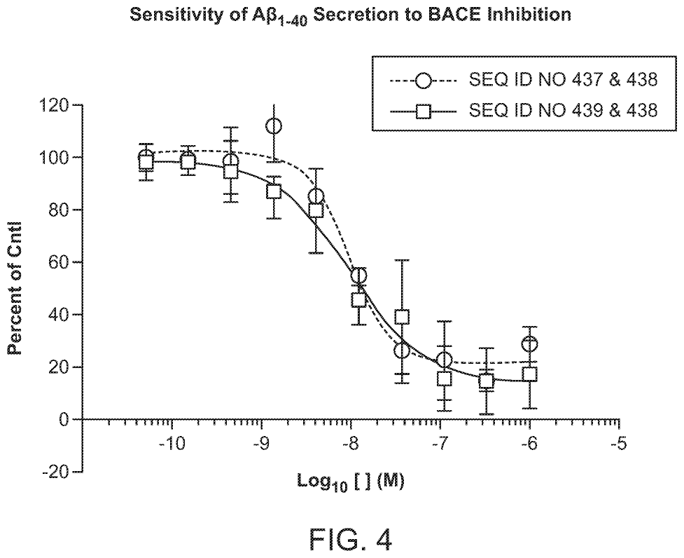

This example describes the bioactivity of an anti-BACE1 peptide-antibody complex. FIG. 4 illustrates amyloid β-protein (Aβ₁₋₄₀) secretion in response to inhibition of BACE1 by an anti-BACE1 peptide-antibody complex of the present disclosure. Bioactivity of the anti-BACE1 peptide-antibody complex was compared to the anti-BACE1 antibody itself. A peptide of SEQ ID NO: 1 was fused to an anti-BACE1 antibody heavy chain of SEQ ID NO: 427 to give a peptide-heavy chain fusion of SEQ ID NO: 429. This peptide-heavy chain fusion was further assembled with an anti-BACE1 antibody light chain of SEQ ID NO: 428 to give the anti-BACE1 peptide-antibody complex. Bioactivity of the anti-BACE1 peptide-antibody complex was compared to an anti-BACE1 antibody comprising an anti-BACE1 antibody heavy chain of SEQ ID NO: 427 and an anti-BACE1 antibody light chain of SEQ ID NO: 428. The peptide-heavy chain fusion of SEQ ID NO: 429 was produced and purified with the anti-BACE1 antibody light chain of SEQ ID NO: 428 and incubated with HEK/hAPP, which is the human embryonic kidney (HEK) cell line expressing the amyloid precursor protein (APP). As a control, HEK/hAPP cells were also incubated with the anti-BACE1 antibody comprising SEQ ID NO: 428 and SEQ ID NO: 427, which together formed the anti-BACE1 antibody without the peptide. The incubation was for 24 hours at 37° C.

In both experimental and control conditions, hAPP expressed by the cells was cleaved by endogenous BACE-1 enzyme resulting in fragments including Aβ1-40. An HTRF assay was used to measure Aβ1-40 processed and released from the cells into the culture supernatants. Levels were quantified in the presence of the peptide-antibody complex or the control antibody. The secretion profile of Aβ1-40 decreased with increasing amounts of the peptide-antibody complex, indicating that the bioactivity of the control anti-BACE1 antibody was comparable with the bioactivity of the peptide-antibody complex.

Peptide-antibody complexes manufactured and tested as described above include peptide-antibody complexes of SEQ ID NO: 429 and SEQ ID NO: 428 or SEQ ID NO: 430 and SEQ ID NO: 427. These peptide-antibody complexes of SEQ ID NO: 429 and SEQ ID NO: 428 or SEQ ID NO: 430 and SEQ ID NO: 427 are administered to a subject to treat Alzheimer's disease.

Similarly, such peptide-antibody complexes including peptides SEQ ID NO: 124, SEQ ID NO: 141, SEQ ID NO: 147, SEQ ID NO: 179, SEQ ID NO: 2, SEQ ID NO: 3, SEQ ID NO: 37, SEQ ID NO: 35, SEQ ID NO: 36, or SEQ ID NO: 39, or SEQ ID NO: 333, SEQ ID NO: 350, SEQ ID NO: 356, SEQ ID NO: 388, SEQ ID NO: 211, SEQ ID NO: 212, SEQ ID NO: 246, SEQ ID NO: 244, SEQ ID NO: 245, or SEQ ID NO: 248 can be administered to a subject to treat Alzheimer's disease.

TABLE 6
SEQ ID NO: 1 and anti-BACE1 Antibody Fusions

| SEQ ID NO: | Description | Amino Acid Sequence |
|---|---|---|
| SEQ ID NO: 429 | Human anti-BACE1 | MEFGLSWVFLVALFRGVQSEVQLVESGG |
| | antibody heavy chain | GLVQPGGSLRLSCAASGFTFLGYGIHWVR |
| | linked to peptide SEQ | QAPGKGLEWVGWISPAGGSTDYADSVKG |
| | ID NO: 1 | RFTISADTSKNTAYLQMNSLRAEDTAVYY |
| | | CARGPFSPWVMDYWGQGTLVTVSSASTK |
| | | GPSVFPLAPSSKSTSGGTAALGCLVKDYF |
| | | PEPVTVSWNSGALTSGVHTFPAVLQSSGL |
| | | YSLSSVVTVPSSSLGTQTYICNVNHKPSNT |
| | | KVDKKVEPKSCDKTHTCPPCPAPELLGGP |
| | | SVFLFPPKPKDTLMISRTPEVTCVVVDVSH |
| | | EDPEVKFNWYVDGVEVHNAKTKPREEQ |
| | | YNSTYRVVSVLTVLHQDWLNGKEYKCK |
| | | VSNKALPAPIEKTISKAKGQPREPQVYTLP |
| | | PSRDELTKNQVSLTCLVKGFYPSDIAVEW |
| | | ESNGQPENNYKTTPPVLDSDGSFFLYSKL |
| | | TVDKSRWQQGNVFSCSVMHEALHNHYT |
| | | QKSLSLSPGKGGGSGGGSMCIPCFTTNPN |
| | | MAAKCNACCGSRRGSCRGPQCIC |
| SEQ ID NO: 430 | Human anti-BACE1 | METDTLLLWVLLLWVPGSTGDIQMTQSP |
| | antibody light chain | SSLSASVGDRVTITCRASQDVSTAVAWYQ |
| | linked to peptide SEQ | QKPGKAPKLLIYSASFLYSGVPSRFSGSGS |
| | ID NO: 1 | GTDFTLTISSLQPEDFATYYCQQFPTYLPT |
| | | FGQGTKVEIKRTVAAPSVFIFPPSDEQLKS |
| | | GTASVVCLLNNFYPREAKVQWKVDNAL |
| | | QSGNSQESVTEQDSKDSTYSLSSTLTLSKA |
| | | DYEKHKVYACEVTHQGLSSPVTKSFNRG |
| | | ECGGGSGGGSMCIPCFTTNPNMAAKCNA |
| | | CCGSRRGSCRGPQCIC |

TABLE 7
Human anti-BACE1 Antibody Chains

| SEQ ID NO: | Description | Amino Acid Sequence |
|---|---|---|
| SEQ ID NO: 427 | Human anti-BACE1 | MEFGLSWVFLVALFRGVQSEVQLVESGG |
| | antibody heavy chain | GLVQPGGSLRLSCAASGFTFLGYGIHWVR |
| | | QAPGKGLEWVGWISPAGGSTDYADSVKG |
| | | RFTISADTSKNTAYLQMNSLRAEDTAVYY |
| | | CARGPFSPWVMDYWGQGTLVTVSSASTK |
| | | GPSVFPLAPSSKSTSGGTAALGCLVKDYF |
| | | PEPVTVSWNSGALTSGVHTFPAVLQSSGL |
| | | YSLSSVVTVPSSSLGTQTYICNVNHKPSNT |
| | | KVDKKVEPKSCDKTHTCPPCPAPELLGGP |
| | | SVFLFPPKPKDTLMISRTPEVTCVVVDVSH |
| | | EDPEVKFNWYVDGVEVHNAKTKPREEQ |
| | | YNSTYRVVSVLTVLHQDWLNGKEYKCK |
| | | VSNKALPAPIEKTISKAKGQPREPQVYTLP |
| | | PSRDELTKNQVSLTCLVKGFYPSDIAVEW |
| | | ESNGQPENNYKTTPPVLDSDGSFFLYSKL |
| | | TVDKSRWQQGNVFSCSVMHEALHNHYT |
| | | QKSLSLSPGK |
| SEQ ID NO: 428 | Human anti-BACE1 | METDTLLLWVLLLWVPGSTGDIQMTQSP |
| | antibody light chain | SSLSASVGDRVTITCRASQDVSTAVAWYQ |
| | | QKPGKAPKLLIYSASFLYSGVPSRFSGSGS |
| | | GTDFTLTISSLQPEDFATYYCQQFPTYLPT |
| | | FGQGTKVEIKRTVAAPSVFIFPPSDEQLKS |
| | | GTASVVCLLNNFYPREAKVQWKVDNAL |
| | | QSGNSQESVTEQDSKDSTYSLSSTLTLSKA |
| | | DYEKHKVYACEVTHQGLSSPVTKSFNRG |
| | | EC |

### Example 4

### Peptide-Antibody Fusions for Targeting Cancerous Cells

This example describes the use of peptide-antibody fusions for treating glioblastoma in a subject. SEQ ID NO: 37 is fused to the anti-cancer antibody, bevacizumab, using linker GGGSGGGS (SEQ ID NO: 444) to treat or target glioblastoma cells. SEQ ID NO: 37 is a tumor homing peptide that can also cross the BBB. The peptide is fused to the heavy chain, the light chain, or both chains, or in multiple locations.

Additional peptide-anti-cancer fusions are prepared using any one of the peptide sequences in TABLE 2, SEQ ID NO: 1-SEQ ID NO: 426, or SEQ ID NO: 1, SEQ ID NO: 2, SEQ ID NO: 141, SEQ ID NO: 179, SEQ ID NO: 35, SEQ ID NO: 36, SEQ ID NO: 39, SEQ ID NO: 244, SEQ ID NO: 245, SEQ ID NO: 248 fused to an anti-cancer antibody described herein, including: antibodies described in TABLE 4, Urelumab, Utomilumab, Ensituximab, Tacatuzumab tetraxetan, Nesvacumab, Vanucizumab, Atezolizumab, Avelumab, Durvalumab, Duligotumab, Elgemtumab, Lumretuzumab, Patritumab, Seribantumab, Pogalizumab, Nerelimomab, Ozoralizumab, Mapatumumab, or Conatumumab, or an antibody that binds to, modulates, or targets any of the following antigens associated with cancer cells: CD20, CD30, CD33, CD52, EpCAM, gpA33, CEA, mucins, TAG-72, carbonic anhydrase IX, PSMA, folate binding protein, gangliosides (e.g., GD2, GD3, GM2), Lewis-Y², VEGF, VEGFR, αVβ3, α5β1, ErbB1/EGFR, ErbB2/HER2, ErbB3, c-MET, IGF1R, EphA3, TRAIL-R1, TRAI-R2, RANKL, FAP, and tenascin, using a linker sequence of GGGSGGGS (SEQ ID NO: 444). The resulting peptide-antibody fusion is administered to a subject to treat glioblastoma

### Example 5

### Peptide-Antibody Fusions for Targeting CNS Disorder

This example describes the use of peptide-antibody fusions for the treatment of Parkinson's disease in a subject. SEQ ID NO: 124, SEQ ID NO: 141, or SEQ ID NO: 179, or SEQ ID NO: 147, SEQ ID NO: 2, SEQ ID NO: 3, SEQ ID NO: 356, SEQ ID NO: 211, SEQ ID NO: 212, SEQ ID NO: 1, SEQ ID NO: 210, SEQ ID NO: 5, SEQ ID NO: 214, SEQ ID NO: 37, SEQ ID NO: 246, SEQ ID NO: 36, SEQ ID NO: 245, which can penetrate the BBB, is fused to an anti-Tau or anti-alpha-synuclein antibody using linker sequence GGGSGGGS (SEQ ID NO: 444). Any one of these peptide sequences can be fused to the heavy chain or the light chain, or both chains of the antibody to yield a peptide-antibody fusion protein that can cross the BBB to block Tau or alpha-synuclein in the CNS. The resulting peptide-antibody fusion is administered to a subject for the treatment of Parkinson's disease.

Additional peptide-antibody fusions for treating Parkinson's disease include those prepared from the peptide sequences in TABLE 2, SEQ ID NO: 1-SEQ ID NO: 426, or SEQ ID NO: 1, SEQ ID NO: 2, SEQ ID NO: 5, SEQ ID NO: 37, SEQ ID NO: 141, or SEQ ID NO: 179 conjugated to an antibody against an antigen, a neuron cell, alpha-synuclein, or a CNS biomarker described herein, including those listed in TABLE 4, using a linker sequence of GGGSGGGS (SEQ ID NO: 444). The resulting peptide-antibody fusions are administered to a subject for the treatment of Parkinson's disease.

### Example 6

### Peptide-Antibody Fusions for Targeting Brain Tumors

This example describes the use of peptide-antibody fusions for treating brain tumor in a subject. SEQ ID NO: 5, SEQ ID NO: 44, or SEQ ID NO: 46, target tumors and can cross the BBB, is fused to nivolumab (anti-PD-1 antibody) using linker sequence GGGSGGGS (SEQ ID NO: 444). The resulting peptide-antibody fusion is administered to a subject for the treatment of a brain tumor.

Additional peptide-antibody fusions for treating brain tumors include those prepared from the peptide sequences in TABLE 2, SEQ ID NO: 1-SEQ ID NO: 426, or SEQ ID NO: 1, SEQ ID NO: 2, SEQ ID NO: 37, SEQ ID NO: 141, or SEQ ID NO: 179 fused to an anti-brain cancer antibody, or an anti-brain tumor biomarker described herein, including those listed in TABLE 4, or an antibody to a checkpoint inhibitor such as PD-L1, PD-1, or CTLA4, or a cytokine such as interleukin-2, or an interferon, or alemtuzumab, atezolizumab, ipilimumab, ofatumumab, nivolumab, pembrolizumab, rituximab, alemtuzumab, amphiphysin, or an antibody that recognizes any of the follows target molecules in the brain: CV2/CRMP5, GAD65, GAD67, gephyrin, Hu(ANNA1), Ma, recoverin, Ri/ANNA2, Tr/PCA1, Yo/PCA1, AMPAR, mGluR1, mGluR2, and mGluR5, GABA, NMDAR, NR2A, NR2B, and Caspr1, using a linker sequence of GGGSGGGS (SEQ ID NO: 444). The resulting peptide-antibody fusions are administered to a subject for the treatment of a brain tumor.

### Example 7

### Treatment of Glioblastoma Using Peptide-Antibody Fusions

This example describes the use of peptide-antibody fusions for treating glioblastoma in a subject. SEQ ID NO: 1, SEQ ID NO: 5, SEQ ID NO: 37, SEQ ID NO: 46, SEQ ID NO: 124, SEQ ID NO: 333, SEQ ID NO: 141, SEQ ID NO: 350, SEQ ID NO: 147, SEQ ID NO: 356, or SEQ ID NO: 179, SEQ ID NO: 388 is fused to the anti-cancer antibody, bevacizumab, using linker sequence GGGSGGGS (SEQ ID NO: 444). The resulting peptide-antibody fusion is administered to a subject for the treatment of glioblastoma.

Additional peptide-antibody fusions for treating glioblastoma include those prepared from peptide sequences in TABLE 2, SEQ ID NO: 1-SEQ ID NO: 426, or SEQ ID NO: 1, SEQ ID NO: 2, SEQ ID NO: 141, or SEQ ID NO: 179, fused to an anti-brain cancer antibody, or a brain tumor biomarker described herein, including those listed in TABLE 4, or an antibody that binds to or modulates a target molecule selected from the group consisting of: SCdc42, extracellular signal-regulated kinase (ERK), mammalian target of rapamycin (mTOR), phosphatidylinositol 3-kinase (PI3K), epidermal growth factor receptor (EGFR), growth factor receptor-bound protein 2 (Grb2), c-Jun N-terminal kinase (JNK), mitogen-activated protein kinase kinases (MEK/MKK), platelet derived growth factor receptor (PDGFR), son of sevenless (SOS), TGFβ-activated kinase (TAK), transforming growth factor (TGF), or vascular endothelial growth factor receptor (VEGFR), using a linker sequence of GGGSGGGS (SEQ ID NO: 444). The resulting peptide-antibody fusions are administered to a subject for the treatment of glioblastoma.

### Example 8

### Peptide-Antibody Conjugates for Targeting Triple Negative Breast Cancer

This example describes the use of peptide-antibody conjugates for treating triple negative breast cancer in a subject. SEQ ID NO: 5, SEQ ID NO: 37, SEQ ID NO: 246, SEQ ID NO: 35, SEQ ID NO: 244, SEQ ID NO: 36, SEQ ID NO: 245, SEQ ID NO: 39, SEQ ID NO: 248 are peptides that target tumors, and are conjugated to the anti-cancer antibody, nivolumab, using linker GGGSGGGS (SEQ ID NO: 444). The resulting otpide-antibody conjugate is administered to treat or target triple negative breast cancer cells.

Additional peptide-anti-cancer conjugates or fusions are prepared using any one of the peptide sequences in TABLE 2, SEQ ID NO: 1-SEQ ID NO: 426, expressed as a fusion with an anti-cancer antibody described herein, or expressed separately and then conjugated to an antibody, including: antibodies described in TABLE 4, Urelumab, Utomilumab, Ensituximab, Tacatuzumab tetraxetan, Nesvacumab, Vanucizumab, Atezolizumab, Avelumab, Durvalumab, Duligotumab, Elgemtumab, Lumretuzumab, Patritumab, Seribantumab, Pogalizumab, Nerelimomab, Ozoralizumab, Mapatumumab, or Conatumumab, or an antibody that binds to, modulates, or targets any of the following antigens associated with cancer cells: CD20, CD30, CD33, CD52, EpCAM, gpA33, CEA, mucins, TAG-72, carbonic anhydrase IX, PSMA, folate binding protein, gangliosides (e.g., GD2, GD3, GM2), Lewis-Y², VEGF, VEGFR, αVβ3, α5β1, ErbB/EGFR, ErbB2/HER2, ErbB3, c-MET, IGF1R, EphA3, TRAIL-R1, TRAI-R2, RANKL, FAP, and tenascin, using a linker sequence of GGGSGGGS (SEQ ID NO: 444). The resulting peptide-antibody fusion is administered to a subject to treat triple negative breast cancer.

### Example 9

### Peptide-Antibody Conjugates for Targeting Colon Cancer

This example describes the use of peptide-antibody conjugate for treating colon cancer in a subject. SEQ ID NO: 5 SEQ ID NO: 37, SEQ ID NO: 246, SEQ ID NO: 35, SEQ ID NO: 244, SEQ ID NO: 36, SEQ ID NO: 245, SEQ ID NO: 39, or SEQ ID NO: 248 is conjugated to the anti-cancer antibody, cetuximab, using linker GGGSGGGS (SEQ ID NO: 444) to treat or target colon cancer cells. Conjugation is to lysine or cysteine residues in the antibody, optionally with a cleavable linker, using amine, carboxyl, or thiol residues in the peptide, using methods known in the art (reference Pharm Res. 2015 November; 32(11):3526-40. Current ADC Linker Chemistry. Jain Ni, Smith SW2, Ghone S2, Tomczuk B2. And Pharm Res. 2015 November; 32(11):3541-71. Antibody-Drug Conjugates: Design, Formulation and Physicochemical Stability. Singh SK1, Luisi DL2, Pak RH3). Diafiltration was performed to remove the free reducing agents or other reactants or small molecules. The resulting peptide-antibody conjugate is administered to a subject to treat colon cancer.

Additional peptide-anti-cancer conjugates are prepared using any one of the peptide sequences in TABLE 2, SEQ ID NO: 1-SEQ ID NO: 426, conjugated to an anti-cancer antibody described herein, including: antibodies described in TABLE 4, Urelumab, Utomilumab, Ensituximab, Tacatuzumab tetraxetan, Nesvacumab, Vanucizumab, Atezolizumab, Avelumab, Durvalumab, Duligotumab, Elgemtumab, Lumretuzumab, Patritumab, Seribantumab, Pogalizumab, Nerelimomab, Ozoralizumab, Mapatumumab, or Conatumumab, or an antibody that binds to, modulates, or targets any of the following antigens associated with cancer cells: CD20, CD30, CD33, CD52, EpCAM, gpA33, CEA, mucins, TAG-72, carbonic anhydrase IX, PSMA, folate binding protein, gangliosides (e.g., GD2, GD3, GM2), Lewis-Y², VEGF, VEGFR, αVβ3, α5β1, ErbB1/EGFR, ErbB2/HER2, ErbB3, c-MET, IGF1R, EphA3, TRAIL-R1, TRAI-R2, RANKL, FAP, and tenascin, using a linker sequence of GGGSGGGS (SEQ ID NO: 444).

The peptide can be conjugated to cytotoxic drug cetuximab that targets epidermal growth factor receptor. Additional peptide-antibody and therapeutic agent conjugations are prepared using any one of the therapeutic agents described herein, including: chemotherapeutic, anti-cancer drug, or anti-cancer agent selected from, but are not limited to: radioisotopes, toxins, enzymes, sensitizing drugs, nucleic acids, including interfering RNAs, and cisplatin. The resulting conjugates can be administered to treat colon cancer.

### Example 10

### Peptide-Antibody Conjugates for Changing Pharmacological Parameters

This example describes the use of peptide-antibody conjugate to modify the excretion, biodistribution, metabolism or other pharmacokinetic properties. SEQ ID NO: 1, SEQ ID NO: 2, SEQ ID NO: 3, SEQ ID NO: 5, SEQ ID NO: 37, SEQ ID NO: 46, SEQ ID NO: 124, SEQ ID NO: 141, or SEQ ID NO: 147, or SEQ ID NO: 179 was conjugated to an antibody Fc fragment (DKTHTCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSRDELTKNQVSLTCLVKGFYPSDIAVEWESNGQPENN YKTTPPVLDSDGSFFLYSKLTVDKSRWQQGNVFSCSVMHEALHNHYTQKSLSLSPG K, SEQ ID NO: 442) of the human IgG1 antibody using linker GGGSGGGS (SEQ ID NO: 444) in such a way that the antibody fragment can bind to FcR and be recycled in vivo extending circulating plasma levels and half-life as illustrated in FIG. 5Q. The resulting peptide-antibody Fc fragment conjugate can be administrered to a subject in order to treat disease that requires drug levels to be maintained for longer periods compared to those achieved by peptide alone.

Additional peptide-antibody fragment conjugates are prepared using any of the peptide sequences in TABLE 2 conjugated to Fc fragments from alternative immunoglobulins, Fab fragment as illustrated in FIG. 5O or scFv fragments as illustrated in FIG. 5P with appropriate targeting capabilities. Each of these exemplified peptide-antibody fragment conjugates may provide improved pharmacokinetic properties for the treatment of disease. Optionally, these conjugates may also display Fc receptor function in activating the immune system.

A peptide of SEQ ID NO: 37 was expressed as a fusion with the Fc portion of IgG1 by the methods of EXAMPLE 1 and EXAMPLE 2 to obtain the following fusion sequence:

SEQ ID NO: 443
GSMCMPCFTTDTQMQERCDRCCGGGGRGRCWGPQCLCIGGSGGSDKTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCS
VMHEALHNHYTQKSLSLSPGK,).

A peptide of SEQ ID NO: 37 (underlined) is followed by a linker (shown in bold), and is followed by the Fc region (shown in italics). This polypeptide fusion was then radiolabeled by reductive methylation with ¹⁴C formaldehyde and sodium cyanoborohydride with standard techniques and then purified.

The polypeptide comprising SEQ ID NO: 37, fused to the Fc portion of IgG, as described above, was then administered to Female Harlan athymic nude mice, weighing 20 g-25 g, via tail vein injection (n=2 mice per polypeptide). The kidneys were ligated to prevent renal filtration of the peptides or the kidneys were left intact and functioning. Each peptide (target dose of 50 nmol; 16-18 μCi/mouse) was allowed to freely circulate within the animal for a target of three-four hours before the animals were euthanized and sectioned for whole body autoradiography. At the end of the dosing period, mice were frozen in a hexane/dry ice bath and then frozen in a block of carboxymethylcellulose. Thin, frozen sections of whole animal sagittal slices were obtained with a microtome, allowed to dessicate in a freezer, and exposed to phosphoimager plates for about ten days.

These plates were developed, and the signal (densitometry) within the blood was measured in the heart chamber for each subject.

The detected level of radioactivity (quantity of polypeptide) in the blood was significantly higher for the peptide-Fc fusion as compared to the peptide alone (78-fold higher) indicating that the Fc portion reduced the excretion extending the circulating levels of the polypeptide. These data indicate that a peptide-Fc fusion will have significantly longer half-life compared to the peptide alone.

### Example 11

### Measuring CNS Accumulation of Peptide-Antibody Conjugate

This example describes measuring blood brain barrier penetration and CNS accumulation of a peptide-Fc fusion.

A peptide of SEQ ID NO: 37 was expressed as a fusion with the Fc portion of IgG1, as described above in EXAMPLE 10, radiolabeled and administered to Female Harlan athymic nude mice via tail vein injection (n=2 mice per polypeptide), as also described up in EXAMPLE 10. The kidneys were left intact and functioning. Following exposure to the test materials, the mice were euthanized and processed as previously described.

These plates were developed, and the signal (densitometry) from organs was normalized to the signal found in the heart blood of each animal.

The detected level of radioactivity (quantity of polypeptide) in the CNS was significantly higher for the peptide-Fc fusion compared to the peptide alone. Specifically, the radioactive signal in the CNS in adminals dosed with peptide of SEQ ID NO: 37 was below the limit of quantification (LOQ) of 300,000 CPM; whereas, the polypeptide of SEQ ID NO: 37 fused to the Fc portion of IgG1 (SEQ ID NO: 443) produced a signal 5.2-fold higher then the LOQ. These data demonstrate that the peptide-Fc fusion accumulated in the CNS at least 5.2-fold higher levels than the free peptide alone.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-51. (canceled)

52. A peptide-antibody complex comprising:
a) a peptide comprising a sequence having at least 90% sequence identity to:
i) SEQ ID NO: 217 or a functional fragment thereof, or
ii) SEQ ID NO: 250, SEQ ID NO: 249, SEQ ID NO: 214, SEQ ID NO: 268, SEQ ID NO: 296, SEQ ID NO: 215, SEQ ID NO: 216, SEQ ID NO: 4, SEQ ID NO: 37, SEQ ID NO: 36, SEQ ID NO: 7, SEQ ID NO: 55, SEQ ID NO: 83, SEQ ID NO: 2, or SEQ ID NO: 3, or a functional fragment thereof; and
b) an antibody or a functional fragment thereof,
wherein the peptide and the antibody are conjugated, linked, bound together by affinity, or fused.

53. The peptide-antibody complex of claim 52, wherein the peptide further comprises:
a) at least 30 amino acid residues;
b) 6 or more cysteine residues; or
c) a plurality of disulfide bridges formed between the cysteine residues.

54. The peptide-antibody complex of claim 52, wherein the peptide is a knotted peptide.

55. The peptide-antibody complex of claim 52, wherein the peptide is a targeting or homing agent, or selectively accumulates in a target cell or tissue.

56. The peptide-antibody complex of claim 52, wherein the antibody is a human or humanized monoclonal antibody, a monoclonal antibody or Fc fusion protein, an antibody fragment comprising scFv, Fab, Fc, heavy chain, light chain, single chain, or complementarity-determining region, or any combination thereof.

57. The peptide-antibody complex of claim 52, wherein the peptide and the antibody are conjugated or linked by a linker.

58. The peptide-antibody complex of claim 57, wherein the linker comprises a cleavable, non-cleavable, or a pH sensitive linker.

59. The peptide-antibody complex of claim 57, wherein the linker comprises a sequence of GGGSGGGS (SEQ ID NO: 444).

60. The peptide-antibody complex of claim 52, wherein the antibody has at least 80% sequence identity with an antibody selected from the group consisting of: Crenezumab, Urelumab, Utomilumab, Ensituximab, Tacatuzumab tetraxetan, Nesvacumab, Vanucizumab, Evinacumab, Vepalimomab, 8H9, Belimumab, Tabalumab, Bapineuzumab, Gantenerumab, Solanezumab, Aducanumab, Detumomab, Nacolomab tafenatox, Igovomab, Oregovomab, Sofituzumab vedotin, Abagovomab, Galcanezumab, Mogamulizumab, PRO 140, Tovetumab, Coltuximab ravtansine, Denintuzumab mafodotin, Inebilizumab, SGN-CD19A, Taplitumomab paptox, Afutuzumab, FBTA05, Moxetumomab pasudotox, Pinatuzumab vedotin, Varlilumab, Atezolizumab, Avelumab, Durvalumab, Enoblituzumab, Lilotomab satetraxetan, Naratuximab emtansine, Otlertuzumab, Tetulomab, Isatuximab, Daratumumab, Ibalizumab, Zanolimumab, Bleselumab, Dacetuzumab, Lucatumumab, Teneliximab, Bivatuzumab mertansine, Abituzumab, Intetumumab, Alemtuzumab, Lorvotuzumab mertansine, Itolizumab, Vorsetuzumab mafodotin, Milatuzumab, Polatuzumab vedotin, Galiximab, Altumomab pentetate, Arcitumomab, Labetuzumab, Besilesomab, Erenumab, Margetuximab, IMAB362, Actoxumab, Bezlotoxumab, Tefibazumab, Tisotumab vedotin, Cabiralizumab, Emactuzumab, Lenzilumab, Namilumab, Ticilimumab, tremelimumab, Ulocuplumab, Sevirumab, Regavirumab, Rovalpituzumab tesirine, Demcizumab, Enoticumab, Navicixizumab, Begelomab, Drozitumab, Parsatuzumab, Cetuximab, Depatuxizumab mafodotin, Futuximab, Imgatuzumab, Laprituximab emtansine, Matuzumab, Necitumumab, Nimotuzumab, Panitumumab, Zalutumumab, Carotuximab, Edobacomab, Nebacumab, Adecatumumab, Citatuzumab bogatox, Edrecolomab, Oportuzumab monatox, Solitomab, Tucotuzumab celmoleukin, Catumaxomab, Fibatuzumab, Epitumomab cituxetan, Sontuzumab, Duligotumab, Elgemtumab, Lumretuzumab, Patritumab, Seribantumab, Radretumab, Pasotuxizumab, Farletuzumab, Mirvetuximab soravtansine, Vantictumab, Crotedumab, 3F8, Ch.14.18, Dinutuximab, Derlotuximab biotin, Suvizumab, Apolizumab, Fasinumab, Ponezumab, Volociximab, Lirilumab, Monalizumab, cBR96-doxorubicin immunoconjugate, Carlumab, Amatuximab, Imalumab, Ublituximab, Anetumab ravtansine, Cantuzumab mertansine, Refanezumab, Fulranumab, Tanezumab, Racotumomab, Ozanezumab, Brontictuzumab, Tarextumab, Vesencumab, Nivolumab, Pidilizumab, Pembrolizumab, Olaratumab, Lifastuzumab vedotin, Bavituximab, Tosatoxumab, Icrucumab, Alacizumab pegol, Ramucirumab, and Pritumumab.

61. The peptide-antibody complex of claim 52, wherein the antibody binds to a target having at least 80% sequence identity with a target selected from the group consisting of: beta amyloid, Tau, alpha synuclein, BACE or BACE fragment (anti-BACE heavy chain, and anti-BACE light chain, or a fragment thereof), IL23, TGF beta, CD137, SAC, alpha-fetoprotein, angiopoietin, anthrax toxin, AOC3, VAP-1, B7-H3, calcitonin, CD4, CD5, CD6, CD20, CD22, CD27, CD30, CD52, CD274, CD276, CD28, CD33, CD37, CD38, CD40, CD44, CD51, CD56, CD70, CD74, CD79B, CD80, CD137, CD140a, CD19, CGRP, ch4D5, CLDN18.2, coagulation factor III, CSF1R, CSF2, CTLA-4, EGFL7, EGFR, endotoxin, EpCAM, gpA3, CD3, ephrin receptor A3, episialin, αVβ3, α5β1, ErbB1/EGFR, ErbB2/HER2, ErbB3, ERBB3, HER3, FAP, fibrin, fibronectin, carbonic anhydrase IX, PSMA, folate binding protein, folate hydrolase, folate receptor, Frizzled receptor, GCGR, gangliosides (e.g., GD2, GD3, GM2) glypican 3, GMCSF receptor, GPNMB, growth differentiation factor 8, GUCY2C, HER1, HER2/neu, HGF, HHGFR, histone complex, HIV-1, HLA-DR, HNGF, scatter factor receptor kinase, TNF, IGF-1 receptor, CD221, IGF1, IGF2, c-MET, IGF1R, IL 17A, IL-13, IL-17, IL2, ILGF2, KIR2D, KLRC1, Lewis-Y antigen, Lewis-Y², MCP-1, mesothelin, MIF, MS4A1, MSLN, NGF, N-glycolylneuraminic acid, NOGO-A, Notch 1, Notch receptor, NRP1, PD-1, PD-L1, PDCD1, PDGF-R, phosphate-sodium co-transporter, RON, RTN4, SDC1, STEAP1, TAG-72, T-cell receptor, TEM1, tenascin, tenascin C, EphA3, TNFR, TNF-α, TRAIL-R1, TRAIL-R2, RANKL, FAP, alpha synuclein tumor-associated calcium signal transducer 2, TWEAK receptor, VEGF, VEGFR1, VEGFR2, vimentin, CD20/MS4A1, 4-1BB, 5T4, activin receptor-like kinase 1, adenocarcinoma antigen, AGS-22M6, B7-H3, BAFF, B-lymphoma cell, C242 antigen, CCR4, CCR5, CEA, CGRP, CLDN18.2, mucins, TAG-72, Clostridium difficile, clumping factor A, coagulation factor III, CSF1R, CSF2, CTLA-4, CXCR4 (CD184), cytomegalovirus, cytomegalovirus glycoprotein B, DLL3, DLL4, DPP4, DR5, EGFL7, EGFR, endoglin, endotoxin, HNGF, integrin α5β1, MSLN, myelin-associated glycoprotein, NGF, phosphatidylserine, and Staphylococcus aureus.

62. The peptide-antibody complex of claim 52, wherein the antibody has at least 90% identity with SEQ ID NO: 427 or SEQ ID NO: 428, or a fragment thereof.

63. The peptide-antibody complex of claim 52, further comprising a therapeutic agent or detectable agent.

64. The peptide-antibody complex of claim 52, wherein the peptide-antibody complex crosses a blood brain barrier or a blood cerebrospinal fluid barrier of a subject.

65. The peptide-antibody complex of claim 52, wherein the peptide crosses a blood brain barrier to target a tumor in a brain.

66. The peptide-antibody complex of claim 52, wherein the peptide-antibody complex homes, targets, is directed to, migrates to, or penetrates a tumor or cancerous cell.

67. The peptide-antibody complex of claim 66, wherein the tumor or cancerous cell is a solid tumor, a brain cancer, a glioblastoma, a primary brain tumor, a metastatic brain tumor, a triple-negative breast cancer, a colon cancer, or a sarcoma.

68. The peptide-antibody complex of claim 52, wherein the antibody inhibits a cellular pathway associated with cancer.

69. The peptide-antibody complex of claim 52, wherein the peptide inhibits a cellular pathway associated with cancer.

70. The peptide-antibody complex of claim 52, wherein the peptide-antibody complex homes, targets, is directed to, or migrates to a specific region in CNS, ventricles, the cerebrospinal fluid, the hippocampus, the meninges, the rostral migratory system, the dentate gyrus, the subventricular zone, or any combination thereof.

71. The peptide-antibody complex of claim 52, wherein the peptide crosses a blood brain barrier to reduce aggregation or plaque in a brain.

72. The peptide-antibody complex of claim 52, further comprising a pharmaceutically acceptable carrier.

73. A method of treating a condition in a subject in need thereof, the method comprising:
administering to the subject a peptide-antibody complex comprising:
a) a peptide comprising a sequence having at least 90% sequence identity to:
i) SEQ ID NO: 217, or a functional fragment thereof, or
ii) SEQ ID NO: 250, SEQ ID NO: 249, SEQ ID NO: 214, SEQ ID NO: 268, SEQ ID NO: 296, SEQ ID NO: 215, SEQ ID NO: 216, SEQ ID NO: 4, SEQ ID NO: 37, SEQ ID NO: 36, SEQ ID NO: 7, SEQ ID NO: 55, SEQ ID NO: 83, SEQ ID NO: 2, or SEQ ID NO: 3, or a functional fragment thereof; and
b) an antibody or a functional fragment thereof,
wherein the peptide and the antibody are conjugated, linked, bound together by affinity, or fused.

74. The method of claim 73, wherein the peptide-antibody complex homes, targets, migrates to, or is directed to a cancerous or diseased region, tissue, structure, or cell of the subject following administration.

75. The method of claim 73, wherein the condition is a tumor or cancer

76. The method of claim 75, wherein the tumor or cancer is a solid tumor, a brain tumor, triple-negative breast cancer, colon cancer, sarcoma, or a metastatic cancer.

77. The method of claim 73, wherein the peptide-antibody complex crosses a blood brain barrier or a blood cerebrospinal fluid barrier of the subject following administration.

78. The method of claim 73, wherein the peptide-antibody complex homes, targets, is directed to or migrates to the ventricles, cerebrospinal fluid, meninges, rostral migratory system, or hippocampus of the subject following administration.

79. The method of claim 73, wherein the condition is a brain disorder.

80. The method of claim 73, wherein the condition is memory loss, Alzheimer's disease, Parkinson's disease, multiple system atrophy (MSA), schizophrenia, epilepsy, progressive multifocal leukoencephalopathy, fungal infection, depression, bipolar disorder, post-traumatic stress disorder, stroke, traumatic brain injury, infection, or multiple sclerosis.


SEQUENCE LISTING (464 sequences)

SEQ ID NO: 1 - protein, 36 aa, Artificial Sequence
  Description of Artificial Sequence Synthetic polypeptide
GSMCIPCFTTNPNMAAKCNACCGSRRGSCRGPQCIC

SEQ ID NO: 2 - protein, 37 aa, Artificial Sequence
  Description of Artificial Sequence Synthetic polypeptide
GSGCLEFWWKCNPNDDKCCRPKLKCSKLFKLCNFSFG

SEQ ID NO: 3 - protein, 38 aa, Artificial Sequence
  Description of Artificial Sequence Synthetic polypeptide
GSECRYWLGTCSKTGDCCSHLSCSPKHGWCVWDWTFRK

SEQ ID NO: 4 - protein, 38 aa, Artificial Sequence
  Description of Artificial Sequence Synthetic polypeptide
GSMCMPCFTTDHQMARRCDDCCGGRGRGRCYGPQCLCR

SEQ ID NO: 5 - protein, 38 aa, Artificial Sequence
  Description of Artificial Sequence Synthetic polypeptide
GSMCMPCFTTHHRMAENCDICCGGDGRGKCYGPQCLCR

SEQ ID NO: 6 - protein, 38 aa, Artificial Sequence
  Description of Artificial Sequence Synthetic polypeptide
GSMCMPCFTTDHRMAENCDICCGGDGRGKCYGPQCLCR

SEQ ID NO: 7 - protein, 38 aa, Artificial Sequence
  Description of Artificial Sequence Synthetic polypeptide
GSMCMPCFTTHHQMAENCDICCGGDGRGKCYGPQCLCR

SEQ ID NO: 8 - protein, 38 aa, Artificial Sequence
  Description of Artificial Sequence Synthetic polypeptide
GSMCMPCFTTHHRMARNCDICCGGDGRGKCYGPQCLCR

SEQ ID NO: 9 - protein, 38 aa, Artificial Sequence
  Description of Artificial Sequence Synthetic polypeptide
GSMCMPCFTTHHRMAERCDICCGGDGRGKCYGPQCLCR

SEQ ID NO: 10 - protein, 38 aa, Artificial Sequence
  Description of Artificial Sequence Synthetic polypeptide
GSMCMPCFTTHHRMAENCDDCCGGDGRGKCYGPQCLCR

SEQ ID NO: 11 - protein, 38 aa, Artificial Sequence
  Description of Artificial Sequence Synthetic polypeptide
GSRCMPCFTTDHQMARRCDDCCGGRGRGKCYGPQCLCR

SEQ ID NO: 12 - protein, 38 aa, Artificial Sequence
  Description of Artificial Sequence Synthetic polypeptide
GSICIPCFTTDHQIARRCDDCCGGRGRGKCYGPQCLCR

SEQ ID NO: 13 - protein, 38 aa, Artificial Sequence
  Description of Artificial Sequence Synthetic polypeptide
GSMCLPCFTTDHQLARRCDDCCGGRGRGKCYGPQCLCR

SEQ ID NO: 14 - protein, 38 aa, Artificial Sequence
  Description of Artificial Sequence Synthetic polypeptide
GSMCMPCFTTEHQMARRCEECCGGRGRGKCYGPQCLCR

SEQ ID NO: 15 - protein, 38 aa, Artificial Sequence
  Description of Artificial Sequence Synthetic polypeptide
GSMCIPCFTTDHQMARRCEECCGGRGRGKCYGPQCLCR

SEQ ID NO: 16 - protein, 38 aa, Artificial Sequence
  Description of Artificial Sequence Synthetic polypeptide
GSICIPCFTTDHQMARRCDDCCGGRGDGKCYGPQCLCR

SEQ ID NO: 17 - protein, 38 aa, Artificial Sequence
  Description of Artificial Sequence Synthetic polypeptide
GSICIPCFTTDHQIARRCDDCCGGRGRGKCYGPQCICR

SEQ ID NO: 18 - protein, 38 aa, Artificial Sequence
  Description of Artificial Sequence Synthetic polypeptide
GSRCMPCFTTDHFMARFCDFCCGGRGRGKCYGPQCLCR

SEQ ID NO: 19 - protein, 38 aa, Artificial Sequence
  Description of Artificial Sequence Synthetic polypeptide
GSRCMPCFTTDHYMARYCDYCCGGRGRGKCYGPQCLCR

SEQ ID NO: 20 - protein, 38 aa, Artificial Sequence
  Description of Artificial Sequence Synthetic polypeptide
GSRCMPCFTTDHRMARRCDRCCGGRGRGKCYGPQCLCR

SEQ ID NO: 21 - protein, 38 aa, Artificial Sequence
  Description of Artificial Sequence Synthetic polypeptide
GSRCMPCFTTDHEMARECDECCGGRGRGKCYGPQCLCR

SEQ ID NO: 22 - protein, 38 aa, Artificial Sequence
  Description of Artificial Sequence Synthetic polypeptide
GSRCMPCFTTDHHMARHCDHCCGGRGRGKCYGPQCLCR

SEQ ID NO: 23 - protein, 38 aa, Artificial Sequence
  Description of Artificial Sequence Synthetic polypeptide
GSLCLPCFTTHHRLADQCDICCGGDGRGKCYGPQCLCR

SEQ ID NO: 24 - protein, 38 aa, Artificial Sequence
  Description of Artificial Sequence Synthetic polypeptide
GSICIPCFTTEHQIARRCEECCGGRGRGKCYGPQCLCR

SEQ ID NO: 25 - protein, 38 aa, Artificial Sequence
  Description of Artificial Sequence Synthetic polypeptide
GSMCMPCFTTDTQMQERCDRCCGGGGRGKCWGPQCLCI

SEQ ID NO: 26 - protein, 38 aa, Artificial Sequence
  Description of Artificial Sequence Synthetic polypeptide
GSMCMPCFTTIYRMAHECDECCGGRGRGKCYGPQCLCR

SEQ ID NO: 27 - protein, 38 aa, Artificial Sequence
  Description of Artificial Sequence Synthetic polypeptide
GSMCMPCFTTGYRMAEYCDICCGGRGRGKCYGPQCLCR

SEQ ID NO: 28 - protein, 38 aa, Artificial Sequence
  Description of Artificial Sequence Synthetic polypeptide
GSMCMPCFTTHRRMANTCDACCGGRSRGKCYGPQCLCR

SEQ ID NO: 29 - protein, 38 aa, Artificial Sequence
  Description of Artificial Sequence Synthetic polypeptide
GSHCMPCFTTDHQMIRRCDDCCGGGSYGKCDGPQCLCF

SEQ ID NO: 30 - protein, 38 aa, Artificial Sequence
  Description of Artificial Sequence Synthetic polypeptide
GSDCMPCFTTDHRMADHCDICCGGDDRGKCYGPQCLCR

SEQ ID NO: 31 - protein, 38 aa, Artificial Sequence
  Description of Artificial Sequence Synthetic polypeptide
GSMCMPCFTTDHEMERRCDDCCGIGGGGKCHGPQCLCG

SEQ ID NO: 32 - protein, 38 aa, Artificial Sequence
  Description of Artificial Sequence Synthetic polypeptide
GSMCMPCFTTEQRMAIICDDCCGGFGRGKCYGPQCLCR

SEQ ID NO: 33 - protein, 38 aa, Artificial Sequence
  Description of Artificial Sequence Synthetic polypeptide
GSMCMPCFTTSEQMFRRCDDCCGGWGDGKCNGPHCLCR

SEQ ID NO: 34 - protein, 39 aa, Artificial Sequence
  Description of Artificial Sequence Synthetic polypeptide
GSGVPINVKCRGSRDCLDPCKKAGMRFGKCINSKCHCTP

SEQ ID NO: 35 - protein, 38 aa, Artificial Sequence
  Description of Artificial Sequence Synthetic polypeptide
GSMCMPCFTTEQRMAIICDDCCGGFGRGRCYGPQCLCR

SEQ ID NO: 36 - protein, 38 aa, Artificial Sequence
  Description of Artificial Sequence Synthetic polypeptide
GSICIPCFTTDHQIARRCDDCCGGRGRGRCYGPQCICR

SEQ ID NO: 37 - protein, 38 aa, Artificial Sequence
  Description of Artificial Sequence Synthetic polypeptide
GSMCMPCFTTDTQMQERCDRCCGGGGRGRCWGPQCLCI

SEQ ID NO: 38 - protein, 37 aa, Artificial Sequence
  Description of Artificial Sequence Synthetic polypeptide
GSMCMPCFTTDTQMQERCDRCCGGGGRGRCWGPQCLC

SEQ ID NO: 39 - protein, 38 aa, Artificial Sequence
  Description of Artificial Sequence Synthetic polypeptide
GSMCMPCFTTDHRMAENCDICCGGDGRGRCYGPQCLCR

SEQ ID NO: 40 - protein, 38 aa, Artificial Sequence
  Description of Artificial Sequence Synthetic polypeptide
GSMCMPCFTTEQRMAIICDDCCGGFGRGKCYGPQCLCI

SEQ ID NO: 41 - protein, 38 aa, Artificial Sequence
  Description of Artificial Sequence Synthetic polypeptide
GSMCMPCFTTEQRMAIICDDCCGGFGRGRCYGPQCLCI

SEQ ID NO: 42 - protein, 38 aa, Artificial Sequence
  Description of Artificial Sequence Synthetic polypeptide
GSICIPCFTTDHQIARRCDDCCGGRGRGKCYGPQCICI

SEQ ID NO: 43 - protein, 38 aa, Artificial Sequence
  Description of Artificial Sequence Synthetic polypeptide
GSICIPCFTTDHQIARRCDDCCGGRGRGRCYGPQCICI

SEQ ID NO: 44 - protein, 38 aa, Artificial Sequence
  Description of Artificial Sequence Synthetic polypeptide
GSMCMPCFTTDTQMQEKCDRCCGGGGRGRCWGPQCLCI

SEQ ID NO: 45 - protein, 38 aa, Artificial Sequence
  Description of Artificial Sequence Synthetic polypeptide
GSMCMPCFTTEQRMAIKCDDCCGGFGRGRCYGPQCLCR

SEQ ID NO: 46 - protein, 38 aa, Artificial Sequence
  Description of Artificial Sequence Synthetic polypeptide
GSICIPCFTTDHQIARKCDDCCGGRGRGRCYGPQCICR

SEQ ID NO: 47 - protein, 38 aa, Artificial Sequence
  Description of Artificial Sequence Synthetic polypeptide
GSMCMPCFTTDHRMAEKCDICCGGDGRGRCYGPQCLCR

SEQ ID NO: 48 - protein, 38 aa, Artificial Sequence
  Description of Artificial Sequence Synthetic polypeptide
GSMCMPCFTTDTQMQERCDRCCGGKGRGRCWGPQCLCI

SEQ ID NO: 49 - protein, 38 aa, Artificial Sequence
  Description of Artificial Sequence Synthetic polypeptide
GSMCMPCFTTEQRMAIICDDCCGGKGRGRCYGPQCLCR

SEQ ID NO: 50 - protein, 38 aa, Artificial Sequence
  Description of Artificial Sequence Synthetic polypeptide
GSICIPCFTTDHQIARRCDDCCGGKGRGRCYGPQCICR

SEQ ID NO: 51 - protein, 38 aa, Artificial Sequence
  Description of Artificial Sequence Synthetic polypeptide
GSMCMPCFTTDHRMAENCDICCGGKGRGRCYGPQCLCR

SEQ ID NO: 52 - protein, 38 aa, Artificial Sequence
  Description of Artificial Sequence Synthetic polypeptide
GSMCMPCFTTDHRMAENCDICCGGDGRGKCYGPQCLCI

SEQ ID NO: 53 - protein, 38 aa, Artificial Sequence
  Description of Artificial Sequence Synthetic polypeptide
GSMCMPCFTTDHRMAENCDICCGGDGRGRCYGPQCLCI

SEQ ID NO: 54 - protein, 38 aa, Artificial Sequence
  Description of Artificial Sequence Synthetic polypeptide
GSMCMPCFTTHHRMAENCDICCGGDGRGRCYGPQCLCR

SEQ ID NO: 55 - protein, 30 aa, Artificial Sequence
  Description of Artificial Sequence Synthetic polypeptide
GSVGCEECPMHCKGKNANPTCDDGVCNCNV

SEQ ID NO: 56 - protein, 30 aa, Artificial Sequence
  Description of Artificial Sequence Synthetic polypeptide
GSVGCEECPMHCKGKNAKPTCDDGVCNCNV

SEQ ID NO: 57 - protein, 30 aa, Artificial Sequence
  Description of Artificial Sequence Synthetic polypeptide
GSVGCEECPMHCKGKNAKPTCDNGVCNCNV

SEQ ID NO: 58 - protein, 30 aa, Artificial Sequence
  Description of Artificial Sequence Synthetic polypeptide
GSVGCEECPMHCKGKHAVPTCDDGVCNCNV

SEQ ID NO: 59 - protein, 30 aa, Artificial Sequence
  Description of Artificial Sequence Synthetic polypeptide
GSVGCEECPAHCKGKNAKPTCDDGVCNCNV

SEQ ID NO: 60 - protein, 30 aa, Artificial Sequence
  Description of Artificial Sequence Synthetic polypeptide
GSVGCEECPAHCKGKNAIPTCDDGVCNCNV

SEQ ID NO: 61 - protein, 30 aa, Artificial Sequence
  Description of Artificial Sequence Synthetic polypeptide
GSVGCEECPMHCKGKMAKPTCDDGVCNCNV

SEQ ID NO: 62 - protein, 30 aa, Artificial Sequence
  Description of Artificial Sequence Synthetic polypeptide
GSVGCEECPMHCKGKNAVPTCDNGVCNCNA

SEQ ID NO: 63 - protein, 30 aa, Artificial Sequence
  Description of Artificial Sequence Synthetic polypeptide
GSVGCEECPMHCKGKMAKPTCYDGVCNCNV

SEQ ID NO: 64 - protein, 30 aa, Artificial Sequence
  Description of Artificial Sequence Synthetic polypeptide
GSVGCEECPMYCKGKNAVPTCDGGVCNCNA

SEQ ID NO: 65 - protein, 30 aa, Artificial Sequence
  Description of Artificial Sequence Synthetic polypeptide
GSVGCEECPKYCKGKNAVPTCDGGVCNCNA

SEQ ID NO: 66 - protein, 30 aa, Artificial Sequence
  Description of Artificial Sequence Synthetic polypeptide
GSVGCEECPVYCKGKKALPTCDGGVCNCNA

SEQ ID NO: 67 - protein, 30 aa, Artificial Sequence
  Description of Artificial Sequence Synthetic polypeptide
GSVGCEEDPMHCKGKQAKPTCCNGVCNCNV

SEQ ID NO: 68 - protein, 34 aa, Artificial Sequence
  Description of Artificial Sequence Synthetic polypeptide
GSVGCAECPMHCKGKMAKPTCENEVCKCNIGKKD

SEQ ID NO: 69 - protein, 29 aa, Artificial Sequence
  Description of Artificial Sequence Synthetic peptide
GSVGCEECPMHCKGKKALPTCDYGCECND

SEQ ID NO: 70 - protein, 33 aa, Artificial Sequence
  Description of Artificial Sequence Synthetic polypeptide
GSIVCKVCKIICGMQGKKVNICKAPIKCKCKKG

SEQ ID NO: 71 - protein, 31 aa, Artificial Sequence
  Description of Artificial Sequence Synthetic polypeptide
GSVSCEDCPDHCSTQKARAKCDNDKCVCEPK

SEQ ID NO: 72 - protein, 31 aa, Artificial Sequence
  Description of Artificial Sequence Synthetic polypeptide
GSVSCEDCPEHCSTQKARAKCDNDKCVCESV

SEQ ID NO: 73 - protein, 31 aa, Artificial Sequence
  Description of Artificial Sequence Synthetic polypeptide
GSVSCEDCPEHCSTQKAQAKCDNDKCVCEPI

SEQ ID NO: 74 - protein, 31 aa, Artificial Sequence
  Description of Artificial Sequence Synthetic polypeptide
GSATCEDCPEHCATQNARAKCDNDKCVCEPK

SEQ ID NO: 75 - protein, 31 aa, Artificial Sequence
  Description of Artificial Sequence Synthetic polypeptide
GSVSCEDCPEHCATKDQRAKCDNDKCVCEPK

SEQ ID NO: 76 - protein, 31 aa, Artificial Sequence
  Description of Artificial Sequence Synthetic polypeptide
GSVGCEDCPEHCSQQNARAKCENDKCVCEPK

SEQ ID NO: 77 - protein, 31 aa, Artificial Sequence
  Description of Artificial Sequence Synthetic polypeptide
GSVSCEDCPEHCATKDQRAKCDNDRCVCEPK

SEQ ID NO: 78 - protein, 31 aa, Artificial Sequence
  Description of Artificial Sequence Synthetic polypeptide
GSVSCEDCPPHCATKDQRAKCENDKCVCEPK

SEQ ID NO: 79 - protein, 31 aa, Artificial Sequence
  Description of Artificial Sequence Synthetic polypeptide
GSVSCEDCPEHCSTQKARAKCDNDKCVCEAI

SEQ ID NO: 80 - protein, 37 aa, Artificial Sequence
  Description of Artificial Sequence Synthetic polypeptide
GSMCMPCFTTEQRMAIICDDCCGGFGRGRCYGPQCLC

SEQ ID NO: 81 - protein, 37 aa, Artificial Sequence
  Description of Artificial Sequence Synthetic polypeptide
GSICIPCFTTDHQIARRCDDCCGGRGRGRCYGPQCIC

SEQ ID NO: 82 - protein, 37 aa, Artificial Sequence
  Description of Artificial Sequence Synthetic polypeptide
GSMCMPCFTTDHRMAENCDICCGGDGRGRCYGPQCLC

SEQ ID NO: 83 - protein, 30 aa, Artificial Sequence
  Description of Artificial Sequence Synthetic polypeptide
GSVGCEECPMHCRGRNANPTCDDGVCNCNV

SEQ ID NO: 84 - protein, 28 aa, Artificial Sequence
  Description of Artificial Sequence Synthetic peptide
GSVGCEECPMHCRGRNANPTCDDGVCNC

SEQ ID NO: 85 - protein, 36 aa, Artificial Sequence
  Description of Artificial Sequence Synthetic polypeptide
GSCGPCFTTDHQMEQKCAECCGGIGKCYGPQCLCNR

SEQ ID NO: 86 - protein, 40 aa, Artificial Sequence
  Description of Artificial Sequence Synthetic polypeptide
GSRCGPCFTTDPQTQAKCSECCGRKGGVCKGPQCICGIQY

SEQ ID NO: 87 - protein, 37 aa, Artificial Sequence
  Description of Artificial Sequence Synthetic polypeptide
GSMCMPCFTTDPNMAKKCRDCCGGNGKCFGPQCLCNR

SEQ ID NO: 88 - protein, 36 aa, Artificial Sequence
  Description of Artificial Sequence Synthetic polypeptide
GSMCMPCFTTDHNMAKKCNDCCGGYGKCFGPQCLCR

SEQ ID NO: 89 - protein, 38 aa, Artificial Sequence
  Description of Artificial Sequence Synthetic polypeptide
GSRCPPCFTTNPNMEADCRKCCGGRGYCASYQCICPGG

SEQ ID NO: 90 - protein, 37 aa, Artificial Sequence
  Description of Artificial Sequence Synthetic polypeptide
GSMCMPCFTTDPNMANKCRDCCGGGKKCFGPQCLCNR

SEQ ID NO: 91 - protein, 64 aa, Artificial Sequence
  Description of Artificial Sequence Synthetic polypeptide
GSMKFLYGVILIALFLTVMTATLSEARCGPCFTTDPQTQAKCSECCGRKGGVCKGPQCICGIQY

SEQ ID NO: 92 - protein, 38 aa, Artificial Sequence
  Description of Artificial Sequence Synthetic polypeptide
GSMCMPCFTTRPDMAQQCRACCKGRGKCFGPQCLCGYD

SEQ ID NO: 93 - protein, 62 aa, Artificial Sequence
  Description of Artificial Sequence Synthetic polypeptide
GSMKFLYGIVFIALFLTVMTATLSDAMCMPCFTTDHNMAKKCRDCCGGNGKCFGPQCLCNRG

SEQ ID NO: 94 - protein, 37 aa, Artificial Sequence
  Description of Artificial Sequence Synthetic polypeptide
GSMCMPCFTTDHNMAKKCRDCCGGNGKCFGPQCLCNR

SEQ ID NO: 95 - protein, 62 aa, Artificial Sequence
  Description of Artificial Sequence Synthetic polypeptide
GSMKFLYGIVFITLFLTVMIATHTEAMCMPCFTTRPNMAQQCRDCCRGRGKCFGPQCLCGYD

SEQ ID NO: 96 - protein, 62 aa, Artificial Sequence
  Description of Artificial Sequence Synthetic polypeptide
GSMKFLYGIVFIALFLTVMIATHTEAMCMPCFTTRPNMAQQCRDCCRGRGKCFGPQCLCGYD

SEQ ID NO: 97 - protein, 37 aa, Artificial Sequence
  Description of Artificial Sequence Synthetic polypeptide
GSRCKPCFTTDPQMSKKCADCCGGKGKGKCYGPQCLC

SEQ ID NO: 98 - protein, 63 aa, Artificial Sequence
  Description of Artificial Sequence Synthetic polypeptide
GSMKFLYGIVFITLFLTVMIATHTEAAMCMPCFTTNLNMEQECRDCCGGTGRCFGPQCLCGYD

SEQ ID NO: 99 - protein, 40 aa, Artificial Sequence
  Description of Artificial Sequence Synthetic polypeptide
GSRCSPCFTTDQQMTKKCYDCCGGKGKGKCYGPQCICAPY

SEQ ID NO: 100 - protein, 37 aa, Artificial Sequence
  Description of Artificial Sequence Synthetic polypeptide
GSCGPCFTTDPYTESKCATCCGGRGKCVGPQCLCNRI

SEQ ID NO: 101 - protein, 41 aa, Artificial Sequence
  Description of Artificial Sequence Synthetic polypeptide
GSTEAMCMPCFTTDHNMAKKCRDCCGGNGKCFGYQCLCNRG

SEQ ID NO: 102 - protein, 61 aa, Artificial Sequence
  Description of Artificial Sequence Synthetic polypeptide
GSMKFLYGIVFIALFLTVMFATQTDGCGPCFTTDANMARKCRECCGGIGKCFGPQCLCNRI

SEQ ID NO: 103 - protein, 61 aa, Artificial Sequence
  Description of Artificial Sequence Synthetic polypeptide
GSMKFLYGIVFIALFLTVMFATQTDGCGPCFTTDANMARKCRECCGGNGKCFGPQCLCNRE

SEQ ID NO: 104 - protein, 62 aa, Artificial Sequence
  Description of Artificial Sequence Synthetic polypeptide
GSMKFLYGTILIAFFLTVMIATHSEARCPPCFTTNPNMEADCRKCCGGRGYCASYQCICPGG

SEQ ID NO: 105 - protein, 41 aa, Artificial Sequence
  Description of Artificial Sequence Synthetic polypeptide
GSTEAMCMPCFTTRPDMAQQCRDCCGGNGKCFGYQCLCNRG

SEQ ID NO: 106 - protein, 62 aa, Artificial Sequence
  Description of Artificial Sequence Synthetic polypeptide
GSMKFLYGIVFIALFLTVMIATLTEAMCMPCFTTRPDMAQQCRDCCGGNGKCFGYQCLCNRG

SEQ ID NO: 107 - protein, 62 aa, Artificial Sequence
  Description of Artificial Sequence Synthetic polypeptide
GSMKFLYGIVFIALFLTVMIATHTEAMCMPCFTTRPDMAQQCRDCCGGNGKCFGYQCLCNRG

SEQ ID NO: 108 - protein, 63 aa, Artificial Sequence
  Description of Artificial Sequence Synthetic polypeptide
GSMKFLYGIILIALFLTVMIATHSEARCPNCFTTNPNAEADCKKCCGNRWGKCAGYQCVCPMK

SEQ ID NO: 109 - protein, 69 aa, Artificial Sequence
  Description of Artificial Sequence Synthetic polypeptide
GSMKFLYGIVFIALFLTGMIATHTEAMCMPCFTTRPDMAQQCRDCCGGNGKCFGYQCLCNRGRIVIMYT

SEQ ID NO: 110 - protein, 37 aa, Artificial Sequence
  Description of Artificial Sequence Synthetic polypeptide
GSMCMPCFTTRPGMAQQCRDCCGGNGKCFGYQCLCNR

SEQ ID NO: 111 - protein, 37 aa, Artificial Sequence
  Description of Artificial Sequence Synthetic polypeptide
GSMCIPCFTTNPNMAAKCNACCGSRRGSCRGPQCICR

SEQ ID NO: 112 - protein, 37 aa, Artificial Sequence
  Description of Artificial Sequence Synthetic polypeptide
GSMCIPCFTTNPNMAAKCNACCGSRRGSCRGPQCICN

SEQ ID NO: 113 - protein, 36 aa, Artificial Sequence
  Description of Artificial Sequence Synthetic polypeptide
GSMCIPCFTTNPNMAAKCNACCGGNGSCRGPQCICN

SEQ ID NO: 114 - protein, 38 aa, Artificial Sequence
  Description of Artificial Sequence Synthetic polypeptide
GSMCIPCFTTNPNMAAKCNACCGSRGRGSCRGPQCICN

SEQ ID NO: 115 - protein, 38 aa, Artificial Sequence
  Description of Artificial Sequence Synthetic polypeptide
GSMCIPCFTTNPNMAAKCNACCGSRGRGKCRGPQCICN

SEQ ID NO: 116 - protein, 37 aa, Artificial Sequence
  Description of Artificial Sequence Synthetic polypeptide
GSMCIPCFTTDHQMAAKCNACCGSRRGSCRGPQCICN

SEQ ID NO: 117 - protein, 37 aa, Artificial Sequence
  Description of Artificial Sequence Synthetic polypeptide
GSMCIPCFTTNHQMAAKCNACCGSRRGSCRGPQCICN

SEQ ID NO: 118 - protein, 38 aa, Artificial Sequence
  Description of Artificial Sequence Synthetic polypeptide
GSMCIPCFTTNPNMARKCNACCGSRGRGSCRGPQCICN

SEQ ID NO: 119 - protein, 38 aa, Artificial Sequence
  Description of Artificial Sequence Synthetic polypeptide
GSMCIPCFTTNPNMAAKCNACCGGKGRGSCRGPQCICN

SEQ ID NO: 120 - protein, 37 aa, Artificial Sequence
  Description of Artificial Sequence Synthetic polypeptide
GSMCIPCFTTNPNMAAKCNACCGSRRGSCFGPQCICN

SEQ ID NO: 121 - protein, 38 aa, Artificial Sequence
  Description of Artificial Sequence Synthetic polypeptide
GSMCIPCFTTNPNMAAKCNACCGSRGRGKCFGPQCICN

SEQ ID NO: 122 - protein, 38 aa, Artificial Sequence
  Description of Artificial Sequence Synthetic polypeptide
GSMCIPCFTTNPNMAAKCNACCGSRGRGSCFGPQCICN

SEQ ID NO: 123 - protein, 38 aa, Artificial Sequence
  Description of Artificial Sequence Synthetic polypeptide
GSMCIPCFTTNPNMAAKCNACCGSRGRGSCYGPQCICN

SEQ ID NO: 124 - protein, 37 aa, Artificial Sequence
  Description of Artificial Sequence Synthetic polypeptide
GSMCIPCFTTNPNMAAKCDACCGSRRGSCRGPQCICN

SEQ ID NO: 125 - protein, 37 aa, Artificial Sequence
  Description of Artificial Sequence Synthetic polypeptide
GSMCIPCFTTNHQMAAKCDACCGSRRGSCRGPQCICN

SEQ ID NO: 126 - protein, 38 aa, Artificial Sequence
  Description of Artificial Sequence Synthetic polypeptide
GSMCIPCFTTNHNMAAKCDACCGGRGRGSCRGPQCICN

SEQ ID NO: 127 - protein, 38 aa, Artificial Sequence
  Description of Artificial Sequence Synthetic polypeptide
GSMCIPCFTTNPNMAAKCDACCGSRGRGSCRGPQCICN

SEQ ID NO: 128 - protein, 38 aa, Artificial Sequence
  Description of Artificial Sequence Synthetic polypeptide
GSMCIPCFTTNPNMAAKCDACCGGKGRGSCRGPQCICN

SEQ ID NO: 129 - protein, 38 aa, Artificial Sequence
  Description of Artificial Sequence Synthetic polypeptide
GSMCIPCFTTNHNMAAKCDACCGSRGRGSCRGPQCICN

SEQ ID NO: 130 - protein, 36 aa, Artificial Sequence
  Description of Artificial Sequence Synthetic polypeptide
GSMCIPCFTTNPNMAAKCRDCCGGRGSCRGPQCICN

SEQ ID NO: 131 - protein, 38 aa, Artificial Sequence
  Description of Artificial Sequence Synthetic polypeptide
GSMCMPCFTTNPNMAAKCDDCCGSRGRGSCRGPQCICN

SEQ ID NO: 132 - protein, 36 aa, Artificial Sequence
  Description of Artificial Sequence Synthetic polypeptide
GSMCIPCFTTNPNMAARCNACCGSRRGSCRGPQCIC

SEQ ID NO: 133 - protein, 37 aa, Artificial Sequence
  Description of Artificial Sequence Synthetic polypeptide
GSMCIPCFTTNPNMAAKCNACCGSRRGSCRGPQCICI

SEQ ID NO: 134 - protein, 38 aa, Artificial Sequence
  Description of Artificial Sequence Synthetic polypeptide
GSGCLQFMWKCNPDNDKCCRPNLKCNTYHKWCEFVTGK

SEQ ID NO: 135 - protein, 36 aa, Artificial Sequence
  Description of Artificial Sequence Synthetic polypeptide
GSDCLGFLWKCNPSNDKCCRPNLVCSRKDKWCKYQI

SEQ ID NO: 136 - protein, 38 aa, Artificial Sequence
  Description of Artificial Sequence Synthetic polypeptide
GSDCLGFMRKCIPDNDKCCRPNLVCSRTHKWCKYVFGK

SEQ ID NO: 137 - protein, 37 aa, Artificial Sequence
  Description of Artificial Sequence Synthetic polypeptide
GSECLEIFKACNPSNDQCCKSSKLVCSRKTRACKYQI

SEQ ID NO: 138 - protein, 36 aa, Artificial Sequence
  Description of Artificial Sequence Synthetic polypeptide
GSECGGFWWKCGSGKPACCPKYVCSPKWGLCNFPMP

SEQ ID NO: 139 - protein, 37 aa, Artificial Sequence
  Description of Artificial Sequence Synthetic polypeptide
GSGCLERWWKCNPNDDKCCRPKLKCSKLFKLCNRSRG

SEQ ID NO: 140 - protein, 37 aa, Artificial Sequence
  Description of Artificial Sequence Synthetic polypeptide
GSGCLEEWWKCNPNDDKCCRPKLKCSKLFKLCNESEG

SEQ ID NO: 141 - protein, 37 aa, Artificial Sequence
  Description of Artificial Sequence Synthetic polypeptide
GSGCLEIWWKCNPNDDKCCRPKLKCSKLFKLCNYSIG

SEQ ID NO: 142 - protein, 37 aa, Artificial Sequence
  Description of Artificial Sequence Synthetic polypeptide
GSGCLEFWWKCNPNDDKCCRPKLKCSKLGKLCNFSFG

SEQ ID NO: 143 - protein, 37 aa, Artificial Sequence
  Description of Artificial Sequence Synthetic polypeptide
GSGCLEFWWKCNPNDDKCCRPKLKCSPLGKLCNFSFG

SEQ ID NO: 144 - protein, 37 aa, Artificial Sequence
  Description of Artificial Sequence Synthetic polypeptide
GSGCLEFWWKCNPNDDKCCRPKLKCSPNGKLCNFSFG

SEQ ID NO: 145 - protein, 37 aa, Artificial Sequence
  Description of Artificial Sequence Synthetic polypeptide
GSGCLEFWWKCNPNDDKCCRPKLKCSRKTKLCNFSFG

SEQ ID NO: 146 - protein, 37 aa, Artificial Sequence
  Description of Artificial Sequence Synthetic polypeptide
GSGCLEFWWKCNPNDDKCCRPKLKCGSNFKLCNFSFG

SEQ ID NO: 147 - protein, 37 aa, Artificial Sequence
  Description of Artificial Sequence Synthetic polypeptide
GSGCLEFWWKCNPNDDKCCRPKLKCSTKHKLCNFSFG

SEQ ID NO: 148 - protein, 37 aa, Artificial Sequence
  Description of Artificial Sequence Synthetic polypeptide
GSGCLEFWWKCNPNDDKCCRPKLKCSNDGKLCNFSFG

SEQ ID NO: 149 - protein, 37 aa, Artificial Sequence
  Description of Artificial Sequence Synthetic polypeptide
GSGCLEFWWKCNPNDDKCCRPKLKCSKKTKLCNFSFG

SEQ ID NO: 150 - protein, 37 aa, Artificial Sequence
  Description of Artificial Sequence Synthetic polypeptide
GSGCLEFWWKCNPNDDKCCRPKLKCHSNFKLCNFSFG

SEQ ID NO: 151 - protein, 37 aa, Artificial Sequence
  Description of Artificial Sequence Synthetic polypeptide
GSGCLEFWWKCNPNDDKCCRPKLKCSKKFTACNFSFG

SEQ ID NO: 152 - protein, 37 aa, Artificial Sequence
  Description of Artificial Sequence Synthetic polypeptide
GSGCLEIFKACNPNDDKCCRPKLKCSKLFKLCNFSFG

SEQ ID NO: 153 - protein, 37 aa, Artificial Sequence
  Description of Artificial Sequence Synthetic polypeptide
GSGCLKFGWKCNPNDDKCCRPKLKCSKLFKLCNFSFG

SEQ ID NO: 154 - protein, 38 aa, Artificial Sequence
  Description of Artificial Sequence Synthetic polypeptide
GSGCLEFWWKCNPNDDKCCKSSKLKCSKLFKLCNFSFG

SEQ ID NO: 155 - protein, 37 aa, Artificial Sequence
  Description of Artificial Sequence Synthetic polypeptide
GSGCLEFWWKCNPNDDKCCRPKLKCNKLFKLCNISIG

SEQ ID NO: 156 - protein, 35 aa, Artificial Sequence
  Description of Artificial Sequence Synthetic polypeptide
GSGCLEFWWKCNPNDDCCRKLKCSKLFKLCNFSFG

SEQ ID NO: 157 - protein, 37 aa, Artificial Sequence
  Description of Artificial Sequence Synthetic polypeptide
GSGCLEFWWKCNPSNDQCCRPKLKCSKLFKLCNFSFG

SEQ ID NO: 158 - protein, 38 aa, Artificial Sequence
  Description of Artificial Sequence Synthetic polypeptide
GSGCLEFWWKCNPNDDKCCRPSKLVCSKLFKLCNFSFG

SEQ ID NO: 159 - protein, 37 aa, Artificial Sequence
  Description of Artificial Sequence Synthetic polypeptide
GSGCLEFLGECNPNDDKCCRPKLKCSKLFKLCNFSFG

SEQ ID NO: 160 - protein, 37 aa, Artificial Sequence
  Description of Artificial Sequence Synthetic polypeptide
GSGCLWYLWKCNPNDDKCCRPKLKCSKLFKLCNFSFG

SEQ ID NO: 161 - protein, 37 aa, Artificial Sequence
  Description of Artificial Sequence Synthetic polypeptide
GSGCLEFWWRCNPNDDRCCRPRLRCSRLFRLCNFSFG

SEQ ID NO: 162 - protein, 32 aa, Artificial Sequence
  Description of Artificial Sequence Synthetic polypeptide
GSGCLEFWWRCNPNDDRCCRPRLRCSRLFRLC

SEQ ID NO: 163 - protein, 37 aa, Artificial Sequence
  Description of Artificial Sequence Synthetic polypeptide
GSCRYFLGECKKTSECCEHLACHDKHKWCAWDWTIGK

SEQ ID NO: 164 - protein, 36 aa, Artificial Sequence
  Description of Artificial Sequence Synthetic polypeptide
GSECRYWLGGCSAGQTCCKHLVCSRRHGWCVWDGTF

SEQ ID NO: 165 - protein, 38 aa, Artificial Sequence
  Description of Artificial Sequence Synthetic polypeptide
GSECRWYLGECSQDGDCCKHLQCHSNYEWCIWDGTFSK

SEQ ID NO: 166 - protein, 38 aa, Artificial Sequence
  Description of Artificial Sequence Synthetic polypeptide
GSECRWYLGGCSQDGDCCKHLQCHSNYEWCVWDGTFSK

SEQ ID NO: 167 - protein, 36 aa, Artificial Sequence
  Description of Artificial Sequence Synthetic polypeptide
GSDCRKFLGACTQTSDCCKHLACHNKHKWCAWDWTI

SEQ ID NO: 168 - protein, 39 aa, Artificial Sequence
  Description of Artificial Sequence Synthetic polypeptide
GSECRYLMGGCSKDGDCCEHLVCRTKWPYHCVWDWTFGK

SEQ ID NO: 169 - protein, 38 aa, Artificial Sequence
  Description of Artificial Sequence Synthetic polypeptide
GSECRYRLGTCSKTGDCCSHLSCSPKHGWCVRDRTFRK

SEQ ID NO: 170 - protein, 38 aa, Artificial Sequence
  Description of Artificial Sequence Synthetic polypeptide
GSECRYELGTCSKTGDCCSHLSCSPKHGWCVEDETFRK

SEQ ID NO: 171 - protein, 38 aa, Artificial Sequence
  Description of Artificial Sequence Synthetic polypeptide
GSECRYILGTCSKTGDCCSHLSCSPKHGWCVYDITFRK

SEQ ID NO: 172 - protein, 38 aa, Artificial Sequence
  Description of Artificial Sequence Synthetic polypeptide
GSECRYWLGTCSKTGDCCSHLSCSPKGGWCVWDWTFRK

SEQ ID NO: 173 - protein, 38 aa, Artificial Sequence
  Description of Artificial Sequence Synthetic polypeptide
GSECRYWLGTCSKTGDCCSHLSCSPNHGWCVWDWTFRK

SEQ ID NO: 174 - protein, 38 aa, Artificial Sequence
  Description of Artificial Sequence Synthetic polypeptide
GSECRYWLGTCSKTGDCCSHLSCSRKTGWCVWDWTFRK

SEQ ID NO: 175 - protein, 38 aa, Artificial Sequence
  Description of Artificial Sequence Synthetic polypeptide
GSECRYWLGTCSKTGDCCSHLSCGSNHGWCVWDWTFRK

SEQ ID NO: 176 - protein, 38 aa, Artificial Sequence
  Description of Artificial Sequence Synthetic polypeptide
GSECRYWLGTCSKTGDCCSHLSCSTKHGWCVWDWTFRK

SEQ ID NO: 177 - protein, 38 aa, Artificial Sequence
  Description of Artificial Sequence Synthetic polypeptide
GSECRYWLGTCSKTGDCCSHLSCSSKHGWCVWDWTFRK

SEQ ID NO: 178 - protein, 38 aa, Artificial Sequence
  Description of Artificial Sequence Synthetic polypeptide
GSECRYWLGTCSKTGDCCSHLSCSNDGGWCVWDWTFRK

SEQ ID NO: 179 - protein, 38 aa, Artificial Sequence
  Description of Artificial Sequence Synthetic polypeptide
GSECRYWLGTCSKTGDCCSHLSCSPKTRACVWDWTFRK

SEQ ID NO: 180 - protein, 38 aa, Artificial Sequence
  Description of Artificial Sequence Synthetic polypeptide
GSECRYWLGTCSKTGDCCSHLSCHSNHGWCVWDWTFRK

SEQ ID NO: 181 - protein, 38 aa, Artificial Sequence
  Description of Artificial Sequence Synthetic polypeptide
GSECRYWLGTCSKTGDCCSHLSCSRKHRACVWDWTFRK

SEQ ID NO: 182 - protein, 41 aa, Artificial Sequence
  Description of Artificial Sequence Synthetic polypeptide
GSECRYWLGTCSKTGDQCCKSSHLSCSPKHGWCVWDWTFRK

SEQ ID NO: 183 - protein, 38 aa, Artificial Sequence
  Description of Artificial Sequence Synthetic polypeptide
GSECRYWLGTCSAGQDCCSHLSCSPKHGWCVWDWTFRK

SEQ ID NO: 184 - protein, 38 aa, Artificial Sequence
  Description of Artificial Sequence Synthetic polypeptide
GSECRYWLGGCSATGDCCSHLSCSPKHGWCVWDWTFRK

SEQ ID NO: 185 - protein, 40 aa, Artificial Sequence
  Description of Artificial Sequence Synthetic polypeptide
GSECRYWLGTCSKTGDCCKSSHLVCSPKHGWCVWDWTFRK

SEQ ID NO: 186 - protein, 38 aa, Artificial Sequence
  Description of Artificial Sequence Synthetic polypeptide
GSECLEILGTCSKTGDCCSHLSCSPKHGWCVWDWTFRK

SEQ ID NO: 187 - protein, 38 aa, Artificial Sequence
  Description of Artificial Sequence Synthetic polypeptide
GSECRYWFKACSKTGDCCSHLSCSPKHGWCVWDWTFRK

SEQ ID NO: 188 - protein, 38 aa, Artificial Sequence
  Description of Artificial Sequence Synthetic polypeptide
GSECRYWLGTCSKTGDCCSHLSCSDGHGWCVWDWTFRK

SEQ ID NO: 189 - protein, 38 aa, Artificial Sequence
  Description of Artificial Sequence Synthetic polypeptide
GSECRYWLGTCSKTGDCCSHLSCSKLHGWCVWDWTFRK

SEQ ID NO: 190 - protein, 38 aa, Artificial Sequence
  Description of Artificial Sequence Synthetic polypeptide
GSECRYWLGTCSKTGDCCSHLQCHSKHGWCVWDWTFRK

SEQ ID NO: 191 - protein, 38 aa, Artificial Sequence
  Description of Artificial Sequence Synthetic polypeptide
GSECRYWLGTCSRTGDCCSHLSCSPRHGWCVWDWTFRR

SEQ ID NO: 192 - protein, 30 aa, Artificial Sequence
  Description of Artificial Sequence Synthetic polypeptide
GSECRYWLGTCSRTGDCCSHLSCSPRHGWC

SEQ ID NO: 193 - protein, 37 aa, Artificial Sequence
  Description of Artificial Sequence Synthetic polypeptide
GSMCIPCFTTDHQMAAKCNACCGGKRGSCRGPQCICN

SEQ ID NO: 194 - protein, 36 aa, Artificial Sequence
  Description of Artificial Sequence Synthetic polypeptide
GSMCIPCFTTDHQMAAKCNACCGGKGSCRGPQCICN

SEQ ID NO: 195 - protein, 37 aa, Artificial Sequence
  Description of Artificial Sequence Synthetic polypeptide
GSMCIPCFTTDHQMAAKCNACCGGKRGSCRGPQCICI

SEQ ID NO: 196 - protein, 38 aa, Artificial Sequence
  Description of Artificial Sequence Synthetic polypeptide
GSGCLEIWWKCNPNDDKCCRPKLKCSTKHFKLCNYSIG

SEQ ID NO: 197 - protein, 36 aa, Artificial Sequence
  Description of Artificial Sequence Synthetic polypeptide
GSDCLGILWKCNPSNDKCCRPNLVCSTKHKWCKYQI

SEQ ID NO: 198 - protein, 34 aa, Artificial Sequence
  Description of Artificial Sequence Synthetic polypeptide
GSGCLEIWWKCNPNDDKCCRPKLKCSTKHFKLCN

SEQ ID NO: 199 - protein, 37 aa, Artificial Sequence
  Description of Artificial Sequence Synthetic polypeptide
GSDCLGILWKCNPSNDKCCRPNLVCSTKHKWCKYSIG

SEQ ID NO: 200 - protein, 38 aa, Artificial Sequence
  Description of Artificial Sequence Synthetic polypeptide
GSGCLEIWWKCSKTGDCCSHLSCSPKTRACVWDWTFRK

SEQ ID NO: 201 - protein, 42 aa, Artificial Sequence
  Description of Artificial Sequence Synthetic polypeptide
GSECRYWLGTCSKTGDCCSHLSCSPKTRACSKLFKLCNYSIG

SEQ ID NO: 202 - protein, 38 aa, Artificial Sequence
  Description of Artificial Sequence Synthetic polypeptide
  MOD_RES (3)..(3): Any amino acid or amino acid analogue
  MOD_RES (5)..(5): Any amino acid or amino acid analogue
  MOD_RES (11)..(17): Any amino acid or amino acid analogue
  MOD_RES (19)..(20): Any amino acid or amino acid analogue
  MOD_RES (24)..(27): Any amino acid or amino acid analogue
  MOD_RES (29)..(29): Any amino acid or amino acid analogue
  MOD_RES (31)..(31): Any amino acid or amino acid analogue
  MOD_RES (34)..(34): Any amino acid or amino acid analogue
  MOD_RES (36)..(36): Any amino acid or amino acid analogue
  MOD_RES (38)..(38): Any amino acid or amino acid analogue
GSXCXPCFTTXXXXXXXCXXCCGXXXXGXCXGPXCXCX

SEQ ID NO: 203 - protein, 38 aa, Artificial Sequence; identical to SEQ ID NO: 202
  Description of Artificial Sequence Synthetic polypeptide
  MOD_RES (3)..(3): Met, Arg, Ile, Asp, His or Leu
  MOD_RES (5)..(5): Met, Ile or Leu
  MOD_RES (11)..(11): Asp, His, Glu, Ser, Gly or Ile
  MOD_RES (12)..(12): His, Glu, Gln, Arg, Tyr or Thr
  MOD_RES (13)..(13): Gln, Arg, His, Glu, Tyr or Phe
  MOD_RES (14)..(14): Met, Ile or Leu
  MOD_RES (15)..(15): Ala, Phe, Glu, Ile or Gln
  MOD_RES (16)..(16): Arg, Glu, Ile, Asp, Asn or His
  MOD_RES (17)..(17): Arg, Asn, His, Glu, Tyr, Phe, Ile, Thr or Gln
  MOD_RES (19)..(19): Asp or Glu
  MOD_RES (20)..(20): Asp, Ile, His, Glu, Arg, Tyr, Phe or Ala
  MOD_RES (24)..(24): Gly or Ile
  MOD_RES (25)..(25): Arg, Asp, Trp, Phe or Gly
  MOD_RES (26)..(26): Gly, Asp or Ser
  MOD_RES (27)..(27): Arg, Asp, Gly or Tyr
  MOD_RES (29)..(29): Lys or Arg
  MOD_RES (31)..(31): Tyr, Asn, His, Asp or Trp
  MOD_RES (34)..(34): Gln or His
  MOD_RES (36)..(36): Leu or Ile
  MOD_RES (38)..(38): Arg, Gly, Phe or Ile

SEQ ID NO: 204 - protein, 30 aa, Artificial Sequence
  Description of Artificial Sequence Synthetic polypeptide
  MOD_RES (10)..(10): Any amino acid or amino acid analogue
  MOD_RES (13)..(13): Any amino acid or amino acid analogue
  MOD_RES (15)..(16): Any amino acid or amino acid analogue
  MOD_RES (18)..(18): Any amino acid or amino acid analogue
  MOD_RES (23)..(23): Any amino acid or amino acid analogue
GSVGCEECPXHCXGXXAXPTCDXGVCNCNV

SEQ ID NO: 205 - protein, 30 aa, Artificial Sequence; identical to SEQ ID NO: 204
  Description of Artificial Sequence Synthetic polypeptide
  MOD_RES (10)..(10): Met, Ala, Val, Ile or Leu
  MOD_RES (13)..(13): Lys or Arg
  MOD_RES (15)..(15): Lys or Arg
  MOD_RES (16)..(16): Asn, His, Met, Lys or Gln
  MOD_RES (18)..(18): Asn, Lys, Val, Ile, Leu, Arg or Gln
  MOD_RES (23)..(23): Asp, Asn, Gly, Tyr or Glu

SEQ ID NO: 206 - protein, 30 aa, Artificial Sequence
  Description of Artificial Sequence Synthetic polypeptide
  MOD_RES (6)..(6): Any amino acid or amino acid analogue
  MOD_RES (8)..(8): Any amino acid or amino acid analogue
  MOD_RES (10)..(11): Any amino acid or amino acid analogue
  MOD_RES (16)..(16): Any amino acid or amino acid analogue
  MOD_RES (18)..(18): Any amino acid or amino acid analogue
  MOD_RES (22)..(25): Any amino acid or amino acid analogue
  MOD_RES (27)..(27): Any amino acid or amino acid analogue
  MOD_RES (30)..(30): Any amino acid or amino acid analogue
GSVGCXEXPXXCKGKXAXPTCXXXXCXCNX

SEQ ID NO: 207 - protein, 30 aa, Artificial Sequence
  Description of Artificial Sequence Synthetic polypeptide
  MOD_RES (6)..(6): Ala or Glu
  MOD_RES (8)..(8): Cys or Asp
  MOD_RES (10)..(10): Met, Ala, Lys or Val
  MOD_RES (11)..(11): His or Tyr
  MOD_RES (16)..(16): Asn, His, Met, Lys or Gln
  MOD_RES (18)..(18): Asn, Lys, Val, Ile or Leu
  MOD_RES (22)..(22): Asp, Tyr, Cys or Glu
  MOD_RES (23)..(23): Asp, Asn, Gly or Tyr
  MOD_RES (24)..(24): Gly or Glu
  MISC_FEATURE (25)..(25): May or may not be present
  MOD_RES (27)..(27): Asn, Lys or Glu
  MOD_RES (30)..(30): Val, Ala, Ile or Asp
GSVGCXEXPXXCKGKXAXPTCXXXVCXCNX

SEQ ID NO: 208 - protein, 31 aa, Artificial Sequence
  Description of Artificial Sequence Synthetic polypeptide
  MOD_RES (3)..(4): Any amino acid or amino acid analogue
  MOD_RES (10)..(10): Any amino acid or amino acid analogue
  MOD_RES (13)..(18): Any amino acid or amino acid analogue
  MOD_RES (22)..(22): Any amino acid or amino acid analogue
  MOD_RES (25)..(25): Any amino acid or amino acid analogue
  MOD_RES (30)..(31): Any amino acid or amino acid analogue
GSXXCEDCPXHCXXXXXXAKCXNDXCVCEXX

SEQ ID NO: 209 - protein, 31 aa, Artificial Sequence; identical to SEQ ID NO: 208
  Description of Artificial Sequence Synthetic polypeptide
  MOD_RES (3)..(3): Val or Ala
  MOD_RES (4)..(4): Ser, Thr or Gly
  MOD_RES (10)..(10): Asp or Glu
  MOD_RES (13)..(13): Ser or Ala
  MOD_RES (14)..(14): Thr or Gln
  MOD_RES (15)..(15): Gln or Lys
  MOD_RES (16)..(16): Lys, Asn or Asp
  MOD_RES (17)..(17): Ala or Gln
  MOD_RES (18)..(18): Arg or Gln
  MOD_RES (22)..(22): Asp or Glu
  MOD_RES (25)..(25): Lys or Arg
  MOD_RES (30)..(30): Pro, Ser or Ala
  MOD_RES (31)..(31): Lys, Val or Ile

SEQ ID NO: 210 - protein, 38 aa, Artificial Sequence
  Description of Artificial Sequence Synthetic polypeptide
  MOD_RES (3)..(3): Any amino acid or amino acid analogue except Pro or Cys
  MOD_RES (5)..(5): Ala, Leu, Val, Ile or Met
  MOD_RES (14)..(14): Ala, Leu, Val, Ile or Met
  MOD_RES (29)..(29): Lys or Arg
  MOD_RES (36)..(36): Ala, Leu, Val, Ile or Met
  MOD_RES (38)..(38): Any amino acid or amino acid analogue except Cys
GSXCXPCFTTDHQXARRCDDCCGGRGRGXCYGPQCXCX

SEQ ID NO: 211 - protein, 38 aa, Artificial Sequence
  Description of Artificial Sequence Synthetic polypeptide
  MOD_RES (29)..(29): Arg or Lys
GSMCMPCFTTDHRMAENCDICCGGDGRGXCYGPQCLCR

SEQ ID NO: 212 - protein, 38 aa, Artificial Sequence
  Description of Artificial Sequence Synthetic polypeptide
  MOD_RES (3)..(3): Any amino acid or amino acid analogue
  MOD_RES (11)..(13): Any amino acid or amino acid analogue
  MOD_RES (15)..(17): Any amino acid or amino acid analogue
  MOD_RES (20)..(20): Any amino acid or amino acid analogue
  MOD_RES (24)..(27): Any amino acid or amino acid analogue
  MOD_RES (29)..(29): Any amino acid or amino acid analogue
  MOD_RES (31)..(31): Any amino acid or amino acid analogue
  MOD_RES (34)..(34): Any amino acid or amino acid analogue
  MOD_RES (38)..(38): Any amino acid or amino acid analogue
GSXCMPCFTTXXXMXXXCDXCCGXXXXGXCXGPXCLCX

SEQ ID NO: 213 - protein, 36 aa, Artificial Sequence
  Description of Artificial Sequence Synthetic polypeptide
  MOD_RES (4)..(5): Any amino acid or amino acid analogue
  MOD_RES (7)..(16): Any amino acid or amino acid analogue
  MOD_RES (18)..(19): Any amino acid or amino acid analogue
  MOD_RES (22)..(28): Any amino acid or amino acid analogue
  MOD_RES (30)..(33): Any amino acid or amino acid analogue
  MOD_RES (35)..(35): Any amino acid or amino acid analogue
  MISC_FEATURE (1)..(36): At least some or all of the cysteine residues form intramolecular disulfide bridges
  See specification as filed for detailed description of substitutions and preferred embodiments
GSCXXCXXXXXXXXXXCXXCCXXXXXXXCXXXXCXC

SEQ ID NO: 214 - protein, 34 aa, Artificial Sequence
  Description of Artificial Sequence Synthetic polypeptide
MCIPCFTTNPNMAAKCNACCGSRRGSCRGPQCIC

SEQ ID NO: 215 - protein, 35 aa, Artificial Sequence
  Description of Artificial Sequence Synthetic polypeptide
GCLEFWWKCNPNDDKCCRPKLKCSKLFKLCNFSFG

SEQ ID NO: 216 - protein, 36 aa, Artificial Sequence
  Description of Artificial Sequence Synthetic polypeptide
ECRYWLGTCSKTGDCCSHLSCSPKHGWCVWDWTFRK

SEQ ID NO: 217 - protein, 36 aa, Artificial Sequence
  Description of Artificial Sequence Synthetic polypeptide
MCMPCFTTDHQMARRCDDCCGGRGRGRCYGPQCLCR

SEQ ID NO: 218 - protein, 36 aa, Artificial Sequence
  Description of Artificial Sequence Synthetic polypeptide
MCMPCFTTHHRMAENCDICCGGDGRGKCYGPQCLCR

SEQ ID NO: 219 - protein, 36 aa, Artificial Sequence
  Description of Artificial Sequence Synthetic polypeptide
MCMPCFTTDHRMAENCDICCGGDGRGKCYGPQCLCR

SEQ ID NO: 220 - protein, 36 aa, Artificial Sequence
  Description of Artificial Sequence Synthetic polypeptide
MCMPCFTTHHQMAENCDICCGGDGRGKCYGPQCLCR

SEQ ID NO: 221 - protein, 36 aa, Artificial Sequence
  Description of Artificial Sequence Synthetic polypeptide
MCMPCFTTHHRMARNCDICCGGDGRGKCYGPQCLCR

SEQ ID NO: 222 - protein, 36 aa, Artificial Sequence
  Description of Artificial Sequence Synthetic polypeptide
MCMPCFTTHHRMAERCDICCGGDGRGKCYGPQCLCR

SEQ ID NO: 223 - protein, 36 aa, Artificial Sequence
  Description of Artificial Sequence Synthetic polypeptide
MCMPCFTTHHRMAENCDDCCGGDGRGKCYGPQCLCR

SEQ ID NO: 224 - protein, 36 aa, Artificial Sequence
  Description of Artificial Sequence Synthetic polypeptide
RCMPCFTTDHQMARRCDDCCGGRGRGKCYGPQCLCR

SEQ ID NO: 225 - protein, 36 aa, Artificial Sequence
  Description of Artificial Sequence Synthetic polypeptide
ICIPCFTTDHQIARRCDDCCGGRGRGKCYGPQCLCR

SEQ ID NO: 226 - protein, 36 aa, Artificial Sequence
  Description of Artificial Sequence Synthetic polypeptide
MCLPCFTTDHQLARRCDDCCGGRGRGKCYGPQCLCR

SEQ ID NO: 227 - protein, 36 aa, Artificial Sequence
  Description of Artificial Sequence Synthetic polypeptide
MCMPCFTTEHQMARRCEECCGGRGRGKCYGPQCLCR

SEQ ID NO: 228 - protein, 36 aa, Artificial Sequence
  Description of Artificial Sequence Synthetic polypeptide
MCIPCFTTDHQMARRCEECCGGRGRGKCYGPQCLCR

SEQ ID NO: 229 - protein, 36 aa, Artificial Sequence
  Description of Artificial Sequence Synthetic polypeptide
ICIPCFTTDHQMARRCDDCCGGRGDGKCYGPQCLCR

SEQ ID NO: 230 - protein, 36 aa, Artificial Sequence
  Description of Artificial Sequence Synthetic polypeptide
ICIPCFTTDHQIARRCDDCCGGRGRGKCYGPQCICR

SEQ ID NO: 231 - protein, 36 aa, Artificial Sequence
  Description of Artificial Sequence Synthetic polypeptide
RCMPCFTTDHFMARFCDFCCGGRGRGKCYGPQCLCR

SEQ ID NO: 232 - protein, 36 aa, Artificial Sequence
  Description of Artificial Sequence Synthetic polypeptide
RCMPCFTTDHYMARYCDYCCGGRGRGKCYGPQCLCR

SEQ ID NO: 233 - protein, 36 aa, Artificial Sequence
  Description of Artificial Sequence Synthetic polypeptide
RCMPCFTTDHRMARRCDRCCGGRGRGKCYGPQCLCR

SEQ ID NO: 234 - protein, 36 aa, Artificial Sequence
  Description of Artificial Sequence Synthetic polypeptide
RCMPCFTTDHEMARECDECCGGRGRGKCYGPQCLCR

SEQ ID NO: 235 - protein, 36 aa, Artificial Sequence
  Description of Artificial Sequence Synthetic polypeptide
RCMPCFTTDHHMARHCDHCCGGRGRGKCYGPQCLCR

SEQ ID NO: 236 - protein, 36 aa, Artificial Sequence
  Description of Artificial Sequence Synthetic polypeptide
LCLPCFTTHHRLADQCDICCGGDGRGKCYGPQCLCR

SEQ ID NO: 237 - protein, 36 aa, Artificial Sequence
  Description of Artificial Sequence Synthetic polypeptide
ICIPCFTTEHQIARRCEECCGGRGRGKCYGPQCLCR

SEQ ID NO: 238 - protein, 36 aa, Artificial Sequence
  Description of Artificial Sequence Synthetic polypeptide
MCMPCFTTDTQMQERCDRCCGGGGRGKCWGPQCLCI

SEQ ID NO: 239 - protein, 36 aa, Artificial Sequence
  Description of Artificial Sequence Synthetic polypeptide
MCMPCFTTIYRMAHECDECCGGRGRGKCYGPQCLCR

SEQ ID NO: 240 - protein, 36 aa, Artificial Sequence
  Description of Artificial Sequence Synthetic polypeptide
MCMPCFTTGYRMAEYCDICCGGRGRGKCYGPQCLCR

SEQ ID NO: 241 - protein, 36 aa, Artificial Sequence
  Description of Artificial Sequence Synthetic polypeptide
MCMPCFTTHRRMANTCDACCGGRSRGKCYGPQCLCR

SEQ ID NO: 242 - protein, 36 aa, Artificial Sequence
  Description of Artificial Sequence Synthetic polypeptide
HCMPCFTTDHQMIRRCDDCCGGGSYGKCDGPQCLCF

SEQ ID NO: 243 - protein, 36 aa, Artificial Sequence
  Description of Artificial Sequence Synthetic polypeptide
DCMPCFTTDHRMADHCDICCGGDDRGKCYGPQCLCR

SEQ ID NO: 244 - protein, 36 aa, Artificial Sequence
  Description of Artificial Sequence Synthetic polypeptide
MCMPCFTTDHEMERRCDDCCGIGGGGKCHGPQCLCG

SEQ ID NO: 245 - protein, 36 aa, Artificial Sequence
  Description of Artificial Sequence Synthetic polypeptide
MCMPCFTTEQRMAIICDDCCGGFGRGKCYGPQCLCR

SEQ ID NO: 246 - protein, 36 aa, Artificial Sequence
  Description of Artificial Sequence Synthetic polypeptide
MCMPCFTTSEQMFRRCDDCCGGWGDGKCNGPHCLCR

SEQ ID NO: 247 - protein, 37 aa, Artificial Sequence
  Description of Artificial Sequence Synthetic polypeptide
GVPINVKCRGSRDCLDPCKKAGMRFGKCINSKCHCTP

SEQ ID NO: 248 - protein, 36 aa, Artificial Sequence
  Description of Artificial Sequence Synthetic polypeptide
MCMPCFTTEQRMAIICDDCCGGFGRGRCYGPQCLCR

SEQ ID NO: 249 - protein, 36 aa, Artificial Sequence
  Description of Artificial Sequence Synthetic polypeptide
ICIPCFTTDHQIARRCDDCCGGRGRGRCYGPQCICR

SEQ ID NO: 250 - protein, 36 aa, Artificial Sequence
  Description of Artificial Sequence Synthetic polypeptide
MCMPCFTTDTQMQERCDRCCGGGGRGRCWGPQCLCI

SEQ ID NO: 251 - protein, 35 aa, Artificial Sequence
  Description of Artificial Sequence Synthetic polypeptide
MCMPCFTTDTQMQERCDRCCGGGGRGRCWGPQCLC

SEQ ID NO: 252 - protein, 36 aa, Artificial Sequence
  Description of Artificial Sequence Synthetic polypeptide
MCMPCFTTDHRMAENCDICCGGDGRGRCYGPQCLCR

SEQ ID NO: 253 - protein, 36 aa, Artificial Sequence
  Description of Artificial Sequence Synthetic polypeptide
MCMPCFTTEQRMAIICDDCCGGFGRGKCYGPQCLCI

SEQ ID NO: 254 - protein, 36 aa, Artificial Sequence
  Description of Artificial Sequence Synthetic polypeptide
MCMPCFTTEQRMAIICDDCCGGFGRGRCYGPQCLCI

SEQ ID NO: 255 - protein, 36 aa, Artificial Sequence
  Description of Artificial Sequence Synthetic polypeptide
ICIPCFTTDHQIARRCDDCCGGRGRGKCYGPQCICI

SEQ ID NO: 256 - protein, 36 aa, Artificial Sequence
  Description of Artificial Sequence Synthetic polypeptide
ICIPCFTTDHQIARRCDDCCGGRGRGRCYGPQCICI

SEQ ID NO: 257 - protein, 36 aa, Artificial Sequence
  Description of Artificial Sequence Synthetic polypeptide
MCMPCFTTDTQMQEKCDRCCGGGGRGRCWGPQCLCI

SEQ ID NO: 258 - protein, 36 aa, Artificial Sequence
  Description of Artificial Sequence Synthetic polypeptide
MCMPCFTTEQRMAIKCDDCCGGFGRGRCYGPQCLCR

SEQ ID NO: 259 - protein, 36 aa, Artificial Sequence
  Description of Artificial Sequence Synthetic polypeptide
ICIPCFTTDHQIARKCDDCCGGRGRGRCYGPQCICR

SEQ ID NO: 260 - protein, 36 aa, Artificial Sequence
  Description of Artificial Sequence Synthetic polypeptide
MCMPCFTTDHRMAEKCDICCGGDGRGRCYGPQCLCR

SEQ ID NO: 261 - protein, 36 aa, Artificial Sequence
  Description of Artificial Sequence Synthetic polypeptide
MCMPCFTTDTQMQERCDRCCGGKGRGRCWGPQCLCI

SEQ ID NO: 262 - protein, 36 aa, Artificial Sequence
  Description of Artificial Sequence Synthetic polypeptide
MCMPCFTTEQRMAIICDDCCGGKGRGRCYGPQCLCR

SEQ ID NO: 263 - protein, 36 aa, Artificial Sequence
  Description of Artificial Sequence Synthetic polypeptide
ICIPCFTTDHQIARRCDDCCGGKGRGRCYGPQCICR

SEQ ID NO: 264 - protein, 36 aa, Artificial Sequence
  Description of Artificial Sequence Synthetic polypeptide
MCMPCFTTDHRMAENCDICCGGKGRGRCYGPQCLCR

SEQ ID NO: 265 - protein, 36 aa, Artificial Sequence
  Description of Artificial Sequence Synthetic polypeptide
MCMPCFTTDHRMAENCDICCGGDGRGKCYGPQCLCI

SEQ ID NO: 266 - protein, 36 aa, Artificial Sequence
  Description of Artificial Sequence Synthetic polypeptide
MCMPCFTTDHRMAENCDICCGGDGRGRCYGPQCLCI

SEQ ID NO: 267 - protein, 36 aa, Artificial Sequence
  Description of Artificial Sequence Synthetic polypeptide
MCMPCFTTHHRMAENCDICCGGDGRGRCYGPQCLCR

SEQ ID NO: 268 - protein, 28 aa, Artificial Sequence
  Description of Artificial Sequence Synthetic peptide
VGCEECPMHCKGKNANPTCDDGVCNCNV

SEQ ID NO: 269 - protein, 28 aa, Artificial Sequence
  Description of Artificial Sequence Synthetic peptide
VGCEECPMHCKGKNAKPTCDDGVCNCNV

SEQ ID NO: 270 - protein, 28 aa, Artificial Sequence
  Description of Artificial Sequence Synthetic peptide
VGCEECPMHCKGKNAKPTCDNGVCNCNV

SEQ ID NO: 271 - protein, 28 aa, Artificial Sequence
  Description of Artificial Sequence Synthetic peptide
VGCEECPMHCKGKHAVPTCDDGVCNCNV

SEQ ID NO: 272 - protein, 28 aa, Artificial Sequence
  Description of Artificial Sequence Synthetic peptide
VGCEECPAHCKGKNAKPTCDDGVCNCNV

SEQ ID NO: 273 - protein, 28 aa, Artificial Sequence
  Description of Artificial Sequence Synthetic peptide
VGCEECPAHCKGKNAIPTCDDGVCNCNV

SEQ ID NO: 274 - protein, 28 aa, Artificial Sequence
  Description of Artificial Sequence Synthetic peptide
VGCEECPMHCKGKMAKPTCDDGVCNCNV

SEQ ID NO: 275 - protein, 28 aa, Artificial Sequence
  Description of Artificial Sequence Synthetic peptide
VGCEECPMHCKGKNAVPTCDNGVCNCNA

SEQ ID NO: 276 - protein, 28 aa, Artificial Sequence
  Description of Artificial Sequence Synthetic peptide
VGCEECPMHCKGKMAKPTCYDGVCNCNV

SEQ ID NO: 277 - protein, 28 aa, Artificial Sequence
  Description of Artificial Sequence Synthetic peptide
VGCEECPMYCKGKNAVPTCDGGVCNCNA

SEQ ID NO: 278 - protein, 28 aa, Artificial Sequence
  Description of Artificial Sequence Synthetic peptide
VGCEECPKYCKGKNAVPTCDGGVCNCNA

SEQ ID NO: 279 - protein, 28 aa, Artificial Sequence
  Description of Artificial Sequence Synthetic peptide
VGCEECPVYCKGKKALPTCDGGVCNCNA

SEQ ID NO: 280 - protein, 28 aa, Artificial Sequence
  Description of Artificial Sequence Synthetic peptide
VGCEEDPMHCKGKQAKPTCCNGVCNCNV

SEQ ID NO: 281 - protein, 32 aa, Artificial Sequence
  Description of Artificial Sequence Synthetic polypeptide
VGCAECPMHCKGKMAKPTCENEVCKCNIGKKD

SEQ ID NO: 282 - protein, 27 aa, Artificial Sequence
  Description of Artificial Sequence Synthetic peptide
VGCEECPMHCKGKKALPTCDYGCECND

SEQ ID NO: 283 - protein, 31 aa, Artificial Sequence
  Description of Artificial Sequence Synthetic polypeptide
IVCKVCKIICGMQGKKVNICKAPIKCKCKKG

SEQ ID NO: 284 - protein, 29 aa, Artificial Sequence
  Description of Artificial Sequence Synthetic peptide
VSCEDCPDHCSTQKARAKCDNDKCVCEPK

SEQ ID NO: 285 - protein, 29 aa, Artificial Sequence
  Description of Artificial Sequence Synthetic peptide
VSCEDCPEHCSTQKARAKCDNDKCVCESV

SEQ ID NO: 286 - protein, 29 aa, Artificial Sequence
  Description of Artificial Sequence Synthetic peptide
VSCEDCPEHCSTQKAQAKCDNDKCVCEPI

SEQ ID NO: 287 - protein, 29 aa, Artificial Sequence
  Description of Artificial Sequence Synthetic peptide
ATCEDCPEHCATQNARAKCDNDKCVCEPK

SEQ ID NO: 288 - protein, 29 aa, Artificial Sequence
  Description of Artificial Sequence Synthetic peptide
VSCEDCPEHCATKDQRAKCDNDKCVCEPK

SEQ ID NO: 289 - protein, 29 aa, Artificial Sequence
  Description of Artificial Sequence Synthetic peptide
VGCEDCPEHCSQQNARAKCENDKCVCEPK

SEQ ID NO: 290 - protein, 29 aa, Artificial Sequence
  Description of Artificial Sequence Synthetic peptide
VSCEDCPEHCATKDQRAKCDNDRCVCEPK

SEQ ID NO: 291 - protein, 29 aa, Artificial Sequence
  Description of Artificial Sequence Synthetic peptide
VSCEDCPPHCATKDQRAKCENDKCVCEPK

SEQ ID NO: 292 - protein, 29 aa, Artificial Sequence
  Description of Artificial Sequence Synthetic peptide
VSCEDCPEHCSTQKARAKCDNDKCVCEAI

SEQ ID NO: 293 - protein, 35 aa, Artificial Sequence
  Description of Artificial Sequence Synthetic polypeptide
MCMPCFTTEQRMAIICDDCCGGFGRGRCYGPQCLC

SEQ ID NO: 294 - protein, 35 aa, Artificial Sequence
  Description of Artificial Sequence Synthetic polypeptide
ICIPCFTTDHQIARRCDDCCGGRGRGRCYGPQCIC

SEQ ID NO: 295 - protein, 35 aa, Artificial Sequence
  Description of Artificial Sequence Synthetic polypeptide
MCMPCFTTDHRMAENCDICCGGDGRGRCYGPQCLC

SEQ ID NO: 296 - protein, 28 aa, Artificial Sequence
  Description of Artificial Sequence Synthetic peptide
VGCEECPMHCRGRNANPTCDDGVCNCNV

SEQ ID NO: 297 - protein, 26 aa, Artificial Sequence
  Description of Artificial Sequence Synthetic peptide
VGCEECPMHCRGRNANPTCDDGVCNC

SEQ ID NO: 298 - protein, 34 aa, Artificial Sequence
  Description of Artificial Sequence Synthetic polypeptide
CGPCFTTDHQMEQKCAECCGGIGKCYGPQCLCNR

SEQ ID NO: 299 - protein, 38 aa, Artificial Sequence
  Description of Artificial Sequence Synthetic polypeptide
RCGPCFTTDPQTQAKCSECCGRKGGVCKGPQCICGIQY

SEQ ID NO: 300 - protein, 35 aa, Artificial Sequence
  Description of Artificial Sequence Synthetic polypeptide
MCMPCFTTDPNMAKKCRDCCGGNGKCFGPQCLCNR

SEQ ID NO: 301 - protein, 34 aa, Artificial Sequence
  Description of Artificial Sequence Synthetic polypeptide
MCMPCFTTDHNMAKKCNDCCGGYGKCFGPQCLCR

SEQ ID NO: 302 - protein, 36 aa, Artificial Sequence
  Description of Artificial Sequence Synthetic polypeptide
RCPPCFTTNPNMEADCRKCCGGRGYCASYQCICPGG

SEQ ID NO: 303 - protein, 35 aa, Artificial Sequence
  Description of Artificial Sequence Synthetic polypeptide
MCMPCFTTDPNMANKCRDCCGGGKKCFGPQCLCNR

SEQ ID NO: 304 - protein, 62 aa, Artificial Sequence
  Description of Artificial Sequence Synthetic polypeptide
MKFLYGVILIALFLTVMTATLSEARCGPCFTTDPQTQAKCSECCGRKGGVCKGPQCICGIQY

SEQ ID NO: 305 - protein, 36 aa, Artificial Sequence
  Description of Artificial Sequence Synthetic polypeptide
MCMPCFTTRPDMAQQCRACCKGRGKCFGPQCLCGYD

SEQ ID NO: 306 - protein, 60 aa, Artificial Sequence
  Description of Artificial Sequence Synthetic polypeptide
MKFLYGIVFIALFLTVMTATLSDAMCMPCFTTDHNMAKKCRDCCGGNGKCFGPQCLCNRG

SEQ ID NO: 307 - protein, 35 aa, Artificial Sequence
  Description of Artificial Sequence Synthetic polypeptide
MCMPCFTTDHNMAKKCRDCCGGNGKCFGPQCLCNR

SEQ ID NO: 308 - protein, 60 aa, Artificial Sequence
  Description of Artificial Sequence Synthetic polypeptide
MKFLYGIVFITLFLTVMIATHTEAMCMPCFTTRPNMAQQCRDCCRGRGKCFGPQCLCGYD

SEQ ID NO: 309 - protein, 60 aa, Artificial Sequence
  Description of Artificial Sequence Synthetic polypeptide
MKFLYGIVFIALFLTVMIATHTEAMCMPCFTTRPNMAQQCRDCCRGRGKCFGPQCLCGYD

SEQ ID NO: 310 - protein, 35 aa, Artificial Sequence
  Description of Artificial Sequence Synthetic polypeptide
RCKPCFTTDPQMSKKCADCCGGKGKGKCYGPQCLC

SEQ ID NO: 311 - protein, 61 aa, Artificial Sequence
  Description of Artificial Sequence Synthetic polypeptide
MKFLYGIVFITLFLTVMIATHTEAAMCMPCFTTNLNMEQECRDCCGGTGRCFGPQCLCGYD

SEQ ID NO: 312 - protein, 38 aa, Artificial Sequence
  Description of Artificial Sequence Synthetic polypeptide
RCSPCFTTDQQMTKKCYDCCGGKGKGKCYGPQCICAPY

SEQ ID NO: 313 - protein, 35 aa, Artificial Sequence
  Description of Artificial Sequence Synthetic polypeptide
CGPCFTTDPYTESKCATCCGGRGKCVGPQCLCNRI

SEQ ID NO: 314 - protein, 39 aa, Artificial Sequence
  Description of Artificial Sequence Synthetic polypeptide
TEAMCMPCFTTDHNMAKKCRDCCGGNGKCFGYQCLCNRG

SEQ ID NO: 315 - protein, 59 aa, Artificial Sequence
  Description of Artificial Sequence Synthetic polypeptide
MKFLYGIVFIALFLTVMFATQTDGCGPCFTTDANMARKCRECCGGIGKCFGPQCLCNRI

SEQ ID NO: 316 - protein, 59 aa, Artificial Sequence
  Description of Artificial Sequence Synthetic polypeptide
MKFLYGIVFIALFLTVMFATQTDGCGPCFTTDANMARKCRECCGGNGKCFGPQCLCNRE

SEQ ID NO: 317 - protein, 60 aa, Artificial Sequence
  Description of Artificial Sequence Synthetic polypeptide
MKFLYGTILIAFFLTVMIATHSEARCPPCFTTNPNMEADCRKCCGGRGYCASYQCICPGG

SEQ ID NO: 318 - protein, 39 aa, Artificial Sequence
  Description of Artificial Sequence Synthetic polypeptide
TEAMCMPCFTTRPDMAQQCRDCCGGNGKCFGYQCLCNRG

SEQ ID NO: 319 - protein, 60 aa, Artificial Sequence
  Description of Artificial Sequence Synthetic polypeptide
MKFLYGIVFIALFLTVMIATLTEAMCMPCFTTRPDMAQQCRDCCGGNGKCFGYQCLCNRG

SEQ ID NO: 320 - protein, 60 aa, Artificial Sequence
  Description of Artificial Sequence Synthetic polypeptide
MKFLYGIVFIALFLTVMIATHTEAMCMPCFTTRPDMAQQCRDCCGGNGKCFGYQCLCNRG

SEQ ID NO: 321 - protein, 61 aa, Artificial Sequence
  Description of Artificial Sequence Synthetic polypeptide
MKFLYGIILIALFLTVMIATHSEARCPNCFTTNPNAEADCKKCCGNRWGKCAGYQCVCPMK

SEQ ID NO: 322 - protein, 67 aa, Artificial Sequence
  Description of Artificial Sequence Synthetic polypeptide
MKFLYGIVFIALFLTGMIATHTEAMCMPCFTTRPDMAQQCRDCCGGNGKCFGYQCLCNRGRIVIMYT

SEQ ID NO: 323 - protein, 35 aa, Artificial Sequence
  Description of Artificial Sequence Synthetic polypeptide
MCMPCFTTRPGMAQQCRDCCGGNGKCFGYQCLCNR

SEQ ID NO: 324 - protein, 35 aa, Artificial Sequence
  Description of Artificial Sequence Synthetic polypeptide
MCIPCFTTNPNMAAKCNACCGSRRGSCRGPQCICR

SEQ ID NO: 325 - protein, 35 aa, Artificial Sequence
  Description of Artificial Sequence Synthetic polypeptide
MCIPCFTTNPNMAAKCNACCGSRRGSCRGPQCICN

SEQ ID NO: 326 - protein, 34 aa, Artificial Sequence
  Description of Artificial Sequence Synthetic polypeptide
MCIPCFTTNPNMAAKCNACCGGNGSCRGPQCICN

SEQ ID NO: 327 - protein, 36 aa, Artificial Sequence
  Description of Artificial Sequence Synthetic polypeptide
MCIPCFTTNPNMAAKCNACCGSRGRGSCRGPQCICN

SEQ ID NO: 328 - protein, 36 aa, Artificial Sequence
  Description of Artificial Sequence Synthetic polypeptide
MCIPCFTTNPNMAAKCNACCGSRGRGKCRGPQCICN

SEQ ID NO: 329 - protein, 35 aa, Artificial Sequence
  Description of Artificial Sequence Synthetic polypeptide
MCIPCFTTDHQMAAKCNACCGSRRGSCRGPQCICN

SEQ ID NO: 330 - protein, 35 aa, Artificial Sequence
  Description of Artificial Sequence Synthetic polypeptide
MCIPCFTTNHQMAAKCNACCGSRRGSCRGPQCICN

SEQ ID NO: 331 - protein, 36 aa, Artificial Sequence
  Description of Artificial Sequence Synthetic polypeptide
MCIPCFTTNPNMARKCNACCGSRGRGSCRGPQCICN

SEQ ID NO: 332 - protein, 36 aa, Artificial Sequence
  Description of Artificial Sequence Synthetic polypeptide
MCIPCFTTNPNMAAKCNACCGGKGRGSCRGPQCICN

SEQ ID NO: 333 - protein, 35 aa, Artificial Sequence
  Description of Artificial Sequence Synthetic polypeptide
MCIPCFTTNPNMAAKCNACCGSRRGSCFGPQCICN

SEQ ID NO: 334 - protein, 36 aa, Artificial Sequence
  Description of Artificial Sequence Synthetic polypeptide
MCIPCFTTNPNMAAKCNACCGSRGRGKCFGPQCICN

SEQ ID NO: 335 - protein, 36 aa, Artificial Sequence
  Description of Artificial Sequence Synthetic polypeptide
MCIPCFTTNPNMAAKCNACCGSRGRGSCFGPQCICN

SEQ ID NO: 336 - protein, 36 aa, Artificial Sequence
  Description of Artificial Sequence Synthetic polypeptide
MCIPCFTTNPNMAAKCNACCGSRGRGSCYGPQCICN

SEQ ID NO: 337 - protein, 35 aa, Artificial Sequence
  Description of Artificial Sequence Synthetic polypeptide
MCIPCFTTNPNMAAKCDACCGSRRGSCRGPQCICN

SEQ ID NO: 338 - protein, 35 aa, Artificial Sequence
  Description of Artificial Sequence Synthetic polypeptide
MCIPCFTTNHQMAAKCDACCGSRRGSCRGPQCICN

SEQ ID NO: 339 - protein, 36 aa, Artificial Sequence
  Description of Artificial Sequence Synthetic polypeptide
MCIPCFTTNHNMAAKCDACCGGRGRGSCRGPQCICN

SEQ ID NO: 340 - protein, 36 aa, Artificial Sequence
  Description of Artificial Sequence Synthetic polypeptide
MCIPCFTTNPNMAAKCDACCGSRGRGSCRGPQCICN

SEQ ID NO: 341 - protein, 36 aa, Artificial Sequence
  Description of Artificial Sequence Synthetic polypeptide
MCIPCFTTNPNMAAKCDACCGGKGRGSCRGPQCICN

SEQ ID NO: 342 - protein, 36 aa, Artificial Sequence
  Description of Artificial Sequence Synthetic polypeptide
MCIPCFTTNHNMAAKCDACCGSRGRGSCRGPQCICN

SEQ ID NO: 343 - protein, 34 aa, Artificial Sequence
  Description of Artificial Sequence Synthetic polypeptide
MCIPCFTTNPNMAAKCRDCCGGRGSCRGPQCICN

SEQ ID NO: 344 - protein, 36 aa, Artificial Sequence
  Description of Artificial Sequence Synthetic polypeptide
MCMPCFTTNPNMAAKCDDCCGSRGRGSCRGPQCICN

SEQ ID NO: 345 - protein, 34 aa, Artificial Sequence
  Description of Artificial Sequence Synthetic polypeptide
MCIPCFTTNPNMAARCNACCGSRRGSCRGPQCIC

SEQ ID NO: 346 - protein, 35 aa, Artificial Sequence
  Description of Artificial Sequence Synthetic polypeptide
MCIPCFTTNPNMAAKCNACCGSRRGSCRGPQCICI

SEQ ID NO: 347 - protein, 36 aa, Artificial Sequence
  Description of Artificial Sequence Synthetic polypeptide
GCLQFMWKCNPDNDKCCRPNLKCNTYHKWCEFVTGK

SEQ ID NO: 348 - protein, 34 aa, Artificial Sequence
  Description of Artificial Sequence Synthetic polypeptide
DCLGFLWKCNPSNDKCCRPNLVCSRKDKWCKYQI

SEQ ID NO: 349 - protein, 36 aa, Artificial Sequence
  Description of Artificial Sequence Synthetic polypeptide
DCLGFMRKCIPDNDKCCRPNLVCSRTHKWCKYVFGK

SEQ ID NO: 350 - protein, 35 aa, Artificial Sequence
  Description of Artificial Sequence Synthetic polypeptide
ECLEIFKACNPSNDQCCKSSKLVCSRKTRACKYQI

SEQ ID NO: 351 - protein, 34 aa, Artificial Sequence
  Description of Artificial Sequence Synthetic polypeptide
ECGGFWWKCGSGKPACCPKYVCSPKWGLCNFPMP

SEQ ID NO: 352 - protein, 35 aa, Artificial Sequence
  Description of Artificial Sequence Synthetic polypeptide
GCLERWWKCNPNDDKCCRPKLKCSKLFKLCNRSRG

SEQ ID NO: 353 - protein, 35 aa, Artificial Sequence
  Description of Artificial Sequence Synthetic polypeptide
GCLEEWWKCNPNDDKCCRPKLKCSKLFKLCNESEG

SEQ ID NO: 354 - protein, 35 aa, Artificial Sequence
  Description of Artificial Sequence Synthetic polypeptide
GCLEIWWKCNPNDDKCCRPKLKCSKLFKLCNYSIG

SEQ ID NO: 355 - protein, 35 aa, Artificial Sequence
  Description of Artificial Sequence Synthetic polypeptide
GCLEFWWKCNPNDDKCCRPKLKCSKLGKLCNFSFG

SEQ ID NO: 356 - protein, 35 aa, Artificial Sequence
  Description of Artificial Sequence Synthetic polypeptide
GCLEFWWKCNPNDDKCCRPKLKCSPLGKLCNFSFG

SEQ ID NO: 357 - protein, 35 aa, Artificial Sequence
  Description of Artificial Sequence Synthetic polypeptide
GCLEFWWKCNPNDDKCCRPKLKCSPNGKLCNFSFG

SEQ ID NO: 358 - protein, 35 aa, Artificial Sequence
  Description of Artificial Sequence Synthetic polypeptide
GCLEFWWKCNPNDDKCCRPKLKCSRKTKLCNFSFG

SEQ ID NO: 359 - protein, 35 aa, Artificial Sequence
  Description of Artificial Sequence Synthetic polypeptide
GCLEFWWKCNPNDDKCCRPKLKCGSNFKLCNFSFG

SEQ ID NO: 360 - protein, 35 aa, Artificial Sequence
  Description of Artificial Sequence Synthetic polypeptide
GCLEFWWKCNPNDDKCCRPKLKCSTKHKLCNFSFG

SEQ ID NO: 361 - protein, 35 aa, Artificial Sequence
  Description of Artificial Sequence Synthetic polypeptide
GCLEFWWKCNPNDDKCCRPKLKCSNDGKLCNFSFG

SEQ ID NO: 362 - protein, 35 aa, Artificial Sequence
  Description of Artificial Sequence Synthetic polypeptide
GCLEFWWKCNPNDDKCCRPKLKCSKKTKLCNFSFG

SEQ ID NO: 363 - protein, 35 aa, Artificial Sequence
  Description of Artificial Sequence Synthetic polypeptide
GCLEFWWKCNPNDDKCCRPKLKCHSNFKLCNFSFG

SEQ ID NO: 364 - protein, 35 aa, Artificial Sequence
  Description of Artificial Sequence Synthetic polypeptide
GCLEFWWKCNPNDDKCCRPKLKCSKKFTACNFSFG

SEQ ID NO: 365 - protein, 35 aa, Artificial Sequence
  Description of Artificial Sequence Synthetic polypeptide
GCLEIFKACNPNDDKCCRPKLKCSKLFKLCNFSFG

SEQ ID NO: 366 - protein, 35 aa, Artificial Sequence
  Description of Artificial Sequence Synthetic polypeptide
GCLKFGWKCNPNDDKCCRPKLKCSKLFKLCNFSFG

SEQ ID NO: 367 - protein, 36 aa, Artificial Sequence
  Description of Artificial Sequence Synthetic polypeptide
GCLEFWWKCNPNDDKCCKSSKLKCSKLFKLCNFSFG

SEQ ID NO: 368 - protein, 35 aa, Artificial Sequence
  Description of Artificial Sequence Synthetic polypeptide
GCLEFWWKCNPNDDKCCRPKLKCNKLFKLCNISIG

SEQ ID NO: 369 - protein, 33 aa, Artificial Sequence
  Description of Artificial Sequence Synthetic polypeptide
GCLEFWWKCNPNDDCCRKLKCSKLFKLCNFSFG

SEQ ID NO: 370 - protein, 35 aa, Artificial Sequence
  Description of Artificial Sequence Synthetic polypeptide
GCLEFWWKCNPSNDQCCRPKLKCSKLFKLCNFSFG

SEQ ID NO: 371 - protein, 36 aa, Artificial Sequence
  Description of Artificial Sequence Synthetic polypeptide
GCLEFWWKCNPNDDKCCRPSKLVCSKLFKLCNFSFG

SEQ ID NO: 372 - protein, 35 aa, Artificial Sequence
  Description of Artificial Sequence Synthetic polypeptide
GCLEFLGECNPNDDKCCRPKLKCSKLFKLCNFSFG

SEQ ID NO: 373 - protein, 35 aa, Artificial Sequence
  Description of Artificial Sequence Synthetic polypeptide
GCLWYLWKCNPNDDKCCRPKLKCSKLFKLCNFSFG

SEQ ID NO: 374 - protein, 35 aa, Artificial Sequence
  Description of Artificial Sequence Synthetic polypeptide
GCLEFWWRCNPNDDRCCRPRLRCSRLFRLCNFSFG

SEQ ID NO: 375 - protein, 30 aa, Artificial Sequence
  Description of Artificial Sequence Synthetic polypeptide
GCLEFWWRCNPNDDRCCRPRLRCSRLFRLC

SEQ ID NO: 376 - protein, 35 aa, Artificial Sequence
  Description of Artificial Sequence Synthetic polypeptide
CRYFLGECKKTSECCEHLACHDKHKWCAWDWTIGK

SEQ ID NO: 377 - protein, 34 aa, Artificial Sequence
  Description of Artificial Sequence Synthetic polypeptide
ECRYWLGGCSAGQTCCKHLVCSRRHGWCVWDGTF

SEQ ID NO: 378 - protein, 36 aa, Artificial Sequence
  Description of Artificial Sequence Synthetic polypeptide
ECRWYLGECSQDGDCCKHLQCHSNYEWCIWDGTFSK

SEQ ID NO: 379 - protein, 36 aa, Artificial Sequence
  Description of Artificial Sequence Synthetic polypeptide
ECRWYLGGCSQDGDCCKHLQCHSNYEWCVWDGTFSK

SEQ ID NO: 380 - protein, 34 aa, Artificial Sequence
  Description of Artificial Sequence Synthetic polypeptide
DCRKFLGACTQTSDCCKHLACHNKHKWCAWDWTI

SEQ ID NO: 381 - protein, 37 aa, Artificial Sequence
  Description of Artificial Sequence Synthetic polypeptide
ECRYLMGGCSKDGDCCEHLVCRTKWPYHCVWDWTFGK

SEQ ID NO: 382 - protein, 36 aa, Artificial Sequence
  Description of Artificial Sequence Synthetic polypeptide
ECRYRLGTCSKTGDCCSHLSCSPKHGWCVRDRTFRK

SEQ ID NO: 383 - protein, 36 aa, Artificial Sequence
  Description of Artificial Sequence Synthetic polypeptide
ECRYELGTCSKTGDCCSHLSCSPKHGWCVEDETFRK

SEQ ID NO: 384 - protein, 36 aa, Artificial Sequence
  Description of Artificial Sequence Synthetic polypeptide
ECRYILGTCSKTGDCCSHLSCSPKHGWCVYDITFRK

SEQ ID NO: 385 - protein, 36 aa, Artificial Sequence
  Description of Artificial Sequence Synthetic polypeptide
ECRYWLGTCSKTGDCCSHLSCSPKGGWCVWDWTFRK

SEQ ID NO: 386 - protein, 36 aa, Artificial Sequence
  Description of Artificial Sequence Synthetic polypeptide
ECRYWLGTCSKTGDCCSHLSCSPNHGWCVWDWTFRK

SEQ ID NO: 387 - protein, 36 aa, Artificial Sequence
  Description of Artificial Sequence Synthetic polypeptide
ECRYWLGTCSKTGDCCSHLSCSRKTGWCVWDWTFRK

SEQ ID NO: 388 - protein, 36 aa, Artificial Sequence
  Description of Artificial Sequence Synthetic polypeptide
ECRYWLGTCSKTGDCCSHLSCGSNHGWCVWDWTFRK

SEQ ID NO: 389 - protein, 36 aa, Artificial Sequence
  Description of Artificial Sequence Synthetic polypeptide
ECRYWLGTCSKTGDCCSHLSCSTKHGWCVWDWTFRK

SEQ ID NO: 390 - protein, 36 aa, Artificial Sequence
  Description of Artificial Sequence Synthetic polypeptide
ECRYWLGTCSKTGDCCSHLSCSSKHGWCVWDWTFRK

SEQ ID NO: 391 - protein, 36 aa, Artificial Sequence
  Description of Artificial Sequence Synthetic polypeptide
ECRYWLGTCSKTGDCCSHLSCSNDGGWCVWDWTFRK

SEQ ID NO: 392 - protein, 36 aa, Artificial Sequence
  Description of Artificial Sequence Synthetic polypeptide
ECRYWLGTCSKTGDCCSHLSCSPKTRACVWDWTFRK

SEQ ID NO: 393 - protein, 36 aa, Artificial Sequence
  Description of Artificial Sequence Synthetic polypeptide
ECRYWLGTCSKTGDCCSHLSCHSNHGWCVWDWTFRK

SEQ ID NO: 394 - protein, 36 aa, Artificial Sequence
  Description of Artificial Sequence Synthetic polypeptide
ECRYWLGTCSKTGDCCSHLSCSRKHRACVWDWTFRK

SEQ ID NO: 395 - protein, 39 aa, Artificial Sequence
  Description of Artificial Sequence Synthetic polypeptide
ECRYWLGTCSKTGDQCCKSSHLSCSPKHGWCVWDWTFRK

SEQ ID NO: 396 - protein, 36 aa, Artificial Sequence
  Description of Artificial Sequence Synthetic polypeptide
ECRYWLGTCSAGQDCCSHLSCSPKHGWCVWDWTFRK

SEQ ID NO: 397 - protein, 36 aa, Artificial Sequence
  Description of Artificial Sequence Synthetic polypeptide
ECRYWLGGCSATGDCCSHLSCSPKHGWCVWDWTFRK

SEQ ID NO: 398 - protein, 38 aa, Artificial Sequence
  Description of Artificial Sequence Synthetic polypeptide
ECRYWLGTCSKTGDCCKSSHLVCSPKHGWCVWDWTFRK

SEQ ID NO: 399 - protein, 36 aa, Artificial Sequence
  Description of Artificial Sequence Synthetic polypeptide
ECLEILGTCSKTGDCCSHLSCSPKHGWCVWDWTFRK

SEQ ID NO: 400 - protein, 36 aa, Artificial Sequence
  Description of Artificial Sequence Synthetic polypeptide
ECRYWFKACSKTGDCCSHLSCSPKHGWCVWDWTFRK

SEQ ID NO: 401 - protein, 36 aa, Artificial Sequence
  Description of Artificial Sequence Synthetic polypeptide
ECRYWLGTCSKTGDCCSHLSCSDGHGWCVWDWTFRK

SEQ ID NO: 402 - protein, 36 aa, Artificial Sequence
  Description of Artificial Sequence Synthetic polypeptide
ECRYWLGTCSKTGDCCSHLSCSKLHGWCVWDWTFRK

SEQ ID NO: 403 - protein, 36 aa, Artificial Sequence
  Description of Artificial Sequence Synthetic polypeptide
ECRYWLGTCSKTGDCCSHLQCHSKHGWCVWDWTFRK

SEQ ID NO: 404 - protein, 36 aa, Artificial Sequence
  Description of Artificial Sequence Synthetic polypeptide
ECRYWLGTCSRTGDCCSHLSCSPRHGWCVWDWTFRR

SEQ ID NO: 405 - protein, 28 aa, Artificial Sequence
  Description of Artificial Sequence Synthetic peptide
ECRYWLGTCSRTGDCCSHLSCSPRHGWC

SEQ ID NO: 406 - protein, 35 aa, Artificial Sequence
  Description of Artificial Sequence Synthetic polypeptide
MCIPCFTTDHQMAAKCNACCGGKRGSCRGPQCICN

SEQ ID NO: 407 - protein, 34 aa, Artificial Sequence
  Description of Artificial Sequence Synthetic polypeptide
MCIPCFTTDHQMAAKCNACCGGKGSCRGPQCICN

SEQ ID NO: 408 - protein, 35 aa, Artificial Sequence
  Description of Artificial Sequence Synthetic polypeptide
MCIPCFTTDHQMAAKCNACCGGKRGSCRGPQCICI

SEQ ID NO: 409 - protein, 36 aa, Artificial Sequence
  Description of Artificial Sequence Synthetic polypeptide
GCLEIWWKCNPNDDKCCRPKLKCSTKHFKLCNYSIG

SEQ ID NO: 410 - protein, 34 aa, Artificial Sequence
  Description of Artificial Sequence Synthetic polypeptide
DCLGILWKCNPSNDKCCRPNLVCSTKHKWCKYQI

SEQ ID NO: 411 - protein, 32 aa, Artificial Sequence
  Description of Artificial Sequence Synthetic polypeptide
GCLEIWWKCNPNDDKCCRPKLKCSTKHFKLCN

SEQ ID NO: 412 - protein, 35 aa, Artificial Sequence
  Description of Artificial Sequence Synthetic polypeptide
DCLGILWKCNPSNDKCCRPNLVCSTKHKWCKYSIG

SEQ ID NO: 413 - protein, 36 aa, Artificial Sequence
  Description of Artificial Sequence Synthetic polypeptide
GCLEIWWKCSKTGDCCSHLSCSPKTRACVWDWTFRK

SEQ ID NO: 414 - protein, 40 aa, Artificial Sequence
  Description of Artificial Sequence Synthetic polypeptide
ECRYWLGTCSKTGDCCSHLSCSPKTRACSKLFKLCNYSIG

SEQ ID NO: 415 - protein, 36 aa, Artificial Sequence
  Description of Artificial Sequence Synthetic polypeptide
  MOD_RES (1)..(1): Any amino acid or amino acid analogue
  MOD_RES (3)..(3): Any amino acid or amino acid analogue
  MOD_RES (9)..(15): Any amino acid or amino acid analogue
  MOD_RES (17)..(18): Any amino acid or amino acid analogue
  MOD_RES (22)..(25): Any amino acid or amino acid analogue
  MOD_RES (27)..(27): Any amino acid or amino acid analogue
  MOD_RES (29)..(29): Any amino acid or amino acid analogue
  MOD_RES (32)..(32): Any amino acid or amino acid analogue
  MOD_RES (34)..(34): Any amino acid or amino acid analogue
  MOD_RES (36)..(36): Any amino acid or amino acid analogue
XCXPCFTTXXXXXXXCXXCCGXXXXGXCXGPXCXCX

SEQ ID NO: 416 - protein, 36 aa, Artificial Sequence; identical to SEQ ID NO: 415
  Description of Artificial Sequence Synthetic polypeptide
  MOD_RES (1)..(1): Met, Arg, Ile, Asp, His or Leu
  MOD_RES (3)..(3): Met, Ile or Leu
  MOD_RES (9)..(9): Asp, His, Glu, Ser, Gly or Ile
  MOD_RES (10)..(10): His, Glu, Gln, Arg, Tyr or Thr
  MOD_RES (11)..(11): Gln, Arg, His, Glu, Tyr or Phe
  MOD_RES (12)..(12): Met, Ile or Leu
  MOD_RES (13)..(13): Ala, Phe, Glu, Ile or Gln
  MOD_RES (14)..(14): Arg, Glu, Ile, Asp, Asn or His
  MOD_RES (15)..(15): Arg, Asn, His, Glu, Tyr, Phe, Ile, Thr or Gln
  MOD_RES (17)..(17): Asp or Glu
  MOD_RES (18)..(18): Asp, Ile, His, Glu, Arg, Tyr, Phe or Ala
  MOD_RES (22)..(22): Gly or Ile
  MOD_RES (23)..(23): Arg, Asp, Trp, Phe or Gly
  MOD_RES (24)..(24): Gly, Asp or Ser
  MOD_RES (25)..(25): Arg, Asp, Gly or Tyr
  MOD_RES (27)..(27): Lys or Arg
  MOD_RES (29)..(29): Tyr, Asn, His, Asp or Trp
  MOD_RES (32)..(32): Gln or His
  MOD_RES (34)..(34): Leu or Ile
  MOD_RES (36)..(36): Arg, Gly, Phe or Ile

SEQ ID NO: 417 - protein, 28 aa, Artificial Sequence
  Description of Artificial Sequence Synthetic peptide
  MOD_RES (8)..(8): Any amino acid or amino acid analogue
  MOD_RES (11)..(11): Any amino acid or amino acid analogue
  MOD_RES (13)..(14): Any amino acid or amino acid analogue
  MOD_RES (16)..(16): Any amino acid or amino acid analogue
  MOD_RES (21)..(21): Any amino acid or amino acid analogue
VGCEECPXHCXGXXAXPTCDXGVCNCNV

SEQ ID NO: 418 - protein, 28 aa, Artificial Sequence
  Description of Artificial Sequence Synthetic peptide
  MOD_RES (8)..(8): Met, Ala, Val, Ile or Leu
  MOD_RES (11)..(11): Lys or Arg
  MOD_RES (13)..(13): Lys or Arg
  MOD_RES (14)..(14): Asn, His, Met, Lys or Gln
  MOD_RES (16)..(16): Asn, Lys, Val, Ile, Leu, Arg or Gln
  MOD_RES (21)..(21): Asp, Asn, Gly, Tyr or Glu
VGCEECPXHCXGXXAXPTCDXGVCNCNV

SEQ ID NO: 419 - protein, 28 aa, Artificial Sequence
  Description of Artificial Sequence Synthetic peptide
  MOD_RES (4)..(4): Any amino acid or amino acid analogue
  MOD_RES (6)..(6): Any amino acid or amino acid analogue
  MOD_RES (8)..(9): Any amino acid or amino acid analogue
  MOD_RES (14)..(14): Any amino acid or amino acid analogue
  MOD_RES (16)..(16): Any amino acid or amino acid analogue
  MOD_RES (20)..(23): Any amino acid or amino acid analogue
  MOD_RES (25)..(25): Any amino acid or amino acid analogue
  MOD_RES (28)..(28): Any amino acid or amino acid analogue
VGCXEXPXXCKGKXAXPTCXXXXCXCNX

SEQ ID NO: 420 - protein, 28 aa, Artificial Sequence
  Description of Artificial Sequence Synthetic peptide
  MOD_RES (4)..(4): Ala or Glu
  MOD_RES (6)..(6): Cys or Asp
  MOD_RES (8)..(8): Met, Ala, Lys or Val
  MOD_RES (9)..(9): His or Tyr
  MOD_RES (14)..(14): Asn, His, Met, Lys or Gln
  MOD_RES (16)..(16): Asn, Lys Val, Ile or Leu
  MOD_RES (20)..(20): Asp, Tyr, Cys or Glu
  MOD_RES (21)..(21): Asp, Asn, Gly or Tyr
  MOD_RES (22)..(22): Gly or Glu
  MISC_FEATURE (23)..(23): May or may not be present
  MOD_RES (25)..(25): Asn, Lys or Glu
  MOD_RES (28)..(28): Val, Ala, Ile or Asp
VGCXEXPXXCKGKXAXPTCXXXVCXCNX

SEQ ID NO: 421 - protein, 29 aa, Artificial Sequence
  Description of Artificial Sequence Synthetic peptide
  MOD_RES (1)..(2): Any amino acid or amino acid analogue
  MOD_RES (8)..(8): Any amino acid or amino acid analogue
  MOD_RES (11)..(16): Any amino acid or amino acid analogue
  MOD_RES (20)..(20): Any amino acid or amino acid analogue
  MOD_RES (23)..(23): Any amino acid or amino acid analogue
  MOD_RES (28)..(29): Any amino acid or amino acid analogue
XXCEDCPXHCXXXXXXAKCXNDXCVCEXX

SEQ ID NO: 422 - protein, 29 aa, Artificial Sequence
  Description of Artificial Sequence Synthetic peptide
  MOD_RES (1)..(1): Val or Ala
  MOD_RES (2)..(2): Ser, Thr or Gly
  MOD_RES (8)..(8): Asp or Glu
  MOD_RES (11)..(11): Ser or Ala
  MOD_RES (12)..(12): Thr or Gln
  MOD_RES (13)..(13): Gln or Lys
  MOD_RES (14)..(14): Lys, Asn or Asp
  MOD_RES (15)..(15): Ala or Gln
  MOD_RES (16)..(16): Arg or Gln
  MOD_RES (20)..(20): Asp or Glu
  MOD_RES (23)..(23): Lys or Arg
  MOD_RES (28)..(28): Pro, Ser or Ala
  MOD_RES (29)..(29): Lys, Val or Ile
XXCEDCPXHCXXXXXXAKCXNDXCVCEXX

SEQ ID NO: 423 - protein, 36 aa, Artificial Sequence
  Description of Artificial Sequence Synthetic polypeptide
  MOD_RES (1)..(1): Any amino ccid or amino acid analogue except Pro or Cys
  MOD_RES (3)..(3): Ala, Leu, Val, Ile or Met
  MOD_RES (12)..(12): Ala, Leu, Val, Ile or Met
  MOD_RES (27)..(27): Lys or Arg
  MOD_RES (34)..(34): Ala, Leu, Val, Ile or Met
  MOD_RES (36)..(36): Any amino acid or amino acid analogue except Cys
XCXPCFTTDHQXARRCDDCCGGRGRGXCYGPQCXCX

SEQ ID NO: 424 - protein, 36 aa, Artificial Sequence
  Description of Artificial Sequence Synthetic polypeptide
  MOD_RES (27)..(27): Arg or Lys
MCMPCFTTDHRMAENCDICCGGDGRGXCYGPQCLCR

SEQ ID NO: 425 - protein, 36 aa, Artificial Sequence
  Description of Artificial Sequence Synthetic polypeptide
  MOD_RES (1)..(1): Any amino acid or amino acid analogue
  MOD_RES (9)..(11): Any amino acid or amino acid analogue
  MOD_RES (13)..(15): Any amino acid or amino acid analogue
  MOD_RES (18)..(18): Any amino acid or amino acid analogue
  MOD_RES (22)..(25): Any amino acid or amino acid analogue
  MOD_RES (27)..(27): Any amino acid or amino acid analogue
  MOD_RES (29)..(29): Any amino acid or amino acid analogue
  MOD_RES (32)..(32): Any amino acid or amino acid analogue
  MOD_RES (36)..(36): Any amino acid or amino acid analogue
XCMPCFTTXXXMXXXCDXCCGXXXXGXCXGPXCLCX

SEQ ID NO: 426 - protein, 34 aa, Artificial Sequence
  Description of Artificial Sequence Synthetic polypeptide
  MOD_RES (2)..(3): Any amino acid or amino acid analogue
  MOD_RES (5)..(14): Any amino acid or amino acid analogue
  MOD_RES (16)..(17): Any amino acid or amino acid analogue
  MOD_RES (20)..(26): Any amino acid or amino acid analogue
  MOD_RES (28)..(31): Any amino acid or amino acid analogue
  MOD_RES (33)..(33): Any amino acid or amino acid analogue
  MISC_FEATURE (1)..(34): At least some or all of the cysteine residues form intramolecular disulfide bridges
  See specification as filed for detailed description of substitutions and preferred embodiments
CXXCXXXXXXXXXXCXXCCXXXXXXXCXXXXCXC

SEQ ID NO: 427 - protein, 468 aa, Artificial Sequence
  Description of Artificial Sequence Synthetic polypeptide
MEFGLSWVFLVALFRGVQSEVQLVESGGGLVQPGGSLRLSCAASGFTFLGYGIHWVRQAPGKGLEWVGWISPAGGSTDYADSVKGRFTISADTSKNTAYLQMNSLRAEDTAVYYCARGPFSPWVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 428 - protein, 234 aa, Artificial Sequence
  Description of Artificial Sequence Synthetic polypeptide
METDTLLLWVLLLWVPGSTGDIQMTQSPSSLSASVGDRVTITCRASQDVSTAVAWYQQKPGKAPKLLIYSASFLYSGVPSRFSGSGSGTDFTLTISSLQPEDFATYYCQQFPTYLPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 429 - protein, 510 aa, Artificial Sequence
  Description of Artificial Sequence Synthetic polypeptide
MEFGLSWVFLVALFRGVQSEVQLVESGGGLVQPGGSLRLSCAASGFTFLGYGIHWVRQAPGKGLEWVGWISPAGGSTDYADSVKGRFTISADTSKNTAYLQMNSLRAEDTAVYYCARGPFSPWVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSMCIPCFTTNPNMAAKCNACCGSRRGSCRGPQCIC

SEQ ID NO: 430 - protein, 276 aa, Artificial Sequence
  Description of Artificial Sequence Synthetic polypeptide
METDTLLLWVLLLWVPGSTGDIQMTQSPSSLSASVGDRVTITCRASQDVSTAVAWYQQKPGKAPKLLIYSASFLYSGVPSRFSGSGSGTDFTLTISSLQPEDFATYYCQQFPTYLPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSGGGSMCIPCFTTNPNMAAKCNACCGSRRGSCRGPQCIC

SEQ ID NO: 431 - protein, 46 aa, Artificial Sequence
  Description of Artificial Sequence Synthetic polypeptide
GSRCLPPGRPCYGATQRIPCCGVCSHNNCTGSSELYENKPRRPYIL

SEQ ID NO: 432 - protein, 46 aa, Artificial Sequence
  Description of Artificial Sequence Synthetic polypeptide
GSVGCEECPMHCRGRNANPTCDDGVCNCNVGSSELYENKPRRPYIL

SEQ ID NO: 433 - protein, 54 aa, Artificial Sequence
  Description of Artificial Sequence Synthetic polypeptide
GSMCMPCFTTDTQMQERCDRCCGGGGRGRCWGPQCLCIGSSELYENKPRRPYIL

SEQ ID NO: 434 - protein, 53 aa, Artificial Sequence
  Description of Artificial Sequence Synthetic polypeptide
GSMCMPCFTTDPNMAKKCRDCCGGNGKCFGPQCLCNRGSSELYENKPRRPYIL

SEQ ID NO: 435 - protein, 53 aa, Artificial Sequence
  Description of Artificial Sequence Synthetic polypeptide
GSSEKDCIKHLQRCRENKDCCSKKCSRRGTNPEKRCRGSSELYENKPRRPYIL

SEQ ID NO: 436 - protein, 41 aa, Artificial Sequence
  Description of Artificial Sequence Synthetic polypeptide
RCLPPGRPCYGATQRIPCCGVCSHNNCTELYENKPRRPYIL

SEQ ID NO: 437 - protein, 41 aa, Artificial Sequence
  Description of Artificial Sequence Synthetic polypeptide
VGCEECPMHCRGRNANPTCDDGVCNCNVELYENKPRRPYIL

SEQ ID NO: 438 - protein, 49 aa, Artificial Sequence
  Description of Artificial Sequence Synthetic polypeptide
MCMPCFTTDTQMQERCDRCCGGGGRGRCWGPQCLCIELYENKPRRPYIL

SEQ ID NO: 439 - protein, 48 aa, Artificial Sequence
  Description of Artificial Sequence Synthetic polypeptide
MCMPCFTTDPNMAKKCRDCCGGNGKCFGPQCLCNRELYENKPRRPYIL

SEQ ID NO: 440 - protein, 47 aa, Artificial Sequence
  Description of Artificial Sequence Synthetic polypeptide
EKDCIKHLQRCRENKDCCSKKCSRRGTNPEKRCRELYENKPRRPYIL

SEQ ID NO: 441 - protein, 13 aa, Unknown
  Description of Unknown Neurotensin peptide
ELYENKPRRPYIL

SEQ ID NO: 442 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 443 - protein, 271 aa, Artificial Sequence
  Description of Artificial Sequence Synthetic polypeptide
GSMCMPCFTTDTQMQERCDRCCGGGGRGRCWGPQCLCIGGSGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 444 - protein, 8 aa, Artificial Sequence
  Description of Artificial Sequence Synthetic peptide
GGGSGGGS

SEQ ID NO: 445 - protein, 110 aa, Artificial Sequence
  Description of Artificial Sequence Synthetic polypeptide
  MISC_FEATURE (2)..(10): May or may not be present
  MISC_FEATURE (13)..(21): May or may not be present
  MISC_FEATURE (24)..(32): May or may not be present
  MISC_FEATURE (35)..(43): May or may not be present
  MISC_FEATURE (46)..(54): May or may not be present
  MISC_FEATURE (57)..(65): May or may not be present
  MISC_FEATURE (68)..(76): May or may not be present
  MISC_FEATURE (79)..(87): May or may not be present
  MISC_FEATURE (90)..(98): May or may not be present
  MISC_FEATURE (101)..(109): May or may not be present
  MISC_FEATURE (1)..(110): This sequence may encompass 1-10 ′Gly(1-10)Ser′ repeats
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 446 - protein, 4 aa, Artificial Sequence
  Description of Artificial Sequence Synthetic peptide
GAGA

SEQ ID NO: 447 - protein, 5 aa, Artificial Sequence
  Description of Artificial Sequence Synthetic peptide
GGGGS

SEQ ID NO: 448 - protein, 18 aa, Artificial Sequence
  Description of Artificial Sequence Synthetic peptide
KESGSVSSEQLAQFRSLD

SEQ ID NO: 449 - protein, 14 aa, Artificial Sequence
  Description of Artificial Sequence Synthetic peptide
EGKSSGSGSESKST

SEQ ID NO: 450 - protein, 8 aa, Artificial Sequence
  Description of Artificial Sequence Synthetic peptide
GGGGGGGG

SEQ ID NO: 451 - protein, 12 aa, Artificial Sequence
  Description of Artificial Sequence Synthetic peptide
GSAGSAAGSGEF

SEQ ID NO: 452 - protein, 5 aa, Artificial Sequence
  Description of Artificial Sequence Synthetic peptide
EAAAK

SEQ ID NO: 453 - protein, 7 aa, Artificial Sequence
  Description of Artificial Sequence Synthetic peptide
AEAAAKA

SEQ ID NO: 454 - protein, 15 aa, Artificial Sequence
  Description of Artificial Sequence Synthetic peptide
GGGGSGGGGSGGGGS

SEQ ID NO: 455 - protein, 6 aa, Artificial Sequence
  Description of Artificial Sequence Synthetic peptide
GGGGGG

SEQ ID NO: 456 - protein, 15 aa, Artificial Sequence
  Description of Artificial Sequence Synthetic peptide
  MISC_FEATURE (1)..(15): This sequence may encompass 1-3 ′Glu Ala Ala Ala Lys′ repeating units
EAAAKEAAAKEAAAK

SEQ ID NO: 457 - protein, 46 aa, Artificial Sequence
  Description of Artificial Sequence Synthetic polypeptide
AEAAAKEAAAKEAAAKEAAAKALEAEAAAKEAAAKEAAAKEAAAKA

SEQ ID NO: 458 - protein, 5 aa, Artificial Sequence
  Description of Artificial Sequence Synthetic peptide
PAPAP

SEQ ID NO: 459 - protein, 34 aa, Artificial Sequence
  Description of Artificial Sequence Synthetic polypeptide
  MISC_FEATURE (1)..(34): This sequence may encompass 5-17 ′Ala Pro′ repeating units
APAPAPAPAPAPAPAPAPAPAPAPAPAPAPAPAP

SEQ ID NO: 460 - protein, 10 aa, Artificial Sequence
  Description of Artificial Sequence Synthetic peptide
AGNRVRRSVG

SEQ ID NO: 461 - protein, 4 aa, Artificial Sequence
  Description of Artificial Sequence Synthetic peptide
GFLG

SEQ ID NO: 462 - protein, 40 aa, Artificial Sequence
  Description of Artificial Sequence Synthetic polypeptide
  MISC_FEATURE (1)..(40): This sequence may encompass 1-10 ′Gly Gly Gly Ser′ repeating units
GGGSGGGSGGGSGGGSGGGSGGGSGGGSGGGSGGGSGGGS

SEQ ID NO: 463 - protein, 4 aa, Artificial Sequence
  Description of Artificial Sequence Synthetic peptide
GGGS

SEQ ID NO: 464 - protein, 200 aa, Artificial Sequence
  Description of Artificial Sequence Synthetic polypeptide
  MISC_FEATURE (2)..(10): May or may not be present
  MISC_FEATURE (12)..(20): May or may not be present
  MISC_FEATURE (22)..(30): May or may not be present
  MISC_FEATURE (32)..(40): May or may not be present
  MISC_FEATURE (42)..(50): May or may not be present
  MISC_FEATURE (52)..(60): May or may not be present
  MISC_FEATURE (62)..(70): May or may not be present
  MISC_FEATURE (72)..(80): May or may not be present
  MISC_FEATURE (82)..(90): May or may not be present
  MISC_FEATURE (92)..(100): May or may not be present
  MISC_FEATURE (102)..(110): May or may not be present
  MISC_FEATURE (112)..(120): May or may not be present
  MISC_FEATURE (122)..(130): May or may not be present
  MISC_FEATURE (132)..(140): May or may not be present
  MISC_FEATURE (142)..(150): May or may not be present
  MISC_FEATURE (152)..(160): May or may not be present
  MISC_FEATURE (162)..(170): May or may not be present
  MISC_FEATURE (172)..(180): May or may not be present
  MISC_FEATURE (182)..(190): May or may not be present
  MISC_FEATURE (192)..(200): May or may not be present
  MISC_FEATURE (1)..(200): This sequence may encompass 1-10 ′Gly(1-10)Ser(1-10)′ repeating units
GGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSS